(19) United States
(12) Patent Application Publication
(10) Pub. No.: US 2019/0375815 A1
(43) Pub. Date: Dec. 12, 2019

(54) TREATMENT OF CANCER USING CHIMERIC T CELL RECEPTOR PROTEINS HAVING MULTIPLE SPECIFICITIES

(71) Applicants: Novartis AG, Basel (CH); The Trustees of the University of Pennsylvania, Philadelphia, PA (US)

(72) Inventors: Boris Engels, Arlington, MA (US); Brian Walter Granda, Salisbury, MA (US); Carla Guimaraes, Boston, MA (US); Andreas Loew, Boston, MA (US); Melissa Ramones, Cambridge, MA (US)

(21) Appl. No.: 16/481,976

(22) PCT Filed: Jan. 31, 2018
(86) PCT No.: PCT/US2018/016139

§ 371 Date: Jul. 30, 2019

Related U.S. Application Data
(60) Provisional application No. 62/452,601, filed on Jan. 31, 2017.

(51) Int. Cl. C07K 14/725; C07K 14/705; C07K 16/30; C07K 16/28; A61K 35/17; A61P 35/00

(52) U.S. Cl. CPC C07K 14/7051; C07K 14/70575; C07K 16/30; C07K 16/2803; C07K 2317/622; A61K 35/17; C07K 14/70517; A61P 35/00; C07K 2319/03; C07K 16/2887

(57) ABSTRACT

The present invention features the use of chimeric CD3 proteins to modulate T cell Receptor (TCR) signaling. Specifically, the invention is based, in part, on the discovery that multiple chimeric CD3 proteins (e.g., CD3delta, CD3gamma, and CD3 epsilon) having all or most of their extracellular domain fused to more than one antigen binding domain can activate the TCR in the presence of one or more cognate antigens. The invention is further based on the observation that the above chimeric proteins can be potentiated through the inclusion of a co-stimulatory domain in the intracellular portion of the chimeric molecule. Thus, the preferred elements of the engineered signaling complexes of the invention include more than one antigen binding domain, an extracellular domain derived from one of the above CD3 proteins, and an intracellular co-stimulatory domain.

### RELATED APPLICATION

This application claims priority to U.S. Ser. No. 62/452,601 filed Jan. 31, 2017, the content of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jan. 31, 2018, is named N2067-7147WO_SL.txt and is 231,229 bytes in size.

### FIELD OF THE INVENTION

The present invention relates generally to the use of immune effector cells (e.g., T cells, NK cells) engineered to express Chimeric Membrane Proteins to treat a disease associated with expression of a tumor antigen.

### BACKGROUND OF THE INVENTION

Adoptive cell transfer (ACT) therapy with autologous T-cells, especially with T-cells transduced with Chimeric Antigen Receptors (CARs), has shown promise in hematologic cancer trials. There is a need in the art for improved chimeric molecules for use in ACT.

### SUMMARY OF THE INVENTION

The present invention pertains, at least in part, to the use of immune effector cells (e.g., T cells, NK cells) engineered to express more than one chimeric polypeptide that binds to a tumor antigen as described herein to treat cancer associated with expression of said tumor antigen(s).

In a first aspect, the invention provides a system including:

A first chimeric membrane protein including an extracellular domain including a first antigen binding domain and a first extracellular domain derived from the extracellular domain of CD3 gamma, delta, or epsilon, a transmembrane domain, and an intracellular domain including a first intracellular co-stimulatory domain derived from a protein other than CD3 gamma, delta or epsilon; and

A second chimeric membrane protein including an extracellular domain including a second antigen binding domain and a second extracellular domain derived from the extracellular domain of CD3 gamma, delta, or epsilon, a transmembrane domain, and, optionally, an intracellular domain including a second intracellular co-stimulatory domain derived from a protein other than CD3 gamma, delta or epsilon;

Wherein the first antigen binding domain and the second antigen binding domain are not identical, and wherein the first extracellular domain of CD3 gamma, delta, or epsilon and the second extracellular domain of CD3 gamma, delta, or epsilon are not identical.

In embodiments, said first CD3 gamma, delta, or epsilon extracellular domain includes the entire CD3 gamma, delta, or epsilon extracellular domain. In embodiments, including in the aforementioned embodiments, said second CD3 gamma, delta, or epsilon extracellular domain the entire CD3 gamma, delta, or epsilon extracellular domain.

In embodiments, including in the aforementioned embodiments, a) the first chimeric protein includes the entire CD3 epsilon extracellular domain, and the second chimeric protein includes the entire CD3 gamma extracellular domain; b) the first chimeric protein includes the entire CD3 epsilon extracellular domain, and the second chimeric protein includes the entire CD3 delta extracellular domain; or c) the first chimeric protein includes the entire CD3 delta extracellular domain, and the second chimeric protein includes the entire CD3 gamma extracellular domain.

In embodiments, including in the aforementioned embodiments, the first chimeric protein includes the entire CD3 gamma, delta or epsilon protein. In embodiments, including in the aforementioned embodiments, the second chimeric protein includes the entire CD3 gamma, delta or epsilon protein. In other embodiments, including in the aforementioned embodiments, the first chimeric protein does not include any intracellular domains derived from the CD3 gamma, delta or epsilon protein. In embodiments, including in the aforementioned embodiments, the second chimeric protein does not include any intracellular domains derived from CD3 gamma, delta or epsilon protein.

In embodiments, including in the aforementioned embodiments, the transmembrane domain of the first chimeric protein and/or second chimeric protein does not include a transmembrane domain of CD3 gamma, delta or epsilon.

In embodiments, including in the aforementioned embodiments, the first antigen binding domain is located N-terminal to said first extracellular domain derived from CD3 gamma, delta, or epsilon. In embodiments, including in the aforementioned embodiments, the second antigen binding domain is located N-terminal to said second extracellular domain derived from CD3 gamma, delta, or epsilon.

In embodiments, including in the aforementioned embodiments, the first chimeric protein, the second chimeric protein, or both the first and second chimeric proteins include a third antigen binding domain located N-terminal to said first and/or second antigen binding domain.

In embodiments, including in the aforementioned embodiments, the first antigen binding domain and said first extracellular domain derived from CD3 gamma, delta, or epsilon are connected by a first linker and/or the second antigen binding domain and said second extracellular domain derived from CD3 gamma, delta, or epsilon are connected by a second linker. In embodiments, said first linker and/or second linker includes, e.g., consists of, (GGGGS)n, e.g., wherein n is an integer from 0 to 10 (SEQ ID NO: 68), e.g., wherein n=4. In embodiments, including in the aforementioned embodiments, said second chimeric membrane protein includes a second intracellular co-stimulatory domain derived from a protein other than CD3 gamma, delta or epsilon. In other embodiments, including in the aforementioned embodiments, said second chimeric membrane protein does not include a second intracellular co-stimulatory domain derived from a protein other than CD3 gamma, delta or epsilon. In embodiments, including in the aforementioned embodiments, the system does not include a second intracellular co-stimulatory domain.

In embodiments, including in the aforementioned embodiments, the system includes both the first intracellular co-stimulatory domain and a second intracellular co-stimulatory domain.

In embodiments, including in the aforementioned embodiments, the first chimeric membrane protein includes a third intracellular co-stimulatory domain derived form a protein other than CD3 gamma, delta or epsilon located C-terminal to the first intracellular co-stimulatory domain.

In embodiments, including in the aforementioned embodiments, one or more of said intracellular co-stimulatory domains (e.g., the first intracellular co-stimulatory domain and/or second intracellular co-stimulatory domain, if present, and/or third intracellular co-stimulatory domain, if present) is a functional signaling domain of a protein selected from the group consisting of: an MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, or a functional variant thereof, e.g., includes a co-stimulatory domain described herein.

In embodiments, including in the aforementioned embodiments, the first antigen binding domain binds a tumor antigen. In embodiments, the first antigen binding domain binds a B-cell antigen, for example, CDS, CD10, CD19, CD20, CD21, CD22, CD23, CD24, CD25, CD27, CD30, CD34, CD37, CD38, CD40, CD53, CD69, CD72, CD73, CD74, CD75, CD77, CD79a, CD79b, CD80, CD81, CD82, CD83, CD84, CD85, CD86, CD123, CD135, CD138, CD179, CD269, Flt3, ROR1, BCMA, FcRn5, FcRn2, CS-1, CXCR4, 5, 7, IL-7/3R, IL7/4/3R, or IL4R, for example, CD19, CD20, CD22, FcRn5, FcRn2, BCMA, CS-1, or CD138.

In embodiments, including in the aforementioned embodiments, the second antigen binding domain binds a tumor antigen. In embodiments, the second antigen binding domain binds a B-cell antigen, for example, the same B-cell antigen as bound by the first antigen binding domain, but at a different binding epitope or region on the antigen. In other embodiments, the second antigen binding domain binds a B-cell antigen, for example, a different B-cell antigen than the B-cell antigen bound by the first antigen binding domain. In embodiments, the B-cell antigen bound by the second antigen binding domain is CD5, CD10, CD19, CD20, CD21, CD22, CD23, CD24, CD25, CD27, CD30, CD34, CD37, CD38, CD40, CD53, CD69, CD72, CD73, CD74, CD75, CD77, CD79a, CD79b, CD80, CD81, CD82, CD83, CD84, CD85, CD86, CD123, CD135, CD138, CD179, CD269, Flt3, ROR1, BCMA, FcRn5, FcRn2, CS-1, CXCR4, 5, 7, IL-7/3R, IL7/4/3R, or IL4R, for example, is CD19, CD20, CD22, FcRn5, FcRn2, BCMA, CS-1, or CD138.

In embodiments, a) the first antigen binding domain binds CD19 and the second antigen binding domain binds CD20; b) the first antigen binding domain binds CD19 and the second antigen binding domain binds CD22; or c) the first antigen binding domain binds CD20 and the second antigen binding domain binds CD22.

In embodiments, the second antigen binding domain binds a solid tumor antigen, for example, as described herein, for example, EGFRvIII, mesothelin, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRa4, and a peptide of any of these antigens presented on MHC, for example, a solid tumor antigen selected from the group consisting of CLDN6, mesothelin and EGFRvIII. In embodiments, a) the first antigen binding domain binds CD19 and the second antigen binding domain binds mesothelin; b) the first antigen binding domain binds CD19 and the second antigen binding domain binds EGFRvIII; or c) the first antigen binding domain binds CD19 and the second antigen binding domain binds CLDN6.

In a second aspect, the invention provides a nucleic acid construct encoding the system of any of the aforementioned aspects and embodiments. In embodiments, the nucleic acid construct is RNA, for example, mRNA. In other embodiments, the nucleic acid construct is DNA.

In a third aspect, the invention provides a vector including the nucleic acid construct of the previous aspect. In embodiments, said vector is a lentiviral, adenoviral, or retroviral vector. In embodiments, upon expression of said first and second chimeric membrane proteins, said proteins are expressed as a single mRNA transcript, for example, wherein the nucleic acid sequences encoding said first and second chimeric membrane proteins are separated by a nucleic acid encoding a self-cleavage site or an internal ribosomal entry site.

In a fourth aspect, the invention provides a cell, e.g., as described herein, including the nucleic acid construct of any of the previous nucleic acid construct aspects and embodiments, the vector of any of the aforementioned vector aspects and embodiments, or the system of any of the aforementioned aspects and embodiments. In embodiments, said cell is selected from an NK cell or T cell.

In a fifth aspect, the invention provides a method of treating a subject with a proliferative disorder, said method including administering the cell of any one of the aforementioned cell aspects and embodiments. In embodiments, said subject has a tumor and said administration provides said subject with immunity against said tumor. In embodiments, said cell is a T cell or NK cell and is autologous to said subject. In other embodiments, said cell is an allogeneic T cell or NK cell. In embodiments, said subject is a human.

While features of the chimeric membrane proteins of the system are described above, additional aspects of the chimeric membrane proteins of the system are described below. Thus, In a related aspect, the invention features a chimeric membrane protein including a CD3 gamma, delta, or epsilon domain and an intracellular co-stimulatory domain, wherein the CD3 domain includes an extracellular domain derived from the extracellular domain of CD3 gamma, delta, or epsilon and the intracellular co-stimulatory domain is not derived from CD3 gamma, delta, or epsilon.

In a related aspect, the invention features a chimeric membrane protein including a CD3 gamma, delta, or epsilon domain and a first intracellular dimerization domain, wherein the CD3 gamma, delta, or epsilon domain includes an extracellular domain derived from the extracellular domain of CD3 gamma, delta, or epsilon. In this aspect, the protein can, optionally, further includes an intracellular co-stimulatory domain.

In yet another aspect, the invention features a chimeric membrane protein including an antigen binding domain and a CD3 gamma, delta, or epsilon domain, wherein the CD3 gamma, delta, or epsilon domain includes an extracellular domain derived from the extracellular domain of CD3 gamma, delta, or epsilon.

In any of the foregoing aspects, CD3 gamma, delta, or epsilon domain includes the entire CD3 gamma, delta, or epsilon extracellular domain (e.g., the entire protein) or a portion of the CD3 gamma, delta, or epsilon domain. In certain aspects where it is only a portion of the extracellular domain, the truncated domain retains the ability to associate with the remaining TCR polypeptides. In certain aspects, the chimeric protein does not include any intracellular and/or transmembrane domains derived from CD3 gamma, delta, or epsilon.

In any of the foregoing aspects, the protein also includes an antigen binding domain located N-terminal to the CD3 gamma, delta, or epsilon domain.

In another aspect, the invention features a cell (e.g., a NK cell or T cell) including any one of the foregoing chimeric membrane proteins.

In another aspect, the invention features a nucleic acid (e.g., a DNA or mRNA) encoding any one of the foregoing chimeric membrane proteins. The invention also feature vectors (e.g., a lentiviral, adenoviral, or retroviral) vector including such nucleic acids.

In certain of any of the foregoing cells, the chimeric membrane protein includes the CD3 gamma, delta, or epsilon domain and intracellular dimerization domain, and the cell further includes a second chimeric protein, the second chimeric protein including an intracellular costimulatory domain and a second intracellular dimerization domain. In certain embodiments, the first and second dimerization domains make up a heterodimerization pair and heterodimerize when expressed in the cell (e.g., p53 and MDM2, mFos and mJun Coils, and VPS36 and VPS28). In other embodiments, the first and second dimerization domains make up a heterodimerization pair and heterodimerize when expressed in the cell only in the presence of a dimerization compound. For example, one of the first and second dimerization domains can include a rapamycin analog binding sequence having at least 85% identity with FKBP, and, optionally, the other of the first and second dimerization domains includes a rapamycin analog binding sequence having at least 85% identity with FRP. In another example, one of the first and second dimerization domains includes a rapamycin analog binding sequence from FKBP. Here, the other of the first and second dimerization domain can optionally include a rapamycin analog binding sequence from FRP. In certain embodiments, the rapamycin analog binding sequence includes an AP21967 binding sequence from FKBP or FRP. Other exemplary heterodimerizatoin pairs include a GyrB-GyrB based switch, a GAI-GID1 based switch, or a Halo-tag/SNAP-tag based switch.

The second chimeric protein can be, e.g., a chimeric membrane protein and can, e.g., further include an extracellular antigen-binding domain. In other aspects, certain of the foregoing cells can, e.g., include the CD3 gamma, delta, or epsilon domain and intracellular dimerization domain, and the cell can, e.g., further include a second chimeric protein (e.g., a chimeric membrane protein), the second chimeric protein including an extracellular antigen binding domain, an intracellular costimulatory domain, and a CD3 gamma, delta, or epsilon binding domain (which, e.g., binds the intracellular or extracellular CD3 domain). Such binding domains can be, e.g., derived from an anti-CD3 gamma, delta, or epsilon antibody (e.g., an scFv or Vhh domain). In certain of these embodiments, the extracellular antigen-binding domain (e.g., the antigen binding domain of an antibody or fragment thereof.) of the second chimeric protein is heterologous to the intracellular costimulatory signaling domain of the second chimeric protein and/or is the extracellular domain of an inhibitory molecule. Alternatively, the extracellular antigen-binding domain of the second chimeric protein is naturally associated with the intracellular costimulatory signaling domain of the second chimeric protein.

In certain of the above aspects, the second chimeric protein can be, e.g., expressed as an intracellular protein.

In certain of the foregoing aspects, the first and second chimeric protein both include an intracellular co-stimulatory domain derived from the same or different endogenous protein. In another aspect, the invention features a nucleic acid encoding any of the foregoing first and second chimeric proteins and a vector including such a nucleic acid. Such vectors can be configure such that, upon expression of the first and second chimeric proteins, the proteins are expressed as a single mRNA transcript, e.g., where the first and second chimeric proteins are separated by a nucleic acid encoding a self-cleavage site or an internal ribosomal entry site.

In any of the foregoing embodiments, one or more of the intracellular co-stimulatory domains is a functional signaling domain of a protein selected from the group including of: an MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, or a functional variant thereof.

In yet another aspect, the invention features the treatment of a subject (e.g., a human) with any of the foregoing cells (e.g., wherein the subject has a proliferative disorder (e.g., cancer). In certain embodiments the subject has a tumor and the administration provides the subject with immunity against the tumor. The cell can be, e.g., a T cell or NK cell autologous or allogeneic to the subject.

Chimeric Protein Encoding Nucleic Acids

Accordingly, in one aspect, the invention pertains to an isolated nucleic acid molecule encoding a chimeric membrane protein that comprises one or more of the following: an antigen binding domain (e.g., antibody or antibody fragment, TCR or TCR fragment) that binds to a tumor antigen as described herein, a transmembrane domain (e.g., a transmembrane domain described herein), and an intracellular signaling domain (e.g., an intracellular signaling domain comprising a costimulatory domain (e.g., a costimulatory domain described herein) and/or a primary signaling domain (e.g., a primary signaling domain described herein). In some embodiments, the tumor antigen is chosen from one or more of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDG1cp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDG1cp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In some embodiments, tumor antigen bound by the encoded molecule is chosen from one or more of: TSHR, CD171, CS-1, CLL-1, GD3, Tn Ag, FLT3, CD38, CD44v6, B7H3, KIT, IL-13Ra2, IL-11Ra, PSCA, PRSS21, VEGFR2, LewisY, CD24, PDGFR-beta, SSEA-4, MUC1, EGFR, NCAM, CAIX, LMP2, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, and IGLL1.

In certain embodiments, the tumor antigen bound by the encoded CAR molecule is chosen from one or more of: TSHR, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, and OR51E2.

In certain embodiments, one or more of the antigen binding domains binds a B-Cell antigen, Exemplary B-cell antigens: CD5, CD10, CD19, CD20, CD21, CD22, CD23, CD24, CD25, CD27, CD30, CD34, CD37, CD38, CD40, CD53, CD69, CD72, CD73, CD74, CD75, CD77, CD79a, CD79b, CD80, CD81, CD82, CD83, CD84, CD85, CD86, CD123, CD135, CD138, CD179, CD269, Flt3, ROR1, BCMA, FcRn5, FcRn2, CS-1, CXCR4, 5, 7, IL-7/3R, IL7/4/3R, and IL4R. Particularly preferred B-Cell antigens include: CD19, CD20, CD22, FcRn5, FcRn2, BCMA, CS-1 and CD138. In embodiments, the B-Cell antigen is CD19. In embodiments, the B-Cell antigen is CD20. In embodiments, the B-Cell antigen is CD22. In embodiments, the B-Cell antigen is BCMA. In embodiments, the B-Cell antigen is FcRn5. In embodiments, the B-Cell antigen is FcRn2. In embodiments, the B-Cell antigen is CS-1. In embodiments, the B-Cell antigen is CD138.

In some embodiments, the antigen binding domain of the encoded molecule comprises an antibody, an antibody fragment, an scFv, a Fv, a Fab, a (Fab′)2, a single domain antibody (SDAB), a VH or VL domain, or a camelid VHH domain.

In some embodiments, the transmembrane domain of the encoded molecule comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C, or a functional variant thereof.

In other embodiments, the nucleic acid molecule encodes an intracellular signaling domain comprising a sequence encoding a primary signaling domain and/or a sequence encoding a costimulatory signaling domain. In some embodiments, the intracellular signaling domain comprises a sequence encoding a primary signaling domain. In some embodiments, the intracellular signaling domain comprises a sequence encoding a costimulatory signaling domain. In some embodiments, the intracellular signaling domain comprises a sequence encoding a primary signaling domain and a sequence encoding a costimulatory signaling domain.

In certain embodiments, the encoded primary signaling domain comprises a functional signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP12.

In one embodiment, the encoded primary signaling domain comprises a functional signaling domain of CD3 zeta.

In certain preferred embodiments, the encoded intracellular signaling domain comprises a sequence encoding a costimulatory signaling domain. For example, the intracellular signaling domain can comprise a sequence encoding a primary signaling domain and a sequence encoding a costimulatory signaling domain. In some embodiments, the encoded costimulatory signaling domain comprises a functional signaling domain of a protein chosen from one or more of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, or NKG2D, or a functional variant thereof.

In some embodiments, the nucleic acid molecule further comprises a leader sequence.

In certain embodiments, the encoded antigen binding domain has a binding affinity KD of 10⁻⁴ M to 10⁻⁸M.

In one embodiment, the encoded antigen binding domain is an antigen binding domain described herein, e.g., an antigen binding domain described herein for a target provided above.

In one embodiment, the encoded molecule comprises an antigen binding domain that has a binding affinity KD of 10⁻⁴ M to 10⁻⁸M, e.g., 10⁻⁵ M to 10⁻⁷ M, e.g., 10⁻⁶M or 10⁻⁷ M, for the target antigen. In one embodiment, the antigen binding domain has a binding affinity that is at least five-fold, 10-fold, 20-fold, 30-fold, 50-fold, 100-fold or 1,000-fold less than a reference antibody, e.g., an antibody described herein. In one embodiment, the encoded antigen binding domain has a binding affinity at least 5-fold less than a reference antibody (e.g., an antibody from which the antigen binding domain is derived).

In another aspect, provided herein is a system comprising:

a first chimeric membrane protein comprising an extracellular domain comprising a first antigen binding domain and a first extracellular domain derived from the extracellular domain of CD3 gamma, delta, or epsilon, a transmembrane domain, and an intracellular domain comprising a first intracellular co-stimulatory domain derived from a protein other than CD3 gamma, delta or epsilon; and

a second chimeric membrane protein comprising an extracellular domain comprising a second antigen binding domain and a second extracellular domain derived from the extracellular domain of CD3 gamma, delta, or epsilon, a transmembrane domain, and, optionally, an intracellular domain comprising a second intracellular co-stimulatory domain derived from a protein other than CD3 gamma, delta or epsilon;

wherein the first antigen binding domain and the second antigen binding domain are not identical, and wherein the first extracellular domain derived from the extracellular domain of CD3 gamma, delta, or epsilon and the second extracellular domain derived from the extracellular domain of CD3 gamma, delta, or epsilon are not identical.

In one embodiment, the first extracellular domain comprises the extracellular domain of CD3 gamma, delta, or epsilon, or a functional variant thereof, optionally wherein the first extracellular domain comprises the amino acid sequence of SEQ ID NO: 88, 83, or 78 (or a sequence at least about 85%, 90%, 95%, 99% or more identical thereto, and/or having one, two, three or more substitutions, insertions or deletions, e.g., conserved substitutions). In one embodiment, the first extracellular domain comprises the amino acid sequence of SEQ ID NO: 88. In one embodiment, the first extracellular domain comprises the amino acid sequence of SEQ ID NO: 83. In one embodiment, the first extracellular domain comprises the amino acid sequence of SEQ ID NO: 78. In one embodiment, the second extracellular domain comprises the extracellular domain of CD3 gamma, delta, or epsilon, or a functional variant thereof, optionally wherein the second extracellular domain comprises the amino acid sequence of SEQ ID NO: 88, 83, or 78 (or a sequence at least about 85%, 90%, 95%, 99% or more identical thereto, and/or having one, two, three or more substitutions, insertions or deletions, e.g., conserved substitutions). In one embodiment, the second extracellular domain comprises the amino acid sequence of SEQ ID NO: 88. In one embodiment, the second extracellular domain comprises the amino acid sequence of SEQ ID NO: 83. In one embodiment, the second extracellular domain comprises the amino acid sequence of SEQ ID NO: 78.

In one embodiment, the first chimeric membrane protein comprises the extracellular domain of CD3 gamma, or a functional variant thereof, and the second chimeric membrane protein comprises the extracellular domain of CD3 delta, or a functional variant thereof. In one embodiment, the first chimeric membrane protein comprises the amino acid sequence of SEQ ID NO: 88 (or a sequence at least about 85%, 90%, 95%, 99% or more identical thereto, and/or having one, two, three or more substitutions, insertions or deletions, e.g., conserved substitutions), and the second chimeric membrane protein comprises the amino acid sequence of SEQ ID NO: 83 (or a sequence at least about 85%, 90%, 95%, 99% or more identical thereto, and/or having one, two, three or more substitutions, insertions or deletions, e.g., conserved substitutions). In one embodiment, the first chimeric membrane protein comprises the amino acid sequence of SEQ ID NO: 88, and the second chimeric membrane protein comprises the amino acid sequence of SEQ ID NO: 83. In one embodiment, the first chimeric membrane protein comprises the extracellular domain of CD3 gamma, or a functional variant thereof, and the second chimeric membrane protein comprises the extracellular domain of CD3 epsilon, or a functional variant thereof. In one embodiment, the first chimeric membrane protein comprises the amino acid sequence of SEQ ID NO: 88 (or a sequence at least about 85%, 90%, 95%, 99% or more identical thereto, and/or having one, two, three or more substitutions, insertions or deletions, e.g., conserved substitutions), and the second chimeric membrane protein comprises the amino acid sequence of SEQ ID NO: 78 (or a sequence at least about 85%, 90%, 95%, 99% or more identical thereto, and/or having one, two, three or more substitutions, insertions or deletions, e.g., conserved substitutions). In one embodiment, the first chimeric membrane protein comprises the amino acid sequence of SEQ ID NO: 88, and the second chimeric membrane protein comprises the amino acid sequence of SEQ ID NO: 78. In one embodiment, the first chimeric membrane protein comprises the extracellular domain of CD3 delta, or a functional variant thereof, and the second chimeric membrane protein comprises the extracellular domain of CD3 gamma, or a functional variant thereof. In one embodiment, the first chimeric membrane protein comprises the amino acid sequence of SEQ ID NO: 83 (or a sequence at least about 85%, 90%, 95%, 99% or more identical thereto, and/or having one, two, three or more substitutions, insertions or deletions, e.g., conserved substitutions), and the second chimeric membrane protein comprises the amino acid sequence of SEQ ID NO: 88 (or a sequence at least about 85%, 90%, 95%, 99% or more identical thereto, and/or having one, two, three or more substitutions, insertions or deletions, e.g., conserved substitutions). In one embodiment, the first chimeric membrane protein comprises the amino acid sequence of SEQ ID NO: 83, and the second chimeric membrane protein comprises the amino acid sequence of SEQ ID NO: 88. In one embodiment, the first chimeric membrane protein comprises the extracellular domain of CD3 delta, or a functional variant thereof, and the second chimeric membrane protein comprises the extracellular domain of CD3 epsilon, or a functional variant thereof. In one embodiment, the first chimeric membrane protein comprises the amino acid sequence of SEQ ID NO: 83 (or a sequence at least about 85%, 90%, 95%, 99% or more identical thereto, and/or having one, two, three or more substitutions, insertions or deletions, e.g., conserved substitutions), and the second chimeric membrane protein comprises the amino acid sequence of SEQ ID NO: 78 (or a sequence at least about 85%, 90%, 95%, 99% or more identical thereto, and/or having one, two, three or more substitutions, insertions or deletions, e.g., conserved substitutions). In one embodiment, the first chimeric membrane protein comprises the amino acid sequence of SEQ ID NO: 83, and the second chimeric membrane protein comprises the amino acid sequence of SEQ ID NO: 78. In one embodiment, the first chimeric membrane protein comprises the extracellular domain of CD3 epsilon, or a functional variant thereof, and the second chimeric membrane protein comprises the extracellular domain of CD3 gamma, or a functional variant thereof. In one embodiment, the first chimeric membrane protein comprises the amino acid sequence of SEQ ID NO: 78 (or a sequence at least about 85%, 90%, 95%, 99% or more identical thereto, and/or having one, two, three or more substitutions, insertions or deletions, e.g., conserved substitutions), and the second chimeric membrane protein comprises the amino acid sequence of SEQ ID NO: 88 (or a sequence at least about 85%, 90%, 95%, 99% or more identical thereto, and/or having one, two, three or more substitutions, insertions or deletions, e.g., conserved substitutions). In one embodiment, the first chimeric membrane protein comprises the amino acid sequence of SEQ ID NO: 78, and the second chimeric membrane protein comprises the amino acid sequence of SEQ ID NO: 88. In one embodiment, the first chimeric membrane protein comprises the extracellular domain of CD3 epsilon, or a functional variant thereof, and the second chimeric membrane protein comprises the extracellular domain of CD3 delta, or a functional variant thereof. In one embodiment, the first chimeric membrane protein comprises the amino acid sequence of SEQ ID NO: 78 (or a sequence at least about 85%, 90%, 95%, 99% or more identical thereto, and/or having one, two, three or more substitutions, insertions or deletions, e.g., conserved substitutions), and the second chimeric membrane protein comprises the amino acid sequence of SEQ ID NO: 83 (or a sequence at least about 85%, 90%, 95%, 99% or more identical thereto, and/or having one, two, three or more substitutions, insertions or deletions, e.g., conserved substitutions). In one embodiment, the first chimeric membrane protein comprises the amino acid sequence of SEQ ID NO: 78, and the second chimeric membrane protein comprises the amino acid sequence of SEQ ID NO: 83.

In one embodiment, the transmembrane domain of the first chimeric membrane protein comprises the transmembrane domain of CD3 gamma, delta, or epsilon, or a functional variant thereof. In one embodiment, the transmembrane domain of the first chimeric membrane protein comprises the amino acid sequence of SEQ ID NO: 89, 84, or 79 (or a sequence at least about 85%, 90%, 95%, 99% or more identical thereto, and/or having one, two, three or more substitutions, insertions or deletions, e.g., conserved substitutions).

In one embodiment, the transmembrane domain of the first chimeric membrane protein does not comprise a transmembrane domain of CD3 gamma, delta or epsilon.

In one embodiment, the transmembrane domain of the second chimeric membrane protein comprises the transmembrane domain of CD3 gamma, delta, or epsilon, or a functional variant thereof. In one embodiment, the transmembrane domain of the second chimeric membrane protein comprises the amino acid sequence of SEQ ID NO: 89, 84, or 79 (or a sequence at least about 85%, 90%, 95%, 99% or more identical thereto, and/or having one, two, three or more substitutions, insertions or deletions, e.g., conserved substitutions).

In one embodiment, the transmembrane domain of the second chimeric membrane protein does not comprise a transmembrane domain of CD3 gamma, delta or epsilon.

In one embodiment, the first chimeric membrane protein comprises the CD3 gamma, delta or epsilon protein, or a functional variant thereof. In one embodiment, the first chimeric membrane protein comprises the amino acid sequence of SEQ ID NO: 90, 85, or 80 (or a sequence at least about 85%, 90%, 95%, 99% or more identical thereto, and/or having one, two, three or more substitutions, insertions or deletions, e.g., conserved substitutions). In one embodiment, the first chimeric membrane protein comprises the amino acid sequence of SEQ ID NO: 90. In one embodiment, the first chimeric membrane protein comprises the amino acid sequence of SEQ ID NO: 85. In one embodiment, the first chimeric membrane protein comprises the amino acid sequence of SEQ ID NO: 80. In one embodiment, the first chimeric membrane protein comprises the amino acid sequence of SEQ ID NO: 87, 82, or 77 (or a sequence at least about 85%, 90%, 95%, 99% or more identical thereto, and/or having one, two, three or more substitutions, insertions or deletions, e.g., conserved substitutions). In one embodiment, the first chimeric membrane protein comprises the amino acid sequence of SEQ ID NO: 87. In one embodiment, the first chimeric membrane protein comprises the amino acid sequence of SEQ ID NO: 82. In one embodiment, the first chimeric membrane protein comprises the amino acid sequence of SEQ ID NO: 77. In one embodiment, the second chimeric membrane protein comprises the CD3 gamma, delta or epsilon protein, or a functional variant thereof. In one embodiment, the second chimeric membrane protein comprises the amino acid sequence of SEQ ID NO: 90, 85, or 80 (or a sequence at least about 85%, 90%, 95%, 99% or more identical thereto, and/or having one, two, three or more substitutions, insertions or deletions, e.g., conserved substitutions). In one embodiment, the second chimeric membrane protein comprises the amino acid sequence of SEQ ID NO: 90. In one embodiment, the second chimeric membrane protein comprises the amino acid sequence of SEQ ID NO: 85. In one embodiment, the second chimeric membrane protein comprises the amino acid sequence of SEQ ID NO: 80. In one embodiment, the second chimeric membrane protein comprises the CD3 gamma, delta or epsilon protein, or a functional variant thereof, optionally wherein the second chimeric membrane protein comprises the amino acid sequence of SEQ ID NO: 87, 82, or 77 (or a sequence at least about 85%, 90%, 95%, 99% or more identical thereto, and/or having one, two, three or more substitutions, insertions or deletions, e.g., conserved substitutions). In one embodiment, the second chimeric membrane protein comprises the amino acid sequence of SEQ ID NO: 87. In one embodiment, the second chimeric membrane protein comprises the amino acid sequence of SEQ ID NO: 82. In one embodiment, the second chimeric membrane protein comprises the amino acid sequence of SEQ ID NO: 77.

In one embodiment, the first chimeric membrane protein does not comprise any intracellular domains derived from the CD3 gamma, delta or epsilon protein. In one embodiment, the second chimeric membrane protein does not comprise any intracellular domains derived from the CD3 gamma, delta or epsilon protein.

In one embodiment, the first antigen binding domain is located N-terminal to said first extracellular domain derived from the extracellular domain of CD3 gamma, delta, or epsilon. In one embodiment, the second antigen binding domain is located N-terminal to said second extracellular domain derived from the extracellular domain of CD3 gamma, delta, or epsilon. In one embodiment, the first chimeric membrane protein, the second chimeric membrane protein, or both the first and second chimeric membrane proteins comprise a third antigen binding domain located N-terminal to said first and/or second antigen binding domain. In one embodiment, the first antigen binding domain and said first extracellular domain derived from the extracellular domain of CD3 gamma, delta, or epsilon are connected by a first linker and/or the second antigen binding domain and said second extracellular domain derived from the extracellular domain of CD3 gamma, delta, or epsilon are connected by a second linker. In one embodiment, the first linker and/or second linker comprises, e.g., consists of, (GGGGS)n, e.g., wherein n is an integer from 0 to 10, e.g., wherein n=1, 2, or 4. In one embodiment, n=1. In one embodiment, n=2. In one embodiment, n=4.

In one embodiment, said second chimeric membrane protein comprises a second intracellular co-stimulatory domain derived from a protein other than CD3 gamma, delta or epsilon. In one embodiment, said second chimeric membrane protein does not comprise a second intracellular co-stimulatory domain derived from a protein other than CD3 gamma, delta or epsilon. In one embodiment, the system does not comprise a second intracellular co-stimulatory domain. In one embodiment, the system comprises both the first intracellular co-stimulatory domain and the second intracellular co-stimulatory domain. In one embodiment, the first chimeric membrane protein comprises a third intracellular co-stimulatory domain derived form a protein other than CD3 gamma, delta or epsilon located C-terminal to the first intracellular co-stimulatory domain.

In one embodiment, one or more of said intracellular co-stimulatory domains (e.g., the first intracellular co-stimulatory domain and/or second intracellular co-stimulatory domain, if present, and/or third intracellular co-stimulatory domain, if present) is a functional signaling domain of a protein selected from the group consisting of: an MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, or a functional variant thereof.

In one embodiment, one or more of said intracellular co-stimulatory domains (e.g., the first intracellular co-stimulatory domain and/or second intracellular co-stimulatory domain, if present, and/or third intracellular co-stimulatory domain, if present) is a functional signaling domain of 4-1BB, or a functional variant thereof, optionally wherein one or more of said intracellular co-stimulatory domains (e.g., the first intracellular co-stimulatory domain and/or second intracellular co-stimulatory domain, if present, and/or third intracellular co-stimulatory domain, if present) comprises the amino acid sequence of SEQ ID NO: 50 (or a sequence at least about 85%, 90%, 95%, 99% or more identical thereto, and/or having one, two, three or more substitutions, insertions or deletions, e.g., conserved substitutions). In one embodiment, one or more of said intracellular co-stimulatory domains (e.g., the first intracellular co-stimulatory domain and/or second intracellular co-stimulatory domain, if present, and/or third intracellular co-stimulatory domain, if present) comprises the amino acid sequence of SEQ ID NO: 50.

In one embodiment, the first chimeric membrane protein comprises the amino acid sequence of SEQ ID NO: 91, 86, or 81 (or a sequence at least about 85%, 90%, 95%, 99% or more identical thereto, and/or having one, two, three or more substitutions, insertions or deletions, e.g., conserved substitutions). In one embodiment, the first chimeric membrane protein comprises the amino acid sequence of SEQ ID NO: 91. In one embodiment, the first chimeric membrane protein comprises the amino acid sequence of SEQ ID NO: 86. In one embodiment, the first chimeric membrane protein comprises the amino acid sequence of SEQ ID NO: 81. In one embodiment, the second chimeric membrane protein comprises the amino acid sequence of SEQ ID NO: 91, 86, or 81 (or a sequence at least about 85%, 90%, 95%, 99% or more identical thereto, and/or having one, two, three or more substitutions, insertions or deletions, e.g., conserved substitutions). In one embodiment, the first chimeric membrane protein comprises the amino acid sequence of SEQ ID NO: 91. In one embodiment, the first chimeric membrane protein comprises the amino acid sequence of SEQ ID NO: 86. In one embodiment, the first chimeric membrane protein comprises the amino acid sequence of SEQ ID NO: 81.

In one embodiment, the first antigen binding domain binds a tumor antigen. In one embodiment, the first antigen binding domain binds a B-cell antigen. In one embodiment, the B-cell antigen bound by the first antigen binding domain is CD5, CD10, CD19, CD20, CD21, CD22, CD23, CD24, CD25, CD27, CD30, CD34, CD37, CD38, CD40, CD53, CD69, CD72, CD73, CD74, CD75, CD77, CD79a, CD79b, CD80, CD81, CD82, CD83, CD84, CD85, CD86, CD123, CD135, CD138, CD179, CD269, Flt3, ROR1, BCMA, FcRn5, FcRn2, CS-1, CXCR4, 5, 7, IL-7/3R, IL7/4/3R, or IL4R. In one embodiment, the B-cell antigen bound by the first antigen binding domain is CD19, CD20, CD22, FcRn5, FcRn2, BCMA, CS-1, or CD138.

In one embodiment, the second antigen binding domain binds a tumor antigen. In one embodiment, the second antigen binding domain binds a B-cell antigen. In one embodiment, the second antigen binding domain binds a different B-cell antigen than the B-cell antigen bound by the first antigen binding domain. In one embodiment, the B-cell antigen bound by the second antigen binding domain is CD5, CD10, CD19, CD20, CD21, CD22, CD23, CD24, CD25, CD27, CD30, CD34, CD37, CD38, CD40, CD53, CD69, CD72, CD73, CD74, CD75, CD77, CD79a, CD79b, CD80, CD81, CD82, CD83, CD84, CD85, CD86, CD123, CD135, CD138, CD179, CD269, Flt3, ROR1, BCMA, FcRn5, FcRn2, CS-1, CXCR4, 5, 7, IL-7/3R, IL7/4/3R, or IL4R. In one embodiment, the B-cell antigen bound by the second antigen binding domain is CD19, CD20, CD22, FcRn5, FcRn2, BCMA, CS-1, or CD138.

In one embodiment, the first antigen binding domain binds CD19 and the second antigen binding domain binds CD20. In one embodiment, the first antigen binding domain binds CD19 and the second antigen binding domain binds CD22. In one embodiment, the first antigen binding domain binds CD20 and the second antigen binding domain binds CD22. In one embodiment, the first antigen binding domain binds CD20 and the second antigen binding domain binds CD19. In one embodiment, the first antigen binding domain binds CD22 and the second antigen binding domain binds CD19. In one embodiment, the first antigen binding domain binds CD22 and the second antigen binding domain binds CD20. In one embodiment, the first antigen binding domain binds CD19 and the second antigen binding domain binds CD22, optionally wherein:

(i) the first chimeric membrane protein comprises the amino acid sequence of SEQ ID NO: 70 (or a sequence at least about 85%, 90%, 95%, 99% or more identical thereto, and/or having one, two, three or more substitutions, insertions or deletions, e.g., conserved substitutions), and the second chimeric membrane protein comprises the amino acid sequence of SEQ ID NO: 75 or 76 (or a sequence at least about 85%, 90%, 95%, 99% or more identical thereto, and/or having one, two, three or more substitutions, insertions or deletions, e.g., conserved substitutions);

(ii) the first chimeric membrane protein comprises the amino acid sequence of SEQ ID NO: 71 (or a sequence at least about 85%, 90%, 95%, 99% or more identical thereto, and/or having one, two, three or more substitutions, insertions or deletions, e.g., conserved substitutions), and the second chimeric membrane protein comprises the amino acid sequence of SEQ ID NO: 73, 74, 75, or 76 (or a sequence at least about 85%, 90%, 95%, 99% or more identical thereto, and/or having one, two, three or more substitutions, insertions or deletions, e.g., conserved substitutions); or

(iii) the first chimeric membrane protein comprises the amino acid sequence of SEQ ID NO: 72 (or a sequence at least about 85%, 90%, 95%, 99% or more identical thereto, and/or having one, two, three or more substitutions, insertions or deletions, e.g., conserved substitutions), and the second chimeric membrane protein comprises the amino acid sequence of SEQ ID NO: 73 or 74 (or a sequence at least about 85%, 90%, 95%, 99% or more identical thereto, and/or having one, two, three or more substitutions, insertions or deletions, e.g., conserved substitutions).

In one embodiment, the first or second antigen binding domain binds a solid tumor antigen. In one embodiment, the solid tumor antigen is EGFRvIII, mesothelin, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRa4, or a peptide of any of these antigens presented on MHC. In one embodiment, said solid tumor antigen is selected from the group consisting of CLDN6, mesothelin and EGFRvIII.

In one embodiment, the first antigen binding domain binds CD19 and the second antigen binding domain binds mesothelin. In one embodiment, the first antigen binding domain binds CD19 and the second antigen binding domain binds EGFRvIII. In one embodiment, the first antigen binding domain binds CD19 and the second antigen binding domain binds CLDN6. In one embodiment, the first antigen binding domain binds mesothelin and the second antigen binding domain binds CD19. In one embodiment, the first antigen binding domain binds EGFRvIII and the second antigen binding domain binds CD19. In one embodiment, the first antigen binding domain binds CLDN6 and the second antigen binding domain binds CD19.

In one aspect, the invention provides a nucleic acid construct encoding the system of any of the aforementioned aspects and embodiments. In embodiments, the nucleic acid construct is RNA, for example, mRNA. In other embodiments, the nucleic acid construct is DNA. In one embodiment, the nucleic acid construct comprises a first nucleic acid molecule encoding the first chimeric membrane protein and a second nucleic acid molecule encoding the second chimeric membrane protein. In one embodiment, the first and second nucleic acid molecules are disposed on a single nucleic acid molecule. In one embodiment, the first and second nucleic acid molecules are disposed on separate nucleic acid molecules.

In one aspect, the invention provides a vector including the nucleic acid construct of the previous aspect. In embodiments, said vector is a lentiviral, adenoviral, or retroviral vector. In embodiments, upon expression of said first and second chimeric membrane proteins, said proteins are expressed as a single mRNA transcript, for example, wherein the nucleic acid sequences encoding said first and second chimeric membrane proteins are separated by a nucleic acid encoding a self-cleavage site or an internal ribosomal entry site.

In one aspect, the invention provides a cell including the nucleic acid construct of any of the aforementioned nucleic acid construct aspects and embodiments, the vector of any of the aforementioned vector aspects and embodiments, or the system of any of the aforementioned aspects and embodiments. In embodiments, the cell is a T cell or an NK cell.

In one embodiment, the cell further comprises a first inhibitor, wherein:

(i) the first chimeric membrane protein comprises a first extracellular domain derived from the extracellular domain of CD3 gamma, and the first inhibitor reduces the expression of endogenous CD3 gamma in the cell;

(ii) the first chimeric membrane protein comprises a first extracellular domain derived from the extracellular domain of CD3 delta, and the first inhibitor reduces the expression of endogenous CD3 delta in the cell; or

(iii) the first chimeric membrane protein comprises a first extracellular domain derived from the extracellular domain of CD3 epsilon, and the first inhibitor reduces the expression of endogenous CD3 epsilon in the cell. In one embodiment, the first inhibitor does not reduce or does not substantially reduce the expression of the first chimeric membrane protein in the cell (e.g., the first inhibitor reduces the expression of the first chimeric membrane protein at a level no more than 2, 5, 10, 15, or 20% compared to the expression of the first chimeric membrane protein in the absence of the first inhibitor).

In one embodiment, the cell further comprises a second inhibitor, wherein:

(i) the second chimeric membrane protein comprises a second extracellular domain derived from the extracellular domain of CD3 gamma, and the second inhibitor reduces the expression of endogenous CD3 gamma in the cell;

(ii) the second chimeric membrane protein comprises a second extracellular domain derived from the extracellular domain of CD3 delta, and the second inhibitor reduces the expression of endogenous CD3 delta in the cell; or

(iii) the second chimeric membrane protein comprises a second extracellular domain derived from the extracellular domain of CD3 epsilon, and the second inhibitor reduces the expression of endogenous CD3 epsilon in the cell. In one embodiment, the second inhibitor does not reduce or does not substantially reduce the expression of the second chimeric membrane protein in the cell (e.g., the second inhibitor reduces the expression of the second chimeric membrane protein at a level no more than 2, 5, 10, 15, or 20% compared to the expression of the second chimeric membrane protein in the absence of the second inhibitor). In one embodiment, the first or second inhibitor is an agent that mediates RNA interference, e.g., an siRNA or shRNA, or a nucleic acid molecule encoding an siRNA or shRNA. In one embodiment, the first or second inhibitor is a gene editing system (e.g., a CRISPR/Cas9 system, a zinc finger nuclease system, a TALEN system, or a meganuclease system) or a nucleic acid molecule encoding one or more components of the gene editing system.

In one aspect, the invention provides a method of treating a subject with a proliferative disorder, said method including administering to the subject the cell of any one of the aforementioned cell aspects and embodiments. In embodiments, said subject has a tumor and said administration provides said subject with immunity against said tumor.

In one aspect, the invention provides a method of providing an anti-cancer immune response in a subject having a cancer, comprising administering to the subject the cell of any one of the aforementioned cell aspects and embodiments.

In one embodiment, said cell is a T cell or NK cell and is autologous to said subject. In other embodiments, said cell is an allogeneic T cell or NK cell. In embodiments, said subject is a human. In one embodiment, the subject has a cancer. In one embodiment, the cancer is chosen from mesothelioma (e.g., malignant pleural mesothelioma), e.g., in a subject who has progressed on at least one prior standard therapy; lung cancer (e.g., non-small cell lung cancer, small cell lung cancer, squamous cell lung cancer, or large cell lung cancer); pancreatic cancer (e.g., pancreatic ductal adenocarcinoma, or metastatic pancreatic ductal adenocarcinoma (PDA), e.g., in a subject who has progressed on at least one prior standard therapy); esophageal adenocarcinoma, ovarian cancer (e.g., serous epithelial ovarian cancer, e.g., in a subject who has progressed after at least one prior regimen of standard therapy), breast cancer, colorectal cancer, bladder cancer or any combination thereof. In one embodiment, the cancer is chosen from chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, acute myeloid leukemia (AML), or unclassifiable lymphoma.

In one embodiment, the first antigen binding domain binds to a first antigen (e.g., a first tumor antigen) and the second antigen binding domain binds to a second antigen (e.g., a second tumor antigen), wherein the cancer exhibits heterogeneous expression of the first antigen (e.g., a first tumor antigen) and/or the second antigen (e.g., a second tumor antigen), e.g., wherein less than 90%, 80%, 70%, 60%, or 50% of cells in the cancer express the first antigen (e.g., a first tumor antigen) and less than 90%, 80%, 70%, 60%, or 50% of cells in the cancer express the second antigen (e.g., a second tumor antigen).

In one aspect, this invention provides a method of making a cell, comprising introducing the vector of the aforementioned vector aspects and embodiments into a cell. In one embodiment, the method comprises transducing a cell with the vector of the aforementioned vector aspects and embodiments.

In one embodiment, the method further comprises introducing a first inhibitor into the cell, wherein:

(i) the first chimeric membrane protein comprises a first extracellular domain derived from the extracellular domain of CD3 gamma, and the first inhibitor reduces the expression of endogenous CD3 gamma in the cell;

(ii) the first chimeric membrane protein comprises a first extracellular domain derived from the extracellular domain of CD3 delta, and the first inhibitor reduces the expression of endogenous CD3 delta in the cell; or

(iii) the first chimeric membrane protein comprises a first extracellular domain derived from the extracellular domain of CD3 epsilon, and the first inhibitor reduces the expression of endogenous CD3 epsilon in the cell. In one embodiment, the first inhibitor does not reduce or does not substantially reduce the expression of the first chimeric membrane protein in the cell (e.g., the first inhibitor reduces the expression of the first chimeric membrane protein at a level no more than 2, 5, 10, 15, or 20% compared to the expression of the first chimeric membrane protein in the absence of the first inhibitor).

In one embodiment, the method further comprises introducing a second inhibitor into the cell, wherein:

(i) the second chimeric membrane protein comprises a second extracellular domain derived from the extracellular domain of CD3 gamma, and the second inhibitor reduces the expression of endogenous CD3 gamma in the cell;

(ii) the second chimeric membrane protein comprises a second extracellular domain derived from the extracellular domain of CD3 delta, and the second inhibitor reduces the expression of endogenous CD3 delta in the cell; or

(iii) the second chimeric membrane protein comprises a second extracellular domain derived from the extracellular domain of CD3 epsilon, and the second inhibitor reduces the expression of endogenous CD3 epsilon in the cell. In one embodiment, the second inhibitor does not reduce or does not substantially reduce the expression of the second chimeric membrane protein in the cell (e.g., the second inhibitor reduces the expression of the second chimeric membrane protein at a level no more than 2, 5, 10, 15, or 20% compared to the expression of the second chimeric membrane protein in the absence of the second inhibitor). In one embodiment, the first or second inhibitor is an agent that mediates RNA interference, e.g., an siRNA or shRNA, or a nucleic acid molecule encoding an siRNA or shRNA. In one embodiment, the first or second inhibitor is a gene editing system (e.g., a CRISPR/Cas9 system, a zinc finger nuclease system, a TALEN system, or a meganuclease system) or a nucleic acid molecule encoding one or more components of the gene editing system.

In one embodiment, the cell is an immune effector cell, e.g., a T cell or an NK cell.

Vectors

In another aspect, the invention pertains to a vector comprising a nucleic acid sequence encoding a chimeric polypeptide described herein. In one embodiment, the vector is chosen from a DNA vector, an RNA vector, a plasmid, a lentivirus vector, adenoviral vector, or a retrovirus vector. In one embodiment, the vector is a lentivirus vector.

In an embodiment, the vector comprises a nucleic acid sequence that encodes a chimeric protein, e.g., as described herein, and a nucleic acid sequence that encodes an inhibitory molecule comprising: an inhKIR cytoplasmic domain; a transmembrane domain, e.g., a KIR transmembrane domain; and an inhibitor cytoplasmic domain, e.g., an ITIM domain, e.g., an inhKIR ITIM domain. In an embodiment the inhibitory molecule is a naturally occurring inhKIR, or a sequence sharing at least 50, 60, 70, 80, 85, 90, 95, or 99% homology with, or that differs by no more than 1, 2, 3, 4, 5, 6, 7, 8, 9, 10, 15, or 20 residues from, a naturally occurring inhKIR.

In an embodiment, the nucleic acid sequence that encodes an inhibitory molecule comprises: a SLAM family cytoplasmic domain; a transmembrane domain, e.g., a SLAM family transmembrane domain; and an inhibitor cytoplasmic domain, e.g., a SLAM family domain, e.g., an SLAM family ITIM domain. In an embodiment the inhibitory molecule is a naturally occurring SLAM family member, or a sequence sharing at least 50, 60, 70, 80, 85, 90, 95, or 99% homology with, or that differs by no more than 1, 2, 3, 4, 5, 6, 7, 8, 9, 10, 15, or 20 residues from, a naturally occurring SLAM family member.

In one embodiment, the vector further comprises a promoter. In some embodiments, the promoter is chosen from an EF-1 promoter, a CMV IE gene promoter, an EF-1α promoter, an ubiquitin C promoter, or a phosphoglycerate kinase (PGK) promoter. In one embodiment, the promoter is an EF-1 promoter.

In one embodiment, the vector is an in vitro transcribed vector, e.g., a vector that transcribes RNA of a nucleic acid molecule described herein. In one embodiment, the nucleic acid sequence in the vector further comprises a poly(A) tail, e.g., a poly A tail described herein, e.g., comprising about 150 adenosine bases. In one embodiment, the nucleic acid sequence in the vector further comprises a 3′UTR, e.g., a 3′ UTR described herein, e.g., comprising at least one repeat of a 3′UTR derived from human beta-globulin. In one embodiment, the nucleic acid sequence in the vector further comprises promoter, e.g., a T2A promoter.

Polypeptides

In another aspect, the invention features one or more isolated polypeptide molecules comprising one or more of an antigen binding domain, a transmembrane domain, and an intracellular signaling domain, wherein said antigen binding domain binds to a tumor antigen chosen from one or more of: CD19, CD123, CD22, CD30, CD171, CS-1, CLL-1 (CLECL1), CD33, EGFRvIII, GD2, GD3, BCMA, Tn Ag, PSMA, ROR1, FLT3, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, FAP, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, legumain, HPV E6,E7, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In some embodiments, the antigen binding domain of the polypeptide molecule binds to a tumor antigen chosen from one or more of: TSHR, CD171, CS-1, CLL-1, GD3, Tn Ag, FLT3, CD38, CD44v6, B7H3, KIT, IL-13Ra2, IL-11Ra, PSCA, PRSS21, VEGFR2, LewisY, CD24, PDGFR-beta, SSEA-4, MUC1, EGFR, NCAM, CAIX, LMP2, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, and IGLL1.

In some embodiments, the antigen binding domain of the polypeptide molecule binds to a tumor antigen chosen from one or more of: TSHR, CLDN6, GPRC5D, CXORF61, CD97, CD179a, ALK, polysialic acid, PLAC1, GloboH, NY-BR-1, UPK2, HAVCR1, ADRB3, PANX3, GPR20, LY6K, and OR51E2.

In some embodiments, the antigen binding domain of the polypeptide molecule comprises an antibody, an antibody fragment, an scFv, a Fv, a Fab, a (Fab′)2, a single domain antibody (SDAB), a VH or VL domain, or a camelid VHH domain.

In some embodiments, the antigen binding domain of the polypeptide molecule comprises a transmembrane domain of a protein chosen from an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C, or a functional variant thereof.

In other embodiments, the intracellular signaling domain of the polypeptide molecule comprises a primary signaling domain and/or a costimulatory signaling domain. In other embodiments, the intracellular signaling domain of the polypeptide molecule comprises a primary signaling domain. In other preferred embodiments, the intracellular signaling domain of the polypeptide molecule comprises a costimulatory signaling domain. In yet other embodiments, the intracellular signaling domain of the polypeptide molecule comprises a primary signaling domain and a costimulatory signaling domain.

In other embodiments, the primary signaling domain of the CAR polypeptide molecule comprises a functional signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP12. In one embodiment, the primary signaling domain comprises a functional signaling domain of CD3 zeta.

In preferred embodiments, the intracellular signaling domain of the CAR polypeptide molecule comprises a sequence encoding a costimulatory signaling domain. For example, the intracellular signaling domain can comprise a sequence encoding a primary signaling domain and a sequence encoding a costimulatory signaling domain. In some embodiments, the encoded costimulatory signaling domain comprises a functional signaling domain of a protein chosen from one or more of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, or NKG2D, or a functional variant thereof.

In some embodiments, the CAR polypeptide molecule further comprises a leader sequence. In certain embodiments, the antigen binding domain of the polypeptide molecule has a binding affinity KD of 10⁻⁴ M to 10⁻⁸ M. In one embodiment, the antigen binding domain is an antigen binding domain described herein, e.g., an antigen binding domain described herein for a target provided above. In one embodiment, the CAR molecule comprises an antigen binding domain that has a binding affinity KD of 10⁻⁴ M to 10⁻⁸ M, e.g., 10⁻⁵ M to 10⁻⁷ M, e.g., 10⁻⁶ M or 10⁻⁷ M, for the target antigen. In one embodiment, the antigen binding domain has a binding affinity that is at least five-fold, 10-fold, 20-fold, 30-fold, 50-fold, 100-fold or 1,000-fold less than a reference antibody, e.g., an antibody described herein. In one embodiment, the encoded antigen binding domain has a binding affinity at least 5-fold less than a reference antibody (e.g., an antibody from which the antigen binding domain is derived).

In another aspect, the invention features an isolated polypeptide molecule comprising an antigen binding domain, a transmembrane domain, and an intracellular signaling domain, wherein said antigen binding domain binds to a tumor-supporting antigen (e.g., a tumor-supporting antigen as described herein). In some embodiments, the tumor-supporting antigen is an antigen present on a stromal cell or a myeloid-derived suppressor cell (MDSC).

Chimeric Protein- and Chimeric Protein System-Expressing Cells

In another aspect, the invention pertains to a cell, e.g., an immune effector cell, (e.g., a population of cells, e.g., a population of immune effector cells) comprising a nucleic acid molecule, one or more chimeric polypeptide molecules, or a vector as described herein.

In one embodiment, the cell is a human T cell. In one embodiment, the cell is a cell described herein, e.g., a human T cell, e.g., a human T cell described herein; or a human NK cell, e.g., a human NK cell described herein. In one embodiment, the human T cell is a CD8+ T cell. In one embodiment, the cell is a T cell and the T cell is diaglycerol kinase (DGK) deficient. In one embodiment, the cell is a T cell and the T cell is Ikaros deficient. In one embodiment, the cell is a T cell and the T cell is both DGK and Ikaros deficient.

In another embodiment, a chimeric protein-expressing immune effector cell described herein can further express another agent, e.g., an agent which enhances the activity of a cell. For example, in one embodiment, the agent can be an agent which inhibits an inhibitory molecule. Examples of inhibitory molecules include PD-1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4 and TGF beta, e.g., as described herein. In one embodiment, the agent that inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD-1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4 or TGF beta, or a fragment of any of these, and a second polypeptide which is an intracellular signaling domain described herein (e.g., comprising a costimulatory domain (e.g., 41BB, CD27 or CD28, e.g., as described herein) and/or a primary signaling domain (e.g., a CD3 zeta signaling domain described herein). In one embodiment, the agent comprises a first polypeptide of PD-1 or a fragment thereof, and a second polypeptide of an intracellular signaling domain described herein (e.g., a CD28, CD27, OX40 or 4-IBB signaling domain described herein and/or a CD3 zeta signaling domain described herein).

In one embodiment, the cell further comprises an inhibitory molecule comprising: an inhKIR cytoplasmic domain; a transmembrane domain, e.g., a KIR transmembrane domain; and an inhibitor cytoplasmic domain, e.g., an ITIM domain, e.g., an inhKIR ITIM domain. In an embodiment the inhibitory molecule is a naturally occurring inhKIR, or a sequence sharing at least 50, 60, 70, 80, 85, 90, 95, or 99% homology with, or that differs by no more than 1, 2, 3, 4, 5, 6, 7, 8, 9, 10, 15, or 20 residues from, a naturally occurring inhKIR.

In one embodiment, the cell further comprises an inhibitory molecule comprising: a SLAM family cytoplasmic domain; a transmembrane domain, e.g., a SLAM family transmembrane domain; and an inhibitor cytoplasmic domain, e.g., a SLAM family domain, e.g., an SLAM family ITIM domain. In an embodiment the inhibitory molecule is a naturally occurring SLAM family member, or a sequence sharing at least 50, 60, 70, 80, 85, 90, 95, or 99% homology with, or that differs by no more than 1, 2, 3, 4, 5, 6, 7, 8, 9, 10, 15, or 20 residues from, a naturally occurring SLAM family member.

In one embodiment, the second CAR in the cell is an inhibitory CAR, wherein the inhibitory CAR comprises an antigen binding domain, a transmembrane domain, and an intracellular domain of an inhibitory molecule. The inhibitory molecule can be chosen from one or more of: PD1, PD-L1, CTLA-4, TIM-3, LAG-3, VISTA, BTLA, TIGIT, LAIR1, CD160, 2B4, TGF beta, CEACAM-1, CEACAM-3, and CEACAM-5. In one embodiment, the second CAR molecule comprises the extracellular domain of PD1 or a fragment thereof. In embodiments, the second CAR molecule in the cell further comprises an intracellular signaling domain comprising a primary signaling domain and/or an intracellular signaling domain.

In other embodiments, the intracellular signaling domain in the cell comprises a primary signaling domain comprising the functional domain of CD3 zeta, or a functional variant thereof, and a costimulatory signaling domain comprising the functional domain of 4-1BB. In certain embodiments, the antigen binding domain of the first chimeric molecule comprises a scFv and the antigen binding domain of the second chimeric molecule does not comprise a scFv. For example, the antigen binding domain of the first chimeric molecule comprises a scFv and the antigen binding domain of the second chimeric molecule comprises a camelid VHH domain.

Methods of Treatment/Combination Therapies

In another aspect, the present invention provides a method comprising administering a polypeptide, e.g., as described herein, or a cell comprising one or more nucleic acids encoding a polypeptide, e.g., as described herein. In one embodiment, the subject has a disorder described herein, e.g., the subject has cancer, e.g., the subject has a cancer which expresses a target antigen described herein. In one embodiment, the subject is a human.

In another aspect, the invention pertains to a method of treating a subject having a disease associated with expression of a cancer associated antigen as described herein comprising administering to the subject an effective amount of a cell comprising a polypeptide, e.g., as described herein.

In yet another aspect, the invention features a method of treating a subject having a disease associated with expression of a tumor antigen, comprising administering to the subject an effective amount of a cell, e.g., an immune effector cell (e.g., a population of immune effector cells) comprising a chimeric molecule as described herein.

In a related aspect, the invention features a method of treating a subject having a disease associated with expression of a tumor antigen. The method comprises administering to the subject an effective amount of a cell, e.g., an immune effector cell (e.g., a population of immune effector cells) comprising a chimeric molecule, in combination with an agent that increases the efficacy of the immune cell, wherein:

In another aspect, the invention features a composition comprising an immune effector cell (e.g., a population of immune effector cells) comprising a polypeptide, e.g., as described herein for use in the treatment of a subject having a disease associated with expression of a tumor antigen, e.g., a disorder as described herein.

In certain embodiments of any of the aforesaid methods or uses, the disease associated with a tumor antigen, e.g., a tumor antigen described herein, is selected from a proliferative disease such as a cancer or malignancy or a precancerous condition such as a myelodysplasia, a myelodysplastic syndrome or a preleukemia, or is a non-cancer related indication associated with expression of a tumor antigen described herein. In one embodiment, the disease is a cancer described herein, e.g., a cancer described herein as being associated with a target described herein. In one embodiment, the disease is a hematologic cancer. In one embodiment, the hematologic cancer is leukemia. In one embodiment, the cancer is selected from the group consisting of one or more acute leukemias including but not limited to B-cell acute lymphoid leukemia (“BALL”), T-cell acute lymphoid leukemia (“TALL”), acute lymphoid leukemia (ALL); one or more chronic leukemias including but not limited to chronic myelogenous leukemia (CML), chronic lymphocytic leukemia (CLL); additional hematologic cancers or hematologic conditions including, but not limited to B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and to disease associated with expression of a tumor antigen described herein include, but not limited to, atypical and/or non-classical cancers, malignancies, precancerous conditions or proliferative diseases expressing a tumor antigen as described herein; and any combination thereof. In another embodiment, the disease associated with a tumor antigen described herein is a solid tumor.

In certain embodiments of any of the aforesaid methods or uses, the tumor antigen associated with the disease is chosen from one or more of: CD19, CD123, CD22, CD30, CD171, CS-1, CLL-1 (CLECL1), CD33, EGFRvIII, GD2, GD3, BCMA, Tn Ag, PSMA, ROR1, FLT3, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, FAP, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, legumain, HPV E6, E7, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In other embodiments of any of the aforesaid methods or uses, the tumor antigen associated with the disease is chosen from one or more of: TSHR, TSHR, CD171, CS-1, CLL-1, GD3, Tn Ag, FLT3, CD38, CD44v6, B7H3, KIT, IL-13Ra2, IL-11Ra, PSCA, PRSS21, VEGFR2, LewisY, CD24, PDGFR-beta, SSEA-4, MUC1, EGFR, NCAM, CAIX, LMP2, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, and IGLL1.

In other embodiments of any of the aforesaid methods or uses, the tumor antigen associated with the disease is chosen from one or more of: TSHR, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, and OR51E2.

In certain embodiments, the methods or uses are carried out in combination with an agent that increases the efficacy of the immune effector cell, e.g., an agent as described herein.

In any of the aforesaid methods or uses, the disease associated with expression of the tumor antigen is selected from the group consisting of a proliferative disease, a precancerous condition, a cancer, and a non-cancer related indication associated with expression of the tumor antigen.

The cancer can be a hematologic cancer, e.g., a cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

The cancer can also be chosen from colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers.

In certain embodiments of the methods or uses described herein, the chimeric molecule is administered in combination with an agent that increases the efficacy of the immune effector cell, e.g., one or more of a protein phosphatase inhibitor, a kinase inhibitor, a cytokine, an inhibitor of an immune inhibitory molecule; or an agent that decreases the level or activity of a T_{REG} cell.

In certain embodiments of the methods or uses described herein, the protein phosphatase inhibitor is a SHP-1 inhibitor and/or an SHP-2 inhibitor.

In other embodiments of the methods or uses described herein, kinase inhibitor is chosen from one or more of a CDK4 inhibitor, a CDK4/6 inhibitor (e.g., palbociclib), a BTK inhibitor (e.g., ibrutinib or RN-486), an mTOR inhibitor (e.g., rapamycin or everolimus (RAD001)), an MNK inhibitor, or a dual P13K/mTOR inhibitor. In one embodiment, the BTK inhibitor does not reduce or inhibit the kinase activity of interleukin-2-inducible kinase (ITK).

In other embodiments of the methods or uses described herein, the agent that inhibits the immune inhibitory molecule comprises an antibody or antibody fragment, an inhibitory nucleic acid, a clustered regularly interspaced short palindromic repeats (CRISPR), a transcription-activator like effector nuclease (TALEN), or a zinc finger endonuclease (ZFN) that inhibits the expression of the inhibitory molecule.

In other embodiments of the methods or uses described herein, the agent that decreases the level or activity of the T_{REG} cells is chosen from cyclophosphamide, anti-GITR antibody, CD25-depletion, or a combination thereof.

In certain embodiments of the methods or uses described herein, the immune inhibitory molecule is selected from the group consisting of PD1, PD-L1, CTLA-4, TIM-3, LAG-3, VISTA, BTLA, TIGIT, LAIR1, CD160, 2B4, TGF beta, CEACAM-1, CEACAM-3, and CEACAM-5.

In other embodiments, the agent that inhibits the inhibitory molecule comprises a first polypeptide comprising an inhibitory molecule or a fragment thereof and a second polypeptide that provides a positive signal to the cell, and wherein the first and second polypeptides are expressed on the CAR-containing immune cells, wherein (i) the first polypeptide comprises PD1, PD-L1, CTLA-4, TIM-3, LAG3, VISTA, BTLA, TIGIT, LAIR1, CD160, 2B4, TGF beta, CEACAM-1, CEACAM-3, and CEACAM-5 or a fragment thereof; and/or (ii) the second polypeptide comprises an intracellular signaling domain comprising a primary signaling domain and/or a costimulatory signaling domain. In one embodiment, the primary signaling domain comprises a functional domain of CD3 zeta; and/or the costimulatory signaling domain comprises a functional domain of a protein selected from 41BB, CD27 and CD28.

In other embodiments, cytokine is chosen from IL-7, IL-15 or IL-21, or both.

In other embodiments, the immune effector cell and a second, e.g., any of the combination therapies disclosed herein (e.g., the agent that that increases the efficacy of the immune effector cell) are administered substantially simultaneously or sequentially.

In one embodiment, lymphocyte infusion, for example allogeneic lymphocyte infusion, is used in the treatment of the cancer, wherein the lymphocyte infusion comprises at least one cell of the present invention. In one embodiment, autologous lymphocyte infusion is used in the treatment of the cancer, wherein the autologous lymphocyte infusion comprises at least one cell described herein.

In one embodiment, the cell is a T cell and the T cell is diaglycerol kinase (DGK) deficient. In one embodiment, the cell is a T cell and the T cell is Ikaros deficient. In one embodiment, the cell is a T cell and the T cell is both DGK and Ikaros deficient.

In one embodiment, the method includes administering a cell expressing the cell as described herein, in combination with an agent which enhances the activity of such a cell, wherein the agent is a cytokine, e.g., IL-7, IL-15, IL-21, or a combination thereof. The cytokine can be delivered in combination with, e.g., simultaneously or shortly after, administration of the cell. Alternatively, the cytokine can be delivered after a prolonged period of time after administration of the cell, e.g., after assessment of the subject's response to the cell. In one embodiment the cytokine is administered to the subject simultaneously (e.g., administered on the same day) with or shortly after administration (e.g., administered 1 day, 2 days, 3 days, 4 days, 5 days, 6 days, or 7 days after administration) of the cell or population of cells of any of claims 61-80. In other embodiments, the cytokine is administered to the subject after a prolonged period of time (e.g., e.g., at least 2 weeks, 3 weeks, 4 weeks, 6 weeks, 8 weeks, 10 weeks, or more) after administration of the cell or population of cells of any of claims 61-80, or after assessment of the subject's response to the cell.

In other embodiments, the cells are administered in combination with an agent that ameliorates one or more side effects associated with administration of a cell. Side effects associated with the cell can be chosen from cytokine release syndrome (CRS) or hemophagocytic lymphohistiocytosis (HLH).

In embodiments of any of the aforeseaid methods or uses, the cells expressing the molecule are administered in combination with an agent that treats the disease associated with expression of the tumor antigen, e.g., any of the second or third therapies disclosed herein. Additional exemplary combinations include one or more of the following.

In another embodiment, the cell expressing the molecule, e.g., as described herein, can be administered in combination with another agent, e.g., a kinase inhibitor and/or checkpoint inhibitor described herein. In an embodiment, a cell can further express another agent, e.g., an agent which enhances the activity of a chimeric protein-expressing cell.

For example, in one embodiment, the agent that enhances the activity of a cell can be an agent which inhibits an inhibitory molecule (e.g., an immune inhibitor molecule). Examples of inhibitory molecules include PD1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4 and TGF beta.

In one embodiment, the agent that inhibits the inhibitory molecule is an inhibitory nucleic acid is a dsRNA, a siRNA, or a shRNA. In embodiments, the inhibitory nucleic acid is linked to the nucleic acid that encodes a component of the chimeric molecule. For example, the inhibitory molecule can be expressed on the cell.

In another embodiment, the agent which inhibits an inhibitory molecule, e.g., is a molecule described herein, e.g., an agent that comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD-1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4 or TGF beta, or a fragment of any of these (e.g., at least a portion of the extracellular domain of any of these), and a second polypeptide which is an intracellular signaling domain described herein (e.g., comprising a costimulatory domain (e.g., 41BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of the extracellular domain of PD1), and a second polypeptide of an intracellular signaling domain described herein (e.g., a CD28 signaling domain described herein and/or a CD3 zeta signaling domain described herein).

In one embodiment, the immune effector cell of the present invention, e.g., T cell or NK cell, is administered to a subject that has received a previous stem cell transplantation, e.g., autologous stem cell transplantation.

In one embodiment, the immune effector cell of the present invention, e.g., T cell or NK cells, is administered to a subject that has received a previous dose of melphalan.

In one embodiment, the cell described herein is administered in combination with an agent that increases the efficacy of a cell, e.g., an agent described herein.

In one embodiment, the cells described herein are administered in combination with a low, immune enhancing dose of an mTOR inhibitor. While not wishing to be bound by theory, it is believed that treatment with a low, immune enhancing, dose (e.g., a dose that is insufficient to completely suppress the immune system but sufficient to improve immune function) is accompanied by a decrease in PD-1 positive T cells or an increase in PD-1 negative cells. PD-1 positive T cells, but not PD-1 negative T cells, can be exhausted by engagement with cells which express a PD-1 ligand, e.g., PD-L1 or PD-L2.

In an embodiment, administration of a low, immune enhancing, dose of an mTOR inhibitor, e.g., an allosteric inhibitor, e.g., RAD001, or a catalytic inhibitor, is initiated prior to administration of a cell described herein, e.g., T cells or NK cells. In an embodiment, the cells are administered after a sufficient time, or sufficient dosing, of an mTOR inhibitor, such that the level of PD1 negative immune effector cells, e.g., T cells or NK cells, or the ratio of PD1 negative immune effector cells, e.g., T cells/PD1 positive immune effector cells, e.g., T cells, has been, at least transiently, increased.

In one embodiment, the cell described herein is administered at a dose and/or dosing schedule described herein.

In another aspect, the invention pertains to the isolated nucleic acid molecule encoding one or more chimeric proteins of the invention, the isolated polypeptide molecule of one or more chimeric proteins of the invention, the vector comprising a nucleic acid encoding one or more chimeric proteins of the invention, and the cell comprising one or more chimeric proteins of the invention for use as a medicament.

In any of the aforesaid methods or uses, the disease associated with expression of the tumor-supporting antigen is selected from the group consisting of a proliferative disease, a precancerous condition, a cancer, and a non-cancer related indication associated with expression of the tumor-supporting antigen. In an embodiment, the disease associated with a tumor-supporting antigen described herein is a solid tumor.

In one embodiment of the methods or uses described herein, the polypeptide described herein is administered in combination with another agent. In one embodiment, the agent can be a kinase inhibitor, e.g., a CDK4/6 inhibitor, a BTK inhibitor, an mTOR inhibitor, a MNK inhibitor, or a dual PI3K/mTOR inhibitor, and combinations thereof. In one embodiment, the kinase inhibitor is a CDK4 inhibitor, e.g., a CDK4 inhibitor described herein, e.g., a CD4/6 inhibitor, such as, e.g., 6-Acetyl-8-cyclopentyl-5-methyl-2-(5-piperazin-1-yl-pyridin-2-ylamino)-8H-pyrido[2,3-d]pyrimidin-7-one, hydrochloride (also referred to as palbociclib or PD0332991). In one embodiment, the kinase inhibitor is a BTK inhibitor, e.g., a BTK inhibitor described herein, such as, e.g., ibrutinib. In one embodiment, the kinase inhibitor is an mTOR inhibitor, e.g., an mTOR inhibitor described herein, such as, e.g., rapamycin, a rapamycin analog, OSI-027. The mTOR inhibitor can be, e.g., an mTORC1 inhibitor and/or an mTORC2 inhibitor, e.g., an mTORC1 inhibitor and/or mTORC2 inhibitor described herein. In one embodiment, the kinase inhibitor is a MNK inhibitor, e.g., a MNK inhibitor described herein, such as, e.g., 4-amino-5-(4-fluoroanilino)-pyrazolo [3,4-d] pyrimidine. The MNK inhibitor can be, e.g., a MNK1a, MNK1b, MNK2a and/or MNK2b inhibitor. The dual PI3K/mTOR inhibitor can be, e.g., PF-04695102.

In one embodiment of the methods or uses described herein, the kinase inhibitor is a CDK4 inhibitor selected from aloisine A; flavopiridol or HMR-1275, 2-(2-chlorophenyl)-5,7-dihydroxy-8-[(3S,4R)-3-hydroxy-1-methyl-4-piperidinyl]-4-chromenone; crizotinib (PF-02341066; 2-(2-Chlorophenyl)-5,7-dihydroxy-8-[(2R,3S)-2-(hydroxymethyl)-1-methyl-3-pyrrolidinyl]-4H-1-benzopyran-4-one, hydrochloride (P276-00); 1-methyl-5-[[2-[5-(trifluoromethyl)-1H-imidazol-2-yl]-4-pyridinyfloxy]-N-[4-(trifluoromethyl)phenyl]-1H-benzimidazol-2-amine (RAF265); indisulam (E7070); roscovitine (CYC202); palbociclib (PD0332991); dinaciclib (SCH727965); N45-[[(5-tert-butyloxazol-2-yl)methyl]thio]thiazol-2-yl]piperidine-4-carboxamide (BMS 387032); 4-[[9-chloro-7-(2,6-difluorophenyl)-5H-pyrimido[5,4-d][2]benzazepin-2-yl]amino]-benzoic acid (MLN8054); 5-[3-(4,6-difluoro-1H-benzimidazol-2-yl)-1H-indazol-5-yl]-N-ethyl-4-methyl-3-pyridinemethanamine (AG-024322); 4-(2,6-dichlorobenzoylamino)-1H-pyrazole-3-carboxylic acid N-(piperidin-4-yl)amide (AT7519); 4-[2-methyl-1-(1-methylethyl)-1H-imidazol-5-yl]-N-[4-(methylsulfonyl)phenyl]-2-pyrimidinamine (AZD5438); and XL281 (BMS908662).

In one embodiment of the methods or uses described herein, the kinase inhibitor is an mTOR inhibitor selected from temsirolimus; ridaforolimus (1R,2R,4S)-4-[(2R)-2 [(1R,9S,12S,15R,16E,18R,19R,21R, 23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.0^{4,9}] hexatriaconta-16,24,26,28-tetraen-12-yl]propyl]-2-methoxycyclohexyl dimethylphosphinate, also known as AP23573 and MK8669; everolimus (RAD001); rapamycin (AY22989); simapimod; (5-{2,4-bis[(3S)-3-methylmorpholin-4-yl]pyrido[2,3-d]pyrimidin-7-yl}-2-methoxyphenyl)methanol (AZD8055); 2-amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-(“RGDS” disclosed as SEQ ID NO: 39), inner salt (SF1126); and XL765.

In one embodiment of the methods or uses described herein, the kinase inhibitor is an MNK inhibitor selected from CGP052088; 4-amino-3-(p-fluorophenylamino)-pyrazolo [3,4-d] pyrimidine (CGP57380); cercosporamide; ETC-1780445-2; and 4-amino-5-(4-fluoroanilino)-pyrazolo [3,4-d] pyrimidine.

In one embodiment of the methods or uses described herein, the kinase inhibitor is a dual phosphatidylinositol 3-kinase (PI3K) and mTOR inhibitor selected from 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF-04691502); N-[4-[[4-(Dimethylamino)-1-piperidinyl]carbonyl]phenyl]-N′-[4-(4,6-di-4-morpholinyl-1,3,5-triazin-2-yl)phenyl]urea (PF-05212384, PKI-587); 2-Methyl-2-{4-[3-methyl-2-oxo-8-(quinolin-3-yl)-2,3-dihydro-1H-imidazo[4,5-c]quinolin-1-yl]phenyl}propanenitrile (BEZ-235); apitolisib (GDC-0980, RG7422); 2,4-Difluoro-N-{2-(methyloxy)-5-[4-(4-pyridazinyl)-6-quinolinyl]-3-pyridinyl}benzenesulfonamide (GSK2126458); 8-(6-methoxypyridin-3-yl)-3-methyl-1-(4-(piperazin-1-yl)-3-(trifluoromethyl)phenyl)-1H-imidazo [4,5-c]quinolin-2(3H)-one Maleic acid (NVP-BGT226); 3-[4-(4-Morpholinylpyrido[3′,2′:4,5]furo[3,2-d]pyrimidin-2-yl]phenol (PI-103); 5-(9-isopropyl-8-methyl-2-morpholino-9H-purin-6-yl)pyrimidin-2-amine (VS-5584, SB2343); and N-[2-[(3,5-Dimethoxyphenyl)amino]quinoxalin-3-yl]-4-[(4-methyl-3-methoxyphenyl)carbonyl]aminophenylsulfonamide (XL765).

In one embodiment of the methods or uses described herein, an immune effector cell described herein is administered to a subject in combination with a protein tyrosine phosphatase inhibitor, e.g., a protein tyrosine phosphatase inhibitor described herein. In one embodiment, the protein tyrosine phosphatase inhibitor is an SHP-1 inhibitor, e.g., an SHP-1 inhibitor described herein, such as, e.g., sodium stibogluconate. In one embodiment, the protein tyrosine phosphatase inhibitor is an SHP-2 inhibitor.

In one embodiment of the methods or uses described herein, the chimeric molecule is administered in combination with another agent, and the agent is a cytokine. The cytokine can be, e.g., IL-7, IL-15, IL-21, or a combination thereof. In another embodiment, the CAR molecule is administered in combination with a checkpoint inhibitor, e.g., a checkpoint inhibitor described herein. For example, in one embodiment, the check point inhibitor inhibits an inhibitory molecule selected from PD-1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4 and TGF beta.

Methods of making chimeric protein- and chimeric protein system-expressing cells In another aspect, the invention pertains to a method of making a cell (e.g., an immune effector cell or population thereof) comprising introducing into (e.g., transducing) a cell, e.g., a T cell or a NK cell described herein, with a vector of comprising a nucleic acid encoding a polypeptide or system, e.g., as described herein; or a nucleic acid encoding a polypeptide or system, e.g., as described herein.

The cell in the methods is an immune effector cell (e.g., a T cell or a NK cell, or a combination thereof). In some embodiments, the cell in the methods is diaglycerol kinase (DGK) and/or Ikaros deficient.

In some embodiment, the introducing the nucleic acid molecule comprises transducing a vector comprising the nucleic acid molecule encoding a polypeptide or system, e.g., as described herein, or transfecting the nucleic acid molecule encoding a polypeptide or system, e.g., as described herein, wherein the nucleic acid molecule is an in vitro transcribed RNA.

In other embodiments, the population of cells is expanded by culturing the cells in the presence of an agent that stimulates a CD3/TCR complex associated signal and/or a ligand that stimulates a costimulatory molecule on the surface of the cells. The agent can be a bead conjugated with anti-CD3 antibody, or a fragment thereof, and/or anti-CD28 antibody, or a fragment thereof.

In other embodiments, the population of cells is expanded in an appropriate media that includes one or more interleukin that result in at least a 200-fold, 250-fold, 300-fold, or 350-fold increase in cells over a 14 day expansion period, as measured by flow cytometry.

In other embodiments, the population of cells is expanded in the presence IL-15 and/or IL-7.

In certain embodiments, the method further includes cryopreserving the population of cells after the appropriate expansion period.

In yet other embodiments, the method of making disclosed herein further comprises contacting the population of immune effector cells with a nucleic acid encoding a telomerase subunit, e.g., hTERT. The the nucleic acid encoding the telomerase subunit can be DNA.

The present invention also provides a method of generating a population of RNA-engineered cells, e.g., cells described herein, e.g., immune effector cells (e.g., T cells, NK cells), transiently expressing exogenous RNA.

In another aspect, the invention pertains to a method of providing an anti-tumor immunity in a subject comprising administering to the subject an effective amount of a cell as described herein. In one embodiment, the cell is an autologous T cell or NK cell. In one embodiment, the cell is an allogeneic T cell or NK cell. In one embodiment, the subject is a human In one aspect, the invention includes a population of autologous cells that are transfected or transduced with a vector comprising a nucleic acid molecule as described herein. In one embodiment, the vector is a retroviral vector. In one embodiment, the vector is a self-inactivating lentiviral vector as described elsewhere herein. In one embodiment, the vector is delivered (e.g., by transfecting or electroporating) to a cell, e.g., a T cell or a NK cell, wherein the vector comprises a nucleic acid molecule encoding a polypeptide as described herein, which is transcribed as an mRNA molecule, and the chimeric proteins of the present invention is translated from the RNA molecule and expressed on the surface of the cell.

In one embodiment, the nucleic acid molecule of the present invention molecule, e.g., as described herein, is expressed as an mRNA molecule. In one embodiment, the present invention-expressing cells, e.g., immune effector cells (e.g., T cells, NK cells), can be generated by transfecting or electroporating an RNA molecule encoding the desired proteins (e.g., without a vector sequence) into the cell. In one embodiment, a chimeric protein of the present invention molecule is translated from the RNA molecule once it is incorporated and expressed on the surface of the recombinant cell.

In certain aspects, the foregoing chimeric proteins (e.g., of a system described herein) are encoded by a single nucleic molecule in the same frame and as a single polypeptide chain. In this aspect, the proteins can, e.g., be separated by one or more peptide cleavage sites. (e.g., an auto-cleavage site or a substrate for an intracellular protease). Examples of peptide cleavage sites include the following, wherein the GSG residues are optional:

T2A: (GSG) E G R G S L L T C G D V E E N P G P (SEQ ID NO: 40)

P2A: (GSG) A T N F S L L K Q A G D V E E N P G P (SEQ ID NO: 41)

E2A: (GSG) Q C T N Y A L L K L A G D V E S N P G P (SEQ ID NO: 42)

F2A: (GSG) V K Q T L N F D L L K L A G D V E S N P G P (SEQ ID NO: 43)

In a related aspect, the invention features a single protein, as described above, encoding a two chimeric polypeptides.

In other aspects, the foregoing polypeptides, e.g., of the system, are encoded by a single, or multiple, nucleic molecules and are not expressed as a single polypeptide. Here, e.g., the polypeptides can be controlled by a common promoter or be separated by an internal ribosomal entry site. Alternatively, the expression of the two proteins can be, e.g., controlled by separate promoters.

In yet another aspect, the invention features one or more vectors (e.g., any of the vectors described above) including the foregoing nucleic acid molecules encoding different chimeric proteins, e.g., of the system.

### BRIEF DESCRIPTION OF THE DRAWINGS

FIG. 1 is a schematic showing constitutively active TCR-based Chimeric Antigen Receptor (TCAR). A targeting and costimulatory domain are embedded into the TCR complex by fusion with an intracellular heterodimerization domain and co-transfection/co-transduction with an endogenous TCR complex member such as CD3 epsilon fused to a second heterodimerization domain.

FIG. 2 is a pair of graphs showing JNL signaling and IL2 expression of antigen activated TCARs with intracellular heterodimerization domains.

FIG. 3 is a pair of graphs showing percentage of the indicated cell killing in cells transfected with the indicated constructs as a function of transfection.

FIG. 4 is a graph showing concentration of IL-2 expression as a function of transfection in the indicated constructs.

FIG. 5 is a schematic showing Constitutively Active TCR-based Chimeric Antigen Receptor (TCAR) with enhanced proliferation. A targeting and costimulatory domain are embedded into the TCR complex by fusion with an intracellular heterodimerization domain and co-transfection/co-transduction with the extracellular and transmembrane domains of an endogenous TCR complex member such as CD3 epsilon fused to a second costimulatory domain and a second heterodimerization domain. Unlike third generation CARs, this orientation provides for both costimulatory members to be membrane proximal and should further enhance proliferative capabilities.

FIG. 6 is a schematic showing constitutively active TCR-based Chimeric Antigen Receptor (TCAR). A costimulatory receptor with or without its natural extracellular domain is embedded into the TCR complex by fusion with an intracellular heterodimerization domain and co-transfection/co-transduction with a targeting domain fused to an endogenous TCR complex member such as CD3 epsilon fused to a second intracellular heterodimerization domain.

FIG. 7 is a schematic showing constitutively active TCR-based Chimeric Antigen Receptor (TCAR). A cytosolic costimulatory domain is embedded into the TCR complex by fusion with an intracellular heterodimerization domain and co-transfection/co-transduction with a targeting domain fused to an endogenous TCR complex member such as CD3 epsilon fused to a second intracellular heterodimerization domain.

FIG. 8 is a schematic showing constitutively active TCR-based Chimeric Antigen Receptor (TCAR). A targeting and costimulatory domain are embedded into the TCR complex by fusion with an intracellular domain which binds to a member of the TCR complex.

FIG. 9 is a schematic showing constitutively active TCR-based Chimeric Antigen Receptor (TCAR). A targeting and costimulatory domain are embedded into the TCR complex by fusion with an extracellular domain which binds to a member of the TCR complex.

FIG. 10 is a schematic showing constitutively active Chimeric Antigen Receptor TCR fusion (fusTCAR). VL and Vh of a targeting domain derived from an antibody are embedded into the TCR complex by direct fusions to the endogenous truncated alpha and beta TCR.

FIG. 11 is a schematic showing Constitutively Active Chimeric Antigen Receptor TCR fusion (fusTCAR). VL and Vh of a targeting domain derived from an antibody are embedded into the TCR complex by direct fusions to the endogenous truncated alpha and beta TCR followed by intracellular fusions of one or more costimulatory domains.

FIG. 12 is a schematic showing constitutively active Chimeric Antigen Receptor TCR fusion (fusTCAR). A targeting domain is embedded into the TCR complex by direct fusion to an endogenous TCR complex member such as CD3 epsilon.

FIG. 13 is a schematic showing constitutively active Chimeric Antigen Receptor TCR fusion (fusTCAR). A targeting domain is embedded into the TCR complex by direct fusion to am endogenous TCR complex member such as CD3 epsilon followed by one or more intracellular co-stimulatory domains such as 4-1BB, or a functional variant thereof.

FIG. 14 is a schematic showing constitutively active Chimeric Antigen Receptor TCR fusion (fusTCAR). A targeting domain is embedded into the TCR complex by direct fusion to the extracellular and transmembrane domains of endogenous TCR complex member such as CD3 epsilon followed by one or more intracellular co-stimulatory domains such as 4-1BB, or a functional variant thereof.

FIG. 15 is a graph showing JNL signaling and IL2 expression of activated fusTCARs.

FIG. 16 is a series of graphs showing percentage of specific killing of the indicated cells by cells transfected with the indicated constructs as a function of transfection.

FIG. 17 is a graph showing expression of IL-2 as a function of transfection with the indicated constructs.

FIG. 18 is a pair of graphs showing percentage of the indicated cell killing in cells transfected with the indicated constructs as a function of transfection.

FIG. 19 is a graph showing concentration of IL-2 expression as a function of transfection in the indicated constructs.

FIG. 20 is a schematic showing regulatable TCR-based Chimeric Antigen Receptor (rTCAR) with enhanced proliferation. A targeting and costimulatory domain are embedded into the TCR complex by fusion with an intracellular heterodimerization switch domain and co-transfection/co-transduction with the extracellular and transmembrane domains of an endogenous TCR complex member such as CD3 epsilon fused to a second costimulatory domain and a second heterodimerization switch domain. Signaling is induced upon addition of a switch molecule such as a rapalogue.

FIG. 21 is a schematic showing regulatable TCR-based Chimeric Antigen Receptor (rTCAR). A costimulatory receptor with or without its natural extracellular domain is embedded into the TCR complex by fusion with an intracellular heterodimerization switch domain and co-transfection/co-transduction with a targeting domain fused to an endogenous TCR complex member such as CD3 epsilon fused to a second intracellular heterodimerization switch domain. Proliferation is induced upon addition of a switch molecule such as a rapalogue.

FIG. 22 is a schematic showing constitutively active TCR-based Chimeric Antigen Receptor (TCAR). A targeting and costimulatory domain are embedded into the TCR complex by fusion with an intracellular heterodimerization switch domain and co-transfection/co-transduction with an extracellular domain which binds to a member of the TCR complex fused to a transmembrane and intracellualr domain of a costimulatory receptor and a second heterodimerization switch domain. Signaling is induced upon addition of a switch molecule such as a rapalogue.

FIG. 23 is a schematic showing constitutively active TCR-based Chimeric Antigen Receptor (TCAR). A targeting and costimulatory domain are embedded into the TCR complex by fusion with an intracellular heterodimerization switch domain and co-transfection/co-transduction with a costimulatory receptor with or without its natural extracellular domain fused to a second heterodimerization switch domain and an intracellular domain which binds to a member of the TCR complex. Signaling is induced upon addition of a switch molecule such as a rapalogue.

FIG. 24 is a schematic showing constitutively active TCR-based Chimeric Antigen Receptor (TCAR). A targeting and costimulatory domain are embedded into the TCR complex by fusion with an intracellular heterodimerization switch domain and co-transfection/co-transduction with a cytosolic costimulatory domain fused to a second heterodimerization switch domain and an intracellular domain which binds to a member of the TCR complex. Signaling is induced upon addition of a switch molecule such as a rapalogue.

FIG. 25 is a schematic showing regulatable TCR-based Chimeric Antigen Receptor (TCAR). A targeting and costimulatory domain are embedded into the TCR complex by fusion with an intracellular heterodimerization switch domain and co-transfection/co-transduction with an endogenous TCR complex member such as CD3 epsilon fused to a second heterodimerization switch domain. Signaling is induced upon addition of a switch molecule such as a rapalogue.

FIG. 26 is a schematic showing regulatable TCR-based Chimeric Antigen Receptor (rTCAR). A targeting and costimulatory domain are embedded into the TCR complex by fusion with an intracellular heterodimerization switch domain and co-transfection/co-transduction with an endogenous TCR complex member such as CD3 epsilon fused to a second heterodimerization switch domain. Signaling is induced upon addition of a switch molecule such as a rapalogue. ITAM domain from the CD3 epilson fusion was mutated to phenylalanine to demonstrate signaling was induced by other members of the TCR complex.

FIG. 27 is a series of graphs showing JNL signaling and IL2 expression of antigen activated FKBP/FRP rTCARs induced with RAD001.

FIG. 28 is a series of graphs showing a comparison of JNL signaling and IL2 expression for Rapalogue-mediated antigen activated FKBP/FRP rTCARs with and without knockout of CD3e ITAM signaling.

FIG. 29 is a pair of graphs showing percentage of the indicated cell killing in cells transfected with the indicated constructs as a function of transfection.

FIG. 30 is a graph showing concentration of IL-2 expression as a function of transfection in the indicated constructs.

FIG. 31 is a pair of graphs showing percentage of the indicated cell killing in cells transfected with the indicated constructs as a function of transfection.

FIG. 32 is a graph showing concentration of IL-2 expression as a function of transfection in the indicated constructs.

FIG. 33 is a graph showing light intensity as generated by an NFAT reporter gene system. The anti-idiotype antibody binds the expressed scFv.

FIG. 34 is a pair of graphs showing percentage of the indicated cell killing in cells transfected with the indicated constructs as a function of transfection.

FIG. 35 is a graph showing concentration of IL-2 expression as a function of transfection in the indicated constructs.

FIG. 36, left panel, is a graph showing percentage of the indicated cell killing in cells transfected with the indicated constructs as a function of transfection. FIG. 36, right panels are a series of graphs showing number of cells expressing the indicated construct under the indicated expression conditions.

FIG. 37 is a graph showing percentage of the indicated cell killing in cells transfected with the indicated constructs as a function of transfection.

FIG. 38 is a graph showing concentration of IL-2 expression as a function of transfection in the indicated constructs.

FIG. 39 is a graph showing percentage of the indicated cell killing in cells transfected with the indicated constructs as a function of transfection.

FIG. 40 is a graph showing concentration of IL-2 expression as a function of transfection in the indicated constructs.

FIG. 41 shows various examples of chimeric membrane proteins for use in the various aspects of the invention. In the system aspects of the invention, two or more chimeric membrane proteins are utilized together, e.g., expressed together in a cell.

FIG. 42 is a schematic showing a TCR-based Chimeric Antigen Receptor (TCAR) assembled from the systems of the present invention. The TCAR has specificity for two antigens by fusion of a first and second antigen binding domain (here depicted as scFv antigen binding domains) to a protein comprising the extracellular portion of the CD3 epsilon protine and to a protein comprising the extracellular portion of the CD3 gamma protein. A co-stimulatory signalling domain is further fused to the intracellular portion of one or more of the chimeric membrane molecules. Co-transfection/co-transduction of both chimeric membrane protein into, e.g., T cells, results in formation of TCR comprising two heterologous chimeric proteins, thereby imparting dual antigen specificity to the TCR/cell as well as both CD3 zeta signaling and co-stimulatory signaling upon antigen engagement.

FIG. 43 is a schematic showing a TCR-based Chimeric Antigen Receptor (TCAR) assembled from the systems of the present invention. The TCAR has specificity for two antigens by fusion of a first and second antigen binding domain (here depicted as scFv antigen binding domains) to a protein comprising the extracellular portion of the CD3 epsilon protine and to a protein comprising the extracellular portion of the CD3 delta protein. A co-stimulatory signalling domain is further fused to the intracellular portion of one or more of the chimeric membrane molecules. Co-transfection/co-transduction of both chimeric membrane protein into, e.g., T cells, results in formation of TCR comprising two heterologous chimeric proteins, thereby imparting dual antigen specificity to the TCR/cell as well as both CD3 zeta signaling and co-stimulatory signaling upon antigen engagement.

FIG. 44 is a schematic showing a TCR-based Chimeric Antigen Receptor (TCAR) assembled from the systems of the present invention. The TCAR has specificity for two antigens by fusion of a first and second antigen binding domain (here depicted as scFv antigen binding domains) to a protein comprising the extracellular portion of the CD3 delta protein and to a protein comprising the extracellular portion of the CD3 gamma protein. A co-stimulatory signalling domain is further fused to the intracellular portion of one or more of the chimeric membrane molecules. Co-transfection/co-transduction of both chimeric membrane protein into, e.g., T cells, results in formation of TCR comprising two heterologous chimeric proteins, thereby imparting dual antigen specificity to the TCR/cell as well as both CD3 zeta signaling and co-stimulatory signaling upon antigen engagement.

FIG. 45 is a schematic showing a TCR-based Chimeric Antigen Receptor (TCAR) assembled from the systems of the present invention. The TCAR has specificity for three antigens by fusion of a first, second and third antigen binding domain (here depicted as scFv antigen binding domains) to a protein comprising the extracellular portion of the CD3 delta protein, a protein comprising the extracellular portion of the CD3 epsilon protein, and to a protein comprising the extracellular portion of the CD3 gamma protein. A co-stimulatory signalling domain is further fused to the intracellular portion of one or more of the chimeric membrane molecules. Co-transfection/co-transduction of both chimeric membrane protein into, e.g., T cells, results in formation of TCR comprising two heterologous chimeric proteins, thereby imparting dual antigen specificity to the TCR/cell as well as both CD3 zeta signaling and co-stimulatory signaling upon antigen engagement.

FIG. 46 is a schematic showing a TCR-based Chimeric Antigen Receptor (TCAR) assembled from the systems of the present invention. The TCAR has specificity for three antigens by fusion of a first and second antigen binding domain (here depicted as scFv antigen binding domains) to a protein comprising the extracellular portion of the CD3 gamma protein (here shown as a tandem scFv fusion), and a third antigen binding domain fused to a protein comprising the extracellular portion of the CD3 delta protein. A co-stimulatory signalling domain is further fused to the intracellular portion of one or more of the chimeric membrane molecules. Co-transfection/co-transduction of both chimeric membrane protein into, e.g., T cells, results in formation of TCR comprising two heterologous chimeric proteins, thereby imparting dual antigen specificity to the TCR/cell as well as both CD3 zeta signaling and co-stimulatory signaling upon antigen engagement.

FIGS. 47A-47D are a panel of flow cytometry plots showing expression of TCARs on JNL cells. Non-transduced JNL (UTD), CD19-TCAR, CD22-TCAR, or CD19-TCAR plus CD22-TCAR (CD19/22 dual TCAR) transduced cells were stained with CD19-CAR anti-idiotype Ab and CD22-Fc and assayed by flow cytometry. The number in the upper left quadrant represents the expression level of CD22-TCAR, and the number in the lower right quadrant represents the expression level of CD19-TCAR (Geometric Mean).

FIGS. 48A-C are a panel of bar graphs showing results from a Jurkat NFAT Luciferase (JNL) reporter assay, testing the function of TCARs. Non-transduced JNL (UTD), CD19-TCAR, CD22-TCAR, or CD19-TCAR plus CD22-TCAR (CD19/22 dual TCAR) transduced cells were co-cultured with a chronic myelogenous leukemia (CML) cell line K562 (K562-WT) or K562 cells engineered to over-express CD19 (K562-CD19) or CD20 (K562-CD20). Luminescence (RLU) is shown for each JNL cell line at indicated tumor:JNL cell ratio.

### DETAILED DESCRIPTION

The present invention features the use of chimeric CD3 proteins to modulate T cell Receptor (TCR) signaling. Specifically, the invention is based, in part, on the discovery that chimeric CD3 proteins (e.g., CD3delta, CD3gamma, and CD3 epsilon) having all or most of their extracellular domain fused to an antigen binding domain can activate the TCR in the presesence of a cognate antigen. The invention is further based on the observation that the above chimeric proteins can be poteniated through the inclusion of a co-stimulatory domain in the intracellular portion of the chimeric molecule. Thus, the preferred elements of the engineered signaling complexes of the invention include an antigen binding domain, an extracellular domain derived from one of the above CD3 proteins, and an intracellular co-stimulatory domain. Interestingly, the invention is further based up on the discovery that these elements need not be present in a single polpeptide in order to achieve antigen based-TCR signaling. Indeed, any of the antigen binding domain and/or costimulatory domain can be engineered into a second chimeric molecule and still effectuate signaling provided that the second chimeric molecule and CD3 molecule are coupled either via an inducible or constitutive dimerization domain, as described herein.

TCR-based Chimeric Antigen Receptors (TCARs) may provide intrinsic advantages versus traditional chimeric antigen receptors. Traditional chimeric antigen receptors are single contiguous chain molecules comprising a targeting domain followed by a hinge, a transmembrane domain, one or more costimulatory domains and a signaling domain such as CD3zeta. By making the targeting domain a part of the TCR complex, signaling induced by the TCAR utilizes the entire pathway of accessory proteins within the TCR complex and is not limited to the exclusive signaling provided by a traditional CAR from, for example, CD3zeta on the CAR chain. In the natural pathways for T-cell activation and proliferation, the responsible intracellular pathway members are membrane proximal; while this is not possible for both the costimulatory and signaling domains in the traditional CAR format, TCARs enable the optimal orientation to be engineered into the T-cell.

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically The term “xenogeneic” refers to a graft derived from an animal of a different species.

The term “cancer” refers to a disease characterized by the uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

The phrase “disease associated with expression of a tumor antigen as described herein” includes, but is not limited to, a disease associated with expression of a tumor antigen as described herein or condition associated with cells which express a tumor antigen as described herein including, e.g., proliferative diseases such as a cancer or malignancy or a precancerous condition such as a myelodysplasia, a myelodysplastic syndrome or a preleukemia; or a noncancer related indication associated with cells which express a tumor antigen as described herein. In one aspect, a cancer associated with expression of a tumor antigen as described herein is a hematological cancer. In one aspect, a cancer associated with expression of a tumor antigen as described herein is a solid cancer. Further diseases associated with expression of a tumor antigen described herein include, but not limited to, e.g., atypical and/or non-classical cancers, malignancies, precancerous conditions or proliferative diseases associated with expression of a tumor antigen as described herein. Non-cancer related indications associated with expression of a tumor antigen as described herein include, but are not limited to, e.g., autoimmune disease, (e.g., lupus), inflammatory disorders (allergy and asthma) and transplantation. In some embodiments, the tumor antigen-expressing cells express, or at any time expressed, mRNA encoding the tumor antigen. In an embodiment, the tumor antigen-expressing cells produce the tumor antigen protein (e.g., wild-type or mutant), and the tumor antigen protein may be present at normal levels or reduced levels. In an embodiment, the tumor antigen-expressing cells produced detectable levels of a tumor antigen protein at one point, and subsequently produced substantially no detectable tumor antigen protein.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR of the invention can be replaced with other amino acid residues from the same side chain family and the altered CAR can be tested using the functional assays described herein.

The term “stimulation,” refers to a primary response induced by binding of a stimulatory molecule (e.g., a TCR/CD3 complex) with its cognate ligand (or tumor antigen in the case of a CAR) thereby mediating a signal transduction event, such as, but not limited to, signal transduction via the TCR/CD3 complex or signal transduction via the appropriate NK receptor or signaling domains. Stimulation can mediate altered expression of certain molecules.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloic-derived phagocytes.

“Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame. The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present invention includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The term “tumor-supporting antigen” or “cancer-supporting antigen” interchangeably refer to a molecule (typically a protein, carbohydrate or lipid) that is expressed on the surface of a cell that is, itself, not cancerous, but supports the cancer cells, e.g., by promoting their growth or survival e.g., resistance to immune cells. Exemplary cells of this type include stromal cells and myeloid-derived suppressor cells (MDSCs). The tumor-supporting antigen itself need not play a role in supporting the tumor cells so long as the antigen is present on a cell that supports cancer cells.

The terms “B cell antigen” or “B-Cell antigen” are used interchangeably, and refer to a molecule (typically a protein, carbohydrate or lipid) that is preferentially and specifically expressed on the surface of a B cell which can be targeted with an agent which binds thereto. The B cell antigen of particular interest is preferentially expressed on B cells compared to other non-B cell tissues of a mammal. The B cell antigen may be expressed on one particular B cell population, e.g., B cell precursors or mature B cells, or on more than one particular B cell population, e.g., both precursor B cells and mature B cells. Exemplary B cell surface markers include: CD5, CD10, CD19, CD20, CD21, CD22, CD23, CD24, CD25, CD27, CD30, CD34, CD37, CD38, CD40, CD53, CD69, CD72, CD73, CD74, CD75, CD77, CD79a, CD79b, CD80, CD81, CD82, CD83, CD84, CD85, CD86, CD123, CD135, CD138, CD179, CD269, Flt3, ROR1, BCMA, FcRn5, FcRn2, CS-1, CXCR4, 5, 7, IL-7/3R, IL7/4/3R, and IL4R. Particularly preferred B-Cell antigens include: CD19, CD20, CD22, FcRn5, FcRn2, BCMA, CS-1 and CD138. In embodiments, the B-Cell antigen is CD19. In embodiments, the B-Cell antigen is CD20. In embodiments, the B-Cell antigen is CD22. In embodiments, the B-Cell antigen is BCMA. In embodiments, the B-Cell antigen is FcRn5. In embodiments, the B-Cell antigen is FcRn2. In embodiments, the B-Cell antigen is CS-1. In embodiments, the B-Cell antigen is CD138.

The terms “solid tumor antigen” or “solid tumor cell antigen” refer to a molecule (typically a protein, carbohydrate or lipid) that is preferentially and specifically expressed on the surface of a solid tumor cell which can be targeted with an agent which binds thereto. The solid tumor antigen of particular interest is preferentially expressed on a solid tumor cell compared to other non-tumor tissues of a mammal. The solid tumor antigen may be expressed on one particular solid tumor cell population, e.g., on mesothelioma tumor cells, or on more than one particular solid tumor cell population, e.g., both mesothelioma tumor cells and ovarian cancer cells. Exemplary solid tumor antigens include: EGFRvIII, mesothelin, GD2, Tn Ag, PSMA, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, GPR20, Ly6k, OR51E2, TARP, GFRa4, and a peptide of any of these antigens presented on MHC. Particularly preferred solid tumor antigens include: CLDN6, mesothelin and EGFRvIII.

The terms “myeloid tumor antigen” or “myeloid tumor cell antigen” refer to a molecule (typically a protein, carbohydrate or lipid) that is preferentially and specifically expressed on the surface of a myeloid tumor cell which can be targeted with an agent which binds thereto. The myeloid tumor antigen of particular interest is preferentially expressed on a myeloid tumor cell compared to other non-tumor tissues of a mammal. The myeloid tumor antigen may be expressed on one particular myeloid tumor cell population, e.g., on acute myeloid leukemia (AML) tumor cells, or on more than one particular myeloid tumor cell population. Exemplary myeloid tumor antigens include: CD123, CD33 and CLL-1.

The term “flexible polypeptide linker” or “linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)n, where n is a positive integer equal to or greater than 1 (SEQ ID NO: 44). For example, n=1, n=2, n=3. n=4, n=5 and n=6, n=7, n=8, n=9 and n=10. In one embodiment, the flexible polypeptide linkers include, but are not limited to, (Gly4 Ser)4 (SEQ ID NO: 45) or (Gly4 Ser)3 (SEQ ID NO: 46). In another embodiment, the linkers include multiple repeats of (Gly2Ser), (GlySer) or (Gly3Ser) (SEQ ID NO: 44). Also included within the scope of the invention are linkers described in WO2012/138475, incorporated herein by reference).

As used herein, a 5′ cap (also termed an RNA cap, an RNA 7-methylguanosine cap or an RNA m⁷G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, that has been synthesized in vitro. Generally, the in vitro transcribed RNA is generated from an in vitro transcription vector. The in vitro transcription vector comprises a template that is used to generate the in vitro transcribed RNA.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies. In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

In the context of the present invention, “tumor antigen” or “hyperproliferative disorder antigen” or “antigen associated with a hyperproliferative disorder” refers to antigens that are common to specific hyperproliferative disorders. In certain aspects, the hyperproliferative disorder antigens of the present invention are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer, ovarian cancer, pancreatic cancer, and the like.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a binding partner (e.g., a tumor antigen) protein present in a sample, but which antibody or ligand does not substantially recognize or bind other molecules in the sample. “Membrane anchor” or “membrane tethering domain”, as that term is used herein, refers to a polypeptide or moiety, e.g., a myristoyl group, sufficient to anchor an extracellular or intracellular domain to the plasma membrane.

By “membrane protein” is meant a protein that comprises a transmembrane domain and, when expressed in a target cell, is anchored in, or traverses the cell membrane.

The term “CD3 epsilon” refers to a T-cell surface glycoprotein CD3 epsilon chain. Swiss-Prot accession number P07766 provides exemplary human CD3 epsilon amino acid sequences. An exemplary human CD3 epsilon amino acid sequence is provided as SEQ ID NO: 77. In embodiments, a CD3 epsilon is a functional variant or fragment of a sequence provided in Swiss-Prot accession number P07766 or the sequence of SEQ ID NO: 77. CD3 epsilon may also be referred to herein as CD3E.

The term “CD3 delta” refers to a T-cell surface glycoprotein CD3 delta chain. Swiss-Prot accession number P04234 provides exemplary human CD3 delta amino acid sequences. An exemplary human CD3 delta amino acid sequence is provided as SEQ ID NO: 82. In embodiments, a CD3 delta is a functional variant or fragment of a sequence provided in Swiss-Prot accession number P04234 or the sequence of SEQ ID NO: 82. CD3 delta may also be referred to herein as CD3D.

The term “CD3 gamma” refers to a T-cell surface glycoprotein CD3 gamma chain. Swiss-Prot accession number P09693 provides exemplary human CD3 gamma amino acid sequences. An exemplary human CD3 gamma amino acid sequence is provided as SEQ ID NO: 87. In embodiments a CD3 gamma is a functional variant or fragment of a sequence provided in Swiss-Prot accession number P09693 or the sequence of SEQ ID NO: 87. CD3 gamma may also be referred to herein as CD3G.

By “CD3 delta, gamma, or epsilon domain” is meant a domain that is derived from, and retains at least one endogenous activity of, CD3 delta, gamma or epsilon.

As used herein, a “system” refers to a set of chimeric membrane proteins, e.g., two chimeric membrane proteins. In some embodiments, each of the chimeric membrane proteins comprises an antigen binding domain, a domain derived from a component of TCR (e.g., a domain derived from CD3 gamma, delta, or epsilon), and a transmembrane domain. In some embodiments, one or more of the chimeric membrane proteins further comprise a costimulatory domain.

The compositions and methods of the present invention encompass polypeptides and nucleic acids having the sequences specified, or sequences substantially identical or similar thereto, e.g., sequences at least 85%, 90%, or 95% identical or higher to the sequence specified. In the context of an amino acid sequence, the term “substantially identical” is used herein to refer to a first amino acid sequence that contains a sufficient or minimum number of amino acid residues that are i) identical to, or ii) conservative substitutions of aligned amino acid residues in a second amino acid sequence such that the first and second amino acid sequences can have a common structural domain and/or common functional activity, for example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of a nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity, for example, nucleotide sequences having at least about 85%, 90%, 91%, 92%, 93%, 94%, 95%, 96%, 97%, 98% or 99% identity to a reference sequence, e.g., a sequence provided herein.

The term “variant” refers to a polypeptide that has a substantially identical amino acid sequence to a reference amino acid sequence, or is encoded by a substantially identical nucleotide sequence. In some embodiments, the variant is a functional variant.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

The term “signaling domain” refers to the functional portion of a protein which acts by transmitting information within the cell to regulate cellular activity via defined signaling pathways by generating second messengers or functioning as effectors by responding to such messengers.

By “intracellular co-stimulatory domain” is meant the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, ICAM-1, lymphocyte function-associated antigen-1 (LFA-1), CD2, CDS, CD7, CD287, LIGHT, NKG2C, NKG2D, SLAMF7, NKp80, NKp30, NKp44, NKp46, CD160, B7-H3, and a ligand that specifically binds with CD83, and the like.

The intracellular signaling domain can comprise the entire intracellular portion, or the entire native intracellular signaling domain, of the molecule from which it is derived, or a functional fragment or derivative thereof.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an extracellular domain that is derived from a CD3epsilon molecule, the extracellular domain retains sufficient CD3epsilon structure such that is has the required function, namely, the ability to generate a signal under the appropriate conditions. It does not connotate or include a limitation to a particular process of producing the extracellular domain, e.g., it does not mean that, to provide the extracellular domain, one must start with a CD3epsilon sequence and delete unwanted sequence, or impose mutations, to arrive at the extracellular domain.

By “extracellular domain” is meant the domain of a transmembrane protein that is expressed outside the cell.

By “dimerization domain” is meant a domain that binds a cognate dimerization domain either constitutively or inducibly. Such cognate dimerization domains may be the same or similar to the initial dimerization domain (“homodimerization domains”) or may be heterologous to the initial dimerization domain (“heterodimerization domains”). In cases where the domains constitutively dimerize, such dimerization will typically occur provided that both domains are expressed in the same cellular compartment. In cases where the domains inducibly dimerize, such dimerization will only occur in the presence of a “dimerization molecule.”

“Dimerization molecule,” as that term is used herein, refers to a molecule that promotes the association of a first dimerization domain with a second dimerization domain. In embodiments, the dimerization molecule does not naturally occur in the subject, or does not occur in concentrations that would result in significant dimerization. In embodiments, the dimerization molecule is a small molecule, e.g., rapamycin or a rapalogue, e.g, RAD001.

As used herein, the term “antigen binding domain” refers to a polypeptide capable of binding a second polypeptide. Such antigen binding domains include antibody molecules. Furthermore, the term “antigen binding domain” also includes polypeptides not derived from an antibody molecule (e.g., polypeptides that natively bind a cognate polypeptide or molecule, including the extracellular domains of receptor proteins).

As used herein, the term “antibody molecule” refers to an immunoglobulin chain or fragment thereof, comprising at least one immunoglobulin variable domain sequence. The term “antibody molecule” encompasses antibodies and antibody fragments. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The portion of the chimeric proteins of the invention comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody, or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a composition of the invention comprises an antibody fragment. In a further aspect, the protein comprises an antibody fragment that comprises a scFv.

The antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of the invention comprises an antibody fragment. In a further aspect, the protein comprises an antibody fragment that comprises a scFv. The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme), or a combination thereof.

The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like. In one aspect, the “4-1BB costimulatory domain” is the sequence provided as herein or the equivalent residues from a non-human species, e.g., mouse, rodent, monkey, ape and the like.

The term “bioequivalent” refers to an amount of an agent other than the reference compound (e.g., RAD001), required to produce an effect equivalent to the effect produced by the reference dose or reference amount of the reference compound (e.g., RAD001). In an embodiment the effect is the level of mTOR inhibition, e.g., as measured by P70 S6 kinase inhibition, e.g., as evaluated in an in vivo or in vitro assay, e.g., as measured by an assay described herein, e.g., the Boulay assay. In an embodiment, the effect is alteration of the ratio of PD-1 positive/PD-1 negative T cells, as measured by cell sorting. In an embodiment a bioequivalent amount or dose of an mTOR inhibitor is the amount or dose that achieves the same level of P70 S6 kinase inhibition as does the reference dose or reference amount of a reference compound. In an embodiment, a bioequivalent amount or dose of an mTOR inhibitor is the amount or dose that achieves the same level of alteration in the ratio of PD-1 positive/PD-1 negative T cells as does the reference dose or reference amount of a reference compound.

The term “low, immune enhancing, dose” when used in conjunction with an mTOR inhibitor, e.g., an allosteric mTOR inhibitor, e.g., RAD001 or rapamycin, or a catalytic mTOR inhibitor, refers to a dose of mTOR inhibitor that partially, but not fully, inhibits mTOR activity, e.g., as measured by the inhibition of P70 S6 kinase activity. Methods for evaluating mTOR activity, e.g., by inhibition of P70 S6 kinase, are discussed herein. The dose is insufficient to result in complete immune suppression but is sufficient to enhance the immune response. In an embodiment, the low, immune enhancing, dose of mTOR inhibitor results in a decrease in the number of PD-1 positive T cells and/or an increase in the number of PD-1 negative T cells, or an increase in the ratio of PD-1 negative T cells/PD-1 positive T cells. In an embodiment, the low, immune enhancing, dose of mTOR inhibitor results in an increase in the number of naive T cells. In an embodiment, the low, immune enhancing, dose of mTOR inhibitor results in one or more of the following:

an increase in the expression of one or more of the following markers: CD62L^{high}, CD127^{high}, CD27⁺, and BCL2, e.g., on memory T cells, e.g., memory T cell precursors;

a decrease in the expression of KLRG1, e.g., on memory T cells, e.g., memory T cell precursors; and

an increase in the number of memory T cell precursors, e.g., cells with any one or combination of the following characteristics: increased CD62L^{high} increased CD127^{high}, increased CD27⁺, decreased KLRG1, and increased BCL2;

wherein any of the changes described above occurs, e.g., at least transiently, e.g., as compared to a non-treated subject.

“Refractory” as used herein refers to a disease, e.g., cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

“Relapsed” as used herein refers to the return of a disease (e.g., cancer) or the signs and symptoms of a disease such as cancer after a period of improvement, e.g., after prior treatment of a therapy, e.g., cancer therapy

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

### Description

Provided herein are compositions of matter and methods of use for the treatment of a disease such as cancer using immune effector cells (e.g., T cells, NK cells) engineered with chimeric proteins of the invention.

Sequences of some examples of various components of the instant invention is listed in Table 1, where aa stands for amino acids, and na stands for nucleic acids that encode the corresponding peptide.

TABLE 1
Sequences of various components of CAR (aa-amino acids, na-nucleic acids
that encodes the corresponding protein)

| description | Sequence |
|---|---|
| EF-1 promoter | CGTGAGGCTCCGGTGCCCGTCAGTGGGCAGAGCGCACA |
| | TCGCCCACAGTCCCCGAGAAGTTGGGGGGAGGGGTCGG |
| | CAATTGAACCGGTGCCTAGAGAAGGTGGCGCGGGGTAA |
| | ACTGGGAAAGTGATGTCGTGTACTGGCTCCGCCTTTTTC |
| | CCGAGGGTGGGGGAGAACCGTATATAAGTGCAGTAGTC |
| | GCCGTGAACGTTCTTTTTCGCAACGGGTTTGCCGCCAGA |
| | ACACAGGTAAGTGCCGTGTGTGGTTCCCGCGGGCCTGG |
| | CCTCTTTACGGGTTATGGCCCTTGCGTGCCTTGAATTAC |
| | TTCCACCTGGCTGCAGTACGTGATTCTTGATCCCGAGCT |
| | TCGGGTTGGAAGTGGGTGGGAGAGTTCGAGGCCTTGCG |
| | CTTAAGGAGCCCCTTCGCCTCGTGCTTGAGTTGAGGCCT |
| | GGCCTGGGCGCTGGGGCCGCCGCGTGCGAATCTGGTGG |
| | CACCTTCGCGCCTGTCTCGCTGCTTTCGATAAGTCTCTA |
| | GCCATTTAAAATTTTTGATGACCTGCTGCGACGCTTTTT |
| | TTCTGGCAAGATAGTCTTGTAAATGCGGGCCAAGATCT |
| | GCACACTGGTATTTCGGTTTTTGGGGCCGCGGGCGGCG |
| | ACGGGGCCCGTGCGTCCCAGCGCACATGTTCGGCGAGG |
| | CGGGGCCTGCGAGCGCGGCCACCGAGAATCGGACGGG |
| | GGTAGTCTCAAGCTGGCCGGCCTGCTCTGGTGCCTGGC |
| | CTCGCGCCGCCGTGTATCGCCCCGCCCTGGGCGGCAAG |
| | GCTGGCCCGGTCGGCACCAGTTGCGTGAGCGGAAAGAT |
| | GGCCGCTTCCCGGCCCTGCTGCAGGGAGCTCAAAATGG |
| | AGGACGCGGCGCTCGGGAGAGCGGGCGGGTGAGTCAC |
| | CCACACAAAGGAAAAGGGCCTTTCCGTCCTCAGCCGTC |
| | GCTTCATGTGACTCCACGGAGTACCGGGCGCCGTCCAG |
| | GCACCTCGATTAGTTCTCGAGCTTTTGGAGTACGTCGTC |
| | TTTAGGTTGGGGGGAGGGGTTTTATGCGATGGAGTTTC |
| | CCCACACTGAGTGGGTGGAGACTGAAGTTAGGCCAGCT |
| | TGGCACTTGATGTAATTCTCCTTGGAATTTGCCCTTTTT |
| | GAGTTTGGATCTTGGTTCATTCTCAAGCCTCAGACAGTG |
| | GTTCAAAGTTTTTTTCTTCCATTTCAGGTGTCGTGA |
| | (SEQ ID NO: 47) |
| Leader (aa) | MALPVTALLLPLALLLHAARP (SEQ ID NO: 48) |
| Leader (na) | ATGGCCCTGCCTGTGACAGCCCTGCTGCTGCCTCTGGCT |
| | CTGCTGCTGCATGCCGCTAGACCC (SEQ ID NO: 49) |
| Exemplary 4-1BB | KRGRKKLLYIFKQPFMRPVQTTQEEDGCSCRFPEEEEGGC |
| intracellular domain (aa) | EL (SEQ ID NO: 50) |
| Exemplary 4-1BB | AAACGGGGCAGAAAGAAACTCCTGTATATATTCAAACA |
| intracellular domain (na) | ACCATTTATGAGACCAGTACAAACTACTCAAGAGGAAG |
| | ATGGCTGTA |
| | GCTGCCGATTTCCAGAAGAAGAAGAAGGAGGATGTGA |
| | ACTG (SEQ ID NO: 51) |
| Exemplary CD8 TM (aa) | IYIWAPLAGTCGVLLLSLVITLYC (SEQ ID NO: 92) |
| Exemplary CD8 TM (na) | ATCTACATCTGGGCGCCCTTGGCCGGGACTTGTGGGGT |
| | CCTTCTCCTGTCACTGGTTATCACCCTTTACTGC |
| | (SEQ ID NO: 93) |
| Exemplary CD27 | QRRKYRSNKGESPVEPAEPCRYSCPREEEGSTIPIQEDYRK |
| intracellular domain (aa) | PEPACSP (SEQ ID NO: 94) |
| Exemplary CD27 | AGGAGTAAGAGGAGCAGGCTCCTGCACAGTGACTACAT |
| intracellular domain (na) | GAACATGACTCCCCGCCGCCCCGGGCCCACCCGCAAGC |
| | ATTACCAGCCCTATGCCCCACCACGCGACTTCGCAGCCT |
| | ATCGCTCC (SEQ ID NO: 95) |
| Exemplary CD3-zeta | RVKFSRSADAPAYKQGQNQLYNELNLGRREEYDVLDKRR |
| intracellular domain (aa) | GRDPEMGGKPRRKNPQEGLYNELQKDKMAEAYSEIGMK |
| | GERRRGKGHDGLYQGLSTATKDTYDALHMQALPPR |
| | (SEQ ID NO: 96) |
| Exemplary CD3-zeta | AGAGTGAAGTTCAGCAGGAGCGCAGACGCCCCCGCGTA |
| intracellular domain (na) | CAAGCAGGGCCAGAACCAGCTCTATAACGAGCTCAATC |
| | TAGGACGAAGAGAGGAGTACGATGTTTTGGACAAGAG |
| | ACGTGGCCGGGACCCTGAGATGGGGGGAAAGCCGAGA |
| | AGGAAGAACCCTCAGGAAGGCCTGTACAATGAACTGCA |
| | GAAAGATAAGATGGCGGAGGCCTACAGTGAGATTGGG |
| | ATGAAAGGCGAGCGCCGGAGGGGCAAGGGGCACGATG |
| | GCCTTTACCAGGGTCTCAGTACAGCCACCAAGGACACC |
| | TACGACGCCCTTCACATGCAGGCCCTGCCCCCTCGC |
| | (SEQ ID NO: 97) |
| Exemplary CD3-zeta | RVKFSRSADAPAYQQGQNQLYNELNLGRREEYDVLDKRR |
| intracellular domain (aa) | GRDPEMGGKPRRKNPQEGLYNELQKDKMAEAYSEIGMK |
| | GERRRGKGHDGLYQGLSTATKDTYDALHMQALPPR |
| | (SEQ ID NO: 98) |
| Exemplary CD3-zeta | AGAGTGAAGTTCAGCAGGAGCGCAGACGCCCCCGCGTA |
| intracellular domain (na) | CCAGCAGGGCCAGAACCAGCTCTATAACGAGCTCAATC |
| | TAGGACGAAGAGAGGAGTACGATGTTTTGGACAAGAG |
| | ACGTGGCCGGGACCCTGAGATGGGGGGAAAGCCGAGA |
| | AGGAAGAACCCTCAGGAAGGCCTGTACAATGAACTGCA |
| | GAAAGATAAGATGGCGGAGGCCTACAGTGAGATTGGG |
| | ATGAAAGGCGAGCGCCGGAGGGGCAAGGGGCACGATG |
| | GCCTTTACCAGGGTCTCAGTACAGCCACCAAGGACACC |
| | TACGACGCCCTTCACATGCAGGCCCTGCCCCCTCGC |
| | (SEQ ID NO: 99) |
| Exemplary CD28 | RSKRSRLLHSDYMNMTPRRPGPTRKHYQPYAPPRDFAAY |
| intracellular domain (aa) | RS (SEQ ID NO: 100) |
| Exemplary CD28 | AGGAGTAAGAGGAGCAGGCTCCTGCACAGTGACTACAT |
| intracellular domain (na) | GAACATGACTCCCCGCCGCCCCGGGCCCACCCGCAAGC |
| | ATTACCAGCCCTATGCCCCACCACGCGACTTCGCAGCCT |
| | ATCGCTCC (SEQ ID NO: 101) |
| Exemplary ICOS | TKKKYSSSVHDPNGEYMFMRAVNTAKKSRLTDVTL |
| intracellular domain (aa) | (SEQ ID NO: 102) |
| Exemplary ICOS | ACAAAAAAGAAGTATTCATCCAGTGTGCACGACCCTAA |
| intracellular domain (na) | CGGTGAATACATGTTCATGAGAGCAGTGAACACAGCCA |
| | AAAAATCCAGACTCACAGATGTGACCCTA |
| | (SEQ ID NO: 103) |

Cancer Associated Antigens

The present invention provides immune effector cells (e.g., T cells, NK cells) that are engineered to contain one or more chimeric proteins that direct the immune effector cells to cancer. This is achieved through an antigen binding domain on the protein that is specific for a cancer associated antigen. There are two classes of cancer associated antigens (tumor antigens) that can be targeted by the proteins of the instant invention: (1) cancer associated antigens that are expressed on the surface of cancer cells; and (2) cancer associated antigens that itself is intracellar, however, a fragment of such antigen (peptide) is presented on the surface of the cancer cells by MHC (major histocompatibility complex).

Accordingly, the present invention provides proteins that target the following cancer associated antigens (tumor antigens): CD19, CD123, CD22, CD30, CD171, CS-1, CLL-1 (CLECL1), CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, VEGFR2, LewisY, CD24, PDGFR-beta, PRSS21, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, legumain, HPV E6,E7, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In embodiments, the present invention provides proteins that target the following B-cell antigens: CD5, CD10, CD19, CD20, CD21, CD22, CD23, CD24, CD25, CD27, CD30, CD34, CD37, CD38, CD40, CD53, CD69, CD72, CD73, CD74, CD75, CD77, CD79a, CD79b, CD80, CD81, CD82, CD83, CD84, CD85, CD86, CD123, CD135, CD138, CD179, CD269, Flt3, ROR1, BCMA, FcRn5, FcRn2, CS-1, CXCR4, 5, 7, IL-7/3R, IL7/4/3R, and IL4R. Particularly preferred B-Cell antigens include: CD19, CD20, CD22, FcRn5, FcRn2, BCMA, CS-1 and CD138.

In embodiments, the present invention provides proteins that target the following solid tumor antigens: EGFRvIII, mesothelin, GD2, Tn Ag, PSMA, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, GPR20, Ly6k, OR51E2, TARP, GFRa4, and a peptide of any of these antigens presented on MHC. Particularly preferred solid tumor antigens include: CLDN6, mesothelin and EGFRvIII.

A chimeric proteins described herein can comprise an antigen binding domain (e.g., antibody or antibody fragment, TCR or TCR fragment) that binds to a tumor-supporting antigen (e.g., a tumor-supporting antigen as described herein). In some embodiments, the tumor-supporting antigen is an antigen present on a stromal cell or a myeloid-derived suppressor cell (MDSC). Stromal cells can secrete growth factors to promote cell division in the microenvironment. MDSC cells can inhibit T cell proliferation and activation.

In embodiments, the stromal cell antigen is chosen from one or more of: bone marrow stromal cell antigen 2 (BST2), fibroblast activation protein (FAP) and tenascin. In an embodiment, the FAP-specific antibody is, competes for binding with, or has the same CDRs as, sibrotuzumab. In embodiments, the MDSC antigen is chosen from one or more of: CD33, CD11b, C14, CD15, and CD66b. Accordingly, in some embodiments, the tumor-supporting antigen is chosen from one or more of: bone marrow stromal cell antigen 2 (BST2), fibroblast activation protein (FAP) or tenascin, CD33, CD11b, C14, CD15, and CD66b.

As will be understood from the present disclosure, the systems, cells and other aspects of the invention comprise more than one antigen binding domain, such that more than one antigen is targeted. Combinations of any of the antigens described herein may be targeted by utilizing systems comprising antigen binding domains targeting said combination of more than one antigen.

Chimeric Proteins of the Invention

The invention features one or more chimeric proteins. Generally, the invention features a first chimeric membrane protein that includes all or a functional portion of the extracellular domain of CD3 delta, gamma, or epsilon. These chimeric proteins can further include one or more of the following; an antigen binding domain, an intracellular co-stimulatory domain, and/or dimerization domain. In certain embodiments, e.g., where the first chimeric molecule does not include an antigen binding domain, the invention features a second chimeric membrane protein: this protein having an extracellular antigen binding domain and a dimerization domain. Optionally, this second protein can further include an intracellular co-stimulatory domain (whether or not the first chimeric protein has such a domain). Alternatively, the second chimeric protein can include a domain which binds a domain (e.g., extracellular or intracellular domain) of the first chimeric protein and a co-stimulatory domain, antigen binding domain, or both.

Antigen Binding Domain

In one aspect, certain chimeric proteins of the invention comprises a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a protein of the invention include those associated with viral, bacterial and parasitic infections, autoimmune disease and cancer cells.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like.

In one embodiment, an antigen binding domain against CD22 is an antigen binding portion, e.g., CDRs, of an antibody described in, e.g., Haso et al., Blood, 121(7): 1165-1174 (2013); Wayne et al., Clin Cancer Res 16(6): 1894-1903 (2010); Kato et al., Leuk Res 37(1):83-88 (2013); Creative BioMart (creativebiomart.net): MOM-18047-S(P).

In one embodiment, an antigen binding domain against CS-1 is an antigen binding portion, e.g., CDRs, of Elotuzumab (BMS), see e.g., Tai et al., 2008, Blood 112(4):1329-37; Tai et al., 2007, Blood. 110(5):1656-63.

In one embodiment, an antigen binding domain against CLL-1 is an antigen binding portion, e.g., CDRs, of an antibody available from R&D, ebiosciences, Abcam, for example, PE-CLL1-hu Cat#353604 (BioLegend); and PE-CLL1 (CLEC12A) Cat#562566 (BD).

In one embodiment, an antigen binding domain against CD33 is an antigen binding portion, e.g., CDRs, of an antibody described in, e.g., Bross et al., Clin Cancer Res 7(6):1490-1496 (2001) (Gemtuzumab Ozogamicin, hP67.6), Caron et al., Cancer Res 52(24):6761-6767 (1992) (Lintuzumab, HuM195), Lapusan et al., Invest New Drugs 30(3):1121-1131 (2012) (AVE9633), Aigner et al., Leukemia 27(5): 1107-1115 (2013) (AMG330, CD33 BiTE), Dutour et al., Adv hematol 2012:683065 (2012), and Pizzitola et al., Leukemia doi:10.1038/Lue.2014.62 (2014).

In one embodiment, an antigen binding domain against GD2 is an antigen binding portion, e.g., CDRs, of an antibody described in, e.g., Mujoo et al., Cancer Res. 47(4):1098-1104 (1987); Cheung et al., Cancer Res 45(6):2642-2649 (1985), Cheung et al., J Clin Oncol 5(9):1430-1440 (1987), Cheung et al., J Clin Oncol 16(9):3053-3060 (1998), Handgretinger et al., Cancer Immunol Immunother 35(3):199-204 (1992). In some embodiments, an antigen binding domain against GD2 is an antigen binding portion of an antibody selected from mAb 14.18, 14G2a, ch14.18, hu14.18, 3F8, hu3F8, 3G6, 8B6, 60C3, 10B8, ME36.1, and 8H9, see e.g., WO2012033885, WO2013040371, WO2013192294, WO2013061273, WO2013123061, WO2013074916, and WO201385552. In some embodiments, an antigen binding domain against GD2 is an antigen binding portion of an antibody described in US Publication No.: 20100150910 or PCT Publication No.: WO 2011160119.

In one embodiment, an antigen binding domain against BCMA is an antigen binding portion, e.g., CDRs, of an antibody described in, e.g., WO2012163805, WO200112812, and WO2003062401.

In one embodiment, an antigen binding domain against Tn antigen is an antigen binding portion, e.g., CDRs, of an antibody described in, e.g., U.S. Pat. No. 8,440,798, Brooks et al., PNAS 107(22):10056-10061 (2010), and Stone et al., OncoImmunology 1(6):863-873 (2012).

In one embodiment, an antigen binding domain against PSMA is an antigen binding portion, e.g., CDRs, of an antibody described in, e.g., Parker et al., Protein Expr Purif 89(2):136-145 (2013), US 20110268656 (J591 ScFv); Frigerio et al, European J Cancer 49(9):2223-2232 (2013) (scFvD2B); WO 2006125481 (mAbs 3/A12, 3/E7 and 3/F11) and single chain antibody fragments (scFv A5 and D7).

In one embodiment, an antigen binding domain against ROR1 is an antigen binding portion, e.g., CDRs, of an antibody described in, e.g., Hudecek et al., Clin Cancer Res 19(12):3153-3164 (2013); WO 2011159847; and US20130101607.

In one embodiment, an antigen binding domain against FLT3 is an antigen binding portion, e.g., CDRs, of an antibody described in, e.g., WO2011076922, U.S. Pat. No. 5,777,084, EP0754230, US20090297529, and several commercial catalog antibodies (R&D, ebiosciences, Abcam). In one embodiment, an antigen binding domain against TAG72 is an antigen binding portion, e.g., CDRs, of an antibody described in, e.g., Hombach et al., Gastroenterology 113(4):1163-1170 (1997); and Abcam ab691.

In one embodiment, an antigen binding domain against FAP is an antigen binding portion, e.g., CDRs, of an antibody described in, e.g., Ostermann et al., Clinical Cancer Research 14:4584-4592 (2008) (FAP5), US Pat. Publication No. 2009/0304718; sibrotuzumab (see e.g., Hofheinz et al., Oncology Research and Treatment 26(1), 2003); and Tran et al., J Exp Med 210(6):1125-1135 (2013).

In one embodiment, an antigen binding domain against CD38 is an antigen binding portion, e.g., CDRs, of daratumumab (see, e.g., Groen et al., Blood 116(21):1261-1262 (2010); MOR202 (see, e.g., U.S. Pat. No. 8,263,746); or antibodies described in U.S. Pat. No. 8,362,211.

In one embodiment, an antigen binding domain against CD44v6 is an antigen binding portion, e.g., CDRs, of an antibody described in, e.g., Casucci et al., Blood 122(20):3461-3472 (2013).

In one embodiment, an antigen binding domain against CEA is an antigen binding portion, e.g., CDRs, of an antibody described in, e.g., Chmielewski et al., Gastoenterology 143(4):1095-1107 (2012).

In one embodiment, an antigen binding domain against EPCAM is an antigen binding portion, e.g., CDRS, of an antibody selected from MT110, EpCAM-CD3 bispecific Ab (see, e.g., clinicaltrials.gov/ct2/show/NCT00635596); Edrecolomab; 3622W94; ING-1; and adecatumumab (MT201).

In one embodiment, an antigen binding domain against PRSS21 is an antigen binding portion, e.g., CDRs, of an antibody described in U.S. Pat. No. 8,080,650.

In one embodiment, an antigen binding domain against B7H3 is an antigen binding portion, e.g., CDRs, of an antibody MGA271 (Macrogenics).

In one embodiment, an antigen binding domain against KIT is an antigen binding portion, e.g., CDRs, of an antibody described in, e.g., U.S. Pat. No. 7,915,391, US20120288506, and several commercial catalog antibodies.

In one embodiment, an antigen binding domain against IL-13Ra2 is an antigen binding portion, e.g., CDRs, of an antibody described in, e.g., WO2008/146911, WO2004087758, several commercial catalog antibodies, and WO2004087758.

In one embodiment, an antigen binding domain against CD30 is an antigen binding portion, e.g., CDRs, of an antibody described in, e.g., U.S. Pat. No. 7,090,843 B1, and EP0805871.

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761; WO2005035577; and U.S. Pat. No. 6,437,098.

In one embodiment, an antigen binding domain against CD171 is an antigen binding portion, e.g., CDRs, of an antibody described in, e.g., Hong et al., J Immunother 37(2):93-104 (2014).

In one embodiment, an antigen binding domain against IL-11Ra is an antigen binding portion, e.g., CDRs, of an antibody available from Abcam (cat#ab55262) or Novus Biologicals (cat#EPR5446). In another embodiment, an antigen binding domain again IL-11Ra is a peptide, see, e.g., Huang et al., Cancer Res 72(1):271-281 (2012).

In one embodiment, an antigen binding domain against PSCA is an antigen binding portion, e.g., CDRs, of an antibody described in, e.g., Morgenroth et al., Prostate 67(10):1121-1131 (2007) (scFv 7F5); Nejatollahi et al., J of Oncology 2013 (2013), article ID 839831 (scFv C5-II); and US Pat Publication No. 20090311181.

In one embodiment, an antigen binding domain against VEGFR2 is an antigen binding portion, e.g., CDRs, of an antibody described in, e.g., Chinnasamy et al., J Clin Invest 120(11):3953-3968 (2010).

In one embodiment, an antigen binding domain against LewisY is an antigen binding portion, e.g., CDRs, of an antibody described in, e.g., Kelly et al., Cancer Biother Radiopharm 23(4):411-423 (2008) (hu3S193 Ab (scFvs)); Dolezal et al., Protein Engineering 16(1):47-56 (2003) (NC10 scFv).

In one embodiment, an antigen binding domain against CD24 is an antigen binding portion, e.g., CDRs, of an antibody described in, e.g., Maliar et al., Gastroenterology 143(5):1375-1384 (2012).

In one embodiment, an antigen binding domain against PDGFR-beta is an antigen binding portion, e.g., CDRs, of an antibody Abcam ab32570.

In one embodiment, an antigen binding domain against SSEA-4 is an antigen binding portion, e.g., CDRs, of antibody MC813 (Cell Signaling), or other commercially available antibodies.

In one embodiment, an antigen binding domain against CD20 is an antigen binding portion, e.g., CDRs, of the antibody Rituximab, Ofatumumab, Ocrelizumab, Veltuzumab, or GA101. In one embodiment, an antigen binding domain against Folate receptor alpha is an antigen binding portion, e.g., CDRs, of the antibody IMGN853, or an antibody described in US20120009181; U.S. Pat. No. 4,851,332, LK26: U.S. Pat. No. 5,952,484.

In one embodiment, an antigen binding domain against ERBB2 (Her2/neu) is an antigen binding portion, e.g., CDRs, of the antibody trastuzumab, or pertuzumab.

In one embodiment, an antigen binding domain against MUC1 is an antigen binding portion, e.g., CDRs, of the antibody SAR566658.

In one embodiment, the antigen binding domain against EGFR is antigen binding portion, e.g., CDRs, of the antibody cetuximab, panitumumab, zalutumumab, nimotuzumab, or matuzumab.

In one embodiment, an antigen binding domain against NCAM is an antigen binding portion, e.g., CDRs, of the antibody clone 2-2B: MAB5324 (EMD Millipore) In one embodiment, an antigen binding domain against Ephrin B2 is an antigen binding portion, e.g., CDRs, of an antibody described in, e.g., Abengozar et al., Blood 119(19):4565-4576 (2012).

In one embodiment, an antigen binding domain against IGF-I receptor is an antigen binding portion, e.g., CDRs, of an antibody described in, e.g., U.S. Pat. No. 8,344,112 B2; EP2322550 A1; WO 2006/138315, or PCT/US2006/022995.

In one embodiment, an antigen binding domain against CAIX is an antigen binding portion, e.g., CDRs, of the antibody clone 303123 (R&D Systems).

In one embodiment, an antigen binding domain against LMP2 is an antigen binding portion, e.g., CDRs, of an antibody described in, e.g., U.S. Pat. No. 7,410,640, or US20050129701.

In one embodiment, an antigen binding domain against gp100 is an antigen binding portion, e.g., CDRs, of the antibody HMB45, NKIbetaB, or an antibody described in WO2013165940, or US20130295007

In one embodiment, an antigen binding domain against tyrosinase is an antigen binding portion, e.g., CDRs, of an antibody described in, e.g., U.S. Pat. No. 5,843,674; or U.S. Ser. No. 19/950504048. In one embodiment, an antigen binding domain against EphA2 is an antigen binding portion, e.g., CDRs, of an antibody described in, e.g., Yu et al., Mol Ther 22(1):102-111 (2014).

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761 A3; 20120276046; WO2005035577; or U.S. Pat. No. 6,437,098.

In one embodiment, an antigen binding domain against fucosyl GM1 is an antigen binding portion, e.g., CDRs, of an antibody described in, e.g., US20100297138; or WO2007/067992.

In one embodiment, an antigen binding domain against sLe is an antigen binding portion, e.g., CDRs, of the antibody G193 (for lewis Y), see Scott A M et al, Cancer Res 60: 3254-61 (2000), also as described in Neeson et al, J Immunol May 2013 190 (Meeting Abstract Supplement) 177.10.

In one embodiment, an antigen binding domain against GM3 is an antigen binding portion, e.g., CDRs, of the antibody CA 2523449 (mAb 14F7).

In one embodiment, an antigen binding domain against HMWMAA is an antigen binding portion, e.g., CDRs, of an antibody described in, e.g., Kmiecik et al., Oncoimmunology 3(1):e27185 (2014) (PMID: 24575382) (mAb9.2.27); U.S. Pat. No. 6,528,481; WO2010033866; or US 20140004124.

In one embodiment, an antigen binding domain against o-acetyl-GD2 is an antigen binding portion, e.g., CDRs, of the antibody 8B6.

In one embodiment, an antigen binding domain against TEM1/CD248 is an antigen binding portion, e.g., CDRs, of an antibody described in, e.g., Marty et al., Cancer Lett 235(2):298-308 (2006); Zhao et al., J Immunol Methods 363(2):221-232 (2011).

In one embodiment, an antigen binding domain against CLDN6 is an antigen binding portion, e.g., CDRs, of the antibody IMAB027 (Ganymed Pharmaceuticals), see e.g., clinicaltrial.gov/show/NCT02054351.

In one embodiment, an antigen binding domain against TSHR is an antigen binding portion, e.g., CDRs, of an antibody described in, e.g., U.S. Pat. Nos. 8,603,466; 8,501,415; or U.S. Pat. No. 8,309,693. In one embodiment, an antigen binding domain against GPRC5D is an antigen binding portion, e.g., CDRs, of the antibody FAB6300A (R&D Systems); or LS-A4180 (Lifespan Biosciences).

In one embodiment, an antigen binding domain against CD97 is an antigen binding portion, e.g., CDRs, of an antibody described in, e.g., U.S. Pat. No. 6,846,911; de Groot et al., J Immunol 183(6):4127-4134 (2009); or an antibody from R&D:MAB3734.

In one embodiment, an antigen binding domain against ALK is an antigen binding portion, e.g., CDRs, of an antibody described in, e.g., Mino-Kenudson et al., Clin Cancer Res 16(5):1561-1571 (2010).

In one embodiment, an antigen binding domain against polysialic acid is an antigen binding portion, e.g., CDRs, of an antibody described in, e.g., Nagae et al., J Biol Chem 288(47):33784-33796 (2013).

In one embodiment, an antigen binding domain against PLAC1 is an antigen binding portion, e.g., CDRs, of an antibody described in, e.g., Ghods et al., Biotechnol Appl Biochem 2013 doi:10.1002/bab.1177.

In one embodiment, an antigen binding domain against GloboH is an antigen binding portion of the antibody VK9; or an antibody described in, e.g., Kudryashov V et al, Glycoconj J. 15(3):243-9 (1998), Lou et al., Proc Natl Acad Sci USA 111(7):2482-2487 (2014); MBr1: Bremer E-G et al. J Biol Chem 259:14773-14777 (1984).

In one embodiment, an antigen binding domain against NY-BR-1 is an antigen binding portion, e.g., CDRs of an antibody described in, e.g., Jager et al., Appl Immunohistochem Mol Morphol 15(1):77-83 (2007).

In one embodiment, an antigen binding domain against WT-1 is an antigen binding portion, e.g., CDRs, of an antibody described in, e.g., Dao et al., Sci Transl Med 5(176):176ra33 (2013); or WO2012/135854.

In one embodiment, an antigen binding domain against MAGE-A1 is an antigen binding portion, e.g., CDRs, of an antibody described in, e.g., Willemsen et al., J Immunol 174(12):7853-7858 (2005) (TCR-like scFv).

In one embodiment, an antigen binding domain against sperm protein 17 is an antigen binding portion, e.g., CDRs, of an antibody described in, e.g., Song et al., Target Oncol 2013 Aug. 14 (PMID: 23943313); Song et al., Med Oncol 29(4):2923-2931 (2012).

In one embodiment, an antigen binding domain against Tie 2 is an antigen binding portion, e.g., CDRs, of the antibody AB33 (Cell Signaling Technology).

In one embodiment, an antigen binding domain against MAD-CT-2 is an antigen binding portion, e.g., CDRs, of an antibody described in, e.g., PMID: 2450952; U.S. Pat. No. 7,635,753.

In one embodiment, an antigen binding domain against Fos-related antigen 1 is an antigen binding portion, e.g., CDRs, of the antibody 12F9 (Novus Biologicals).

In one embodiment, an antigen binding domain against MelanA/MART1 is an antigen binding portion, e.g., CDRs, of an antibody described in, EP2514766 A2; or U.S. Pat. No. 7,749,719. In one embodiment, an antigen binding domain against sarcoma translocation breakpoints is an antigen binding portion, e.g., CDRs, of an antibody described in, e.g., Luo et al, EMBO Mol. Med. 4(6):453-461 (2012).

In one embodiment, an antigen binding domain against TRP-2 is an antigen binding portion, e.g., CDRs, of an antibody described in, e.g., Wang et al, J Exp Med. 184(6):2207-16 (1996).

In one embodiment, an antigen binding domain against CYP1B1 is an antigen binding portion, e.g., CDRs, of an antibody described in, e.g., Maecker et al, Blood 102 (9): 3287-3294 (2003).

In one embodiment, an antigen binding domain against RAGE-1 is an antigen binding portion, e.g., CDRs, of the antibody MAB5328 (EMD Millipore).

In one embodiment, an antigen binding domain against human telomerase reverse transcriptase is an antigen binding portion, e.g., CDRs, of the antibody cat no: LS-B95-100

(Lifespan Biosciences)

In one embodiment, an antigen binding domain against intestinal carboxyl esterase is an antigen binding portion, e.g., CDRs, of the antibody 4F12: cat no: LS-B6190-50 (Lifespan Biosciences).

In one embodiment, an antigen binding domain against mut hsp70-2 is an antigen binding portion, e.g., CDRs, of the antibody Lifespan Biosciences: monoclonal: cat no: LS-C133261-100 (Lifespan Biosciences).

In one embodiment, an antigen binding domain against CD79a is an antigen binding portion, e.g., CDRs, of the antibody Anti-CD79a antibody [HM47/A9] (ab3121), available from Abcam; antibody CD79A Antibody #3351 available from Cell Signalling Technology; or antibody HPA017748—Anti-CD79A antibody produced in rabbit, available from Sigma Aldrich.

In one embodiment, an antigen binding domain against CD79b is an antigen binding portion, e.g., CDRs, of the antibody polatuzumab vedotin, anti-CD79b described in Dornan et al., “Therapeutic potential of an anti-CD79b antibody-drug conjugate, anti-CD79b-vc-MMAE, for the treatment of non-Hodgkin lymphoma” Blood. 2009 Sep. 24; 114(13):2721-9. doi: 10.1182/blood-2009-02-205500. Epub 2009 Jul. 24, or the bispecific antibody Anti-CD79b/CD3 described in “4507 Pre-Clinical Characterization of T Cell-Dependent Bispecific Antibody Anti-CD79b/CD3 As a Potential Therapy for B Cell Malignancies” Abstracts of 56^{th} ASH Annual Meeting and Exposition, San Francisco, Calif. December 6-9 2014.

In one embodiment, an antigen binding domain against CD72 is an antigen binding portion, e.g., CDRs, of the antibody J3-109 described in Myers, and Uckun, “An anti-CD72 immunotoxin against therapy-refractory B-lineage acute lymphoblastic leukemia.” Leuk Lymphoma. 1995 June; 18(1-2):119-22, or anti-CD72 (10D6.8.1, mIgG1) described in Polson et al., “Antibody-Drug Conjugates for the Treatment of Non-Hodgkin's Lymphoma: Target and Linker-Drug Selection” Cancer Res Mar. 15, 2009 69; 2358.

In one embodiment, an antigen binding domain against LAIR1 is an antigen binding portion, e.g., CDRs, of the antibody ANT-301 LAIR1 antibody, available from ProSpec; or anti-human CD305 (LAIR1) Antibody, available from BioLegend.

In one embodiment, an antigen binding domain against FCAR is an antigen binding portion, e.g., CDRs, of the antibody CD89/FCARAntibody (Catalog#10414-H08H), available from Sino Biological Inc.

In one embodiment, an antigen binding domain against LILRA2 is an antigen binding portion, e.g., CDRs, of the antibody LILRA2 monoclonal antibody (M17), clone 3C7, available from Abnova, or Mouse Anti-LILRA2 antibody, Monoclonal (2D7), available from Lifespan Biosciences.

In one embodiment, an antigen binding domain against CD300LF is an antigen binding portion, e.g., CDRs, of the antibody Mouse Anti-CMRF35-like molecule 1 antibody, Monoclonal[UP-D2], available from BioLegend, or Rat Anti-CMRF35-like molecule 1 antibody, Monoclonal[234903], available from R&D Systems.

In one embodiment, an antigen binding domain against CLEC12A is an antigen binding portion, e.g., CDRs, of the antibody Bispecific T cell Engager (BiTE) scFv-antibody and ADC described in Noordhuis et al., “Targeting of CLEC12A In Acute Myeloid Leukemia by Antibody-Drug-Conjugates and Bispecific CLL-1xCD3 BiTE Antibody” 53^{rd} ASH Annual Meeting and Exposition, Dec. 10-13, 2011, and MCLA-117 (Merus).

In one embodiment, an antigen binding domain against BST2 (also called CD317) is an antigen binding portion, e.g., CDRs, of the antibody Mouse Anti-CD317 antibody, Monoclonal[3H4], available from Antibodies-Online or Mouse Anti-CD317 antibody, Monoclonal[696739], available from R&D Systems.

In one embodiment, an antigen binding domain against EMR2 (also called CD312) is an antigen binding portion, e.g., CDRs, of the antibody Mouse Anti-CD312 antibody, Monoclonal[LS-B8033] available from Lifespan Biosciences, or Mouse Anti-CD312 antibody, Monoclonal[494025] available from R&D Systems.

In one embodiment, an antigen binding domain against LY75 is an antigen binding portion, e.g., CDRs, of the antibody Mouse Anti-Lymphocyte antigen 75 antibody, Monoclonal[HD30] available from EMD Millipore or Mouse Anti-Lymphocyte antigen 75 antibody, Monoclonal[A15797] available from Life Technologies.

In one embodiment, an antigen binding domain against GPC3 is an antigen binding portion, e.g., CDRs, of the antibody hGC33 described in Nakano K, Ishiguro T, Konishi H, et al. Generation of a humanized anti-glypican 3 antibody by CDR grafting and stability optimization. Anticancer Drugs. 2010 November; 21(10):907-916, or MDX-1414, HN3, or YP7, all three of which are described in Feng et al., “Glypican-3 antibodies: a new therapeutic target for liver cancer.” FEBS Lett. 2014 Jan. 21; 588(2):377-82.

In one embodiment, an antigen binding domain against FCRL5 is an antigen binding portion, e.g., CDRs, of the anti-FcRL5 antibody described in Elkins et al., “FcRL5 as a target of antibody-drug conjugates for the treatment of multiple myeloma” Mol Cancer Ther. 2012 October; 11(10):2222-32.

In one embodiment, an antigen binding domain against IGLL1 is an antigen binding portion, e.g., CDRs, of the antibody Mouse Anti-Immunoglobulin lambda-like polypeptide 1 antibody, Monoclonal[AT1G4] available from Lifespan Biosciences, Mouse Anti-Immunoglobulin lambda-like polypeptide 1 antibody, Monoclonal[HSL11] available from BioLegend.

In one embodiment, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed above.

In another aspect, the antigen binding domain comprises a humanized antibody or an antibody fragment. In some aspects, a non-human antibody is humanized, where specific sequences or regions of the antibody are modified to increase similarity to an antibody naturally produced in a human or fragment thereof. In one aspect, the antigen binding domain is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is nonhuman. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Nicholson et al. Mol. Immun 34 (16-17): 1157-1165 (1997); Carter et al., Proc. Natl. Acad. Sci. USA, 89:4285 (1992); Presta et al., J. Immunol., 151:2623 (1993), the contents of which are incorporated herein by reference herein in their entirety). In some embodiments, the framework region, e.g., all four framework regions, of the heavy chain variable region are derived from a VH4_4-59 germline sequence. In one embodiment, the framework region can comprise, one, two, three, four or five modifications, e.g., substitutions, e.g., from the amino acid at the corresponding murine sequence. In one embodiment, the framework region, e.g., all four framework regions of the light chain variable region are derived from a VK3_1.25 germline sequence. In one embodiment, the framework region can comprise, one, two, three, four or five modifications, e.g., substitutions, e.g., from the amino acid at the corresponding murine sequence.

In some aspects, the antibody fragment is humanized with retention of high affinity for the target antigen and other favorable biological properties. According to one aspect of the invention, humanized antibodies and antibody fragments are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind the target antigen. In this way, FR residues can be selected and combined from the recipient and import sequences so that the desired antibody or antibody fragment characteristic, such as increased affinity for the target antigen, is achieved. In general, the CDR residues are directly and most substantially involved in influencing antigen binding.

A humanized antibody or antibody fragment may retain a similar antigenic specificity as the original antibody, e.g., in the present invention, the ability to bind human a cancer associated antigen as described herein. In some embodiments, a humanized antibody or antibody fragment may have improved affinity and/or specificity of binding to human a cancer associated antigen as described herein.

In one aspect, the antigen binding domain of the invention is characterized by particular functional features or properties of an antibody or antibody fragment. For example, in one aspect, the antigen binding domain specifically binds a tumor antigen as described herein. In one aspect, the anti-cancer associated antigen as described herein binding domain is a fragment, e.g., a single chain variable fragment (scFv). In one aspect, the anti-cancer associated antigen as described herein binding domain is a Fv, a Fab, a (Fab′)2, or a bi-functional (e.g. bi-specific) hybrid antibody (e.g., Lanzavecchia et al., Eur. J. Immunol. 17, 105 (1987)). In one aspect, the antibodies and fragments thereof of the invention binds a cancer associated antigen as described herein protein with wild-type or enhanced affinity. In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)n, where n is a positive integer equal to or greater than 1 (SEQ ID NO: 52). In one embodiment, the linker can be (Gly₄Ser)₄ (SEQ ID NO: 45) or (Gly₄Ser)₃ (SEQ ID NO: 46). Variation in the linker length may retain or enhance activity, giving rise to superior efficacy in activity studies.

In another aspect, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a T cell clone linked by a linker (e.g., a flexible peptide). This approach is very useful to cancer associated target that itself is intracellar, however, a fragment of such antigen (peptide) is presented on the surface of the cancer cells by MHC.

CD19 Antigen Binding Domain

In some embodiments, the antigen binding domain disclosed herein binds to CD19 (e.g., human CD19) (“CD19 antigen binding domain”).

In one embodiment, the CD19 antigen binding domain has the same or a similar binding specificity as the FMC63 scFv fragment described in Nicholson et al. Mol. Immun 34 (16-17): 1157-1165 (1997). In one embodiment, the CD19 antigen binding domain includes the scFv fragment described in Nicholson et al. Mol. Immun. 34 (16-17): 1157-1165 (1997), which is incorporated herein by reference.

In one embodiment, the CD19 antigen binding domain comprises an antigen binding domain (e.g., the antigen binding domain of the CAR19 construct) described in PCT publication WO 2012/079000, which is incorporated herein by reference, or a sequence at least about 85%, 90%, 95%, 99% or more identical thereto, and/or having one, two, three or more substitutions, insertions or deletions, e.g., conserved substitutions.

In one embodiment, the CD19 antigen binding domain comprises an antigen binding domain (e.g., a humanized antigen binding domain) according to Table 3 of WO2014/153270, incorporated herein by reference, or a sequence at least about 85%, 90%, 95%, 99% or more identical thereto, and/or having one, two, three or more substitutions, insertions or deletions, e.g., conserved substitutions. Humanization of murine CD19 antibody is desired for the clinical setting, where the mouse-specific residues may induce a human-anti-mouse antigen (HAMA) response in patients who receive CART19 treatment, i.e., treatment with T cells transduced with the CAR19 construct. The production, characterization, and efficacy of humanized CD19 CAR sequences is described in International Application WO2014/153270 which is herein incorporated by reference in its entirety, including Examples 1-5 (p. 115-159). WO2014/153270 also describes methods of assaying the binding and efficacy of various CD19 antigen binding domain constructs.

In one embodiment, the CD19 antigen binding domain comprises the amino acid sequence of SEQ ID NO: 104 (or a sequence at least about 85%, 90%, 95%, 99% or more identical thereto, and/or having one, two, three or more substitutions, insertions or deletions, e.g., conserved substitutions).

(SEQ ID NO: 104)
DIQMTQTTSSLSASLGDRVTISCRASQDISKYLNWYQQKPDGTVKLL
IYHTSRLHSGVPSRFSGSGSGTDYSLTISNLEQEDIATYFCQQGNTL
PYTFGGGTKLEITGGGGSGGGGSGGGGSEVKLQESGPGLVAPSQSLS
VTCTVSGVSLPDYGVSWIRQPPRKGLEWLGVIWGSETTYYNSALKSR
LTIIKDNSKSQVFLKMNSLQTDDTAIYYCAKHYYYGGSYAMDYWGQG
TSVTVSS

BCMA Antigen Binding Domain

In some embodiments, the antigen binding domain disclosed herein binds to BCMA (e.g., human BCMA) (“BCMA antigen binding domain”).

Exemplary BCMA antigen binding domain can include sequences disclosed in Table 1 or 16 of WO2016/014565, incorporated herein by reference, or a sequence at least about 85%, 90%, 95%, 99% or more identical thereto, and/or having one, two, three or more substitutions, insertions or deletions, e.g., conserved substitutions. In one embodiment, the BCMA antigen binding domain comprises one or more CDRs, VH, VL, or scFv of BCMA-1, BCMA-2, BCMA-3, BCMA-4, BCMA-5, BCMA-6, BCMA-7, BCMA-8, BCMA-9, BCMA-10, BCMA-11, BCMA-12, BCMA-13, BCMA-14, BCMA-15, 149362, 149363, 149364, 149365, 149366, 149367, 149368, 149369, BCMA_EBB-C1978-A4, BCMA_EBB-C1978-G1, BCMA_EBB-C1979-C1, BCMA_EBB-C1978-C7, BCMA_EBB-C1978-D10, BCMA_EBB-C1979-C12, BCMA_EBB-C1980-G4, BCMA_EBB-C1980-D2, BCMA_EBB-C1978-A10, BCMA_EBB-C1978-D4, BCMA_EBB-C1980-A2, BCMA_EBB-C1981-C3, BCMA_EBB-C1978-G4, A7D12.2, C11D5.3, C12A3.2, or C13F12.1, disclosed in WO2016/014565, or a sequence at least about 85%, 90%, 95%, 99% or more identical thereto, and/or having one, two, three or more substitutions, insertions or deletions, e.g., conserved substitutions.

Additional exemplary BCMA antigen binding domains are disclosed in WO 2017/021450, WO 2017/011804, WO 2017/025038, WO 2016/090327, WO 2016/130598, WO 2016/210293, WO 2016/090320, WO 2016/014789, WO 2016/094304, WO 2016/154055, WO 2015/166073, WO 2015/188119, WO 2015/158671, U.S. Pat. Nos. 9,243,058, 8,920,776, 9,273,141, 7,083,785, 9,034,324, US 2007/0049735, US 2015/0284467, US 2015/0051266, US 2015/0344844, US 2016/0131655, US 2016/0297884, US 2016/0297885, US 2017/0051308, US 2017/0051252, US 2017/0051252, WO 2016/020332, WO 2016/087531, WO 2016/079177, WO 2015/172800, WO 2017/008169, U.S. Pat. No. 9,340,621, US 2013/0273055, US 2016/0176973, US 2015/0368351, US 2017/0051068, US 2016/0368988, and US 2015/0232557, herein incorporated by reference in their entirety. In some embodiments, additional exemplary BCMA antigen binding domains are generated using the VH and VL sequences from PCT Publication WO2012/0163805 (the contents of which are hereby incorporated by reference in its entirety).

CD20 Antigen Binding Domain

In some embodiments, the antigen binding domain disclosed herein binds to CD20 (e.g., human CD20) (“CD20 antigen binding domain”). In some embodiments, the CD20 antigen binding domain includes an antigen binding domain according to WO2016/164731 and PCT/US2017/055627, incorporated herein by reference. Exemplary CD20 antigen binding domains are disclosed in, e.g., Tables 1-5 of PCT/US2017/055627. In some embodiments, the CD20 antigen binding domain comprises a CDR, variable region, or scFv sequence of a CD20 antigen binding domain disclosed in PCT/US2017/055627 or WO2016/164731, or a sequence at least about 85%, 90%, 95%, 99% or more identical thereto, and/or having one, two, three or more substitutions, insertions or deletions, e.g., conserved substitutions.

CD22 Antigen Binding Domain

In some embodiments, the antigen binding domain disclosed herein binds to CD22 (e.g., human CD22) (“CD22 antigen binding domain”). In some embodiments, the CD22 antigen binding domain includes an antigen binding domain according to WO2016/164731 and PCT/US2017/055627, incorporated herein by reference. Exemplary CD22 antigen binding domains are disclosed in, e.g., Tables 6A, 6B, 7A, 7B, 7C, 8A, 8B, 9A, 9B, 10A, and 10B of WO2016/164731 and Tables 6-10 of PCT/US2017/055627. In some embodiments, the CD22 antigen binding domain comprise a CDR, variable region, or scFv sequence of a CD22 antigen binding domain disclosed in PCT/US2017/055627 or WO2016/164731, or a sequence at least about 85%, 90%, 95%, 99% or more identical thereto, and/or having one, two, three or more substitutions, insertions or deletions, e.g., conserved substitutions.

EGFR Antigen Binding Domain

In some embodiments, the antigen binding domain disclosed herein binds to EGFR (e.g., human EGFR, e.g., EGFRvIII) (“EGFRvIII antigen binding domain”). In some embodiments, the EGFRvIII antigen binding domain includes an antigen binding domain according to WO2014/130657, incorporated herein by reference. Exemplary EGFRvIII antigen binding domains are disclosed in, e.g., Table 2 of WO2014/130657. In some embodiments, the EGFRvIII antigen binding domain comprises a CDR, variable region, or scFv sequence of an EGFRvIII antigen binding domain disclosed in WO2014/130657, or a sequence at least about 85%, 90%, 95%, 99% or more identical thereto, and/or having one, two, three or more substitutions, insertions or deletions, e.g., conserved substitutions.

Mesothelin Antigen Binding Domain

In some embodiments, the antigen binding domain disclosed herein binds to mesothelin (e.g., human mesothelin) (“mesothelin antigen binding domain”). In some embodiments, the mesothelin antigen binding domain includes an antigen binding domain according to WO2015090230 and WO2017112741, incorporated herein by reference. Exemplary mesothelin antigen binding domains are disclosed in, e.g., Tables 2, 3, 4, and 5 of WO2017112741. In some embodiments, the mesothelin antigen binding domain comprises a CDR, variable region, or scFv sequence of a mesothelin antigen binding domain disclosed in WO2015090230 and WO2017112741, or a sequence at least about 85%, 90%, 95%, 99% or more identical thereto, and/or having one, two, three or more substitutions, insertions or deletions, e.g., conserved substitutions.

Transmembrane Domain

With respect to the transmembrane domain, in various embodiments, a chimeric protein can be designed to comprise a transmembrane domain that is attached to the extracellular domain of the chimeric protein. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the chimeric molecule, e.g., in one embodiment, the transmembrane domain may be from the same protein that the signaling domain, costimulatory domain, the hinge domain, or the extracellular domain is derived from. In another aspect, the transmembrane domain is not derived from the same protein that any other domain of the chimeric protein is derived from.

In one aspect, the transmembrane domain may be recombinant, in which case it will comprise predominantly hydrophobic residues such as leucine and valine. In one aspect a triplet of phenylalanine, tryptophan and valine can be found at each end of a recombinant transmembrane domain.

Cytoplasmic Domain

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12. In one embodiment, a CAR of the invention comprises an intracellular signaling domain, e.g., a primary signaling domain of CD3-zeta.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a.

Regulatable Chimeric Antigen Receptors

In some embodiments, a regulatable CAR (RCAR) where the CAR activity can be controlled is desirable to optimize the safety and efficacy of a CAR therapy. There are many ways CAR activities can be regulated. For example, inducible apoptosis using, e.g., a caspase fused to a dimerization domain (see, e.g., Di et al., N Egnl. J. Med. 2011 Nov. 3; 365(18):1673-1683), can be used as a safety switch in the CAR therapy of the instant invention. In an aspect, a RCAR comprises a set of polypeptides, typically two in the simplest embodiments, in which the components of a standard CAR described herein, e.g., an antigen binding domain and an intracellular signaling domain, are partitioned on separate polypeptides or members. In some embodiments, the set of polypeptides include a dimerization switch that, upon the presence of a dimerization molecule, can couple the polypeptides to one another, e.g., can couple an antigen binding domain to an intracellular signaling domain.

Dimerization Switches

Dimerization switches can be non-covalent or covalent. In a non-covalent dimerization switch, the dimerization molecule promotes a non-covalent interaction between the switch domains. In a covalent dimerization switch, the dimerization molecule promotes a covalent interaction between the switch domains.

In an embodiment, the RCAR comprises a FKBP/FRAP, or FKBP/FRB, -based dimerization switch. FKBP12 (FKBP, or FK506 binding protein) is an abundant cytoplasmic protein that serves as the initial intracellular target for the natural product immunosuppressive drug, rapamycin. Rapamycin binds to FKBP and to the large PI3K homolog FRAP (RAFT, mTOR). FRB is a 93 amino acid portion of FRAP, that is sufficient for binding the FKBP-rapamycin complex (Chen, J., Zheng, X. F., Brown, E. J. & Schreiber, S. L. (1995) Identification of an 11-kDa FKBP12-rapamycin-binding domain within the 289-kDa FKBP12-rapamycin-associated protein and characterization of a critical serine residue. Proc Natl Acad Sci USA 92: 4947-51.)

In embodiments, an FKBP/FRAP, e.g., an FKBP/FRB, based switch can use a dimerization molecule, e.g., rapamycin or a rapamycin analog.
The amino acid sequence of FKBP is as follows:

(SEQ ID NO: 53)
DVPDYASLGGPSSPKKKRKVSRGVQVETISPGDGRTFPKRG
QTCVVHYTGMLEDGKKFDSSRDRNKPFKFMLGKQEVIRGW
EEGVAQMSVGQRAKLTISPDYAYGATGHPGIIPPHATLVFD
VELLKLETSY

In embodiments, an FKBP switch domain can comprise a fragment of FKBP having the ability to bind with FRB, or a fragment or analog thereof, in the presence of rapamycin or a rapalog, e.g., the underlined portion, which is:

(SEQ ID NO: 54)
VQVETISPGDGRTFPKRGQTCVVHYTGMLEDGKKFDSSRD
RNKPFKFMLGKQEVIRGWEEGVAQMSVGQRAKLTISPDYA
YGATGHPGIIPPHATLVFDVELLKLETS

The amino acid sequence of FRB is as follows:

ILWHEMWHEG LEEASRLYFG ERNVKGMFEV LEPLHAMMER GPQTLKETSF NQAYGRDLME AQEWCRKYMK SGNVKDLTQA WDLYYHVFRR ISK (SEQ ID NO: 55)

In embodiments, the FKBP/FRB dimerization switch comprises a modified FRB switch domain that exhibits altered, e.g., enhanced, complex formation between an FRB-based switch domain, e.g., the modified FRB switch domain, a FKBP-based switch domain, and the dimerization molecule, e.g., rapamycin or a rapalogue, e.g., RAD001. In an embodiment, the modified FRB switch domain comprises one or more mutations, e.g., 2, 3, 4, 5, 6, 7, 8, 9, 10 or more, selected from mutations at amino acid position(s) L2031, E2032, S2035, R2036, F2039, G2040, T2098, W2101, D2102, Y2105, and F2108, where the wild-type amino acid is mutated to any other naturally-occurring amino acid. In an embodiment, a mutant FRB comprises a mutation at E2032, where E2032 is mutated to phenylalanine (E2032F), methionine (E2032M), arginine (E2032R), valine (E2032V), tyrosine (E2032Y), isoleucine (E20321), or leucine (E2032L). In an embodiment, a mutant FRB comprises a mutation at T2098, where T2098 is mutated to phenylalanine (T2098F) or leucine (T2098L). In an embodiment, a mutant FRB comprises a mutation at E2032 and at T2098, where E2032 is mutated to any amino acid, and where T2098 is mutated to any amino acid. In an embodiment, a mutant FRB comprises an E20321 and a T2098L mutation. In an embodiment, a mutant FRB comprises an E2032L and a T2098L mutation.

TABLE 10
Exemplary mutant FRB having increased
affinity for a dimerization molecule.

| FRB | |
|---|---|
| mutant | Amino Acid Sequence |
| E2032I | ILWHEMWHEGLIEASRLYFGERNVKGMFEVLEPLHAMMER |
| mutant | GPQTLKETSFNQAYGRDLMEAQEWCRKYMKSGNVKDLTQA |
| | WDLYYHVFRRISKTS |
| | (SEQ ID NO: 56) |
| E2032L | ILWHEMWHEGLLEASRLYFGERNVKGMFEVLEPLHAMMER |
| mutant | GPQTLKETSFNQAYGRDLMEAQEWCRKYMKSGNVKDLTQA |
| | WDLYYHVFRRISKTS |
| | (SEQ ID NO: 57) |
| T2098L | ILWHEMWHEGLEEASRLYFGERNVKGMFEVLEPLHAMMER |
| mutant | GPQTLKETSFNQAYGRDLMEAQEWCRKYMKSGNVKDLLQA |
| | WDLYYHVFRRISKTS |
| | (SEQ ID NO: 58) |
| E2032, | ILWHEMWHEGLXEASRLYFGERNVKGMFEVLEPLHAMMER |
| T2098 | GPQTLKETSFNQAYGRDLMEAQEWCRKYMKSGNVKDLXQA |
| mutant | WDLYYHVFRRISKTS |
| | (SEQ ID NO: 59) |
| E2032I, | ILWHEMWHEGLIEASRLYFGERNVKGMFEVLEPLHAMMER |
| T2098L | GPQTLKETSFNQAYGRDLMEAQEWCRKYMKSGNVKDLLQA |
| mutant | WDLYYHVFRRISKTS |
| | (SEQ ID NO: 60) |
| E2032L, | ILWHEMWHEGLLEASRLYFGERNVKGMFEVLEPLHAMMER |
| T2098L | GPQTLKETSFNQAYGRDLMEAQEWCRKYMKSGNVKDLLQA |
| mutant | WDLYYHVFRRISKTS |
| | (SEQ ID NO: 61) |

Other suitable dimerization switches include a GyrB-GyrB based dimerization switch, a Gibberellin-based dimerization switch, a tag/binder dimerization switch, and a halo-tag/snap-tag dimerization switch. Following the guidance provided herein, such switches and relevant dimerization molecules will be apparent to one of ordinary skill.

Dimerization Molecule

Association between the switch domains is promoted by the dimerization molecule. In the presence of dimerization molecule interaction or association between switch domains allows for signal transduction between a polypeptide associated with, e.g., fused to, a first switch domain, and a polypeptide associated with, e.g., fused to, a second switch domain. In the presence of non-limiting levels of dimerization molecule signal transduction is increased by 1.1, 1.2, 1.3, 1.4, 1.5, 1.6, 1.7, 1.8, 1.9, 2, 5, 10, 50, 100 fold, e.g., as measured in a system described herein.

Rapamycin and rapamycin analogs (sometimes referred to as rapalogues), e.g., RAD001, can be used as dimerization molecules in a FKBP/FRB-based dimerization switch described herein. In an embodiment the dimerization molecule can be selected from rapamycin (sirolimus), RAD001 (everolimus), zotarolimus, temsirolimus, AP-23573 (ridaforolimus), biolimus and AP21967. Additional rapamycin analogs suitable for use with FKBP/FRB-based dimerization switches are further described in the section entitled “Combination Therapies”, or in the subsection entitled “Exemplary mTOR inhibitors”.

Systems Comprising More than One Chimeric Membrane Protein

In an aspect, the invention provides systems of chimeric membrane proteins, which, when expressed in a cell, for example, result in formation of TCR that has specificity for more than one antigen, e.g., tumor antigen, e.g., described herein. Such systems are advantageous in that they do not require (though they may include) a dimerization domain described herein, but, because the antigen binding domains are linked to more than one component of the TCR, when the TCR assembles, the TCR has altered specificity towards the antigens of the antigen binding domains. The systems further comprise one or more intracellular co-stimulatory domains. Without being bound by theory, inclusion of one or more intracellular co-stimulatory domains allows for signaling both through the CD3 zeta domain of the TCR as well as through the co-stimulatory domain or domains upon antigen recognition.

Thus, the invention provides: a system comprising:

A first chimeric membrane protein comprising an extracellular domain comprising a first antigen binding domain and a first extracellular domain derived from the extracellular domain of CD3 gamma, delta, or epsilon, a transmembrane domain, and an intracellular domain comprising a first intracellular co-stimulatory domain derived from a protein other than CD3 gamma, delta or epsilon; and

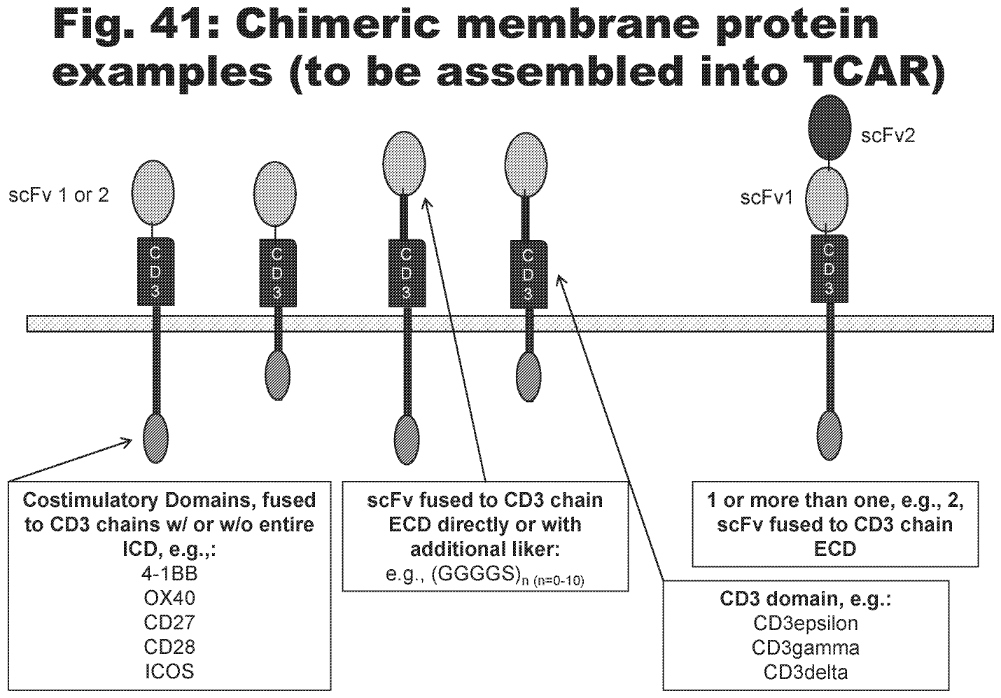

A second chimeric membrane protein comprising an extracellular domain comprising a second antigen binding domain and a second extracellular domain derived from the extracellular domain of CD3 gamma, delta, or epsilon, a transmembrane domain, and, optionally, an intracellular domain comprising a second intracellular co-stimulatory domain derived from a protein other than CD3 gamma, delta or epsilon; Wherein the first antigen binding domain and the second antigen binding domain are not identical, and wherein the first extracellular domain of CD3 gamma, delta, or epsilon and the second extracellular domain of CD3 gamma, delta, or epsilon are not identical. Exemplary embodiments of the chimeric membrane protein(s) are shown in FIG. 41.

In embodiments, the first CD3 gamma, delta, or epsilon extracellular domain comprises the entire CD3 gamma, delta, or epsilon extracellular domain.

In embodiments, the second CD3 gamma, delta, or epsilon extracellular domain the entire CD3 gamma, delta, or epsilon extracellular domain.

In embodiments, a) the first chimeric protein comprises the entire CD3 epsilon extracellular domain, and the second chimeric protein comprises the entire CD3 gamma extracellular domain; b) the first chimeric protein comprises the entire CD3 epsilon extracellular domain, and the second chimeric protein comprises the entire CD3 delta extracellular domain; or c) the first chimeric protein comprises the entire CD3 delta extracellular domain, and the second chimeric protein comprises the entire CD3 gamma extracellular domain.

In embodiments, the first chimeric protein comprises the entire CD3 gamma, delta or epsilon protein, e.g., the extracellular, transmembrane and intracellular domains of the CD3 gamma, delta or epsilon protein.

In embodiments, the second chimeric protein comprises the entire CD3 gamma, delta or epsilon protein, e.g., the extracellular, transmembrane and intracellular domains of the CD3 gamma, delta or epsilon protein.

In other embodiments, the first chimeric protein does not comprise any intracellular domains derived from the CD3 gamma, delta or epsilon protein. In embodiments, the second chimeric protein does not comprise any intracellular domains derived from CD3 gamma, delta or epsilon protein.

In embodiments, the transmembrane domain of the first chimeric protein and/or second chimeric protein does not comprise a transmembrane domain of CD3 gamma, delta or epsilon.

In embodiments, the first antigen binding domain is located N-terminal to said first extracellular domain derived from CD3 gamma, delta, or epsilon. In embodiments, the second antigen binding domain is located N-terminal to said second extracellular domain derived from CD3 gamma, delta, or epsilon. In embodiments, the first chimeric protein, the second chimeric protein, or both the first and second chimeric proteins comprise a third antigen binding domain located N-terminal to said first and/or second antigen binding domain.

In embodiments, the first antigen binding domain and said first extracellular domain derived from CD3 gamma, delta, or epsilon are connected by a first linker, e.g., a linker described herein, e.g., a (GGGGS)n linker where n is an integer from 0 to 10 (SEQ ID NO: 68), e.g., where n is equal to 4; and/or the second antigen binding domain and said second extracellular domain derived from CD3 gamma, delta, or epsilon are connected by a second linker, e.g., a linker described herein, e.g., a (GGGGS)n linker (SEQ ID NO: 68) or (GGGS)n linker (SEQ ID NO: 69), where n is an integer from 0 to 10, e.g., where n is equal to 4. Alternatively, rigid linkers (e.g., proline-rich linkers) may be used, as are known in the art.

In embodiments, only one of the two chimeric membrane proteins of the system comprises an intracellular signaling domain comprising an intracellular co-stimulatory domain, e.g., an intracellular co-stimulatory domain described herein. In embodiments, said chimeric membrane protein consists of only one intracellular co-stimulatory domain. In other embodiments, said membrane protein comprises more than one (e.g., two) intracellular signaling domains. In other embodiments, both the first chimeric membrane protein and the second chimeric membrane protein each comprise an intracellular co-stimulatory domain derived from a protein other than CD3 gamma, delta or epsilon. In embodiments, the intracellular co-stimulatory domains are the same (e.g., both are 4-1BB co-stimultory domains). In other embodiments, they are different (e.g., one is a 4-1BB co-stimulatory domain and the other is a CD28 co-stimulatory domain). The co-stimulatory domains are selected from the co-stimulatory domains described herein. In embodiments, the co-stimulatory domains are disposed immediately adjacent (e.g., immediately C-terminal) to the transmembrane domain. In other embodiments, the co-stimulatory domains are disposed C-terminal to the intracellular portion of the CD3 delta, gamma or epsilon domain, for example, the entire intracellular portion of the CD3 delta, gamma or epsilon, or the truncated portion of the CD3 delta, gamma or epsilon.

In embodiments, the antigen binding domains are as described herein. In embodiments, one or more antigen binding domains is an antibody or antibody-like molecule. In embodiments, one or more of the antigen binding domains (e.g., each of the antigen binding domains that are present in the system) are scFv. In embodiments, both the first and second antigen binding domains bind tumor antigens. In embodiments, both the first and second antigen binding domains bind B-cell antigens, e.g., as described herein. In preferred embodiments, the B-cell antigens are CD19 and CD20, CD20 and CD22, or CD19 and CD22. In other embodiments, one antigen binding domain binds a B-cell antigen, e.g., as described herein, e.g., CD19, CD20 or CD22, and the other binds a solid tumor antigen, e.g., as described herein, e.g., mesothelin or EGFRvIII.

In embodiments, one or more of the chimeric membrane proteins comprises more than one, e.g., two, antigen binding domains. By way of example, such antigen binding domains may be presented as tandem scFv antigen binding domains, optionally with a linker disposed between them. Such tandem scFv arrangements are shown in FIG. 41.

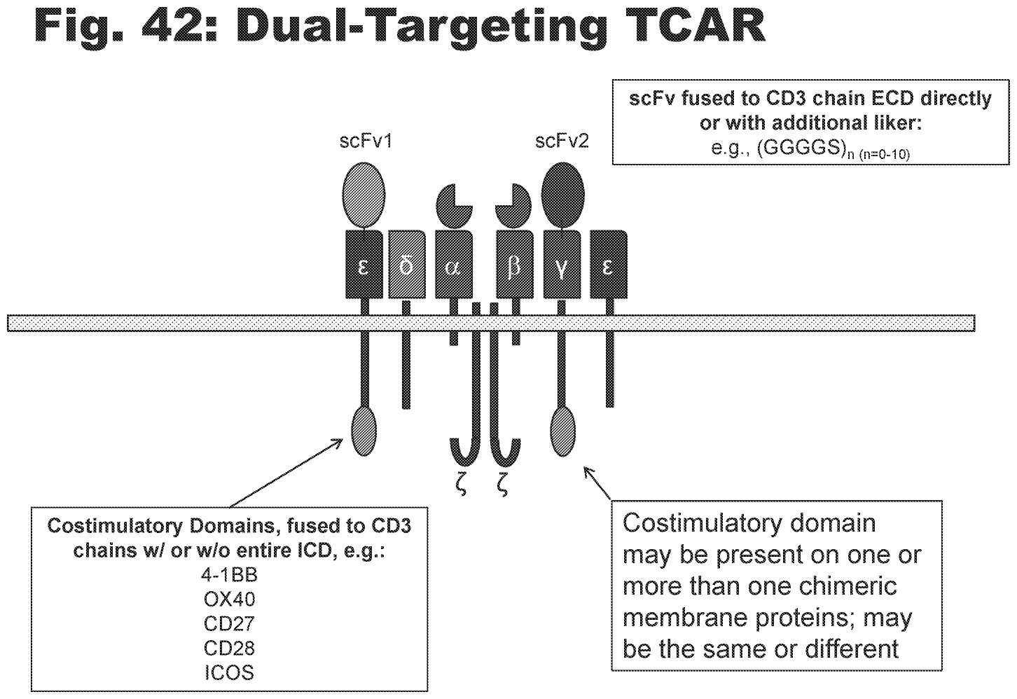

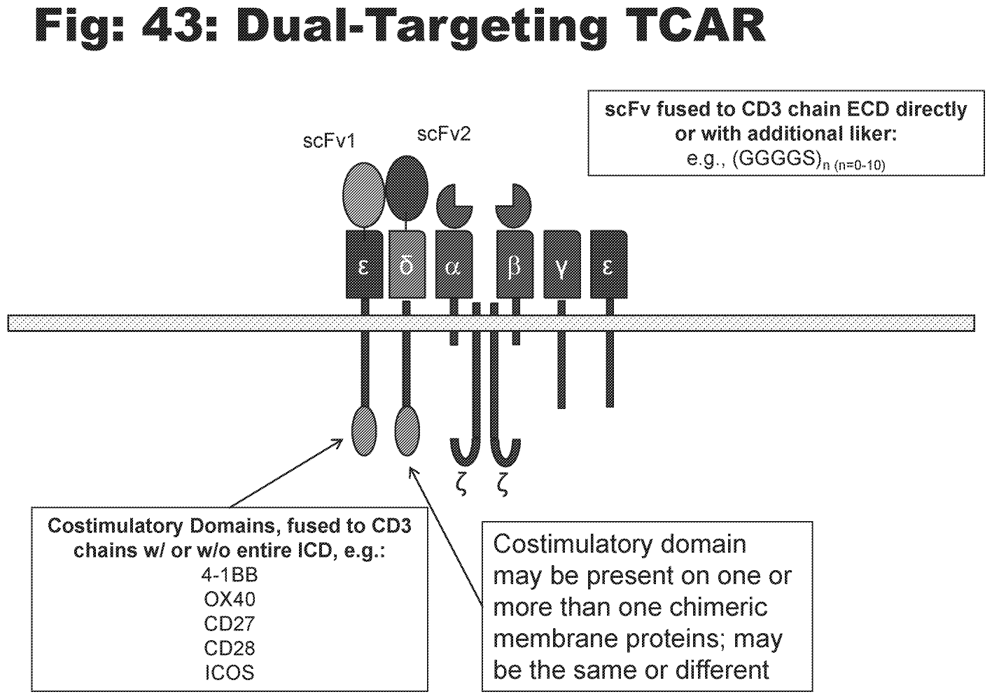

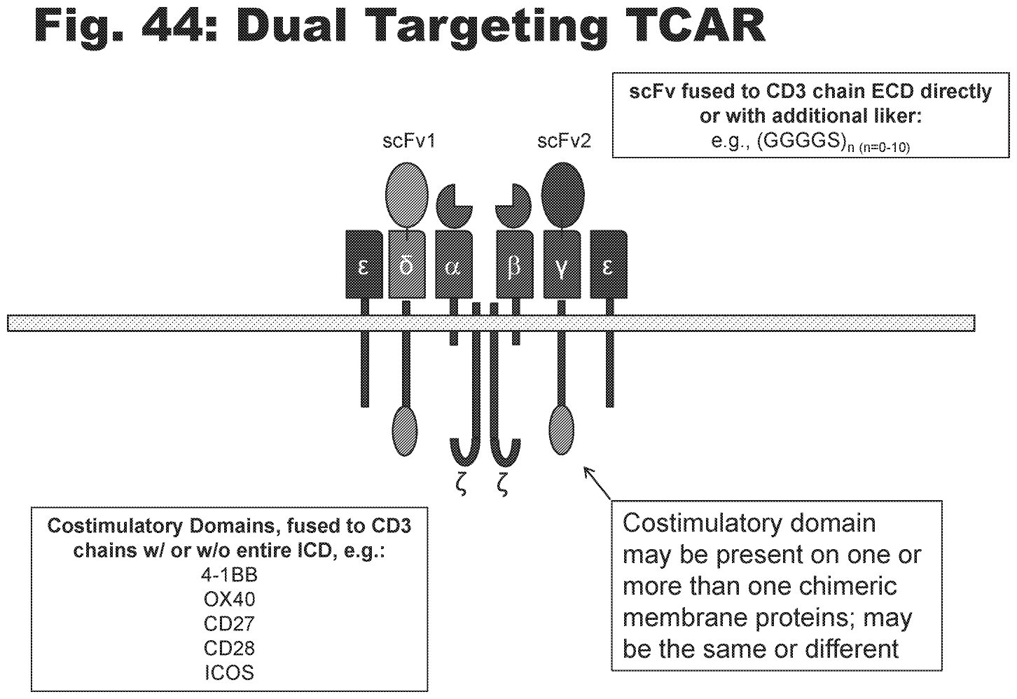

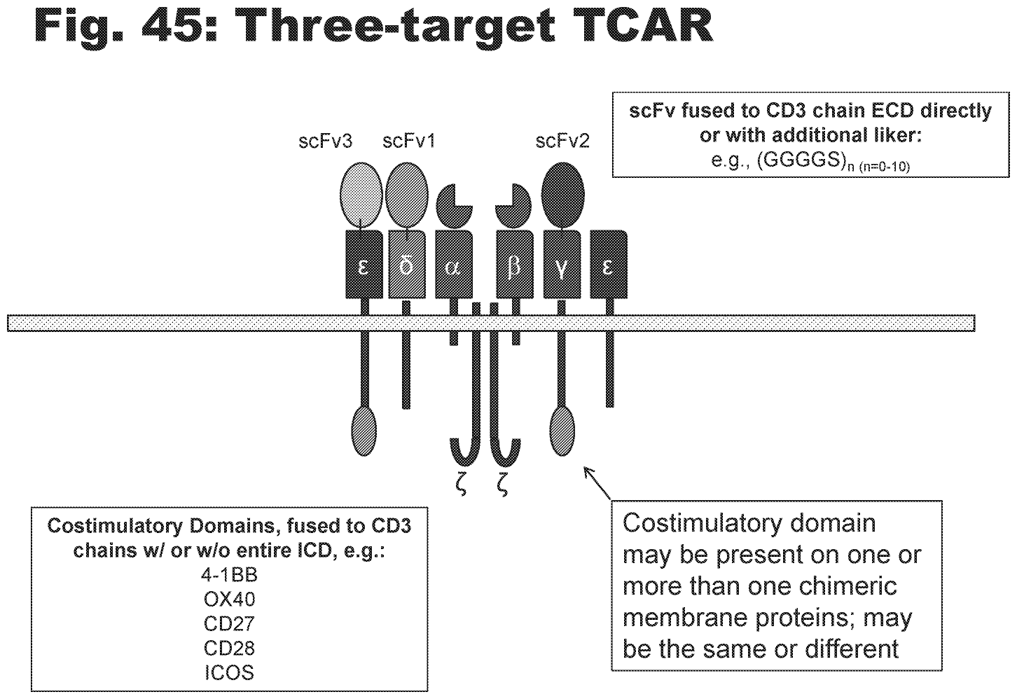

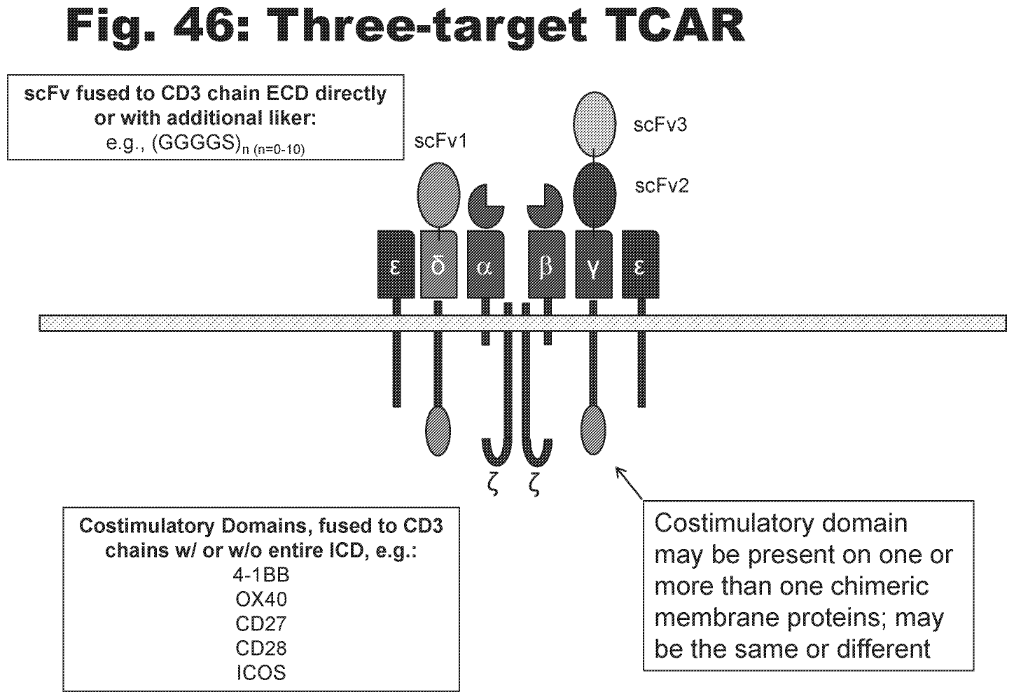

Specific examples of TCRs assembled using the systems contemplated herein are shown in FIG. 42, FIG. 43, FIG. 44, FIG. 45, or FIG. 46.

In embodiments, it can be beneficial to reduce or eliminate expression of the natural homolog (e.g., the natural CD3 epsilon, delta, or gamma homolog) of one or more of the chimeric membrane proteins of the invention. Thus, the invention provides a cell which comprises a system described herein, which additionally has reduced or eliminated expression of endogenous CD3 epsilon, delta and/or gamma proteins where the system comprises chimeric versions of the proteins. Thus, for example, where the system comprises a first chimeric membrane protein comprising all or part of the extracellular domain of CD3 delta and a second chimeric membrane protein comprising all or part of the extracellular domain of CD3 gamma, in embodiments, the cell comprising said system also has reduced or eliminated expression of endogenous CD3 gamma and/or CD3 delta. Without being bound by theory, it is believed that such reduced or eliminated expression of the endogenous counterparts of the chimeric membrane protein will favor TCR formation with the chimeric protein and reduce or eliminate TCR on the cell surface that is formed with only one or none of the chimeric membrane proteins of the system. Molecules and systems useful for reducing or eliminating expression of such one or more endogenous components of the TCR include the siRNA, shRNA, and gene editing (e.g., CRISPR, TALEN and ZFN gene editing) systems described herein.

TABLE 2
Exemplary sequences

| SEQ ID | | |
|---|---|---|
| NO | Comment | Sequence |
| SEQ ID | Exemplary human CD3 | DGNEEMGGITQTPYKVSISGTTVILTCPQYPGSEILWQ |
| NO: 77 | epsilon (P07766) | HNDKNIGGDEDDKNIGSDEDHLSLKEFSELEQSGYYV |
| | (ECD: underlined; | CYPRGSKPEDANFYLYLRARVCENCMEMDVMSVATI |
| | transmembrane: bold) | VIVDICITGGLLLLVYYWSKNRKAKAKPVTRGAGAG |
| | | GRQRGQNKERPPPVPNPDYEPIRKGQRDLYSGLNQRRI |
| SEQ ID | Exemplary human CD3 | DGNEEMGGITQTPYKVSISGTTVILTCPQYPGSEILWQ |
| NO: 78 | epsilon ECD | HNDKNIGGDEDDKNIGSDEDHLSLKEFSELEQSGYYV |
| | | CYPRGSKPEDANFYLYLRARVCENCMEMD |
| SEQ ID | Exemplary human CD3 | VMSVATIVIVDICITGGLLLLVYYWS |
| NO: 79 | epsilon transmembrane | |
| | domain | |
| SEQ ID | Exemplary human CD3 | DGNEEMGGITQTPYKVSISGTTVILTCPQYPGSEILWQ |
| NO: 80 | epsilon ECD and | HNDKNIGGDEDDKNIGSDEDHLSLKEFSELEQSGYYV |
| | transmembrane domain | CYPRGSKPEDANFYLYLRARVCENCMEMDVMSVATI |
| | | VIVDICITGGLLLLVYYWS |
| SEQ ID | Exemplary CD3 | DGNEEMGGITQTPYKVSISGTTVILTCPQYPGSEILWQ |
| NO: 81 | epsilon ECD TM-41BB | HNDKNIGGDEDDKNIGSDEDHLSLKEFSELEQSGYYV |
| | | CYPRGSKPEDANFYLYLRARVCENCMEMDVMSVATI |
| | | VIVDICITGGLLLLVYYWSKRGRKKLLYIFKQPFMRPV |
| | | QTTQEEDGCSCRFPEEEEGGCEL |
| SEQ ID | Exemplary human CD3 | FKIPIEELEDRVFVNCNTSITWVEGTVGTLLSDITRLDL |
| NO: 82 | delta (P04234) (ECD: | GKRILDPRGIYRCNGTDIYKDKESTVQVHYRMCQSCV |
| | underlined; | ELDPATVAGIIVTDVIATLLLALGVFCFAGHETGRLS |
| | transmembrane: bold) | GAADTQALLRNDQVYQPLRDRDDAQYSHLGGNWAR |
| | | NK |
| SEQ ID | Exemplary human CD3 | FKIPIEELEDRVFVNCNTSITWVEGTVGTLLSDITRLDL |
| NO: 83 | delta ECD | GKRILDPRGIYRCNGTDIYKDKESTVQVHYRMCQSCV |
| | | ELDPATVA |
| SEQ ID | Exemplary human CD3 | GIIVTDVIATLLLALGVFCFA |
| NO: 84 | delta transmembrane | |
| | domain | |
| SEQ ID | Exemplary human CD3 | FKIPIEELEDRVFVNCNTSITWVEGTVGTLLSDITRLDL |
| NO: 85 | delta ECD and | GKRILDPRGIYRCNGTDIYKDKESTVQVHYRMCQSCV |
| | transmembrane domain | ELDPATVAGIIVTDVIATLLLALGVFCFA |
| SEQ ID | Exemplary CD3 delta | FKIPIEELEDRVFVNCNTSITWVEGTVGTLLSDITRLDL |
| NO: 86 | ECD TM-41BB | GKRILDPRGIYRCNGTDIYKDKESTVQVHYRMCQSCV |
| | | ELDPATVAGIIVTDVIATLLLALGVFCFAKRGRKKLLYI |
| | | FKQPFMRPVQTTQEEDGCSCRFPEEEEGGCEL |
| SEQ ID | Exemplary human CD3 | QSIKGNHLVKVYDYQEDGSVLLTCDAEAKNITWFKD |
| NO: 87 | gamma (P09693) | GKMIGFLTEDKKKWNLGSNAKDPRGMYQCKGSQNK |
| | (ECD: underlined; | SKPLQVYYRMCQNCIELNAATISGFLFAEIVSIFVLAV |
| | transmembrane: bold) | GVYFIAGQDGVRQSRASDKQTLLPNDQLYQPLKDRE |
| | | DDQYSHLQGNQLRRN |
| SEQ ID | Exemplary human CD3 | QSIKGNHLVKVYDYQEDGSVLLTCDAEAKNITWFKD |
| NO: 88 | gamma ECD | GKMIGFLTEDKKKWNLGSNAKDPRGMYQCKGSQNK |
| | | SKPLQVYYRMCQNCIELNAATIS |
| SEQ ID | Exemplary human CD3 | GFLFAEIVSIFVLAVGVYFIA |
| NO: 89 | gamma transmembrane | |
| | domain | |
| SEQ ID | Exemplary human CD3 | QSIKGNHLVKVYDYQEDGSVLLTCDAEAKNITWFKD |
| NO: 90 | gamma ECD and | GKMIGFLTEDKKKWNLGSNAKDPRGMYQCKGSQNK |
| | transmembrane domain | SKPLQVYYRMCQNCIELNAATISGFLFAEIVSIFVLAVG |
| | | VYFIA |
| SEQ ID | Exemplary CD3 | QSIKGNHLVKVYDYQEDGSVLLTCDAEAKNITWFKD |
| NO: 91 | gamma ECD TM-41BB | GKMIGFLTEDKKKWNLGSNAKDPRGMYQCKGSQNK |
| | | SKPLQVYYRMCQNCIELNAATISGFLFAEIVSIFVLAVG |
| | | VYFIAKRGRKKLLYIFKQPFMRPVQTTQEEDGCSCRFP |
| | | EEEEGGCEL |
| SEQ ID | CD19scFv-G4S- | EIVMTQSPATLSLSPGERATLSCRASQDISKYLNWYQQ |
| NO: 70 | CD3eECDTM-41BB | KPGQAPRLLIYHTSRLHSGIPARFSGSGSGTDYTLTISSL |
| | (linker: underlined) | QPEDFAVYFCQQGNTLPYTFGQGTKLEIKGGGGSGGG |
| | | GSGGGGSQVQLQESGPGLVKPSETLSLTCTVSGVSLPD |
| | | YGVSWIRQPPGKGLEWIGVIWGSETTYYSSSLKSRVTI |
| | | SKDNSKNQVSLKLSSVTAADTAVYYCAKHYYYGGSY |
| | | AMDYWGQGTLVTVSSGGGGSDGNEEMGGITQTPYKV |
| | | SISGTTVILTCPQYPGSEILWQHNDKNIGGDEDDKNIGS |
| | | DEDHLSLKEFSELEQSGYYVCYPRGSKPEDANFYLYL |
| | | RARVCENCMEMDVMSVATIVIVDICITGGLLLLVYYW |
| | | SKRGRKKLLYIFKQPFMRPVQTTQEEDGCSCRFPEEEE |
| | | GGCEL |
| SEQ ID | CD19scFv-G4S- | EIVMTQSPATLSLSPGERATLSCRASQDISKYLNWYQQ |
| NO: 71 | CD3dECDTM-41BB | KPGQAPRLLIYHTSRLHSGIPARFSGSGSGTDYTLTISSL |
| | (linker: underlined) | QPEDFAVYFCQQGNTLPYTFGQGTKLEIKGGGGSGGG |
| | | GSGGGGSQVQLQESGPGLVKPSETLSLTCTVSGVSLPD |
| | | YGVSWIRQPPGKGLEWIGVIWGSETTYYSSSLKSRVTI |
| | | SKDNSKNQVSLKLSSVTAADTAVYYCAKHYYYGGSY |
| | | AMDYWGQGTLVTVSSGGGGSFKIPIEELEDRVFVNCN |
| | | TSITWVEGTVGTLLSDITRLDLGKRILDPRGIYRCNGTD |
| | | IYKDKESTVQVHYRMCQSCVELDPATVAGIIVTDVIAT |
| | | LLLALGVFCFAKRGRKKLLYIFKQPFMRPVQTTQEED |
| | | GCSCRFPEEEEGGCEL |
| SEQ ID | CD19scFv-G4S- | EIVMTQSPATLSLSPGERATLSCRASQDISKYLNWYQQ |
| NO: 72 | CD3gECDTM-41BB | KPGQAPRLLIYHTSRLHSGIPARFSGSGSGTDYTLTISSL |
| | (linker: underlined) | QPEDFAVYFCQQGNTLPYTFGQGTKLEIKGGGGSGGG |
| | | GSGGGGSQVQLQESGPGLVKPSETLSLTCTVSGVSLPD |
| | | YGVSWIRQPPGKGLEWIGVIVVGSETTYYSSSLKSRVTI |
| | | SKDNSKNQVSLKLSSVTAADTAVYYCAKHYYYGGSY |
| | | AMDYWGQGTLVTVSSGGGGSQSIKGNHLVKVYDYQ |
| | | EDGSVLLTCDAEAKNITWFKDGKMIGFLTEDKKKWN |
| | | LGSNAKDPRGMYQCKGSQNKSKPLQVYYRMCQNCIE |
| | | LNAATISGFLFAEIVSIFVLAVGVYFIAKRGRKKLLYIF |
| | | KQPFMRPVQTTQEEDGCSCRFPEEEEGGCEL |
| SEQ ID | CD22-65scFv-G45- | EVQLQQSGPGLVKPSQTLSLTCAISGDSMLSNSDTWN |
| NO: 73 | CD3eECDTM-41BB | WIRQSPSRGLEWLGRTYHRSTWYDDYASSVRGRVSIN |
| | (linker: underlined) | VDTSKNQYSLQLNAVTPEDTGVYYCARVRLQDGNSW |
| | | SDAFDVWGQGTMVTVSSGGGGSGGGGSGGGGSQSAL |
| | | TQPASASGSPGQSVTISCTGTSSDVGGYNYVSWYQQH |
| | | PGKAPKLMIYDVSNRPSGVSNRFSGSKSGNTASLTISG |
| | | LQAEDEADYYCSSYTSSSTLYVFGTGTQLTVLGGGGS |
| | | QDGNEEMGGITQTPYKVSISGTTVILTCPQYPGSEILW |
| | | QHNDKNIGGDEDDKNIGSDEDHLSLKEFSELEQSGYY |
| | | VCYPRGSKPEDANFYLYLRARVCENCMEMDVMSVAT |
| | | IVIVDICITGGLLLLVYYWSKRGRKKLLYIFKQPFMRP |
| | | VQTTQEEDGCSCRFPEEEEGGCEL |
| SEQ ID | CD22-65scFv-2G45- | EVQLQQSGPGLVKPSQTLSLTCAISGDSMLSNSDTWN |
| NO: 74 | CD3eECDTM-41BB | WIRQSPSRGLEWLGRTYHRSTWYDDYASSVRGRVSIN |
| | (linker: underlined) | VDTSKNQYSLQLNAVTPEDTGVYYCARVRLQDGNSW |
| | | SDAFDVWGQGTMVTVSSGGGGSGGGGSGGGGSQSAL |
| | | TQPASASGSPGQSVTISCTGTSSDVGGYNYVSWYQQH |
| | | PGKAPKLMIYDVSNRPSGVSNRFSGSKSGNTASLTISG |
| | | LQAEDEADYYCSSYTSSSTLYVFGTGTQLTVLGGGGS |
| | | GGGGSQDGNEEMGGITQTPYKVSISGTTVILTCPQYPG |
| | | SEILWQHNDKNIGGDEDDKNIGSDEDHLSLKEFSELEQ |
| | | SGYYVCYPRGSKPEDANFYLYLRARVCENCMEMDV |
| | | MSVATIVIVDICITGGLLLLVYYWSKRGRKKLLYIFKQ |
| | | PFMRPVQTTQEEDGCSCRFPEEEEGGCEL |
| SEQ ID | CD22-65scFv-G45- | EVQLQQSGPGLVKPSQTLSLTCAISGDSMLSNSDTWN |
| NO: 75 | CD3gECDTM-41BB | WIRQSPSRGLEWLGRTYHRSTWYDDYASSVRGRVSIN |
| | (linker: underlined) | VDTSKNQYSLQLNAVTPEDTGVYYCARVRLQDGNSW |
| | | SDAFDVWGQGTMVTVSSGGGGSGGGGSGGGGSQSAL |
| | | TQPASASGSPGQSVTISCTGTSSDVGGYNYVSWYQQH |
| | | PGKAPKLMIYDVSNRPSGVSNRFSGSKSGNTASLTISG |
| | | LQAEDEADYYCSSYTSSSTLYVFGTGTQLTVLGGGGS |
| | | QSIKGNHLVKVYDYQEDGSVLLTCDAEAKNITWFKD |
| | | GKMIGFLTEDKKKWNLGSNAKDPRGMYQCKGSQNK |
| | | SKPLQVYYRMCQNCIELNAATISGFLFAEIVSIFVLAVG |
| | | VYFIAKRGRKKLLYIFKQPFMRPVQTTQEEDGCSCRFP |
| | | EEEEGGCEL |
| SEQ ID | CD22-65scFv-2G45- | EVQLQQSGPGLVKPSQTLSLTCAISGDSMLSNSDTWN |
| NO: 76 | CD3gECDTM-41BB | WIRQSPSRGLEWLGRTYHRSTWYDDYASSVRGRVSIN |
| | (linker: underlined) | VDTSKNQYSLQLNAVTPEDTGVYYCARVRLQDGNSW |
| | | SDAFDVWGQGTMVTVSSGGGGSGGGGSGGGGSQSAL |
| | | TQPASASGSPGQSVTISCTGTSSDVGGYNYVSWYQQH |
| | | PGKAPKLMIYDVSNRPSGVSNRFSGSKSGNTASLTISG |
| | | LQAEDEADYYCSSYTSSSTLYVFGTGTQLTVLGGGGS |
| | | GGGGSQSIKGNHLVKVYDYQEDGSVLLTCDAEAKNIT |
| | | WFKDGKMIGFLTEDKKKWNLGSNAKDPRGMYQCKG |
| | | SQNKSKPLQVYYRMCQNCIELNAATISGFLFAEIVSIFV |
| | | LAVGVYFIAKRGRKKLLYIFKQPFMRPVQTTQEEDGC |
| | | SCRFPEEEEGGCEL |

Nucleic Acid Constructs Encoding a CAR

The present invention also provides nucleic acid molecules encoding one or more chimeric protein constructs described herein. In one aspect, the nucleic acid molecule is provided as a messenger RNA transcript. In one aspect, the nucleic acid molecule is provided as a DNA construct.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (w), a primer binding site (PBS), one or more (e.g., two) long terminal repeats (LTR), and a transgene of interest, e.g., a gene encoding a chimeric protein. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR of the invention is an adenoviral vector (A5/35). In another embodiment, the expression of nucleic acids encoding chimeric proteins can be accomplished using of transposons such as sleeping beauty, crisper, CAS9, and zinc finger nucleases. See below June et al. 2009 Nature Reviews Immunology 9.10: 704-716, is incorporated herein by reference.

Sources of Cells

Prior to expansion and genetic modification or other modification, a source of cells, e.g., T cells or natural killer (NK) cells, can be obtained from a subject. The term “subject” is intended to include living organisms in which an immune response can be elicited (e.g., mammals). Examples of subjects include humans, monkeys, chimpanzees, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In certain aspects of the present disclosure, immune effector cells, e.g., T cells, can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.2014.31.

In one aspect, T cells are isolated from peripheral blood lymphocytes by lysing the red blood cells and depleting the monocytes, for example, by centrifugation through a PERCOLL™ gradient or by counterflow centrifugal elutriation.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

In one embodiment, T regulatory cells, e.g., CD25+ T cells, are removed from the population using an anti-CD25 antibody, or fragment thereof, or a CD25-binding ligand, IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein.

In one embodiment, the T regulatory cells, e.g., CD25+ T cells, are removed from the population using CD25 depletion reagent from Miltenyi™. In one embodiment, the ratio of cells to CD25 depletion reagent is 1e7 cells to 20 uL, or 1e7 cells to 15 uL, or 1e7 cells to 10 uL, or 1e7 cells to 5 uL, or 1e7 cells to 2.5 uL, or 1e7 cells to 1.25 uL. In one embodiment, e.g., for T regulatory cells, e.g., CD25+ depletion, greater than 500 million cells/ml is used. In a further aspect, a concentration of cells of 600, 700, 800, or 900 million cells/ml is used. In one embodiment, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In one embodiment, the resulting population T regulatory depleted cells has 2×10⁹ T regulatory cells, e.g., CD25+ cells, or less (e.g., 1×10⁹, 5×10⁸, 1×10⁸, 5×10⁷, 1×10⁷, or less CD25+ cells).

In one embodiment, the T regulatory cells, e.g., CD25+ cells, are removed from the population using the CliniMAC system with a depletion tubing set, such as, e.g., tubing 162-01. In one embodiment, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., T_{REG} cells), in a subject prior to apheresis or during manufacturing of a chimeric protein-expressing cell product can reduce the risk of subject relapse. For example, methods of depleting T_{REG} cells are known in the art. Methods of decreasing T_{REG} cells include, but are not limited to, cyclophosphamide, anti-GITR antibody (an anti-GITR antibody described herein), CD25-depletion, and combinations thereof.

In some embodiments, the manufacturing methods comprise reducing the number of (e.g., depleting) T_{REG} cells prior to manufacturing of the chimeric protein-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the chimeric protein-expressing cell (e.g., T cell, NK cell) product.

In an embodiment, a subject is pre-treated with one or more therapies that reduce T_{REG} cells prior to collection of cells, thereby reducing the risk of subject relapse to cell treatment. In an embodiment, methods of decreasing T_{REG} cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the cell product.

In one embodiment, the population of cells to be removed are neither the regulatory T cells or tumor cells, but cells that otherwise negatively affect the expansion and/or function of cells, e.g. cells expressing CD14, CD11b, CD33, CD15, or other markers expressed by potentially immune suppressive cells. In one embodiment, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory depleted, e.g., CD25+ depleted, and tumor antigen depleted cells that are suitable for expression of a chimeric protein. In one embodiment, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory depleted, e.g., CD25+ depleted cells, and check point inhibitor depleted cells, e.g., PD1+, LAG3+ and/or TIM3+ depleted cells. Exemplary check point inhibitors include B7-H1, B7-1, CD160, P1H, 2B4, PD1, TIM3, CEACAM (e.g., CEACAM-1, CEACAM-3 and/or CEACAM-5), LAG3, TIGIT, CTLA-4, BTLA and LAIR1. In one embodiment, check point inhibitor expressing cells are removed simultaneously with the T regulatory, e.g., CD25+ cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25+ cells, and the removal of the check point inhibitor expressing cells is sequential, and can occur, e.g., in either order.

Methods described herein can include a positive selection step. For example, T cells can be isolated by incubation with anti-CD3/anti-CD28 (e.g., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours, e.g., 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points.

In one embodiment, a T cell population can be selected that expresses one or more of IFN-γ, TNFα, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712. For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 10 billion cells/ml, 9 billion/ml, 8 billion/ml, 7 billion/ml, 6 billion/ml, or 5 billion/ml is used. In one aspect, a concentration of 1 billion cells/ml is used. In yet one aspect, a concentration of cells from 75, 80, 85, 90, 95, or 100 million cells/ml is used. In further aspects, concentrations of 125 or 150 million cells/ml can be used.

Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10⁶/ml. In other aspects, the concentration used can be from about 1×10⁵/ml to 1×10⁶/ml, and any integer value in between.

In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain aspects, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in immune effector cell therapy for any number of diseases or conditions that would benefit from immune effector cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In one embodiment, the immune effector cells expressing a CAR molecule, e.g., a CAR molecule described herein, are obtained from a subject that has received a low, immune enhancing dose of an mTOR inhibitor. In an embodiment, the population of immune effector cells, e.g., T cells, to be engineered to express a CAR, are harvested after a sufficient time, or after sufficient dosing of the low, immune enhancing, dose of an mTOR inhibitor, such that the level of PD1 negative immune effector cells, e.g., T cells, or the ratio of PD1 negative immune effector cells, e.g., T cells/PD1 positive immune effector cells, e.g., T cells, in the subject or harvested from the subject has been, at least transiently, increased.

In other embodiments, population of immune effector cells can be treated ex vivo by contact with an amount of an mTOR inhibitor that increases the number of PD1 negative immune effector cells, e.g., T cells or increases the ratio of PD1 negative immune effector cells, e.g., T cells/PD1 positive immune effector cells, e.g., T cells.

In one embodiment, a T cell population is diaglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In one embodiment, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In an embodiment, the NK cells are obtained from the subject. In another embodiment, the NK cells are an NK cell line, e.g., NK-92 cell line (Conkwest).

Allogeneic Cells

In embodiments described herein, the immune effector cell can be an allogeneic immune effector cell, e.g., T cell or NK cell. For example, the cell can be an allogeneic T cell, e.g., an allogeneic T cell lacking expression of a functional T cell receptor (TCR) and/or human leukocyte antigen (HLA), e.g., HLA class I and/or HLA class II.

A T cell lacking a functional TCR can be, e.g., engineered such that it does not express any functional TCR on its surface, engineered such that it does not express one or more subunits that comprise a functional TCR or engineered such that it produces very little functional TCR on its surface. Alternatively, the T cell can express a substantially impaired TCR, e.g., by expression of mutated or truncated forms of one or more of the subunits of the TCR. The term “substantially impaired TCR” means that this TCR will not elicit an adverse immune reaction in a host.

A T cell described herein can be, e.g., engineered such that it does not express a functional HLA on its surface. For example, a T cell described herein, can be engineered such that cell surface expression HLA, e.g., HLA class 1 and/or HLA class II, is downregulated.

In some embodiments, the T cell can lack a functional TCR and a functional HLA, e.g., HLA class I and/or HLA class II.

Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

In some embodiments, the allogeneic cell can be a cell which does not express or expresses at low levels an inhibitory molecule, e.g. by any method described herein. For example, the cell can be a cell that does not express or expresses at low levels an inhibitory molecule. Examples of inhibitory molecules include PD1, PD-L1, CTLA4, TIM3, CEACAM (e.g., CEACAM-1, CEACAM-3 and/or CEACAM-5), LAG3, VISTA, BTLA, TIGIT, LAIR1, CD160, 2B4 and TGF beta. Inhibition of an inhibitory molecule, e.g., by inhibition at the DNA, RNA or protein level, can optimize a cell performance. In embodiments, an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used.

siRNA and shRNA to Inhibit TCR or HLA

In some embodiments, TCR expression and/or HLA expression can be inhibited using siRNA or shRNA that targets a nucleic acid encoding a TCR and/or HLA in a T cell. Expression of siRNA and shRNAs in T cells can be achieved using any conventional expression system, e.g., such as a lentiviral expression system.

Exemplary shRNAs that downregulate expression of components of the TCR are described, e.g., in US Publication No.: 2012/0321667. Exemplary siRNA and shRNA that downregulate expression of HLA class I and/or HLA class II genes are described, e.g., in U.S. publication No.: US 2007/0036773.

CRISPR to Inhibit TCR or HLA

“CRISPR” or “CRISPR to TCR and/or HLA” or “CRISPR to inhibit TCR and/or HLA” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence or mutate a TCR and/or HLA gene.

Naturally-occurring CRISPR/Cas systems are found in approximately 40% of sequenced eubacteria genomes and 90% of sequenced archaea. Grissa et al. (2007) BMC Bioinformatics 8: 172. This system is a type of prokaryotic immune system that confers resistance to foreign genetic elements such as plasmids and phages and provides a form of acquired immunity. Barrangou et al. (2007) Science 315: 1709-1712; Marragini et al. (2008) Science 322: 1843-1845.

The CRISPR/Cas system can thus be used to edit a TCR and/or HLA gene (adding or deleting a basepair), or introducing a premature stop which thus decreases expression of a TCR and/or HLA. The CRISPR/Cas system can alternatively be used like RNA interference, turning off TCR and/or HLA gene in a reversible fashion. In a mammalian cell, for example, the RNA can guide the Cas protein to a TCR and/or HLA promoter, sterically blocking RNA polymerases.

Artificial CRISPR/Cas systems can be generated which inhibit TCR and/or HLA, using technology known in the art, e.g., that described in U.S. Publication No. 20140068797, and Cong (2013) Science 339: 819-823. Other artificial CRISPR/Cas systems that are known in the art may also be generated which inhibit TCR and/or HLA, e.g., that described in Tsai (2014) Nature Biotechnol., 32:6 569-576, U.S. Pat. Nos. 8,871,445; 8,865,406; 8,795,965; 8,771,945; and 8,697,359.

TALEN to Inhibit TCR and/or HLA

“TALEN” or “TALEN to HLA and/or TCR” or “TALEN to inhibit HLA and/or TCR” refers to a transcription activator-like effector nuclease, an artificial nuclease which can be used to edit the HLA and/or TCR gene.

Zinc Finger Nuclease to Inhibit HLA and/or TCR

“ZFN” or “Zinc Finger Nuclease” or “ZFN to HLA and/or TCR” or “ZFN to inhibit HLA and/or TCR” refer to a zinc finger nuclease, an artificial nuclease which can be used to edit the HLA and/or TCR gene.

Like a TALEN, a ZFN comprises a Fold nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160.

Telomerase Expression

While not wishing to be bound by any particular theory, in some embodiments, a therapeutic T cell has short term persistence in a patient, due to shortened telomeres in the T cell; accordingly, transfection with a telomerase gene can lengthen the telomeres of the T cell and improve persistence of the T cell in the patient. See Carl June, “Adoptive T cell therapy for cancer in the clinic”, Journal of Clinical Investigation, 117:1466-1476 (2007). Thus, in an embodiment, an immune effector cell, e.g., a T cell, ectopically expresses a telomerase subunit, e.g., the catalytic subunit of telomerase, e.g., TERT, e.g., hTERT. In some aspects, this disclosure provides a method of producing a cell, comprising contacting a cell with a nucleic acid encoding a telomerase subunit, e.g., the catalytic subunit of telomerase, e.g., TERT, e.g., hTERT. The cell may be contacted with the nucleic acid before, simultaneous with, or after being contacted with a construct encoding a chimeric protein.

Activation and Expansion of Immune Effector Cells (e.g., T Cells)

Immune effector cells such as T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, a population of immune effector cells e.g., T regulatory cell depleted cells, may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besançon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain aspects, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one aspect, the agent providing the costimulatory signal is bound to a cell surface and the agent providing the primary activation signal is in solution or coupled to a surface. In certain aspects, both agents can be in solution. In one aspect, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one aspect, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In one aspect, a 1:1 ratio of each antibody bound to the beads for CD4+ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular aspect an increase of from about 1 to about 3 fold is observed as compared to the expansion observed using a ratio of 1:1. In one aspect, the ratio of CD3:CD28 antibody bound to the beads ranges from 100:1 to 1:100 and all integer values there between. In one aspect, more anti-CD28 antibody is bound to the particles than anti-CD3 antibody, i.e., the ratio of CD3:CD28 is less than one. In certain aspects, the ratio of anti CD28 antibody to anti CD3 antibody bound to the beads is greater than 2:1. In one particular aspect, a 1:100 CD3:CD28 ratio of antibody bound to beads is used. In one aspect, a 1:75 CD3:CD28 ratio of antibody bound to beads is used. In a further aspect, a 1:50 CD3:CD28 ratio of antibody bound to beads is used. In one aspect, a 1:30 CD3:CD28 ratio of antibody bound to beads is used. In one preferred aspect, a 1:10 CD3:CD28 ratio of antibody bound to beads is used. In one aspect, a 1:3 CD3:CD28 ratio of antibody bound to the beads is used. In yet one aspect, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain aspects the ratio of cells to particles ranges from 1:100 to 100:1 and any integer values in-between and in further aspects the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one aspect, a ratio of particles to cells of 1:1 or less is used. In one particular aspect, a preferred particle: cell ratio is 1:5. In further aspects, the ratio of particles to cells can be varied depending on the day of stimulation. For example, in one aspect, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular aspect, the ratio of particles to cells is 1:1 on the first day of stimulation and adjusted to 1:5 on the third and fifth days of stimulation. In one aspect, particles are added on a daily or every other day basis to a final ratio of 1:1 on the first day, and 1:5 on the third and fifth days of stimulation. In one aspect, the ratio of particles to cells is 2:1 on the first day of stimulation and adjusted to 1:10 on the third and fifth days of stimulation. In one aspect, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type. In one aspect, the most typical ratios for use are in the neighborhood of 1:1, 2:1 and 3:1 on the first day.

In further aspects, the cells, such as T cells, are combined with agent-coated beads, the beads and the cells are subsequently separated, and then the cells are cultured. In an alternative aspect, prior to culture, the agent-coated beads and cells are not separated but are cultured together. In a further aspect, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one aspect the cells (for example, 10⁴ to 10⁹ T cells) and beads (for example, DYNABEADS® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain aspects, it may be desirable to significantly decrease the volume in which particles and cells are mixed together (i.e., increase the concentration of cells), to ensure maximum contact of cells and particles. For example, in one aspect, a concentration of about 10 billion cells/ml, 9 billion/ml, 8 billion/ml, 7 billion/ml, 6 billion/ml, 5 billion/ml, or 2 billion cells/ml is used. In one aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain aspects. For example, using high concentration of cells allows more efficient selection of CD8+ T cells that normally have weaker CD28 expression.

In one embodiment, cells transduced with a nucleic acid described herein, are expanded, e.g., by a method described herein. In one embodiment, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days).

Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In one embodiment, the cells are expanded in an appropriate media (e.g., media described herein) that includes one or more interleukin that result in at least a 200-fold (e.g., 200-fold, 250-fold, 300-fold, 350-fold) increase in cells over a 14 day expansion period, e.g., as measured by a method described herein such as flow cytometry. In one embodiment, the cells are expanded in the presence of IL-15 and/or IL-7 (e.g., IL-15 and IL-7).

In embodiments, methods described herein comprise removing T regulatory cells, e.g., CD25+ T cells, from a cell population, e.g., using an anti-CD25 antibody, or fragment thereof, or a CD25-binding ligand, IL-2. Methods of removing T regulatory cells, e.g., CD25+ T cells, from a cell population are described herein. In embodiments, the methods, e.g., manufacturing methods, further comprise contacting a cell population (e.g., a cell population in which T regulatory cells, such as CD25+ T cells, have been depleted; or a cell population that has previously contacted an anti-CD25 antibody, fragment thereof, or CD25-binding ligand) with IL-15 and/or IL-7. For example, the cell population (e.g., that has previously contacted an anti-CD25 antibody, fragment thereof, or CD25-binding ligand) is expanded in the presence of IL-15 and/or IL-7.

In some embodiments a cell described herein is contacted with a composition comprising a interleukin-15 (IL-15) polypeptide, a interleukin-15 receptor alpha (IL-15Ra) polypeptide, or a combination of both a IL-15 polypeptide and a IL-15Ra polypeptide e.g., hetIL-15, during the manufacturing of the cell, e.g., ex vivo. In embodiments, a cell described herein is contacted with a composition comprising a IL-15 polypeptide during the manufacturing of the cell, e.g., ex vivo. In embodiments, a cell described herein is contacted with a composition comprising a combination of both a IL-15 polypeptide and a IL-15 Ra polypeptide during the manufacturing of the cell, e.g., ex vivo.

In one embodiment the cell described herein is contacted with a composition comprising hetIL-15 during ex vivo expansion. In an embodiment, the cell described herein is contacted with a composition comprising an IL-15 polypeptide during ex vivo expansion. In an embodiment, the cell described herein is contacted with a composition comprising both an IL-15 polypeptide and an IL-15Ra polypeptide during ex vivo expansion. In one embodiment the contacting results in the survival and proliferation of a lymphocyte subpopulation, e.g., CD8+ T cells.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Therapeutic Application

In another aspect, a method of treating a subject, e.g., reducing or ameliorating, a hyperproliferative condition or disorder (e.g., a cancer), e.g., solid tumor, a soft tissue tumor, or a metastatic lesion, in a subject is provided. As used herein, the term “cancer” is meant to include all types of cancerous growths or oncogenic processes, metastatic tissues or malignantly transformed cells, tissues, or organs, irrespective of histopathologic type or stage of invasiveness. Examples of solid tumors include malignancies, e.g., sarcomas, adenocarcinomas, and carcinomas, of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. In one embodiment, the cancer is a melanoma, e.g., an advanced stage melanoma. Metastatic lesions of the aforementioned cancers can also be treated or prevented using the methods and compositions of the invention. Examples of other cancers that can be treated include bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. Treatment of metastatic cancers, e.g., metastatic cancers that express PD-L1 (Iwai et al. (2005) Int. Immunol. 17:133-144) can be effected using the antibody molecules described herein.

Exemplary cancers whose growth can be inhibited include cancers typically responsive to immunotherapy. Non-limiting examples of cancers for treatment include melanoma (e.g., metastatic malignant melanoma), renal cancer (e.g. clear cell carcinoma), prostate cancer (e.g. hormone refractory prostate adenocarcinoma), breast cancer, colon cancer and lung cancer (e.g. non-small cell lung cancer). Additionally, refractory or recurrent malignancies can be treated using the molecules described herein.

In one aspect, the invention pertains to a method of treating cancer in a subject. The method comprises administering to the subject cell of the present invention such that the cancer is treated in the subject. In one aspect, the cancer associated with expression of a cancer associate antigen as described herein is a hematological cancer. In one aspect, the hematological cancer is a leukemia or a lymphoma. In one aspect, a cancer associated with expression of a cancer associate antigen as described herein includes cancers and malignancies including, but not limited to, e.g., one or more acute leukemias including but not limited to, e.g., B-cell acute Lymphoid Leukemia (“BALL”), T-cell acute Lymphoid Leukemia (“TALL”), acute lymphoid leukemia (ALL); one or more chronic leukemias including but not limited to, e.g., chronic myelogenous leukemia (CML), Chronic Lymphoid Leukemia (CLL). Additional cancers or hematologic conditions associated with expression of a cancer associate antigen as described herein include, but are not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further a disease associated with a cancer associate antigen as described herein expression include, but not limited to, e.g., atypical and/or non-classical cancers, malignancies, precancerous conditions or proliferative diseases associated with expression of a cancer associate antigen as described herein.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of immune effector cells (e.g., T cells, NK cells) comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also provides compositions and methods for in vivo immunization to elicit an immune response directed against an antigen in a patient.

Hematologic Cancer

Hematological cancer conditions are the types of cancer such as leukemia, lymphoma, and malignant lymphoproliferative conditions that affect blood, bone marrow and the lymphatic system.

Leukemia can be classified as acute leukemia and chronic leukemia. Acute leukemia can be further classified as acute myelogenous leukemia (AML) and acute lymphoid leukemia (ALL). Chronic leukemia includes chronic myelogenous leukemia (CML) and chronic lymphoid leukemia (CLL). Other related conditions include myelodysplastic syndromes (MDS, formerly known as “preleukemia”) which are a diverse collection of hematological conditions united by ineffective production (or dysplasia) of myeloid blood cells and risk of transformation to AML.

Lymphoma is a group of blood cell tumors that develop from lymphocytes. Exemplary lymphomas include non-Hodgkin lymphoma and Hodgkin lymphoma.

The present invention provides for compositions and methods for treating cancer. In one aspect, the cancer is a hematologic cancer including but is not limited to hematolical cancer is a leukemia or a lymphoma. In one aspect, the cells of the invention may be used to treat cancers and malignancies such as, but not limited to, e.g., acute leukemias including but not limited to, e.g., B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further a disease associated with a cancer associate antigen as described herein expression includes, but not limited to, e.g., atypical and/or non-classical cancers, malignancies, precancerous conditions or proliferative diseases expressing a cancer associate antigen as described herein.

The present invention also provides methods for preventing, treating and/or managing a disease associated with a cancer associated antigen as described herein-expressing cells (e.g., a hematologic cancer or atypical cancer expressing a cancer associated antigen as described herein), the methods comprising administering to a subject in need a T cell or NK cell of the invention that binds to a cancer associated antigen as described herein-expressing cell. In one aspect, the subject is a human. Non-limiting examples of disorders associated with a cancer associated antigen as described herein-expressing cells include autoimmune disorders (such as lupus), inflammatory disorders (such as allergies and asthma) and cancers (such as hematological cancers or atypical cancers expressing a cancer associated antigen as described herein).

The present invention also provides methods for preventing, treating and/or managing a disease associated with a cancer associated antigen as described herein-expressing cells, the methods comprising administering to a subject in need a T cell or NK cell of the invention that binds to a cancer associated antigen as described herein-expressing cell. In one aspect, the subject is a human.

The present invention provides methods for preventing relapse of cancer associated with a cancer associated antigen as described herein-expressing cells, the methods comprising administering to a subject in need thereof a T cell or NK cell of the invention that binds to a cancer associated antigen as described herein-expressing cell. In one aspect, the methods comprise administering to the subject in need thereof an effective amount of a T cell or NK cell described herein that binds to a cancer associated antigen as described herein-expressing cell in combination with an effective amount of another therapy.

Pharmaceutical Compositions and Treatments

Pharmaceutical compositions of the present invention may comprise a chimeric protein-expressing cell, e.g., a plurality of chimeric protein-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A. When “an immunologically effective amount,” “an anti-tumor effective amount,” “a tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the immune effector cells (e.g., T cells, NK cells) described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

In certain aspects, it may be desired to administer activated immune effector cells (e.g., T cells, NK cells) to a subject and then subsequently redraw blood (or have an apheresis performed), activate immune effector cells (e.g., T cells, NK cells) therefrom according to the present invention, and reinfuse the patient with these activated and expanded immune effector cells (e.g., T cells, NK cells). This process can be carried out multiple times every few weeks. In certain aspects, immune effector cells (e.g., T cells, NK cells) can be activated from blood draws of from 10 cc to 400 cc. In certain aspects, immune effector cells (e.g., T cells, NK cells) are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In one aspect, the T cell compositions of the present invention are administered to a patient by intradermal or subcutaneous injection. In one aspect, the T cell compositions of the present invention are administered by i.v. injection. The compositions of immune effector cells (e.g., T cells, NK cells) may be injected directly into a tumor, lymph node, or site of infection.

In a particular exemplary aspect, subjects may undergo leukapheresis, wherein leukocytes are collected, enriched, or depleted ex vivo to select and/or isolate the cells of interest, e.g., T cells. These T cell isolates may be expanded by methods known in the art and treated such that one or more constructs of the invention may be introduced, thereby creating a T cell of the invention. Subjects in need thereof may subsequently undergo standard treatment with high dose chemotherapy followed by peripheral blood stem cell transplantation. In certain aspects, following or concurrent with the transplant, subjects receive an infusion of the expanded T cells of the present invention. In an additional aspect, expanded cells are administered before or following surgery.

The dosage of the above treatments to be administered to a patient will vary with the precise nature of the condition being treated and the recipient of the treatment. The scaling of dosages for human administration can be performed according to art-accepted practices.

The dose for CAMPATH, for example, will generally be in the range 1 to about 100 mg for an adult patient, usually administered daily for a period between 1 and 30 days. The preferred daily dose is 1 to 10 mg per day although in some instances larger doses of up to

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: Constitutively Active TCARs Using Intracellular Heterodimerization Domains

Transient Expression and Activation Assays

Materials and Methods
Synthesis of Constitutively Active TCAR constructs

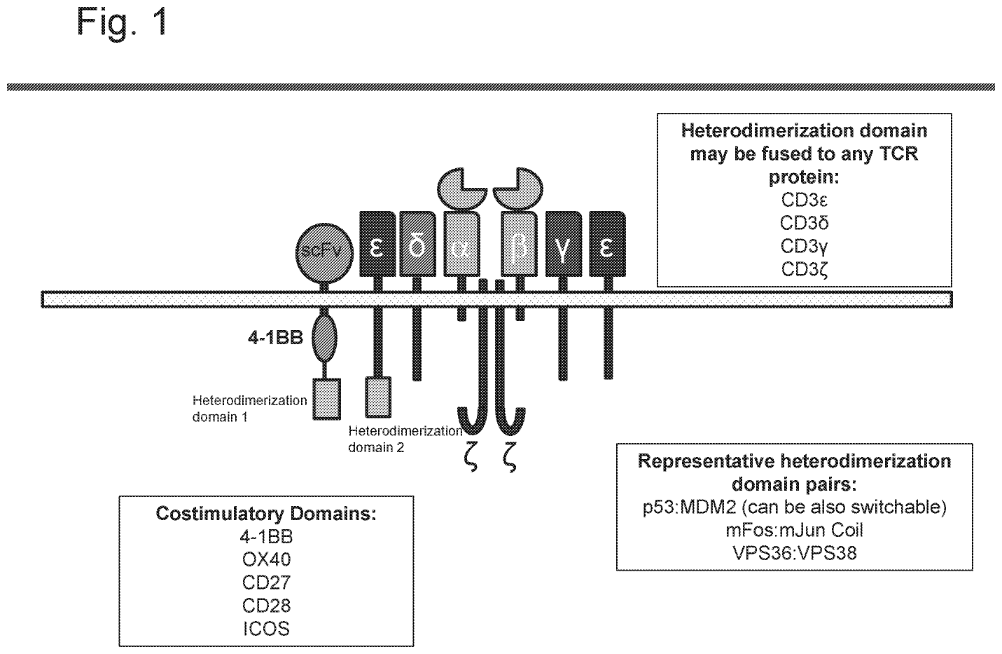

Pairs of plasmid DNA were synthesized externally by DNA2.0. The nominal non-regulatable CAR construct, CD19scFv-BBZ, SEQ ID NO: 1, was used as a control. For the TCAR, various intracellular heterodimerization domains can be linked to different domains of the TCAR constructs as shown in FIG. 1.

“TCAR1” comprises a pair of constructs. In the first construct, the CD19 scFv was cloned with CD8 hinge and transmembrane domain followed by the costimulatory domain 4-1BB and the heterodimerization domain VPS28 at the C-terminus (SEQ ID NO: 2). The corresponding second construct was designed by fusing the heterodimerization domain VPS36 to a linker at the C-terminus of CD3 epsilon (SEQ ID NO: 3). “TCAR2” comprises a pair of constructs. In the first construct, the CD19 scFv was cloned with CD8 hinge and transmembrane domain followed by the costimulatory domain 4-1BB and the heterodimerization domain mJUN at the C-terminus (SEQ ID NO: 4). The corresponding second construct was designed by fusing the heterodimerization domain mFos to a linker at the C-terminus of CD3 epsilon (SEQ ID NO:5).

CD19scFv-BBZ
(SEQ ID NO: 1)
GSATMALPVTALLLPLALLLHAARPEIVMTQSPATLSLSPGERATLSCRA
SQDISKYLNWYQQKPGQAPRLLIYHTSRLHSGIPARFSGSGSGTDYTLTI
SSLQPEDFAVYFCQQGNTLPYTFGQGTKLEIKGGGGSGGGGSGGGGSGGG
GSQVQLQESGPGLVKPSETLSLTCTVSGVSLPDYGVSWIRQPPGKGLEWI
GVIWGSETTYYNSSLKSRVTISKDNSKNQVSLKLSSVTAADTAVYYCAKH
YYYGGSYAMDYWGQGTLVTVSSTTTPAPRPPTPAPTIASQPLSLRPEACR
PAAGGAVHTRGLDFACDIYIWAPLAGTCGVLLLSLVITLYCKRGRKKLLY
IFKQPFMRPVQTTQEEDGCSCRFPEEEEGGCELRVKFSRSADAPAYKQGQ
NQLYNELNLGRREEYDVLDKRRGRDPEMGGKPRRKNPQEGLYNELQKDKM
AEAYSEIGMKGERRRGKGHDGLYQGLSTATKDTYDALHMQALPPR
CD19scFV-BB-VPS28
(SEQ ID NO: 2)
GSATMALPVTALLLPLALLLHAARPEIVMTQSPATLSLSPGERATLSCRA
SQDISKYLNWYQQKPGQAPRLLIYHTSRLHSGIPARFSGSGSGTDYTLTI
SSLQPEDFAVYFCQQGNTLPYTFGQGTKLEIKGGGGSGGGGSGGGGSQVQ
LQESGPGLVKPSETLSLTCTVSGVSLPDYGVSWIRQPPGKGLEWIGVIWG
SETTYYSSSLKSRVTISKDNSKNQVSLKLSSVTAADTAVYYCAKHYYYGG
SYAMDYWGQGTLVTVSSTTTPAPRPPTPAPTIASQPLSLRPEACRPAAGG
AVHTRGLDFACDIYIWAPLAGTCGVLLLSLVITLYCSLKRGRKKLLYIFK
QPFMRPVQTTQEEDGCSCRFPEEEEGGCELMFNAKYVAEATGNFITVMDA
LKLNYNAKDQLHPLLAELLISINRVTRDDFENRSKLIDWIVRINKLSIGD
TLTETQIRELLFDLELAYKSFYALLD
CD3e-VPS36
(SEQ ID NO: 3)
GSMQSGTHWRVLGLCLLSVGVWGQDGNEEMGGITQTPYKVSISGTTVILT
CPQYPGSEILWQHNDKNIGGDEDDKNIGSDEDHLSLKEFSELEQSGYYVC
YPRGSKPEDANFYLYLRARVCENCMEMDVMSVATIVIVDICITGGLLLLV
YYWSKNRKAKAKPVTRGAGAGGRQRGQNKERPPPVPNPDYEPIRKGQRDL
YSGLNQRRIGSGSGGSGSGGGSGSGSSGASADVVSTWVCPICMVSNETQG
EFTKDTLPTPICINCGVPADYELTKSSINCSNAIDPNANPRNQFG
CD19scFV-BB-mJUN
(SEQ ID NO: 4)
GSATMALPVTALLLPLALLLHAARPEIVMTQSPATLSLSPGERATLSCRA
SQDISKYLNWYQQKPGQAPRLLIYHTSRLHSGIPARFSGSGSGTDYTLTI
SSLQPEDFAVYFCQQGNTLPYTFGQGTKLEIKGGGGSGGGGSGGGGSQVQ
LQESGPGLVKPSETLSLTCTVSGVSLPDYGVSWIRQPPGKGLEWIGVIWG
SETTYYSSSLKSRVTISKDNSKNQVSLKLSSVTAADTAVYYCAKHYYYGG
SYAMDYWGQGTLVTVSSTTTPAPRPPTPAPTIASQPLSLRPEACRPAAGG
AVHTRGLDFACDIYIWAPLAGTCGVLLLSLVITLYCSLKRGRKKLLYIFK
QPFMRPVQTTQEEDGCSCRFPEEEEGGCELRIARLEEEVKTLEAQNSELA
STANMLEEQVAQLKQKV
CD3e-mFos
(SEQ ID NO: 5)
GSMQSGTHWRVLGLCLLSVGVWGQDGNEEMGGITQTPYKVSISGTTVILT
CPQYPGSEILWQHNDKNIGGDEDDKNIGSDEDHLSLKEFSELEQSGYYVC
YPRGSKPEDANFYLYLRARVCENCMEMDVMSVATIVIVDICITGGLLLLV
YYWSKNRKAKAKPVTRGAGAGGRQRGQNKERPPPVPNPDYEPIRKGQRDL
YSGLNQRRIGSGSGGSLTDTLQAKTDQLKDEKSALQTKIANLLKEKEKLE
FIL

Generation of Jurkat Reporter Cell Line for Initial Characterization of CAR Function

As an alternative to primary T cell transduction and activation, a Jurkat-NFAT reporter cell line can be used to evaluate the functional activity of CAR constructs. The Jurkat T cell line (E6-1) was transfected with a NFAT-luciferase reporter construct and a stable, clonal cell line Jurket cells with NFAT-LUC reporter (JNL), was selected for further characterization based on strong induction of the NFAT reporter following PMA and ionomycin stimulation.

Transfection of Jurkat Reporter Cell Line and Activation of NFAT.

Jurkat cells with NFAT-LUC reporter (JNL) were grown to the density of 0.5-1.0×10⁶/ml in RPMI-1640 media containing 2 mM glutamine and 10% fetal bovine serum with puromycin at 0.5 μg/ml. For each transfection 2.0×10⁶ cells were spin down at 100 g for 10 minutes. 1 μg DNA each for co-transfection or 2 μg for single transfection of the control CAR were used per transfection. Amaxa Nucleofector solution V and supplement I was mixed and 100 μl was added into the tube with DNA construct. The mixture was then added to the cells and transferred to the electroporation cuvette. Electroporation was done under setting X-001 using Amaxa Nucleofector II Device. 0.4 ml of RPMI-1640 media containing 2 mM glutamine and 10% FBS was added immediately after electroporation and the mixture was transferred into 0.25 ml growth media in one well of the 6-well plate and allowed to recover for at least 3 hours. During cell recovery, white solid bottom tissue culture treated plates were coated with either anti-CD19 idiotype antibody or irrelevant human IgG1-Fc negative control for 2 hours followed by blocking with 5% BSA in FBS for 30 minutes at 37° C., 5% CO₂. The blocking buffer was then aspirated. 100 μL of each of the transfected Jurkat constructs was plated in triplicate. After an overnight incubation 100 μL of One-Glo Luciferase (Promega) reagent was added to each well. To determine the relative-fold activation of the anti-idiotype wells to the negative control wells, the plate was then incubated for 5 min to allow for equilibrium of the luciferase signal and read using an Envision multilabel reader.

IL-2 Expression in Transfected Jurkat (JNL) Cells

Transfection of JNL cells and activation was performed as described above in the JNL RGA assay excepting incubation which was for 40-48 hours at 37° C., 5% CO₂. Supernatant was collected from the cells by centrifuging at 300×g for 10 minutes. Levels of IL-2 expression were measured using Mesoscale Discovery Human IL-2 kit (Mesoscale). All provided reagents were prepared according to manufacturer's instructions. 50 μL of collected supernatant (neat) and prepared standard were added to the pre-coated MSD plate and incubated at room temperature with shaking for two hours. The plate was washed 3× with 300 μL PBS+Tween 20 and 25 μL detection antibody solution was added to each well. The plate was incubated again at room temperature with shaking for 2 hours and washed with the previous conditions and 150 μL 2× Read Buffer T was added to each well. The plate was immediately read on the MSD instrument for human IL-2 levels.

Transient Expression Results

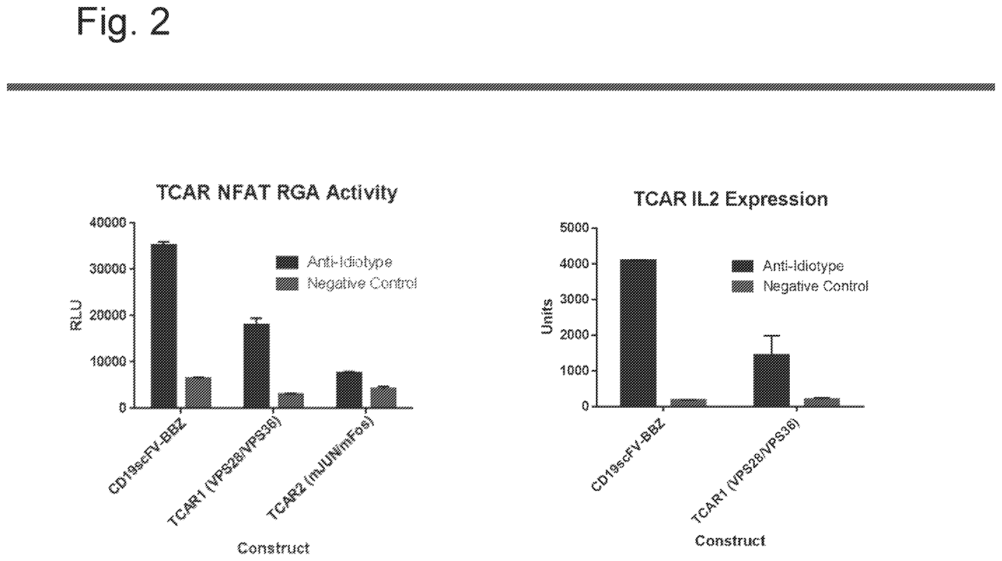

Transient transfection via electroporation of JNL cells of TCAR1 and TCAR2 demonstrated antigen-dependent signaling in the reporter gene assay as shown in FIG. 2. TCAR1, VPS28/VPS36-based heterodimers, demonstrated similar fold over background activation compared to the positive control CAR, CD19scFV-BBZ in the RGA assay. Expression of IL2 after 40 hours of activation was also evaluated. Antigen dependent IL2 expression was also observed for TCAR1; due to the low intrinsic signal in the JNL RGA assay, TCAR2 was not assessed. Further enhancements in signaling and IL2 expression would be expected by optimizing the orientation of the heterodimerization domains via linker length, enhancing the affinity of the heterodimerization domains to one another and/or enhancing the affinity of the CD3 epsilon interface to the remainder of the TCR complex.

Production of Lentiviral Transduced Primary Human T-Cells

TCAR1 was also evaluated in vitro using primary human T-cells produced via lentiviral transduction in comparison to CD19scFv-BBZ.

Lentivirus Production

Lenti-X 293T cells (Clontech), grown in DMEM supplemented with 10% FBS and Non-essential amino acids were co-transfected with lentiviral vector plasmids along with the pRSV.rev, pMDL.g/p.rre and pVSVg packaging plasmids using Lipofectamine 2000 (Invitrogen) transfection reagent. Lentivirus vector containing supernatants were harvested 48 hours after transfection, and concentrated using Lenti-X Concentrator (Clontech) and centrifugation at 1,500×g for 45 minutes. Concentrated vector was stored at −80 C until further use.

Lentivirus vector titers were determined using limited dilution on Sup-T1 cells (ATCC) cultured in RPMI-1640 supplemented with 10% FBS. Vectors were 3-fold serial diluted then 50 uL of diluted vector was added to a flat bottom microtiter plate containing Sup-T1 cells. After 72 hours cells were harvested and analyzed via FACS using Protein-L for scFv expression. The titer in transducing units per mL (TU/mL) was calculated from the vector dilution in which percent positive expression in Sup-T1 cells was less than 20% but greater than 5% using the following equation:

TU/mL=(% Positive/100)×2E⁴×dilution factor×20

T Cell Isolation and Viral Transduction into Primary T Cells

Normal donor T cells were isolated via MACS negative selection (Miltenyi pan T cell isolation kit) from human PBMC obtained from Cellular Technology Limited. Purified T cells were cultured in RPMI supplemented with 10% FBS, 100 U/ml penicillin, 100 μg/ml streptomycin, 10 mM HEPES and 1 mM non-essential amino acids and activated with Dynabeads human T-activator CD3/CD28 beads (Invitrogen) at a bead to cell ratio of 3:1. After 18-24 hours of activation T cells were transduced with lentiviral vectors at a multiplicity of infection (MOI) of 5. Transduced T cells were expanded every 2-3 days for 10 days maintaining a cell density of −0.75 million per mL. Cells were aliquoted and cryogenically frozen.

Cytotoxicity and IL2 Assay

Transduced T cells were analyzed for their ability to kill target expressing cell lines as well as secretion of the cytokine IL2; used as a surrogate for proliferation. The target expressing cell lines Nalm6 (CD19), cultured in RPMI with 10% FBS and K562 (negative control), cultured in IMDM with 10% FBS, were all engineered to stably express firefly luciferase under puromycin selection. Briefly, thawed transduced T cells were analyzed via FACS for percent CAR expression. All constructs were normalized to 10% CAR positive expression by diluting with isolated, expanded, freeze/thawed untransduced T cells. Transduced normalized T cells were then cultured in 200 μL of media at various effector to target ratios, holding target cells constant at 2.5E⁴ cells/well. Target cells were plated alone without the presence of effector cells to determine maximum luminescence. After 18-20 hours 100 μL of culture supernatant was removed for subsequent IL2 analysis and 100 μL of OneGlo (Promega) luciferase substrate was added to the remaining supernatant and cells. Luminescence was measured on an Envison plate reader after a 10 minute incubation. Percent specific lysis was calculated using the following equation:

Specific lysis (%)=(1−(sample luminescence/average maximum1 luminescence))*100

The harvested supernatant was analyzed for the amount of the IL2 via MSD ELISA following the manufacturer's instructions.

Primary Human T-Cell Results

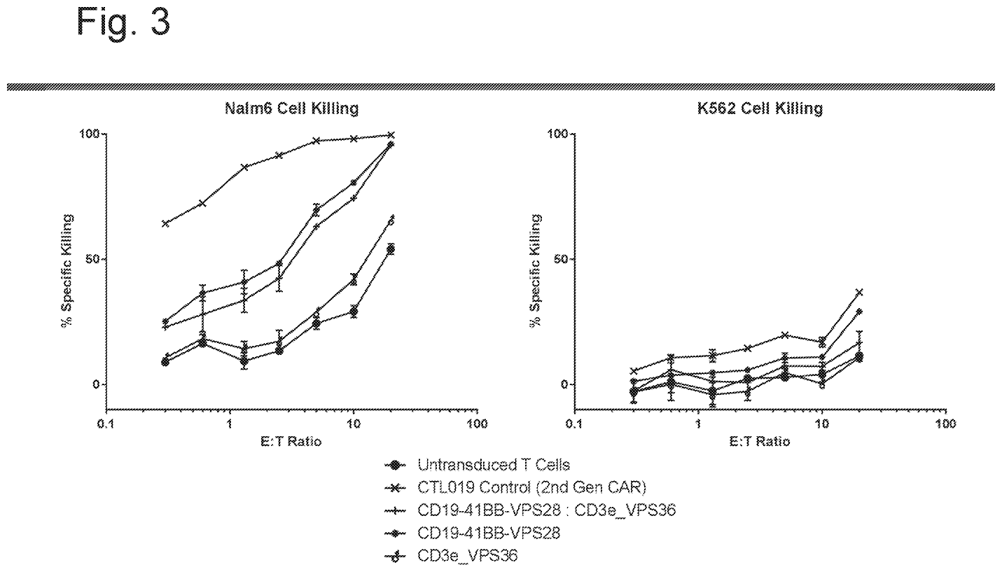

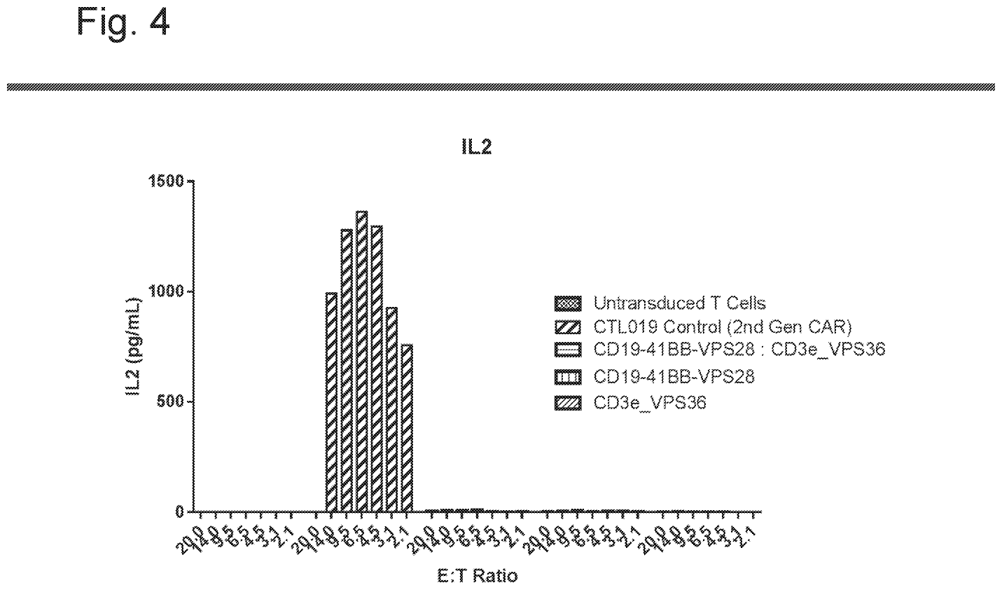

FIG. 3 and FIG. 4 show the functional activity of “TCAR1” relative to the control CD19scFv-BBZ. As can be observed from both the redirected lysis assay and the IL2 expression results, TCAR1 demonstrated reduced functionality. It is not clear if both constructs were expressed in the T-cells under the transduction conditions; further optimizations may be needed to ensure simultaneous expression of both constructs containing both hetoerdimerization domains in the same cell. Additionally, further enhancements may be needed by optimizing the orientation of the heterodimerization domains via linker length or enhancing the affinity of the heterodimerization domains to one another. However, transient signaling results demonstrated the potential for these types of chimeric antigen receptors.

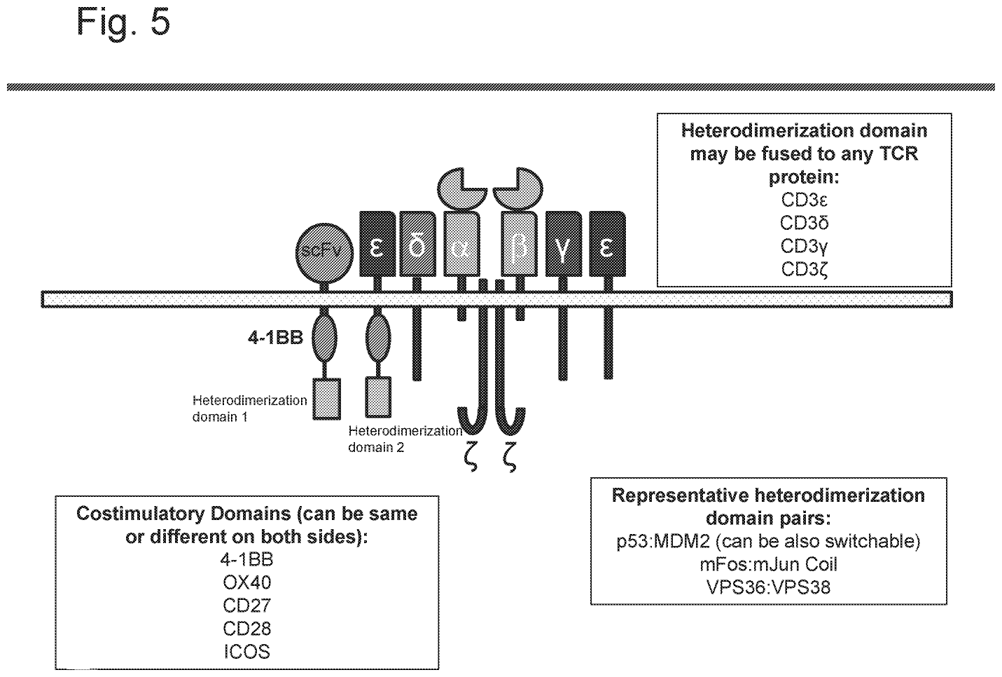

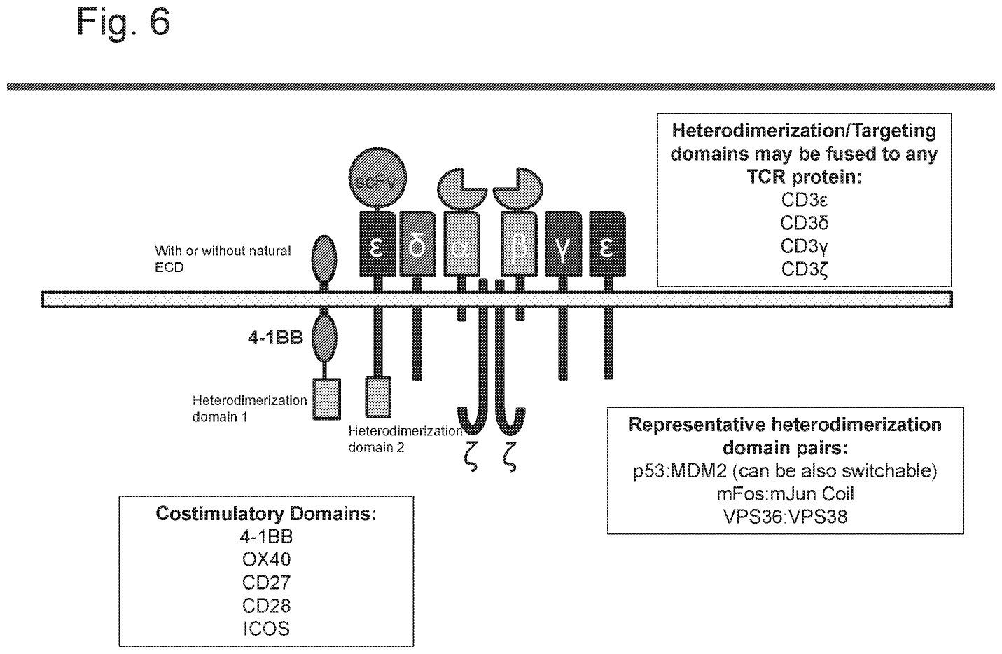

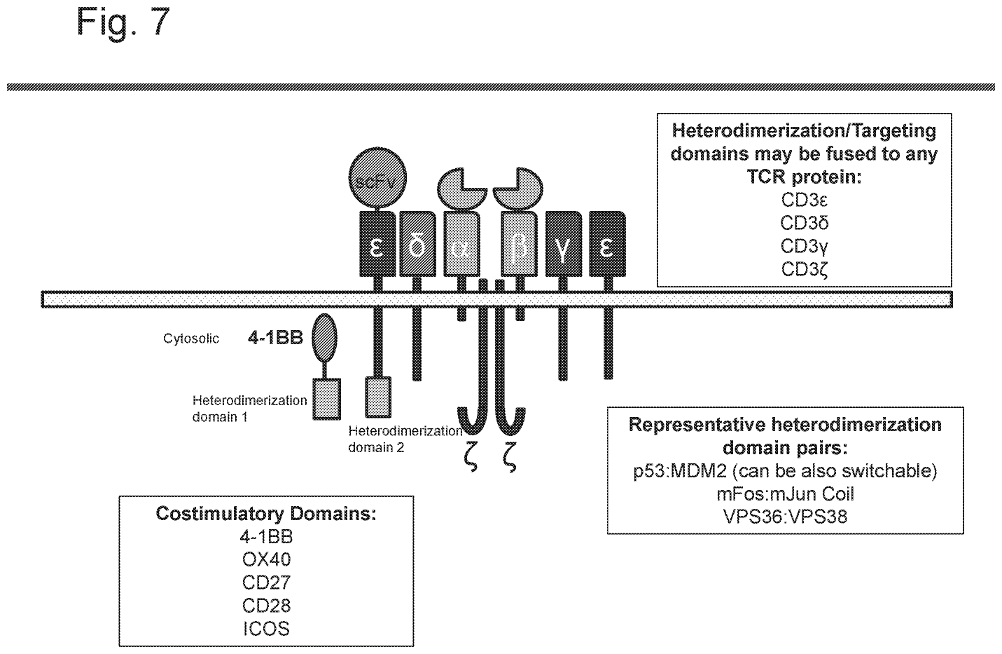

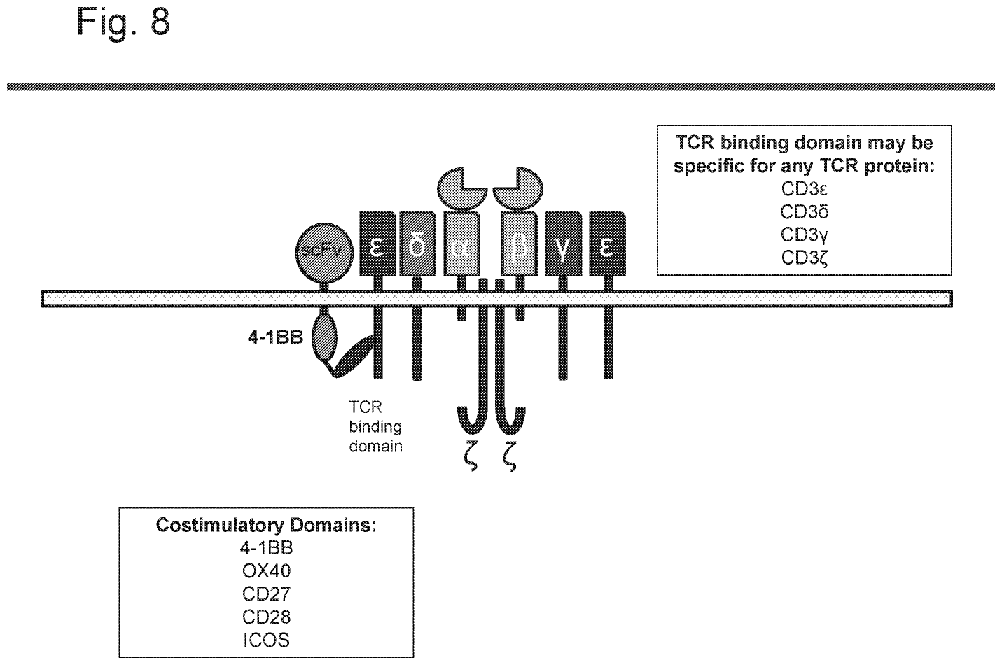

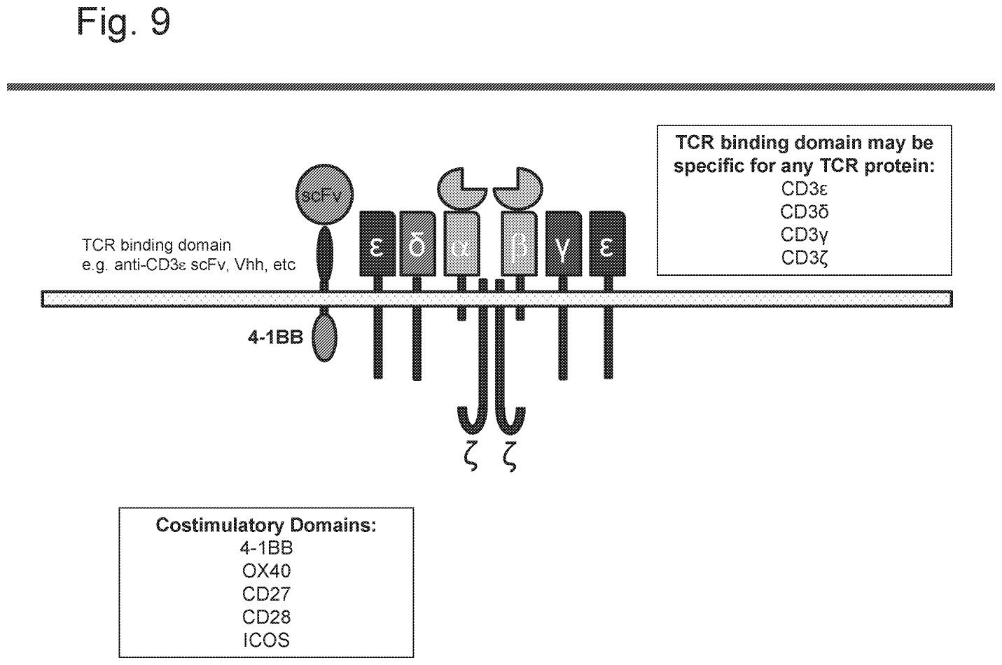

### Example 2: Constitutively Active TCARs with Enhanced Proliferation Using Intracellular Heterodimerization Domains (FIG. 5-9)

Synthesis of Constitutively Active TCAR constructs with multiple costimulatory domains Pairs of plasmid DNA will be synthesized externally by DNA2.0. The nominal non-regulatable CAR construct, CD19scFv-BBZ, SEQ ID NO: 1, will be used as a control. “TCAR1” comprises a pair of constructs. In the first construct, the CD19 scFv was cloned with CD8 hinge and transmembrane domain followed by the costimulatory domain 4-1BB and the heterodimerization domain VPS28 at the C-terminus (SEQ ID NO: 2). The corresponding second construct was designed as above by fusing the heterodimerization domain VPS36 to a linker at the C-terminus of CD3 epsilon (SEQ ID NO:3). “TCAR3” (FIG. 5) comprises a pair of constructs. In the first construct, the CD19 scFv will be cloned with CD8 hinge and transmembrane domain followed by the costimulatory domain 4-1BB and the heterodimerization domain VPS28 at the C-terminus (SEQ ID NO: 2). The corresponding second construct will be designed by fusing the intracellular costimulatory domain of CD28 followed by VPS36 to the C-terminus of CD3 epsilon extracellular and transmembrane domains (SEQ ID NO:6).

CD3eECDTM-CD28-VPS36
(SEQ ID NO: 6)
GSMQSGTHWRVLGLCLLSVGVWGQDGNEEMGGITQTPYKVSISGTTVILT
CPQYPGSEILWQHNDKNIGGDEDDKNIGSDEDHLSLKEFSELEQSGYYVC
YPRGSKPEDANFYLYLRARVCENCMEMDVMSVATIVIVDICITGGLLLLV
YYWSRSKRSRLLHSDYMNMTPRRPGPTRKHYQPYAPPRDFAAYRSGSGSG
GSGSGGGSGSGSSGASADVVSTWVCPICMVSNETQGEFTKDTLPTPICIN
CGVPADYELTKSSINCSNAIDPNANPRNQFG

Transfection of Jurkat Reporter Cell Line and Activation of NFAT.

Activation following target antigen engagement of the antigen binding domain will be measured with the Jurkat cells with NFAT-LUC reporter (JNL) reporter cell line as described in Example 1. The transfected cells will be added to the target plate with 100 μl per well. Luciferase One Glo reagent 100 μl will be added per well. The samples will be incubated for 5 min and then luminescence will be measured as described.

IL-2 expression in transfected Jurkat (JNL) Cells

Transfection of JNL cells and activation will be performed as described above in the JNL RGA assay excepting incubation which will be for 40-48 hours at 37° C., 5% CO₂. Measurement of secreted IL2 will be performed as described in Example 1.

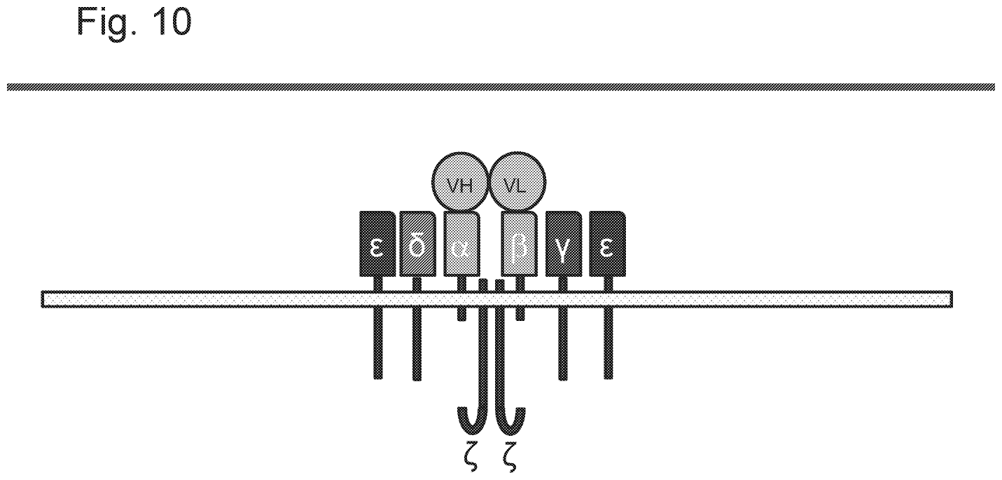

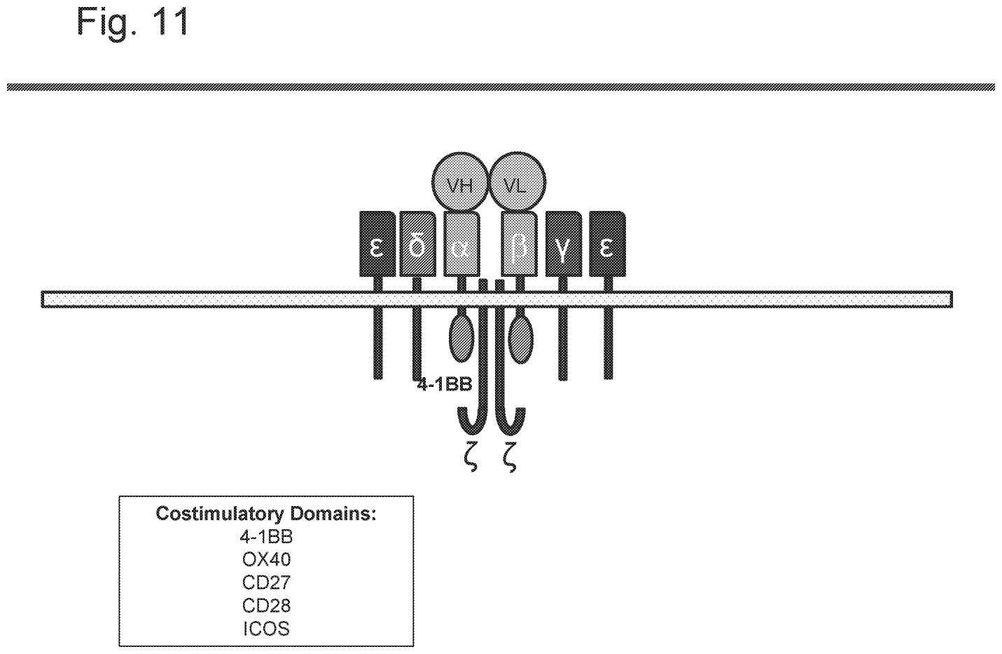

### Example 3: Constitutively Active TCARs Fused into the TCR Complex Via CD3 Epsilon (fusTCAR) (FIGS. 10-11)

Transient Expression and Activation Assays

Synthesis of fusTCAR Constructs

Plasmid DNA were synthesized externally by DNA2.0. The nominal non-regulatable CAR construct, CD19scFv-BBZ, SEQ ID NO: 1, was used as a control. For the fusTCAR, the targeting domain can be fused directly to different members of TCR complex with or without additional intracellular co-stimulatory and signaling domains.

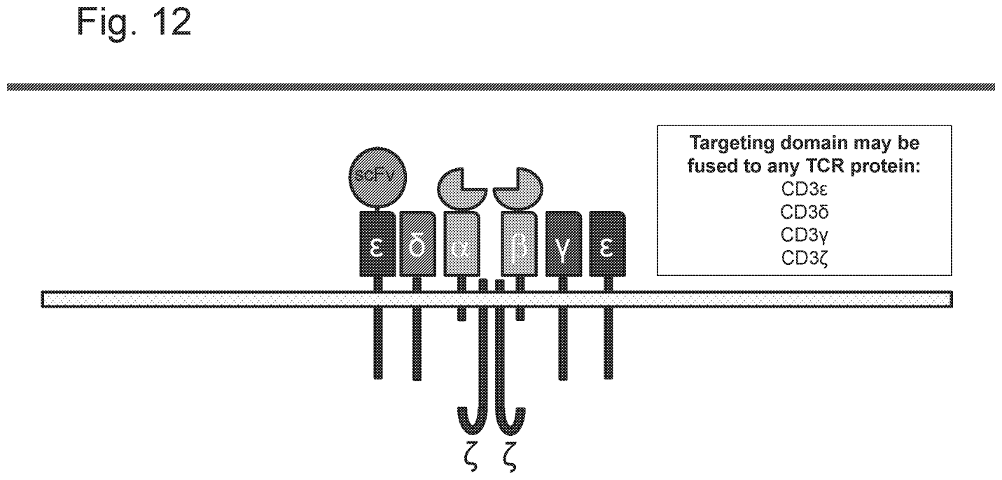

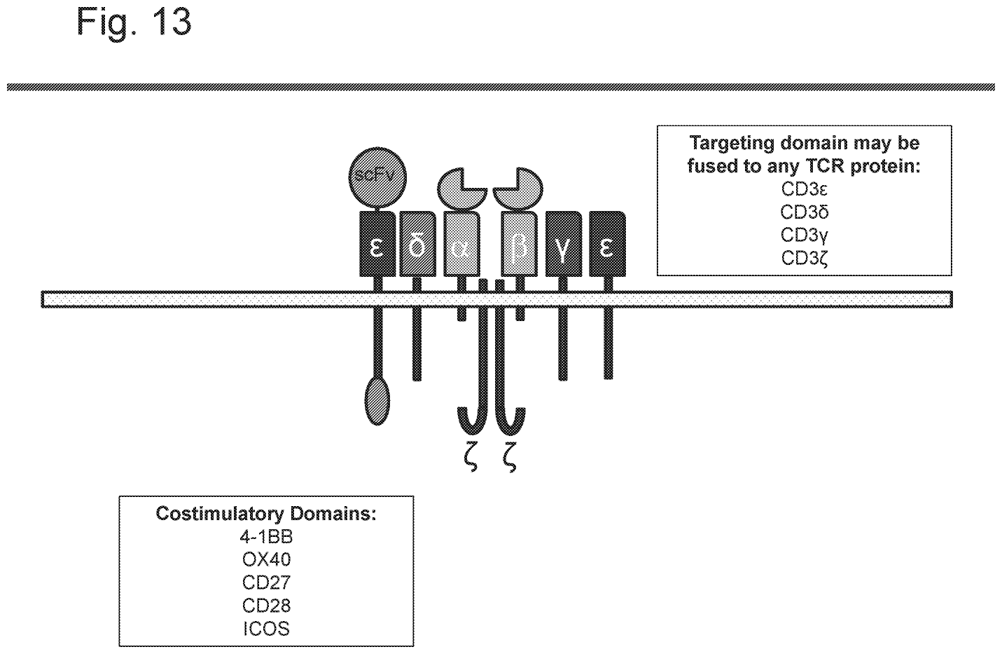

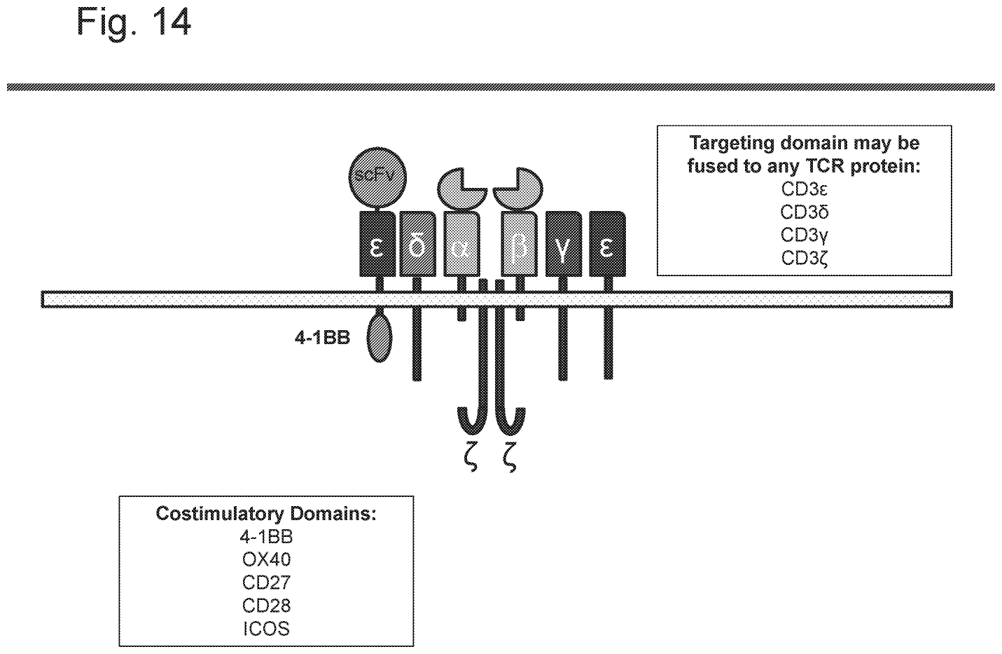

In “fusTCAR1” (FIG. 12) the CD19 scFv was cloned as an N-terminal fusion to the complete CD3 epsilon protein (SEQ ID NO: 7). “FusTCAR2” (FIG. 13) was cloned as an N-terminal fusion to the complete CD3 epsilon protein followed by a C-terminal fusion of the intracellular costimulatory domain of 4-1BB (SEQ ID NO: 8) “FusTCAR3” (FIG. 14) lacks internal endogenous ITAM domains. CD19 scFv was cloned onto the N-terminus of the CD3 extracellular and transmembrane domains followed by the intracellular costimulatory domain 4-1BB (SEQ ID NO: 9).

CD19scFv-CD3e
(Seq ID NO: 7)
GSATMALPVTALLLPLALLLHAARPEIVMTQSPATLSLSPGERATLSCRA
SQDISKYLNWYQQKPGQAPRLLIYHTSRLHSGIPARFSGSGSGTDYTLTI
SSLQPEDFAVYFCQQGNTLPYTFGQGTKLEIKGGGGSGGGGSGGGGSQVQ
LQESGPGLVKPSETLSLTCTVSGVSLPDYGVSWIRQPPGKGLEWIGVIWG
SETTYYSSSLKSRVTISKDNSKNQVSLKLSSVTAADTAVYYCAKHYYYGG
SYAMDYWGQGTLVTVSSGGGGSDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI
CD19scFv-CD3e-41BB
(Seq ID NO: 8)
GSATMALPVTALLLPLALLLHAARPEIVMTQSPATLSLSPGERATLSCRA
SQDISKYLNWYQQKPGQAPRLLIYHTSRLHSGIPARFSGSGSGTDYTLTI
SSLQPEDFAVYFCQQGNTLPYTFGQGTKLEIKGGGGSGGGGSGGGGSQVQ
LQESGPGLVKPSETLSLTCTVSGVSLPDYGVSWIRQPPGKGLEWIGVIWG
SETTYYSSSLKSRVTISKDNSKNQVSLKLSSVTAADTAVYYCAKHYYYGG
SYAMDYWGQGTLVTVSSGGGGSDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRIGSGSGGSKRGRKKLLYIFKQPFMRPVQTTQEEDGCSCRFPEEE
EGGCEL
CD19scFv-CD3eECDTM-41BB
(Seq ID NO: 9)
GSATMALPVTALLLPLALLLHAARPEIVMTQSPATLSLSPGERATLSCRA
SQDISKYLNWYQQKPGQAPRLLIYHTSRLHSGIPARFSGSGSGTDYTLTI
SSLQPEDFAVYFCQQGNTLPYTFGQGTKLEIKGGGGSGGGGSGGGGSQVQ
LQESGPGLVKPSETLSLTCTVSGVSLPDYGVSWIRQPPGKGLEWIGVIWG
SETTYYSSSLKSRVTISKDNSKNQVSLKLSSVTAADTAVYYCAKHYYYGG
SYAMDYWGQGTLVTVSSGGGGSDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKRGRKKLLYIFKQPFMRPVQTTQEEDGCSCRFPEEEEGGCEL

Transfection of Jurkat reporter cell line and activation of NFAT.

Activation following target antigen engagement of the antigen binding domain was measured with the Jurkat cells with NFAT-LUC reporter (JNL) reporter cell line as described in Example 1. The transfected cells was added to the target plate with 100 μl per well. Luciferase One Glo reagent 100 μl was added per well. The samples were incubated for 5 min and then luminescence was measured as described.

IL-2 Expression in Transfected Jurkat (JNL) Cells

Transfection of JNL cells and activation was performed as described above in the JNL RGA assay excepting incubation which was for 40-48 hours at 37° C., 5% CO₂. Measurement of antigen-dependent IL2 expression was performed as described in Example 1.

Transient Expression Results

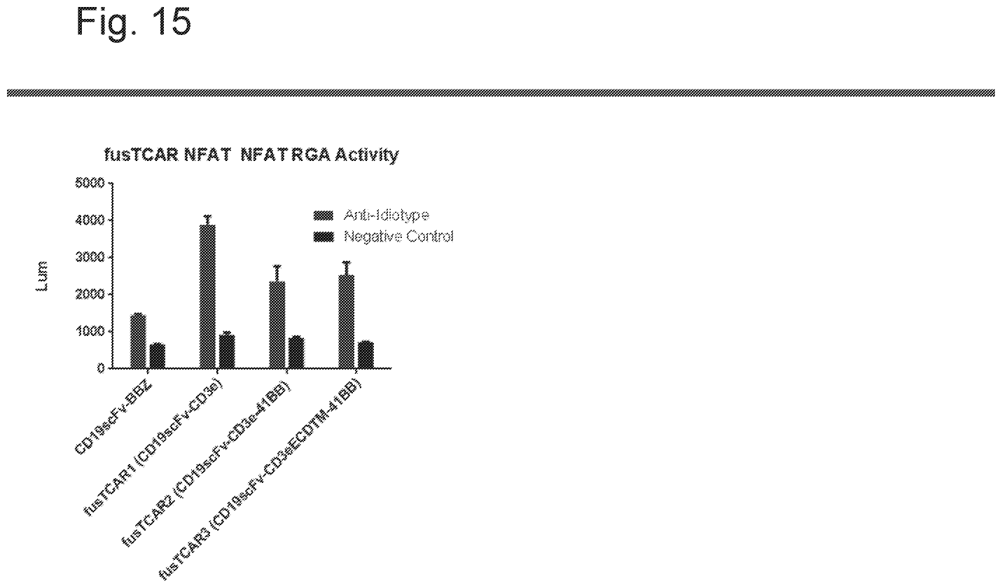

Initial screening of fusTCAR1, fusTCAR2 and fusTCAR3 via transient transfection into JNL cells demonstrated antigen-dependent signaling as shown in FIG. 15. Importantly, signaling was still observed with fusTCAR3, which was truncated and lacked the ITAM signaling domain of CD3 epsilon. Transfection of a construct containing ITAM signaling domains is thus not a prerequisite for activity of fusTCARs. By associating the targeting domain with the TCR complex, signaling is mediated through all members of the complex and is not exclusively limited to that derived from a signaling domain fused to the targeting domain.

Production of Lentiviral Transduced Primary Human T-Cells

FusTCARs were also tested in primary human T-Cells for their activity. Prior to production of lentivirus additional constructs were also designed to confirm the dependence of functional ITAMS for in vitro activity of traditional CAR constructs and the independent activity for TCARs regardless of the presence or absence of functional ITAMS. Plasmid DNA were synthesized externally by DNA2.0. The first generation CAR design construct, CD19scFv-Zeta, SEQ ID No: 10, was synthesized; CD19 scFv was cloned as a N-terminal fusion to the CD8a linker and transmembrane domain followed by the intracellular signaling domain CD3zeta. A second construct (SEQ ID NO: 11) was similarly cloned, excepting that all intracellular tyrosine residues within CD3 zeta annotated to be phosphorylated were switched to phenylalanine in order to abbrogate intracellular phosphotyrosine signaling. As a combination of a intracellular costimulatory domain with intracellular signaling domain has previously been demonstrated to be beneficial for typical CAR constructs, a final construct was cloned whereby CD19 scFv was a N-terminal fusion to the CD8a linker and transmembrane domain followed by 4-1BB; no CD3 zeta signaling domain was included in this construct (SEQ ID NO: 12). Finally, an analogous fusTCAR was synthesized to CD19scFV-Zeta_7 YtoF. “FusTCAR4” lacks internal endogenous ITAM domains. CD19scFv, SEQ ID NO: 13 was cloned as a N-terminal fusion to the complete CD3 epsilon protein except those tyrosines annotated to be phosphorylated were mutated to phenylalanine rendering the instrinic signaling pathways associated with CD3 epsilon ITAMs inactive.

CD19scFv-Zeta
(SEQ ID NO: 10)
GSMALPVTALLLPLALLLHAARPEIVMTQSPATLSLSPGERATLSCRASQ
DISKYLNWYQQKPGQAPRLLIYHTSRLHSGIPARFSGSGSGTDYTLTISS
LQPEDFAVYFCQQGNTLPYTFGQGTKLEIKGGGGSGGGGSGGGGSGGGGS
QVQLQESGPGLVKPSETLSLTCTVSGVSLPDYGVSWIRQPPGKGLEWIGV
IWGSETTYYNSSLKSRVTISKDNSKNQVSLKLSSVTAADTAVYYCAKHYY
YGGSYAMDYWGQGTLVTVSSTTTPAPRPPTPAPTIASQPLSLRPEACRPA
AGGAVHTRGLDFACDIYIWAPLAGTCGVLLLSLVITLYCRVKFSRSADAP
AYKQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPP
R
CD19scFv-Zeta_7YtoF
(SEQ ID NO: 11)
GSMALPVTALLLPLALLLHAARPEIVMTQSPATLSLSPGERATLSCRASQ
DISKYLNWYQQKPGQAPRLLIYHTSRLHSGIPARFSGSGSGTDYTLTISS
LQPEDFAVYFCQQGNTLPYTFGQGTKLEIKGGGGSGGGGSGGGGSGGGGS
QVQLQESGPGLVKPSETLSLTCTVSGVSLPDYGVSWIRQPPGKGLEWIGV
IWGSETTYYNSSLKSRVTISKDNSKNQVSLKLSSVTAADTAVYYCAKHYY
YGGSYAMDYWGQGTLVTVSSTTTPAPRPPTPAPTIASQPLSLRPEACRPA
AGGAVHTRGLDFACDIYIWAPLAGTCGVLLLSLVITLYCRVKFSRSADAP
AFKQGQNQLFNELNLGRREEFDVLDKRRGRDPEMGGKPRRKNPQEGLFNE
LQKDKMAEAFSEIGMKGERRRGKGHDGLFQGLSTATKDTFDALHMQALPP
R
CD19scFv-BB
(SEQ ID NO: 12)
GSMALPVTALLLPLALLLHAARPEIVMTQSPATLSLSPGERATLSCRASQ
DISKYLNWYQQKPGQAPRLLIYHTSRLHSGIPARFSGSGSGTDYTLTISS
LQPEDFAVYFCQQGNTLPYTFGQGTKLEIKGGGGSGGGGSGGGGSGGGGS
QVQLQESGPGLVKPSETLSLTCTVSGVSLPDYGVSWIRQPPGKGLEWIGV
IWGSETTYYNSSLKSRVTISKDNSKNQVSLKLSSVTAADTAVYYCAKHYY
YGGSYAMDYWGQGTLVTVSSTTTPAPRPPTPAPTIASQPLSLRPEACRPA
AGGAVHTRGLDFACDIYIWAPLAGTCGVLLLSLVITLYCKRGRKKLLYIF
KQPFMRPVQTTQEEDGCSCRFPEEEEGGCEL
CD19scFv-CD3e_2YtoF
(SEQ ID NO: 13)
GSMALPVTALLLPLALLLHAARPEIVMTQSPATLSLSPGERATLSCRASQ
DISKYLNWYQQKPGQAPRLLIYHTSRLHSGIPARFSGSGSGTDYTLTISS
LQPEDFAVYFCQQGNTLPYTFGQGTKLEIKGGGGSGGGGSGGGGSQVQLQ
ESGPGLVKPSETLSLTCTVSGVSLPDYGVSWIRQPPGKGLEWIGVIWGSE
TTYYSSSLKSRVTISKDNSKNQVSLKLSSVTAADTAVYYCAKHYYYGGSY
AMDYWGQGTLVTVSSGGGGSDGNEEMGGITQTPYKVSISGTTVILTCPQY
PGSEILWQHNDKNIGGDEDDKNIGSDEDHLSLKEFSELEQSGYYVCYPRG
SKPEDANFYLYLRARVCENCMEMDVMSVATIVIVDICITGGLLLLVYYWS
KNRKAKAKPVTRGAGAGGRQRGQNKERPPPVPNPDFEPIRKGQRDLFSGL
NQRRI

Lentivirus Production and Viral Transduction into Primary T Cells

As described in Example 1, lentivirus were produced and transduced into isolated primary human T-cells. Transduced T-cells and non-transduced control T-cells were expanded and frozen for subsequent analysis.

Cytotoxicity and IL2 Assay

Cytotoxicity and IL2 production induced by cross-linking primary human T-Cells to target tumor cells were assessed as described in Example 1.

Primary Human T-Cell Results

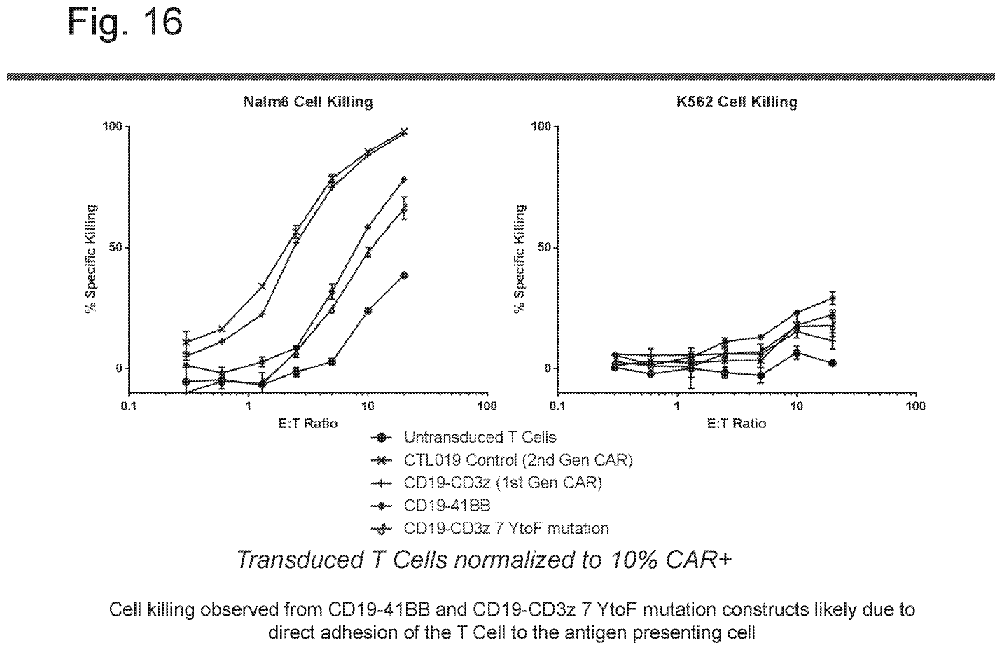

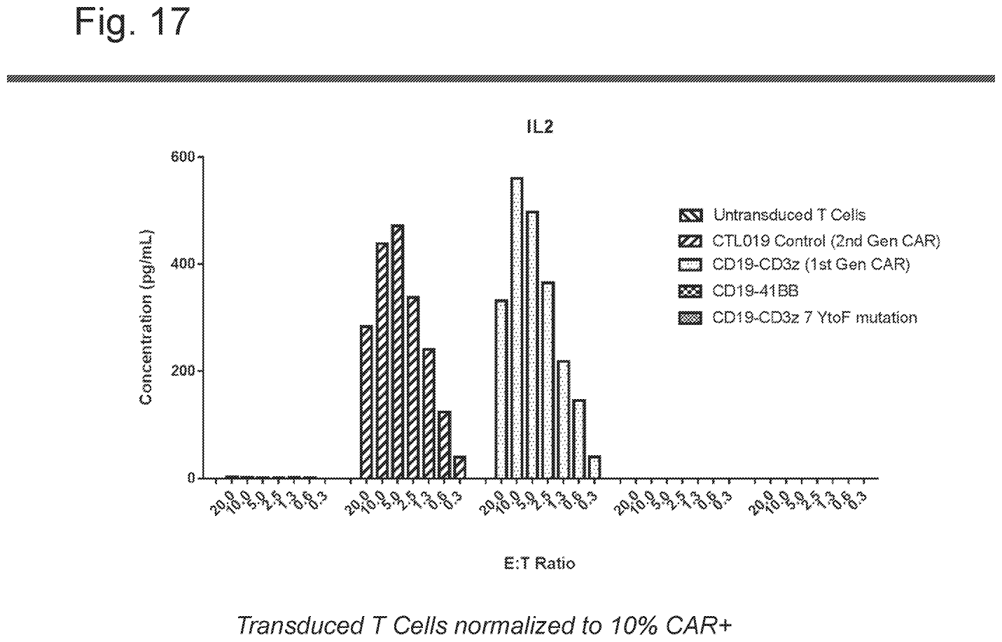

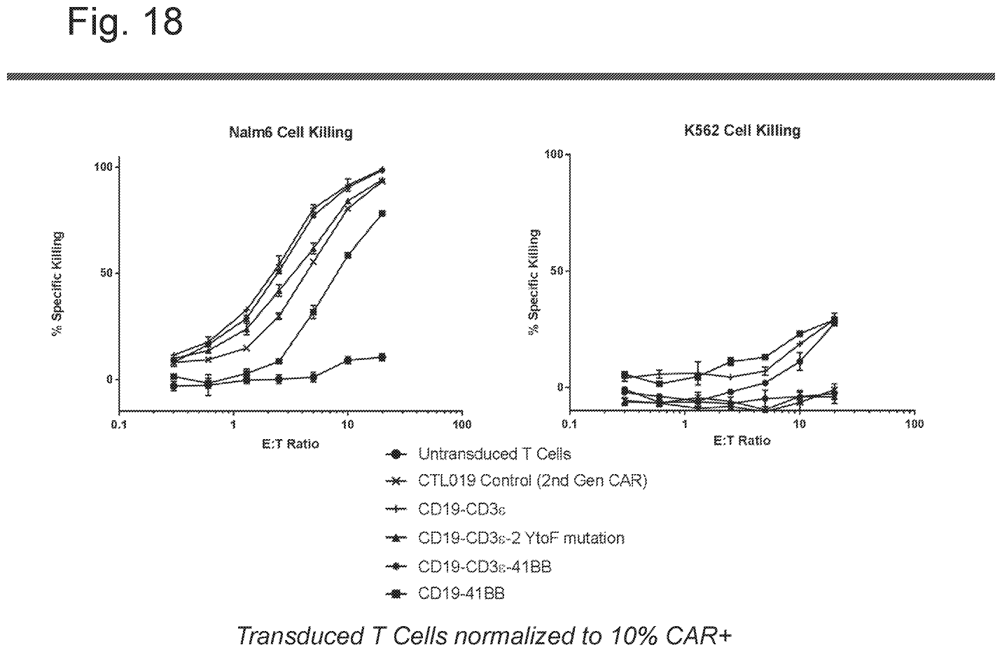

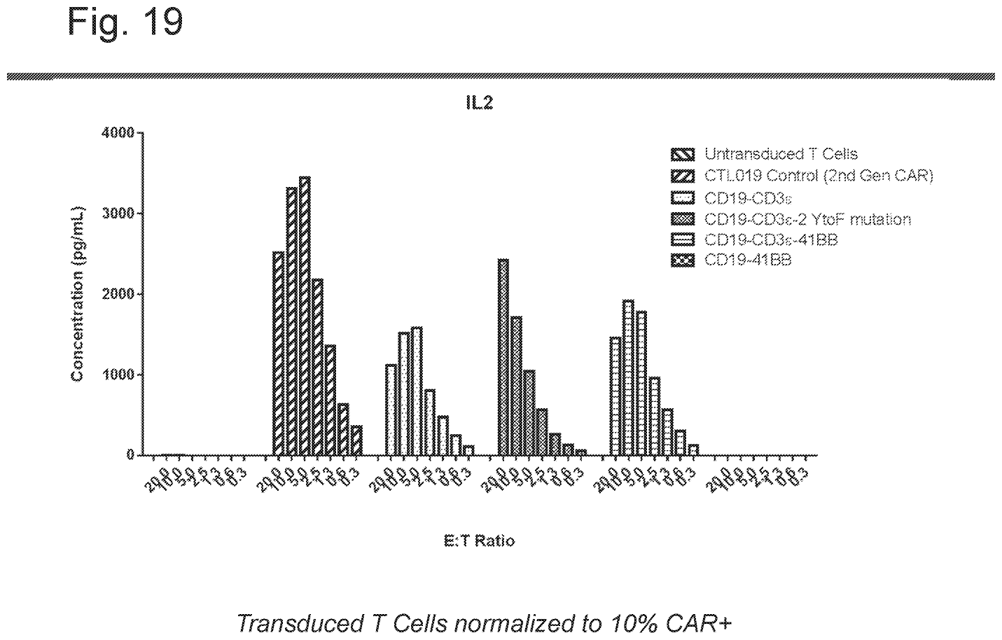

As can be observed in FIG. 16 and FIG. 17, traditional CARs require functional endogenous ITAM signaling domains in order to induce maximal T-cell redirected lysis of the target cells and IL2 production. Constructs in which CD3 zeta was replaced with 41BB or were mutated to inactivate the ITAMS in CD3 zeta were deficient in both elements. In contrast, FIG. 18 and FIG. 19 demonstrate that fusTCAR activity is both specific and independent of endogenous functional ITAMs. Redirected lytic activity and IL2 secretion was observed regardless of the presence or absence or order of costimulatory domains. Furthermore, neither mutation of the key tyrosines involved in signaling nor complete removal of the intracellular domain of CD3 epsilon resulted in appreciable loss in functional in vitro activity of the TCARs.

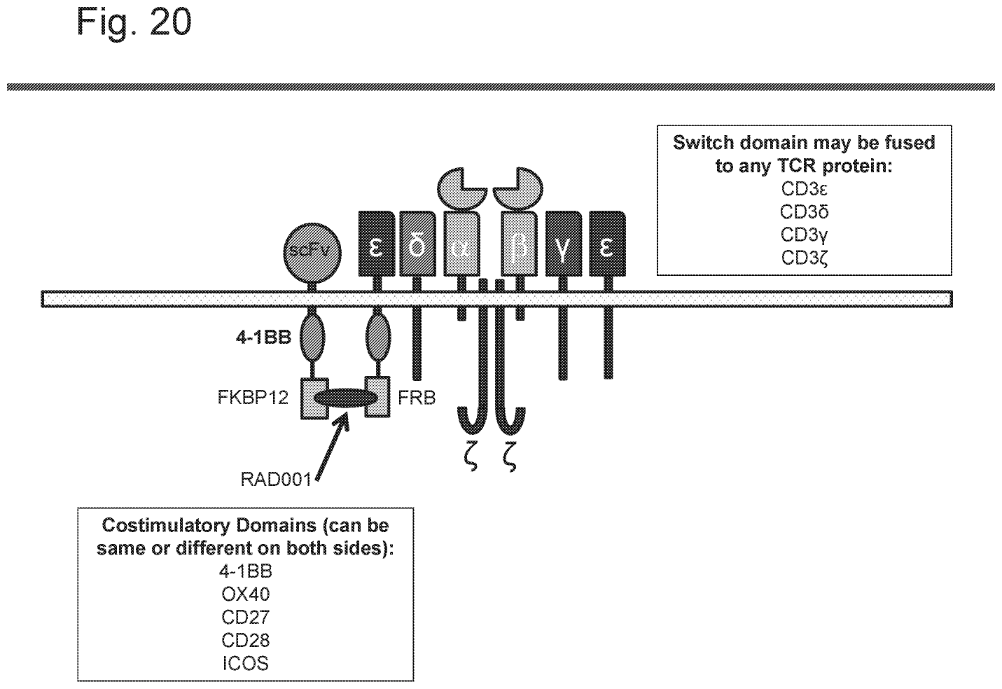

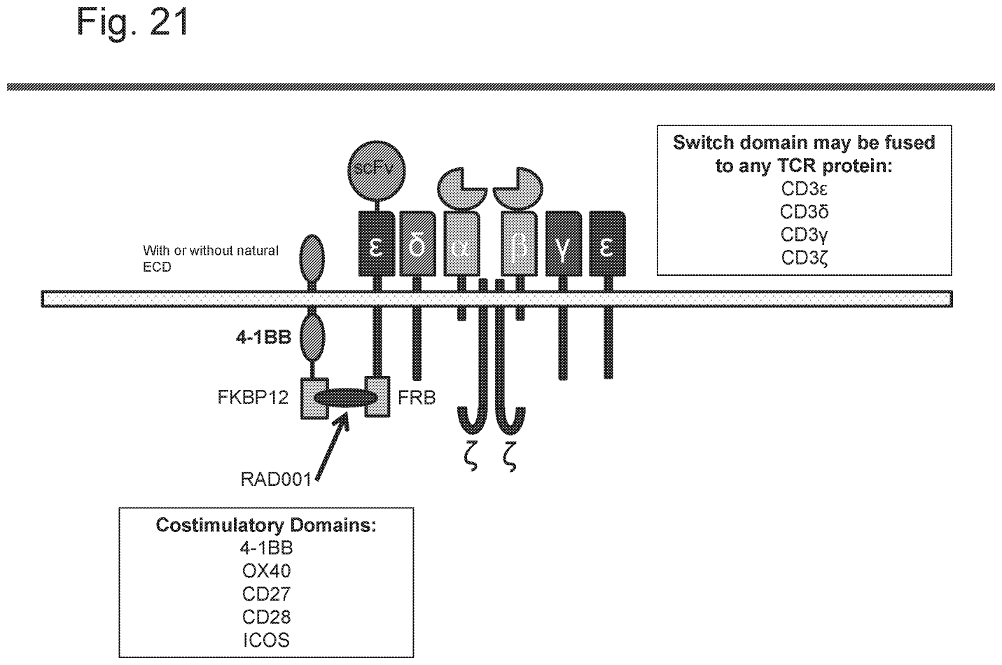

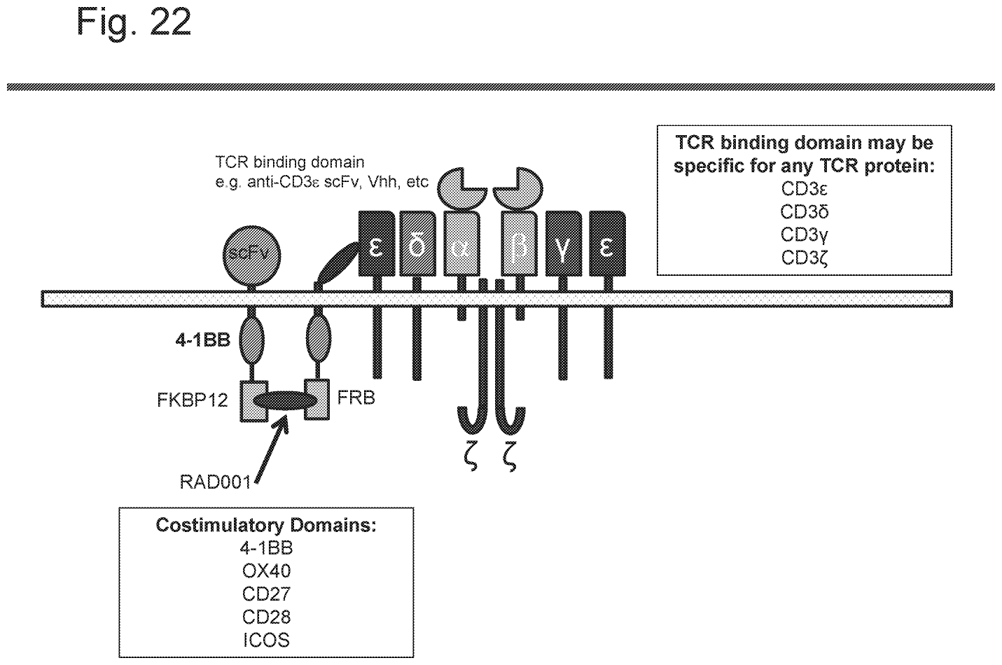

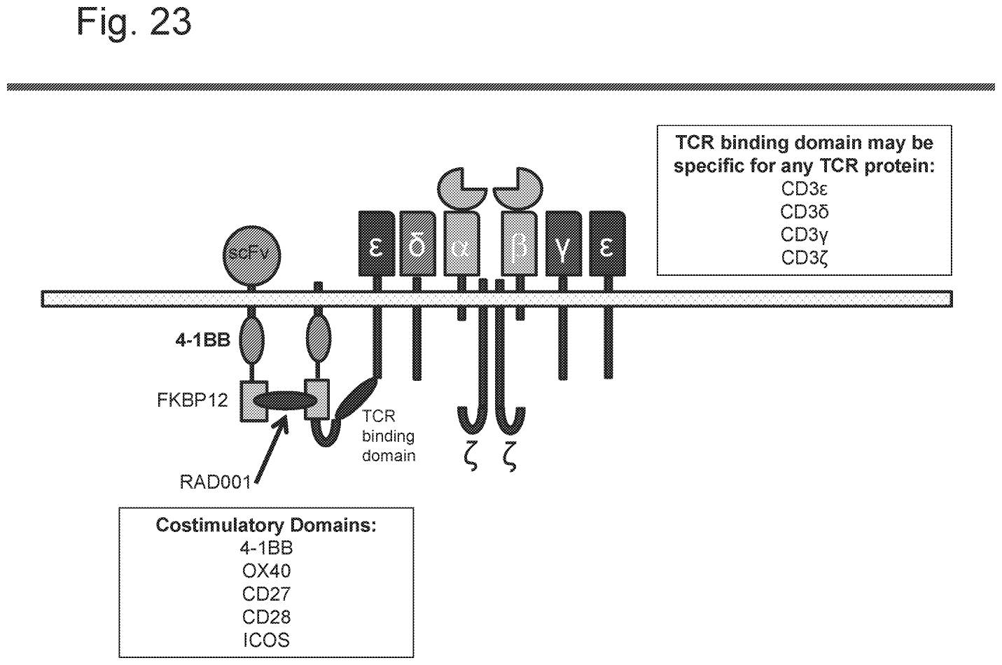

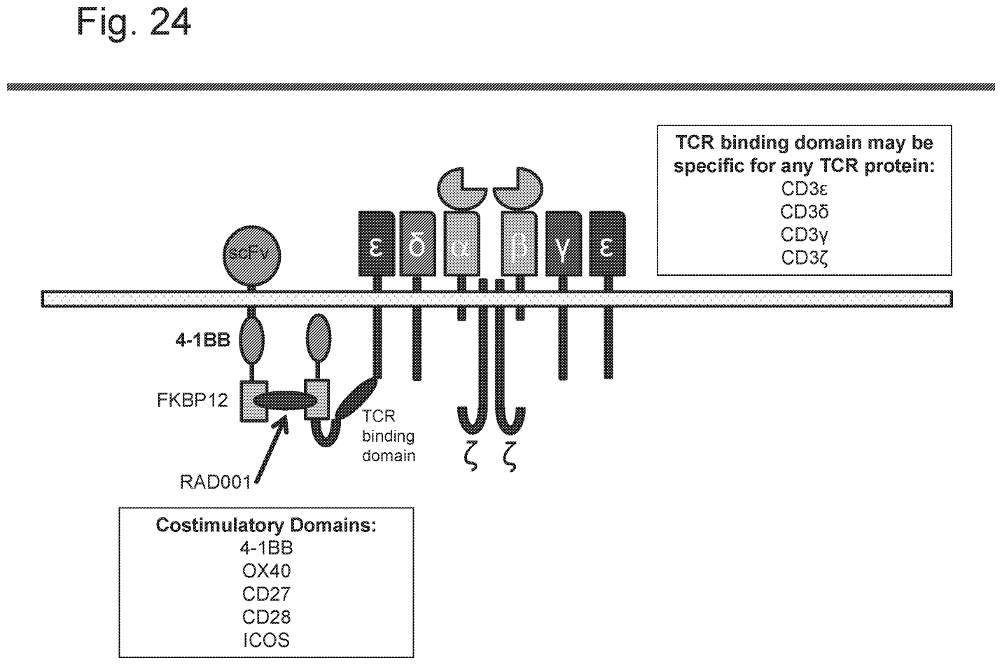

### Example 4: Regulatable TCARs Using Rapalogue Switch (rTCAR) (FIGS. 20-24)

Synthesis of Rapalogue Switch-Mediated rTCAR Constructs

Pairs of plasmid DNA were synthesized externally by DNA2.0. The nominal non-regulatable CAR construct, CD19scFv-BBZ, SEQ ID NO: 1, was used as a control. For the rTCAR, the various heterodimerization domains can be linked to different domains of the rTCAR constructs.

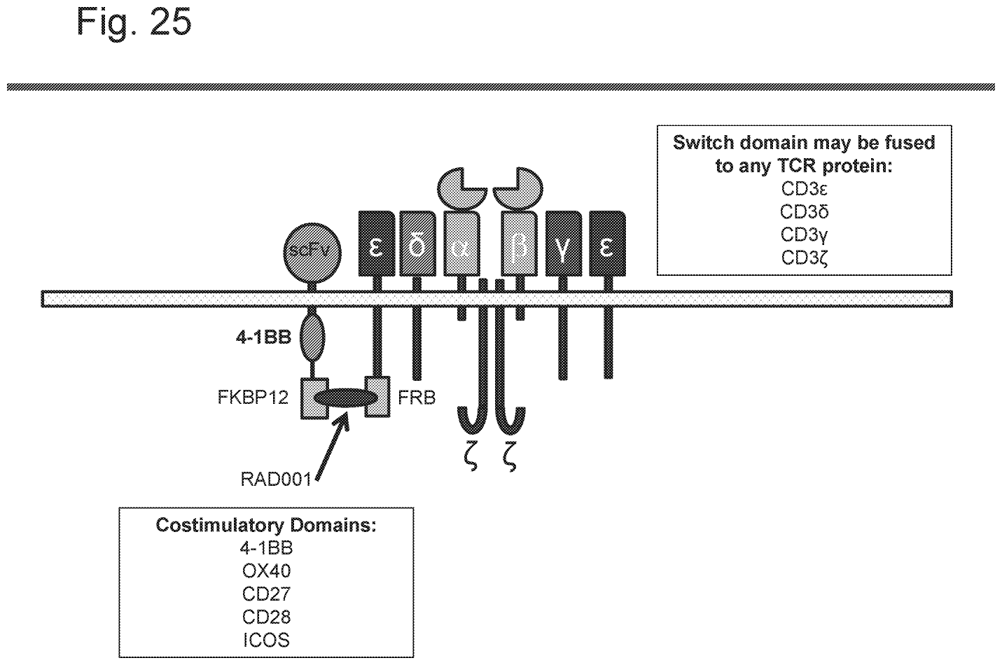

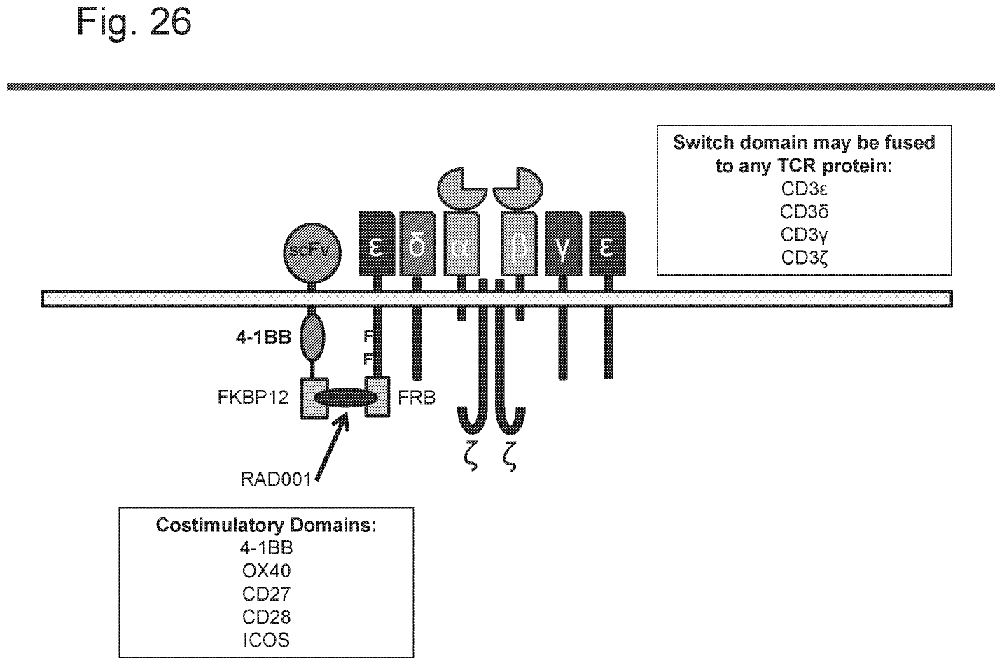

“rTCAR1” (FIG. 25) comprises a pair of constructs. In the first construct, the CD19 scFv was cloned with CD8 hinge and transmembrane domain followed by the costimulatory domain 4-1BB and FKBP at the C-terminus (SEQ ID NO: 14). The corresponding second construct was designed by fusing a mutated FRB domain with enhanced affinity to RAD001 to a linker at the C-terminus of CD3 epsilon (SEQ ID NO:15). “rTCAR2” (FIG. 26) comprises a pair of constructs. In the first construct, the CD19 scFv was cloned with CD8 hinge and transmembrane domain followed by the costimulatory domain 4-1BB and FKBP at the C-terminus (SEQ ID NO: 14). The corresponding second construct was designed by fusing a mutated FRB domain with enhanced affinity to RAD001 to a linker at the C-terminus of CD3 epsilon; additionally the two tyrosines within the ITAM domain of CD3 epsilon were mutated to phenylalanine to remove intrinsic signaling pathway from CD3 epsilon and demonstrate that signaling was mediated from the entire TCR complex (SEQ ID NO: 16).

CD19scFV-BB-FKBP
(SEQ ID NO: 14)
GSATMALPVTALLLPLALLLHAARPEIVMTQSPATLSLSPGERATLSCRA
SQDISKYLNWYQQKPGQAPRLLIYHTSRLHSGIPARFSGSGSGTDYTLTI
SSLQPEDFAVYFCQQGNTLPYTFGQGTKLEIKGGGGSGGGGSGGGGSQVQ
LQESGPGLVKPSETLSLTCTVSGVSLPDYGVSWIRQPPGKGLEWIGVIWG
SETTYYSSSLKSRVTISKDNSKNQVSLKLSSVTAADTAVYYCAKHYYYGG
SYAMDYWGQGTLVTVSSTTTPAPRPPTPAPTIASQPLSLRPEACRPAAGG
AVHTRGLDFACDIYIWAPLAGTCGVLLLSLVITLYCSLKRGRKKLLYIFK
QPFMRPVQTTQEEDGCSCRFPEEEEGGCELMGVQVETISPGDGRTFPKRG
QTCVVHYTGMLEDGKKFDSSRDRNKPFKFMLGKQEVIRGWEEGVAQMSVG
QRAKLTISPDYAYGATGHPGIIPPHATLVFDVELLKLE
CD3e-FRBmutant
(SEQ ID NO: 15)
GSMQSGTHWRVLGLCLLSVGVWGQDGNEEMGGITQTPYKVSISGTTVILT
CPQYPGSEILWQHNDKNIGGDEDDKNIGSDEDHLSLKEFSELEQSGYYVC
YPRGSKPEDANFYLYLRARVCENCMEMDVMSVATIVIVDICITGGLLLLV
YYWSKNRKAKAKPVTRGAGAGGRQRGQNKERPPPVPNPDYEPIRKGQRDL
YSGLNQRRIGSGSGGSILWHEMWHEGLIEASRLYFGERNVKGMFEVLEPL
HAMMERGPQTLKETSFNQAYGRDLMEAQEWCRKYMKSGNVKDLLQAWDLY
YHVFRRISK
CD3e-YtoFdouble-FRBmutant
(SEQ ID NO: 16)
GSMQSGTHWRVLGLCLLSVGVWGQDGNEEMGGITQTPYKVSISGTTVILT
CPQYPGSEILWQHNDKNIGGDEDDKNIGSDEDHLSLKEFSELEQSGYYVC
YPRGSKPEDANFYLYLRARVCENCMEMDVMSVATIVIVDICITGGLLLLV
YYWSKNRKAKAKPVTRGAGAGGRQRGQNKERPPPVPNPDFEPIRKGQRDL
FSGLNQRRIGSGSGGSILWHEMWHEGLIEASRLYFGERNVKGMFEVLEPL
HAMMERGPQTLKETSFNQAYGRDLMEAQEWCRKYMKSGNVKDLLQAWDLY
YHVFRRISK

Dose Response of Rapalogue on NFAT Activation

The ability of RCAR constructs to demonstrate rapalogue-dependent signal activation following target antigen engagement of the antigen binding domain was measured with the Jurkat cells with NFAT-LUC reporter (JNL) reporter cell line as described in Example 1. The transfected cells were added to the target plate with 100 μl per well and co-incubated with varying concentrations of RAD001 for 18 hrs. Luciferase One Glo reagent 100 μl was added per well. The samples were incubated for 5 min and then luminescence was measured as described.

IL-2 Expression in Transfected Jurkat (JNL) Cells

Transfection of JNL cells and activation were performed as described above in the JNL RGA assay excepting incubation which was for 40 hours at 37° C., 5% CO₂. Measurement of secreted IL2 was performed as described in Example 1.

Results

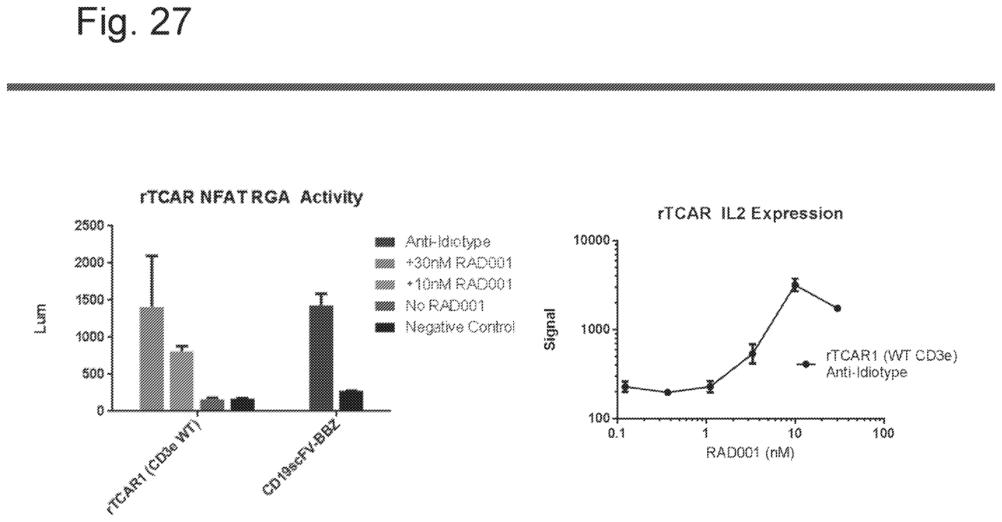

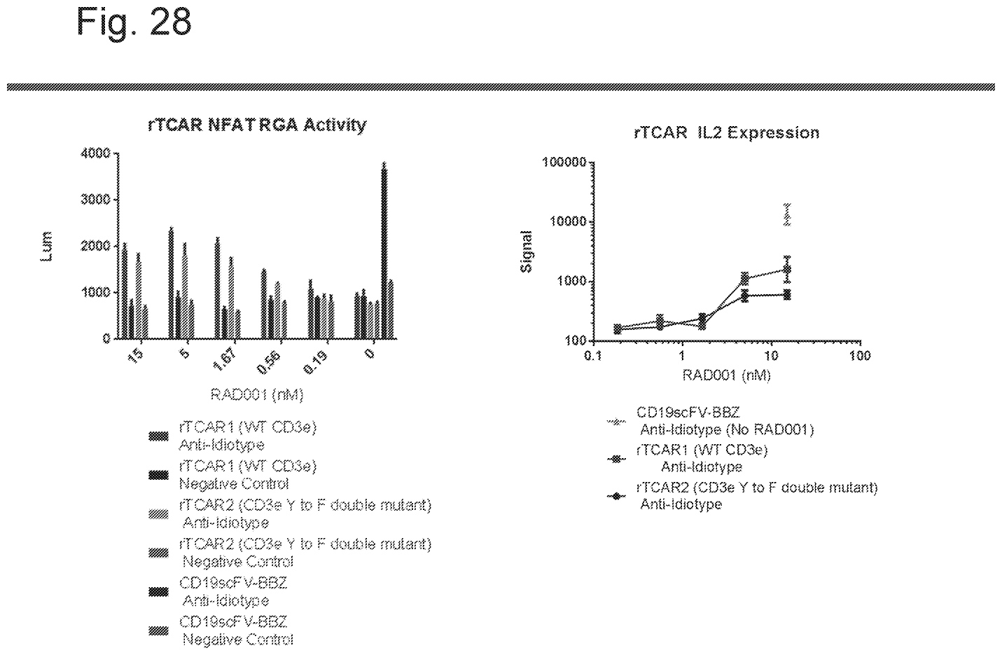

Initial screening of rTCAR1 via transient transfection into JNL cells demonstrated RAD001-mediated and antigen-dependent signaling and IL2 expression as shown in FIG. 27. In a subsequent experiment, rTCAR1 was compared to rTCAR2 in which the ITAM signaling of the transiently transfected CD3 epsilon was abrogated by mutation of the corresponding tyrosines to phenylalanine. A dose response with RAD001 was observed for rTCAR1 and rTCAR2 in both the reporter gene assay as well as in antigen-induced IL2 expression (FIG. 28), albeit with reduced signal and expression for rTCAR2 as would be expected. Transfection of a construct containing ITAM signaling domains is thus not a prerequisite for activity of rTCARs. By associating the targeting domain with the TCR complex, signaling is mediated through all members of the complex and is not exclusively limited to that derived from a signaling domain fused to the targeting domain.

### Example 6: Constitutively Active TCARs Fused into the TCR Complex Via Truncated CD3 Epsilon Extracellular Domains (fusTCAR)

Crystal structures of CD3 epsilon and gamma in complex with the Fab fragment of the anti-CD3 monoclonal antibody OKT3 (Kjer-Nielsen et. al., 2004) and CD3 epsilon and delta in complex with the scFv of the anti-CD3 monoclonal Ab UCHT1 (Arnett et. al., 2004) have been reported. These structures demonstrate that the interaction of epsilon with other accessory proteins is mediated through a beta sheet proximal to the membrane. Thus, fusion of a targeting domain to a truncated form of the CD3 epsilon extracellular domain comprised of the sequence containing the beta sheet may be the only prerequisite for fusTCAR activity.

Transient Expression

Synthesis of fusTCAR Constructs

Plasmid DNA was synthesized externally by DNA2.0. The nominal non-regulatable CAR construct, CD19scFv-BBZ, SEQ ID NO: 1, was used as a control.

In “fusTCAR5” the CD19 scFv was cloned as an N-terminal fusion to an N-terminally truncated form of CD3 epsilon extracellular and transmembrane domains followed by the intracellular costimulatory domain 4-1BB (SEQ ID NO: 17). “FusTCAR 6”, “fusTCAR7” and “fusTCAR8” was cloned similarly to “fusTCAR5” to elucidate the role of the two membrane proximal cysteines in CD3 epsilon which do not appear to be involved in intramolecular disulfide bonding in mediating the interaction with other TCR complex members. For “fusTCAR6” (SEQ ID No: 18), the first cysteine was mutated to serine. In “fusTCAR7” (SEQ ID NO: 19), the second cysteine was mutated to serine. Finally, for “fusTCAR8” (SEQ ID NO: 20), both cysteines were mutated to serine. All four constructs in this example lack intrinsic intracellular ITAM signaling domains.

CD19scFv-CD3e_minimalECDTM-41BB
(Seq ID NO: 17)
GSATMALPVTALLLPLALLLHAARPEIVMTQSPATLSLSPGERATLSCRA
SQDISKYLNWYQQKPGQAPRLLIYHTSRLHSGIPARFSGSGSGTDYTLTI
SSLQPEDFAVYFCQQGNTLPYTFGQGTKLEIKGGGGSGGGGSGGGGSQVQ
LQESGPGLVKPSETLSLTCTVSGVSLPDYGVSWIRQPPGKGLEWIGVIWG
SETTYYSSSLKSRVTISKDNSKNQVSLKLSSVTAADTAVYYCAKHYYYGG
SYAMDYWGQGTLVTVSSGGGGSPEDANFYLYLRARVCENCMEMDVMSVAT
IVIVDICITGGLLLLVYYWSKRGRKKLLYIFKQPFMRPVQTTQEEDGCSC
RFPEEEEGGCEL
CD19scFv-CD3e_minimalECD-1stCystoSer-TM-41BB
(Seq ID NO: 18)
GSATMALPVTALLLPLALLLHAARPEIVMTQSPATLSLSPGERATLSCRA
SQDISKYLNWYQQKPGQAPRLLIYHTSRLHSGIPARFSGSGSGTDYTLTI
SSLQPEDFAVYFCQQGNTLPYTFGQGTKLEIKGGGGSGGGGSGGGGSQVQ
LQESGPGLVKPSETLSLTCTVSGVSLPDYGVSWIRQPPGKGLEWIGVIWG
SETTYYSSSLKSRVTISKDNSKNQVSLKLSSVTAADTAVYYCAKHYYYGG
SYAMDYWGQGTLVTVSSGGGGSPEDANFYLYLRARVSENCMEMDVMSVAT
IVIVDICITGGLLLLVYYWSKRGRKKLLYIFKQPFMRPVQTTQEEDGCSC
RFPEEEEGGCEL
CD19scFv-CD3e_minimalECDTM-2ndCystoSer-TM-41BB
(Seq ID NO: 19)
GSATMALPVTALLLPLALLLHAARPEIVMTQSPATLSLSPGERATLSCRA
SQDISKYLNWYQQKPGQAPRLLIYHTSRLHSGIPARFSGSGSGTDYTLTI
SSLQPEDFAVYFCQQGNTLPYTFGQGTKLEIKGGGGSGGGGSGGGGSQVQ
LQESGPGLVKPSETLSLTCTVSGVSLPDYGVSWIRQPPGKGLEWIGVIWG
SETTYYSSSLKSRVTISKDNSKNQVSLKLSSVTAADTAVYYCAKHYYYGG
SYAMDYWGQGTLVTVSSGGGGSPEDANFYLYLRARVCENSMEMDVMSVAT
IVIVDICITGGLLLLVYYWSKRGRKKLLYIFKQPFMRPVQTTQEEDGCSC
RFPEEEEGGCEL
CD19scFv-CD3e_minimalECDTM-2xCystoSer-TM-41BB
(Seq ID NO: 20)
GSATMALPVTALLLPLALLLHAARPEIVMTQSPATLSLSPGERATLSCRA
SQDISKYLNWYQQKPGQAPRLLIYHTSRLHSGIPARFSGSGSGTDYTLTI
SSLQPEDFAVYFCQQGNTLPYTFGQGTKLEIKGGGGSGGGGSGGGGSQVQ
LQESGPGLVKPSETLSLTCTVSGVSLPDYGVSWIRQPPGKGLEWIGVIWG
SETTYYSSSLKSRVTISKDNSKNQVSLKLSSVTAADTAVYYCAKHYYYGG
SYAMDYWGQGTLVTVSSGGGGSPEDANFYLYLRARVSENSMEMDVMSVAT
IVIVDICITGGLLLLVYYWSKRGRKKLLYIFKQPFMRPVQTTQEEDGCSC
RFPEEEEGGCEL

Transfection of Jurkat Reporter Cell Line and Activation of NFAT.

Activation following target antigen engagement of the antigen binding domain was measured with the Jurkat cells with NFAT-LUC reporter (JNL) reporter cell line as described in Example 1. The transfected cells was added to the target plate with 100 μl per well. Luciferase One Glo reagent 100 μl was added per well. The samples were incubated for 5 min and then luminescence was measured as described.

Transient Expression Results

Target dependent signaling was not observed in transient expressed fusTCAR using the truncated extracellular domains. Subsequent FACs analysis demonstrated that there was no detectable expression of the constructs on the cell surface.

Production of Lentiviral Transduced Primary Human T-Cells To determine if low expression was unique to the reporter cell line, a single representative truncated fusion construct, fusTCAR6, was also tested in primary human T-Cells for activity relative to Cd19scFv-BBZ.

Lentivirus Production and Viral Transduction into Primary T Cells

As described in Example 1, lentivirus were produced and transduced into isolated primary human T-cells. Transduced T-cells and non-transduced control T-cells were expanded and frozen for subsequent analysis.

Cytotoxicity and IL2 Assay

Cytotoxicity and IL2 production induced by cross-linking primary human T-Cells to target tumor cells were assessed as described in Example 1.

Primary Human T-Cell Results

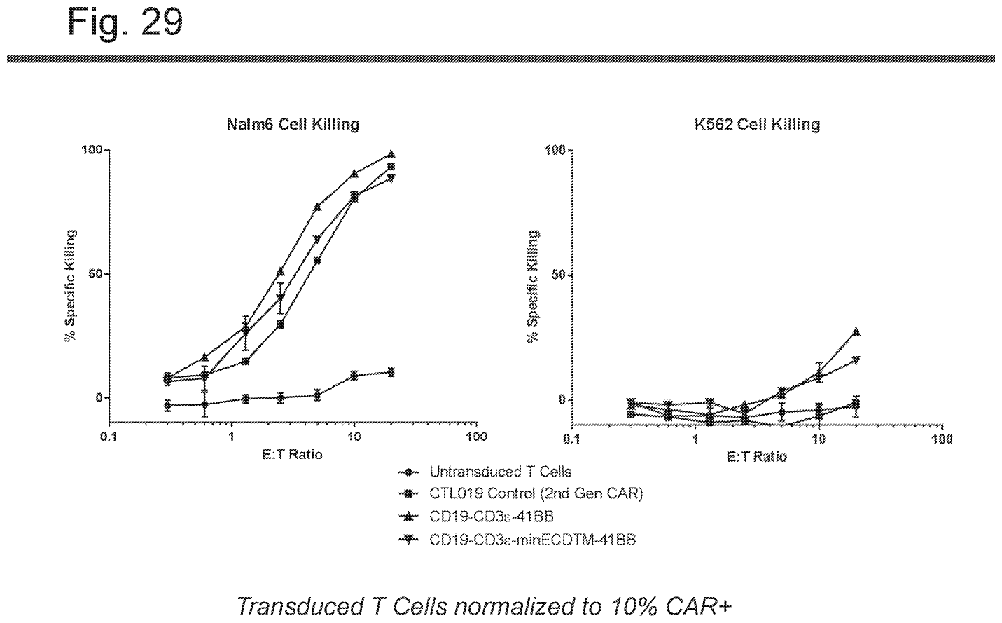

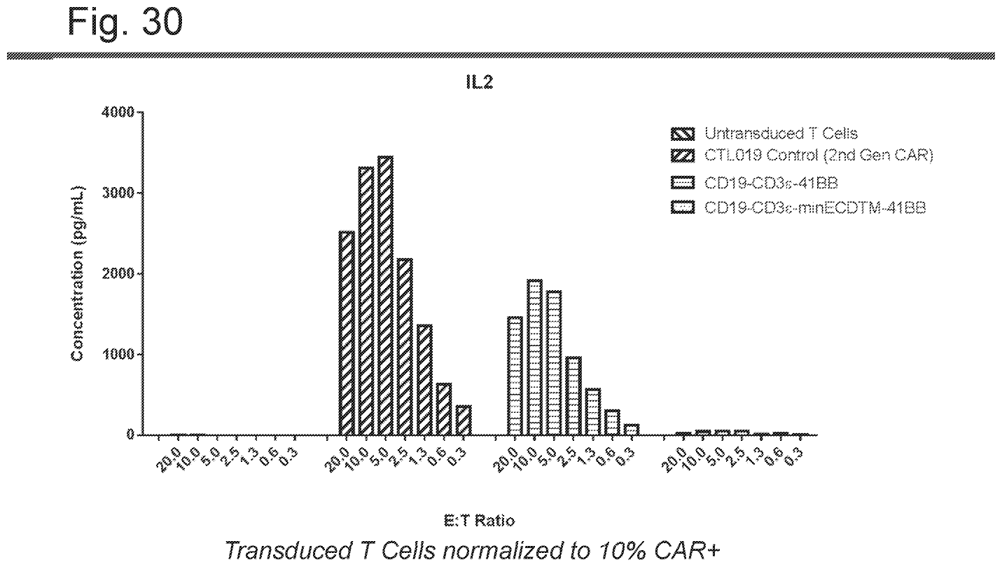

As can be observed in FIG. 29, fusTCAR6 demonstrated comparable cytolytic activity relative to the control CD19scFv-BBZ CAR. Important to note that redirected lytic activity was observed despite the absence of ITAM signaling domains. In contrast, as shown in FIG. 30, fusTCAR6 resulted in reduced IL2 expression. Additional optimization of the construct is necessary to either stabilize the beta sheet or improve the interaction of the truncated CD3 epsilon with the remaining endogenous components of TCR. Nonetheless, the results demonstrated that the entire extracellular domain of the extracellular region of CD3 epsilon is not required to maintain cytolytic activity.

### Example 7: Constitutively Active TCARs Fused into the TCR Complex Via CD3 Epsilon with Alternative Costimulatory Domains

Traditional CARs have demonstrated to be functional with alternative costimulatory domains other than 4-1BB.

Production of Lentiviral Transduced Primary Human T-Cells

Synthesis of fusTCAR Constructs

Plasmid DNA will be synthesized externally by DNA2.0. The nominal non-regulatable CAR construct, CD19scFv-BBZ, SEQ ID NO: 1, will be used as a control and “fusCAR3” will be used as the TCAR control (SEQ ID NO: 9).

In fusTCAR listed in the table below the CD19 scFv will be cloned as an N-terminal fusion to the CD3 epsilon extracellular and transmembrane domains followed by intracellular costimulatory domains as specified. “FusTCAR9” to “fusTCAR13” lack intrinsic intracellular ITAM signaling domains.

| “FusTCAR” | Costimulatory Domain | SEQ ID NO |
|---|---|---|
| fusTCAR9 | CD27 | 21 |
| fusTCAR10 | CD28 | 22 |
| fusTCAR11 | OX40 | 23 |
| fusTCAR12 | ICOS | 24 |
| fusTCAR13 | CD2 | 25 |

CD19scFv-CD3eECDTM-CD27 (SEQ ID NO: 21)
GSMALPVTALLLPLALLLHAARPEIVMTQSPATLSLSPGERATLSCRASQ
DISKYLNWYQQKPGQAPRLLIYHTSRLHSGIPARFSGSGSGTDYTLTISS
LQPEDFAVYFCQQGNTLPYTFGQGTKLEIKGGGGSGGGGSGGGGSQVQLQ
ESGPGLVKPSETLSLTCTVSGVSLPDYGVSWIRQPPGKGLEWIGVIWGSE
TTYYSSSLKSRVTISKDNSKNQVSLKLSSVTAADTAVYYCAKHYYYGGSY
AMDYWGQGTLVTVSSGGGGSDGNEEMGGITQTPYKVSISGTTVILTCPQY
PGSEILWQHNDKNIGGDEDDKNIGSDEDHLSLKEFSELEQSGYYVCYPRG
SKPEDANFYLYLRARVCENCMEMDVMSVATIVIVDICITGGLLLLVYYWS
QRRKYRSNKGESPVEPAEPCHYSCPREEEGSTIPIQEDYRKPEPACSP
CD19scFv-CD3eECDTM-CD28 (SEQ ID NO: 22)
GSMALPVTALLLPLALLLHAARPEIVMTQSPATLSLSPGERATLSCRASQ
DISKYLNWYQQKPGQAPRLLIYHTSRLHSGIPARFSGSGSGTDYTLTISS
LQPEDFAVYFCQQGNTLPYTFGQGTKLEIKGGGGSGGGGSGGGGSQVQLQ
ESGPGLVKPSETLSLTCTVSGVSLPDYGVSWIRQPPGKGLEWIGVIWGSE
TTYYSSSLKSRVTISKDNSKNQVSLKLSSVTAADTAVYYCAKHYYYGGSY
AMDYWGQGTLVTVSSGGGGSDGNEEMGGITQTPYKVSISGTTVILTCPQY
PGSEILWQHNDKNIGGDEDDKNIGSDEDHLSLKEFSELEQSGYYVCYPRG
SKPEDANFYLYLRARVCENCMEMDVMSVATIVIVDICITGGLLLLVYYWS
RSKRSRLLHSDYMNMTPRRPGPTRKHYQPYAPPRDFAAYRS
CD19scFv-CD3eECDTM-OX40 (SEQ ID NO: 23)
GSMALPVTALLLPLALLLHAARPEIVMTQSPATLSLSPGERATLSCRASQ
DISKYLNWYQQKPGQAPRLLIYHTSRLHSGIPARFSGSGSGTDYTLTISS
LQPEDFAVYFCQQGNTLPYTFGQGTKLEIKGGGGSGGGGSGGGGSQVQLQ
ESGPGLVKPSETLSLTCTVSGVSLPDYGVSWIRQPPGKGLEWIGVIWGSE
TTYYSSSLKSRVTISKDNSKNQVSLKLSSVTAADTAVYYCAKHYYYGGSY
AMDYWGQGTLVTVSSGGGGSDGNEEMGGITQTPYKVSISGTTVILTCPQY
PGSEILWQHNDKNIGGDEDDKNIGSDEDHLSLKEFSELEQSGYYVCYPRG
SKPEDANFYLYLRARVCENCMEMDVMSVATIVIVDICITGGLLLLVYYWS
RRDQRLPPDAHKPPGGGSPRTPIQEEQADAHSTLAKI
CD19scFv-CD3eECDTM-ICOS (SEQ ID NO: 24)
GSMALPVTALLLPLALLLHAARPEIVMTQSPATLSLSPGERATLSCRASQ
DISKYLNWYQQKPGQAPRLLIYHTSRLHSGIPARFSGSGSGTDYTLTISS
LQPEDFAVYFCQQGNTLPYTFGQGTKLEIKGGGGSGGGGSGGGGSQVQLQ
ESGPGLVKPSETLSLTCTVSGVSLPDYGVSWIRQPPGKGLEWIGVIWGSE
TTYYSSSLKSRVTISKDNSKNQVSLKLSSVTAADTAVYYCAKHYYYGGSY
AMDYWGQGTLVTVSSGGGGSDGNEEMGGITQTPYKVSISGTTVILTCPQY
PGSEILWQHNDKNIGGDEDDKNIGSDEDHLSLKEFSELEQSGYYVCYPRG
SKPEDANFYLYLRARVCENCMEMDVMSVATIVIVDICITGGLLLLVYYWS
TKKKYSSSVHDPNGEYMFMRAVNTAKKSRLTDVTL
CD19scFv-CD3eECDTM-CD2 (SEQ ID NO: 25)
GSMALPVTALLLPLALLLHAARPEIVMTQSPATLSLSPGERATLSCRASQ
DISKYLNWYQQKPGQAPRLLIYHTSRLHSGIPARFSGSGSGTDYTLTISS
LQPEDFAVYFCQQGNTLPYTFGQGTKLEIKGGGGSGGGGSGGGGSQVQLQ
ESGPGLVKPSETLSLTCTVSGVSLPDYGVSWIRQPPGKGLEWIGVIWGSE
TTYYSSSLKSRVTISKDNSKNQVSLKLSSVTAADTAVYYCAKHYYYGGSY
AMDYWGQGTLVTVSSGGGGSDGNEEMGGITQTPYKVSISGTTVILTCPQY
PGSEILWQHNDKNIGGDEDDKNIGSDEDHLSLKEFSELEQSGYYVCYPRG
SKPEDANFYLYLRARVCENCMEMDVMSVATIVIVDICITGGLLLLVYYWS
KRKKQRSRRNDEELETRAHRVATEERGRKPHQIPASTPQNPATSQHPPPP
PGHRSQAPSHRPPPPGHRVQHQPQKRPPAPSGTQVHQQKGPPLPRPRVQP
KPPHGAAENSLSPSSN

Lentivirus Production and Viral Transduction into Primary T Cells

As described in Example 1, lentivirus were produced and transduced into isolated primary human T-cells. Transduced T-cells and non-transduced control T-cells were expanded and frozen for subsequent analysis.

Cytotoxicity and IL2 Assay

Cytotoxicity and IL2 production induced by cross-linking primary human T-Cells to target tumor cells were assessed as described in Example 1.

Primary Human T-Cell Results

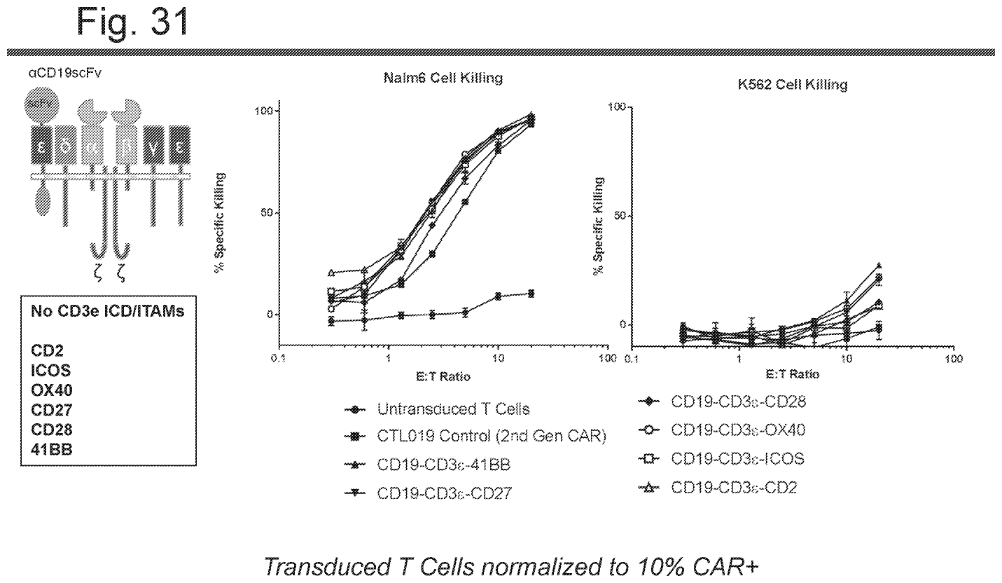

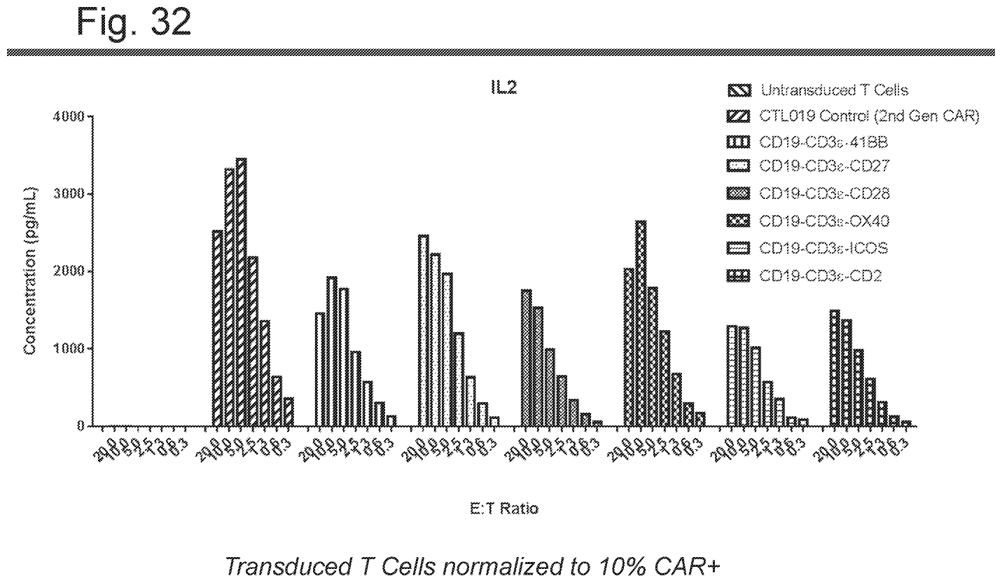

FIG. 31 and FIG. 32 demonstrate that fusTCARs may be employed with any costimulatory domain and still retain target dependent cytolytic activity and induce IL2 expression despite the lack of ITAM domains within the constructs.

### Example 8: Constitutively Active TCARs Fused into the TCR Complex Via CD3 Gamma, CD3 Delta and CD3 Zeta (fusTCAR)

Given the complex multi-protein architecture of the TCR complex, activity for TCARs may not be limited to covalent and non-covalent fusions with CD3 epsilon; non-covalent and covalent fusions with other accessory proteins in the complex such as for examples CD3 gamma, CD3 delta and CD3 zeta may also produce active TCARs. Additionally, as immunological synapse may be mediated by the distance between the target cells and the T-Cells, it may become necessary to mediate the optimal length by using different length linkers between the tumor targeting arm and the fusion with these accessory proteins.

Transient Expression and Activation Assays

Synthesis of fusTCAR Constructs

Plasmid DNA will be synthesized externally by DNA2.0. The nominal non-regulatable CAR construct, CD19scFv-BBZ, SEQ ID NO: 1, will be used as a control.

In “fusTCAR14” the CD19 scFv will be cloned as an N-terminal fusion with 2×G4S linker (SEQ ID NO: 62) to the CD3 delta extracellular and transmembrane domains followed by the intracellular costimulatory domain 4-1BB (SEQ ID NO: 26). Similarly, “fusTCAR15” (SEQ ID NO: 27) will be cloned excepting with 4×G4S linker (SEQ ID NO: 45) between the scFv and the CD3 epsilon extracellular domain.

In “fusTCAR16” the CD19 scFv was cloned as an N-terminal fusion to the CD3 delta extracellular and transmembrane domains followed by the intracellular costimulatory domain 4-1BB (SEQ ID NO: 28). “fusTCAR17” (SEQ ID NO: 29) and “fusCAR18” (SEQ ID NO: 30) were cloned similarly excepting with 2×G4S (SEQ ID NO: 62) and 4×G4S (SEQ ID NO: 45) linkers, respectively, between the scFv and the CD3 delta extracellular domain.

In “fusTCAR19” the CD19 scFv was cloned as an N-terminal fusion to the CD3 gamma extracellular and transmembrane domains followed by the intracellular costimulatory domain 4-1BB (SEQ ID NO: 31). “fusTCAR20” (SEQ ID NO: 32) and “fusTCAR21” (SEQ ID NO: 33) were cloned similarly excepting with 2×G4S(SEQ ID NO: 62) and 4×G4S (SEQ ID NO: 45) linkers, respectively, between the scFv and the CD3 gamma extracellular domain.

CD19scFv-CD3e_2G4S_ECDTM-41BB
(Seq ID NO: 26/“2G4S” disclosed as SEQ ID NO: 62)
GSMALPVTALLLPLALLLHAARPEIVMTQSPATLSLSPGERATLSCRASQ
DISKYLNWYQQKPGQAPRLLIYHTSRLHSGIPARFSGSGSGTDYTLTISS
LQPEDFAVYFCQQGNTLPYTFGQGTKLEIKGGGGSGGGGSGGGGSQVQLQ
ESGPGLVKPSETLSLTCTVSGVSLPDYGVSWIRQPPGKGLEWIGVIWGSE
TTYYSSSLKSRVTISKDNSKNQVSLKLSSVTAADTAVYYCAKHYYYGGSY
AMDYWGQGTLVTVSSGGGGSGGGGSDGNEEMGGITQTPYKVSISGTTVIL
TCPQYPGSEILWQHNDKNIGGDEDDKNIGSDEDHLSLKEFSELEQSGYYV
CYPRGSKPEDANFYLYLRARVCENCMEMDVMSVATIVIVDICITGGLLLL
VYYWSKRGRKKLLYIFKQPFMRPVQTTQEEDGCSCRFPEEEEGGCEL
CD19scFv-CD3e_4G45_ECDTM-41BB
(Seq ID NO: 27/“4G4S” disclosed as SEQ ID NO: 45)
GSMALPVTALLLPLALLLHAARPEIVMTQSPATLSLSPGERATLSCRASQ
DISKYLNWYQQKPGQAPRLLIYHTSRLHSGIPARFSGSGSGTDYTLTISS
LQPEDFAVYFCQQGNTLPYTFGQGTKLEIKGGGGSGGGGSGGGGSQVQLQ
ESGPGLVKPSETLSLTCTVSGVSLPDYGVSWIRQPPGKGLEWIGVIWGSE
TTYYSSSLKSRVTISKDNSKNQVSLKLSSVTAADTAVYYCAKHYYYGGSY
AMDYWGQGTLVTVSSGGGGSGGGGSGGGGSGGGGSDGNEEMGGITQTPYK
VSISGTTVILTCPQYPGSEILWQHNDKNIGGDEDDKNIGSDEDHLSLKEF
SELEQSGYYVCYPRGSKPEDANFYLYLRARVCENCMEMDVMSVATIVIVD
ICITGGLLLLVYYWSKRGRKKLLYIFKQPFMRPVQTTQEEDGCSCRFPEE
EEGGCEL
CD19scFv-CD3d_ECDTM-41BB
(Seq ID NO: 28)
GSATMALPVTALLLPLALLLHAARPEIVMTQSPATLSLSPGERATLSCRA
SQDISKYLNWYQQKPGQAPRLLIYHTSRLHSGIPARFSGSGSGTDYTLTI
SSLQPEDFAVYFCQQGNTLPYTFGQGTKLEIKGGGGSGGGGSGGGGSQVQ
LQESGPGLVKPSETLSLTCTVSGVSLPDYGVSWIRQPPGKGLEWIGVIWG
SETTYYSSSLKSRVTISKDNSKNQVSLKLSSVTAADTAVYYCAKHYYYGG
SYAMDYWGQGTLVTVSSGGGGSFKIPIEELEDRVFVNCNTSITWVEGTVG
TLLSDITRLDLGKRILDPRGIYRCNGTDIYKDKESTVQVHYRMCQSCVEL
DPATVAGIIVTDVIATLLLALGVFCFAKRGRKKLLYIFKQPFMRPVQTTQ
EEDGCSCRFPEEEEGGCEL
CD19scFv-CD3d_2G4S_ECDTM-41BB
(Seq ID NO: 29/“2G4S” disclosed as SEQ ID NO: 62)
GSMALPVTALLLPLALLLHAARPEIVMTQSPATLSLSPGERATLSCRASQ
DISKYLNWYQQKPGQAPRLLIYHTSRLHSGIPARFSGSGSGTDYTLTISS
LQPEDFAVYFCQQGNTLPYTFGQGTKLEIKGGGGSGGGGSGGGGSQVQLQ
ESGPGLVKPSETLSLTCTVSGVSLPDYGVSWIRQPPGKGLEWIGVIWGSE
TTYYSSSLKSRVTISKDNSKNQVSLKLSSVTAADTAVYYCAKHYYYGGSY
AMDYWGQGTLVTVSSGGGGSGGGGSFKIPIEELEDRVFVNCNTSITWVEG
TVGTLLSDITRLDLGKRILDPRGIYRCNGTDIYKDKESTVQVHYRMCQSC
VELDPATVAGIIVTDVIATLLLALGVFCFAKRGRKKLLYIFKQPFMRPVQ
TTQEEDGCSCRFPEEEEGGCEL
CD19scFv-CD3d_4G45_ECDTM-41BB
(Seq ID NO: 30/“4G4S” disclosed as SEQ ID NO: 45)
GSMALPVTALLLPLALLLHAARPEIVMTQSPATLSLSPGERATLSCRASQ
DISKYLNWYQQKPGQAPRLLIYHTSRLHSGIPARFSGSGSGTDYTLTISS
LQPEDFAVYFCQQGNTLPYTFGQGTKLEIKGGGGSGGGGSGGGGSQVQLQ
ESGPGLVKPSETLSLTCTVSGVSLPDYGVSWIRQPPGKGLEWIGVIWGSE
TTYYSSSLKSRVTISKDNSKNQVSLKLSSVTAADTAVYYCAKHYYYGGSY
AMDYWGQGTLVTVSSGGGGSGGGGSGGGGSGGGGSFKIPIEELEDRVFVN
CNTSITWVEGTVGTLLSDITRLDLGKRILDPRGIYRCNGTDIYKDKESTV
QVHYRMCQSCVELDPATVAGIIVTDVIATLLLALGVFCFAKRGRKKLLYI
FKQPFMRPVQTTQEEDGCSCRFPEEEEGGCEL
CD19scFv-CD3gECDTM-41BB
(Seq ID NO: 31)
GSATMALPVTALLLPLALLLHAARPEIVMTQSPATLSLSPGERATLSCRA
SQDISKYLNWYQQKPGQAPRLLIYHTSRLHSGIPARFSGSGSGTDYTLTI
SSLQPEDFAVYFCQQGNTLPYTFGQGTKLEIKGGGGSGGGGSGGGGSQVQ
LQESGPGLVKPSETLSLTCTVSGVSLPDYGVSWIRQPPGKGLEWIGVIWG
SETTYYSSSLKSRVTISKDNSKNQVSLKLSSVTAADTAVYYCAKHYYYGG
SYAMDYWGQGTLVTVSSGGGGSQSIKGNHLVKVYDYQEDGSVLLTCDAEA
KNITWFKDGKMIGFLTEDKKKWNLGSNAKDPRGMYQCKGSQNKSKPLQVY
YRMCQNCIELNAATISGFLFAEIVSIFVLAVGVYFIAKRGRKKLLYIFKQ
PFMRPVQTTQEEDGCSCRFPEEEEGGCEL
CD19scFv-2G4S_CD3gECDTM-41BB
(Seq ID NO: 32/“2G4S” disclosed as SEQ ID NO: 62)
GSMALPVTALLLPLALLLHAARPEIVMTQSPATLSLSPGERATLSCRASQ
DISKYLNWYQQKPGQAPRLLIYHTSRLHSGIPARFSGSGSGTDYTLTISS
LQPEDFAVYFCQQGNTLPYTEGQGTKLEIKGGGGSGGGGSGGGGSQVQLQ
ESGPGLVKPSETLSLTCTVSGVSLPDYGVSWIRQPPGKGLEWIGVIWGSE
TTYYSSSLKSRVTISKDNSKNQVSLKLSSVTAADTAVYYCAKHYYYGGSY
AMDYWGQGTLVTVSSGGGGSGGGGSQSIKGNHLVKVYDYQEDGSVLLTCD
AEAKNITWFKDGKMIGFLTEDKKKWNLGSNAKDPRGMYQCKGSQNKSKPL
QVYYRMCQNCIELNAATISGFLFAEIVSIFVLAVGVYFIAKRGRKKLLYI
FKQPFMRPVQTTQEEDGCSCRFPEEEEGGCEL
CD19scFv-4G45_CD3gECDTM-41BB
(Seq ID NO: 33/“4G4S” disclosed as SEQ ID NO: 45)
GSMALPVTALLLPLALLLHAARPEIVMTQSPATLSLSPGERATLSCRASQ
DISKYLNWYQQKPGQAPRLLIYHTSRLHSGIPARFSGSGSGTDYTLTISS
LQPEDFAVYFCQQGNTLPYTEGQGTKLEIKGGGGSGGGGSGGGGSQVQLQ
ESGPGLVKPSETLSLTCTVSGVSLPDYGVSWIRQPPGKGLEWIGVIWGSE
TTYYSSSLKSRVTISKDNSKNQVSLKLSSVTAADTAVYYCAKHYYYGGSY
AMDYWGQGTLVTVSSGGGGSGGGGSGGGGSGGGGSQSIKGNHLVKVYDYQ
EDGSVLLTCDAEAKNITWFKDGKMIGFLTEDKKKWNLGSNAKDPRGMYQC
KGSQNKSKPLQVYYRMCQNCIELNAATISGFLFAEIVSIFVLAVGVYFIA
KRGRKKLLYIFKQPFMRPVQTTQEEDGCSCRFPEEEEGGCEL

Transfection of Jurkat Reporter Cell Line and Activation of NFAT.

Activation following target antigen engagement of the antigen binding domain was measured with the Jurkat cells with NFAT-LUC reporter (JNL) reporter cell line as described in Example 1. The transfected cells were added to the target plate with 100 μl per well. Luciferase One Glo reagent 100 μl was added per well. The samples were incubated for 5 min and then luminescence was measured as described.

Transient Transfection Results

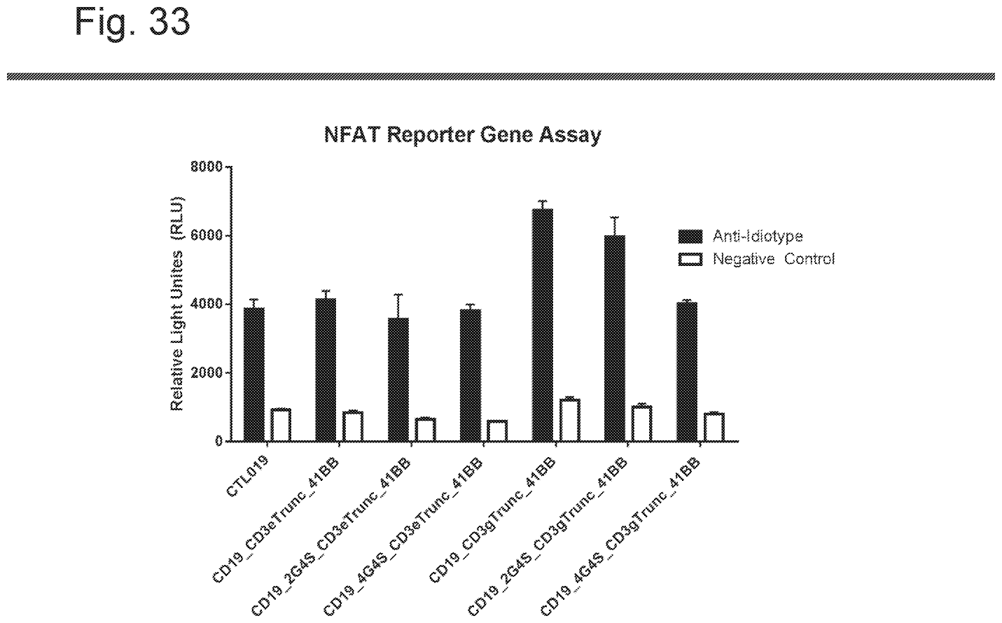

FIG. 33 demonstrates that TCARs are functional and result in signaling via the NFAT pathway regardless of whether CD3 epsilon or CD3 gamma was used for the fusion in the construct. Additionally, linkers of various lengths may be employed to fuse the binding domain to the remainder of the TCAR in order to obtain the desired results. Constructs fused to CD3 delta did not transiently express on the cell surface of the reporter cells based upon FACS so a determination could not be made as to their suitability based upon this approach and evaluation was instead performed in primary human T-cells.

Production of Lentiviral Transduced Primary Human T-Cells

FusTCARs were also tested in primary human T-Cells for their activity. Prior to production of lentivirus an additional construct was also designed to test if the extracellular and transmembrane domains of CD3 zeta could be used in the absence of its intracellular domain. Plasmid DNA was synthesized externally by DNA2.0. In “fusTCAR22” the CD19 scFv was cloned as an N-terminal fusion to the CD3 zeta extracellular and transmembrane domains followed by the intracellular costimulatory domain 4-1BB (SEQ ID NO: 34).

CD19scFv-CD3zECDTM-41BB
(Seq ID NO: 34)
GSMALPVTALLLPLALLLHAARPEIVMTQSPATLSLSPGERATLSCRASQ
DISKYLNWYQQKPGQAPRLLIYHTSRLHSGIPARFSGSGSGTDYTLTISS
LQPEDFAVYFCQQGNTLPYTFGQGTKLEIKGGGGSGGGGSGGGGSQVQLQ
ESGPGLVKPSETLSLTCTVSGVSLPDYGVSWIRQPPGKGLEWIGVIWGSE
TTYYSSSLKSRVTISKDNSKNQVSLKLSSVTAADTAVYYCAKHYYYGGSY
AMDYWGQGTLVTVSSGGGGSQSFGLLDPKLCYLLDGILFIYGVILTALFL
KRGRKKLLYIFKQPFMRPVQTTQEEDGCSCRFPEEEEGGCEL

Lentivirus Production and Viral Transduction into Primary T Cells

As described in Example 1, lentivirus were produced for fusTCAR3, fusTCAR16, fusTCAR19 and fusTCAR22 and transduced into isolated primary human T-cells. Transduced T-cells and non-transduced control T-cells were expanded and frozen for subsequent analysis.

Cytotoxicity and IL2 Assay

Cytotoxicity and IL2 production induced by cross-linking primary human T-Cells to target tumor cells were assessed as described in Example 1.

Primary Human T-Cell Results

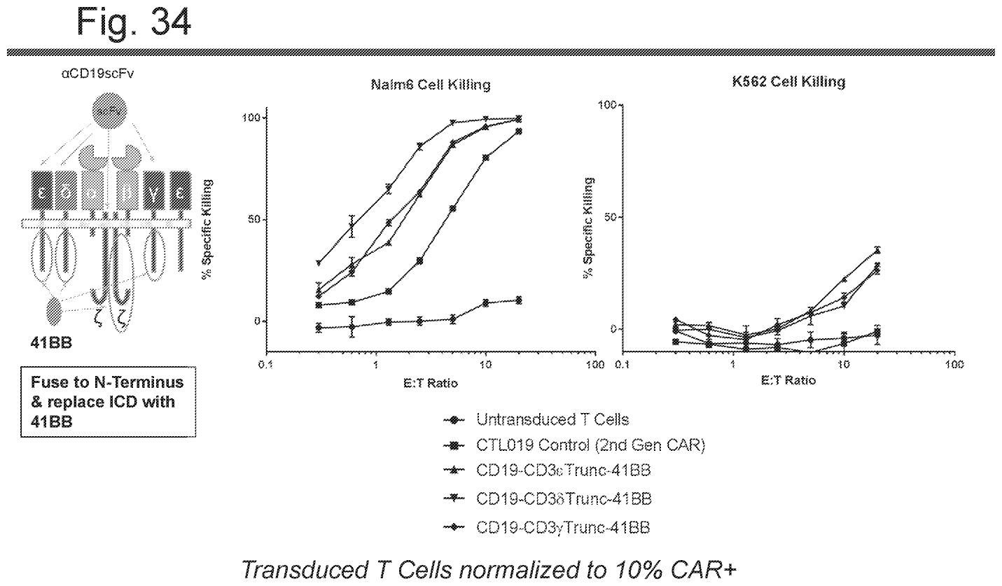

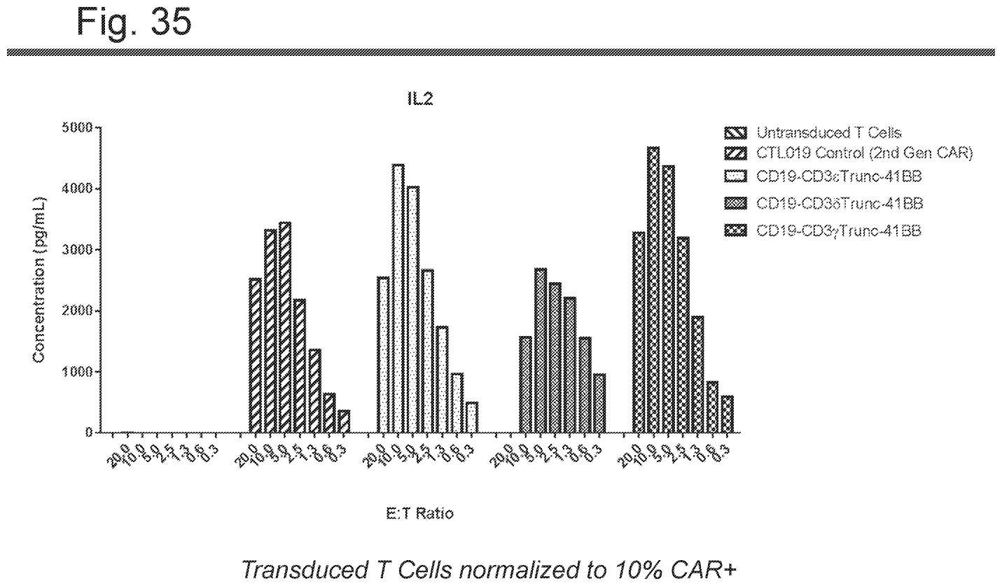

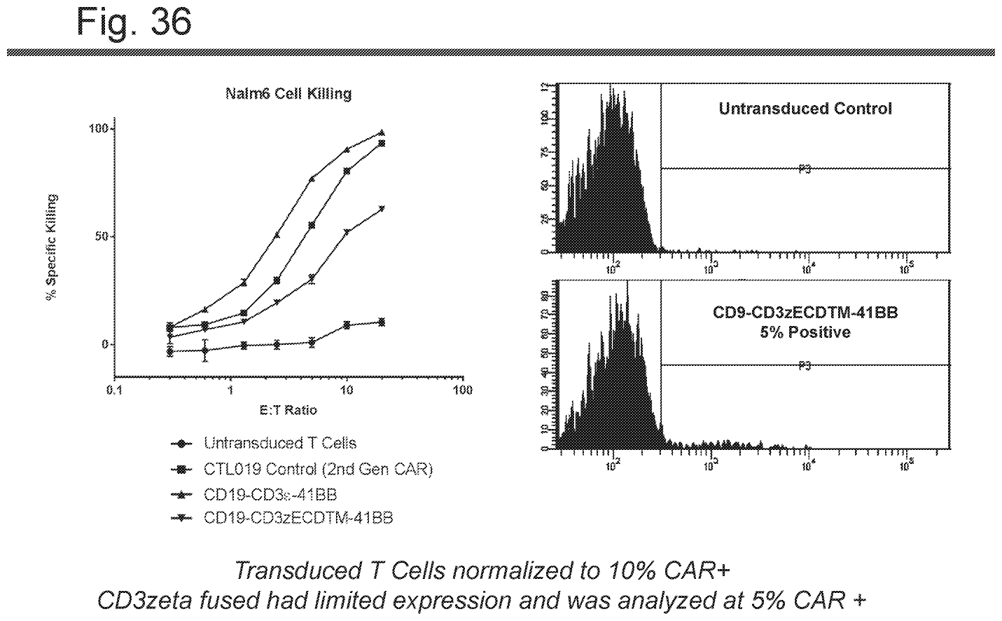

As can be observed in FIG. 34 and FIG. 35, fusTCARs on CD3 epsilon, CD3 gamma and CD3 delta demonstrated appreciable activity relative to the control CD19scFv-BBZ CAR. Important to note that redirected lytic activity and IL2 secretion was observed despite the absence of ITAM signaling domains. In contrast, as shown in FIG. 36, fusTCAR on CD3 zeta resulted in reduced lytic activity. FACS analysis demonstrated low cell surface expression for this construct likely due to the architecture of the TCR and the addition of the tumor targeting domain; additional optimization of the construct design is necessary to improve expression and maximize activity.

### Example 9: Constitutively Active TCARs Fused into the TCR Complex Via CD3 Epsilon with Alternative Binding Domains

TCARs should demonstrate broad applicability against solid as well as hematological tumors using a variety of binding domains and target antigens. Mesothelin is one antigen of interest expressed on a broad range of tumor types.

Synthesis of fusTCAR Constructs

Plasmid DNA will be synthesized externally by DNA2.0. The nominal non-regulatable CAR construct, MSLN5scFv-BBZ, SEQ ID NO: 35, will be used as a control.

In “fusTCAR23” the CD19 scFv will be cloned as an N-terminal fusion with G4S linker (SEQ ID NO: 52) to the CD3 epsilon extracellular and transmembrane domains followed by the intracellular costimulatory domain 4-1BB (SEQ ID NO: 36). “FusTCAR25” was cloned similarly excepting CD8a linker was used to fuse to the N-terminus of CD3 epsilon extracellular and transmembrane domains follower by 4-1BB (SEQ ID NO: 37) “FusTCAR25” was cloned as an N-terminal fusion with G4S linker (SEQ ID NO: 52) to the CD3 epsilon extracellular and transmembrane domains followed by the intracellular costimulatory domain CD27 (SEQ ID NO: 38) “FusTCAR23,” “fusTCAR24” and “fusTCAR25” lack intrinsic intracellular ITAM signaling domains.

MSLN5scFv-BBZ
(SEQ ID NO: 35)
GSMALPVTALLLPLALLLHAARPQVQLVQSGAEVEKPGASVKVSCKASGY
TFTDYYMHWVRQAPGQGLEWMGWINPNSGGTNYAQKFQGRVTMTRDTSIS
TAYMELSRLRSDDTAVYYCASGWDFDYWGQGTLVTVSSGGGGSGGGGSGG
GGSGGGGSDIVMTQSPSSLSASVGDRVTITCRASQSIRYYLSWYQQKPGK
APKLLIYTASILQNGVPSRFSGSGSGTDFTLTISSLQPEDFATYYCLQTY
TTPDFGPGTKVEIKTTTPAPRPPTPAPTIASQPLSLRPEACRPAAGGAVH
TRGLDFACDIYIWAPLAGTCGVLLLSLVITLYCKRGRKKLLYIFKQPFMR
PVQTTQEEDGCSCRFPEEEEGGCELRVKFSRSADAPAYKQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPR
MSLN5scFv-CD3eECDTM-41BB
(SEQ ID NO: 36)
GSMALPVTALLLPLALLLHAARPQVQLVQSGAEVEKPGASVKVSCKASGY
TFTDYYMHWVRQAPGQGLEWMGWINPNSGGTNYAQKFQGRVTMTRDTSIS
TAYMELSRLRSDDTAVYYCASGWDFDYWGQGTLVTVSSGGGGSGGGGSGG
GGSGGGGSDIVMTQSPSSLSASVGDRVTITCRASQSIRYYLSWYQQKPGK
APKLLIYTASILQNGVPSRFSGSGSGTDFTLTISSLQPEDFATYYCLQTY
TTPDFGPGTKVEIKGGGGSDGNEEMGGITQTPYKVSISGTTVILTCPQYP
GSEILWQHNDKNIGGDEDDKNIGSDEDHLSLKEFSELEQSGYYVCYPRGS
KPEDANFYLYLRARVCENCMEMDVMSVATIVIVDICITGGLLLLVYYWSK
RGRKKLLYIFKQPFMRPVQTTQEEDGCSCRFPEEEEGGCEL
MSLN5scFv-CD8hinge-CD3eECDTM-41BB
(SEQ ID NO: 37)
GSMALPVTALLLPLALLLHAARPQVQLVQSGAEVEKPGASVKVSCKASGY
TFTDYYMHWVRQAPGQGLEWMGWINPNSGGTNYAQKFQGRVTMTRDTSIS
TAYMELSRLRSDDTAVYYCASGWDFDYWGQGTLVTVSSGGGGSGGGGSGG
GGSGGGGSDIVMTQSPSSLSASVGDRVTITCRASQSIRYYLSWYQQKPGK
APKWYTASILQNGVPSRFSGSGSGTDFTLTISSLQPEDFATYYCLQTYTT
PDFGPGTKVEIKTTTPAPRPPTPAPTIASQPLSLRPEASRPAAGGAVHTR
GLDTGGGSDGNEEMGGITQTPYKVSISGTTVILTCPQYPGSEILWQHNDK
NIGGDEDDKNIGSDEDHLSLKEFSELEQSGYYVCYPRGSKPEDANFYLYL
RARVCENCMEMDVMSVATIVIVDICITGGLLLLVYYWSKRGRKKLLYIFK
QPFMRPVQTTQEEDGCSCRFPEEEEGGCEL
MSLN5scFv-CD3eECDTM-CD27
(SEQ ID NO: 38)
GSMALPVTALLLPLALLLHAARPQVQLVQSGAEVEKPGASVKVSCKASGY
TFTDYYMHWVRQAPGQGLEWMGWINPNSGGTNYAQKFQGRVTMTRDTSIS
TAYMELSRLRSDDTAVYYCASGWDFDYWGQGTLVTVSSGGGGSGGGGSGG
GGSGGGGSDIVMTQSPSSLSASVGDRVTITCRASQSIRYYLSWYQQKPGK
APKLLIYTASILQNGVPSRFSGSGSGTDFTLTISSLQPEDFATYYCLQTY
TTPDFGPGTKVEIKGGGGSDGNEEMGGITQTPYKVSISGTTVILTCPQYP
GSEILWQHNDKNIGGDEDDKNIGSDEDHLSLKEFSELEQSGYYVCYPRGS
KPEDANFYLYLRARVCENCMEMDVMSVATIVIVDICITGGLLLLVYYWSQ
RRKYRSNKGESPVEPAEPCHYSCPREEEGSTIPIQEDYRKPEPACSP

Lentivirus Production and Viral Transduction into Primary T Cells

As described in Example 1, lentivirus were produced and transduced into isolated primary human T-cells. Transduced T-cells and non-transduced control T-cells were expanded and frozen for subsequent analysis.

Cytotoxicity and IL2 Assay

Cytotoxicity and IL2 production induced by cross-linking primary human T-Cells to target tumor cells were assessed as described in Example 1. OVCAR8, naturally overexpressing mesothelin and transduced with firefly luciferase, was substituted as the target cell line.

Primary Human T-Cell Results

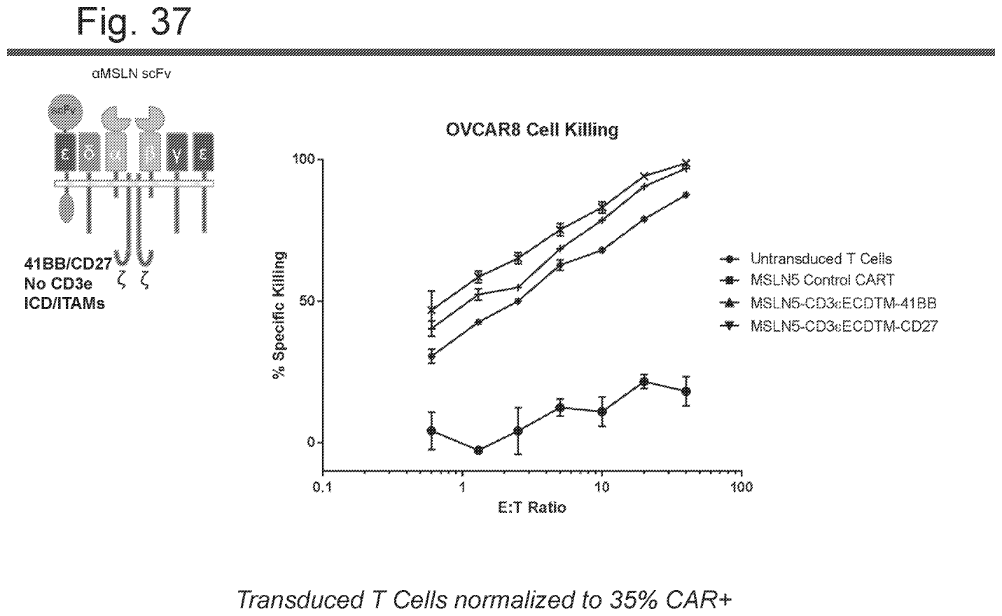

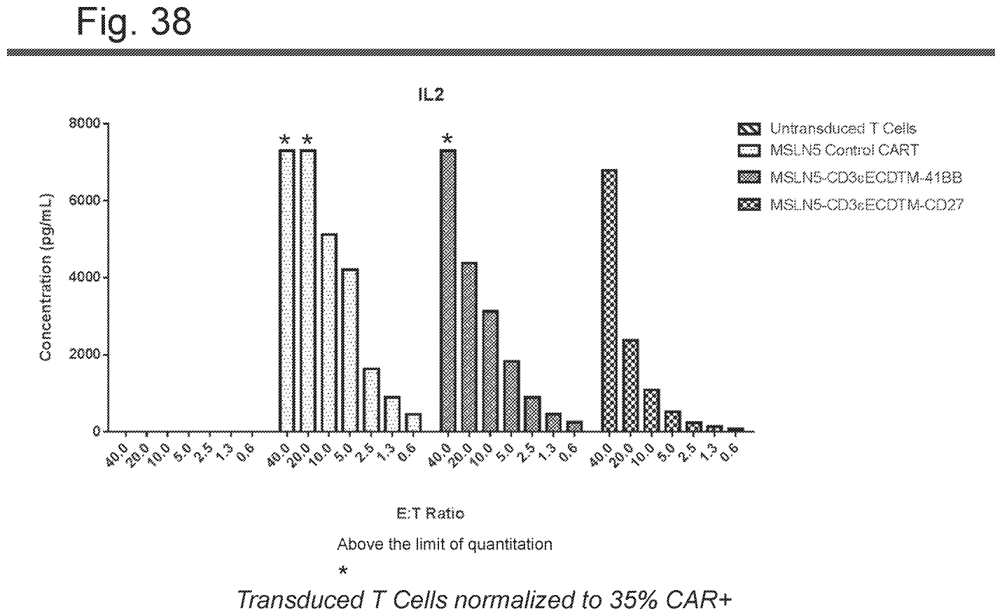

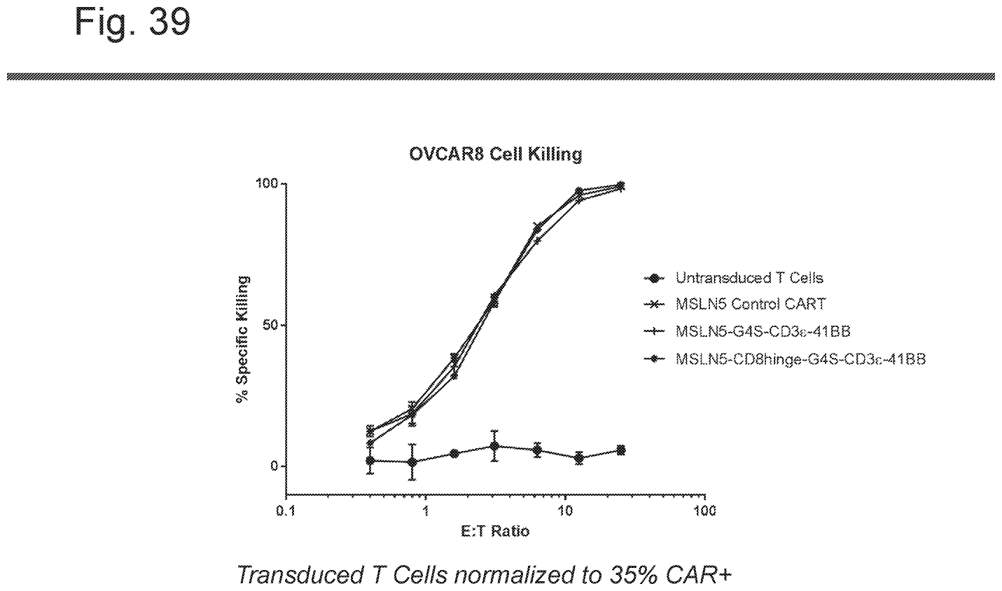

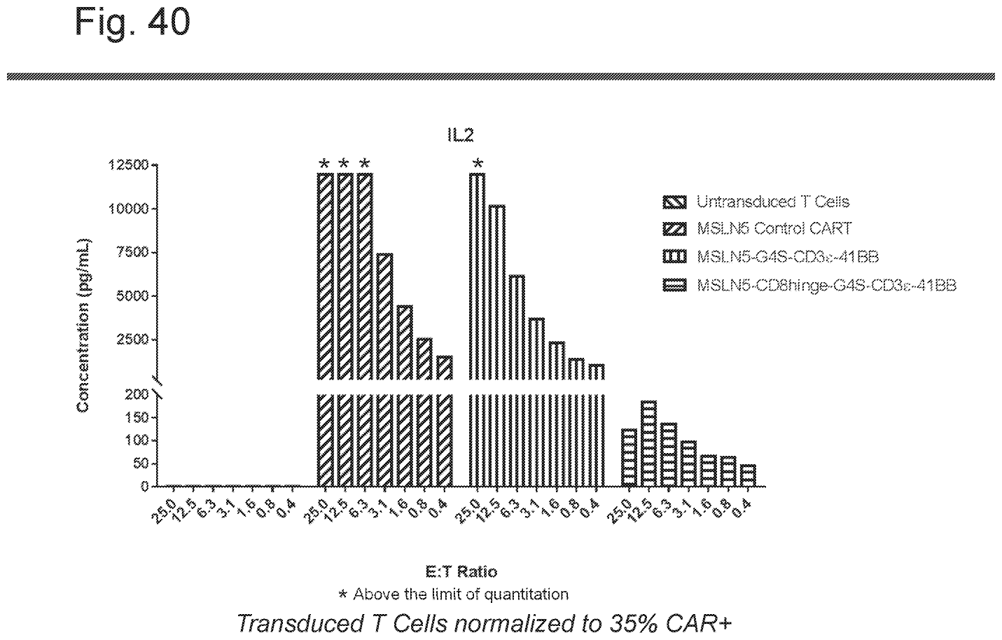

Similar to the other examples utilizing CD19-targeting TCARS, TCARs targeting mesothelin antigen are potent cytotoxic molecules. Cytotoxic activity and IL2 expression upon engagement (FIGS. 37 and 38, respectively) did not require ITAMs as a prerequisite for activity and both CD27 and 4-1BB intracellular costimulatory domains demonstrated good functional activity. As shown in FIGS. 39 and 40, the linker between the tumor targeting domain and the TCR accessory protein can modulate the functional activity of TCARs and can be adjusted to obtain the desired characteristics.

### Example 10: TCAR Targeting Mesothelin and CD19

Synthesis of Chimeric Membrane Protein Constructs Plasmid DNA is Synthesized Externally by DNA2.0. The Nominal Non-Regulatable CAR construct, MSLN5scFv-BBZ, SEQ ID NO: 35, and/or The nominal non-regulatable CAR construct, CD19scFv-BBZ, SEQ ID NO: 1, will be used as controls. In addition to the chimeric membrane proteins described in the previous examples, the following chimeric membrane proteins will also be used.

MSLN5scFv-CD3dECDTM-41BB
(SEQ ID NO: 63)
GSMALPVTALLLPLALLLHAARPQVQLVQSGAEVEKPGASVKVSCKASGY
TFTDYYMHWVRQAPGQGLEWMGWINPNSGGTNYAQKFQGRVTMTRDTSIS
TAYMELSRLRSDDTAVYYCASGWDFDYWGQGTLVTVSSGGGGSGGGGSGG
GGSGGGGSDIVMTQSPSSLSASVGDRVTITCRASQSIRYYLSWYQQKPGK
APKLLIYTASILQNGVPSRFSGSGSGTDFTLTISSLQPEDFATYYCLQTY
TTPDFGPGTKVEIKGGGGSFKIPIEELEDRVFVNCNTSITWVEGTVGTLL
SDITRLDLGKRILDPRGIYRCNGTDIYKDKESTVQVHYRMCQSCVELDPA
TVAGIIVTDVIATLLLALGVFCFAKRGRKKLLYIFKQPFMRPVQTTQEED
GCSCRFPEEEEGGCEL
MSLN5scFv-CD3gECDTM-41BB
(SEQ ID NO: 64)
GSMALPVTALLLPLALLLHAARPQVQLVQSGAEVEKPGASVKVSCKASGY
TFTDYYMHWVRQAPGQGLEWMGWINPNSGGTNYAQKFQGRVTMTRDTSIS
TAYMELSRLRSDDTAVYYCASGWDFDYWGQGTLVTVSSGGGGSGGGGSGG
GGSGGGGSDIVMTQSPSSLSASVGDRVTITCRASQSIRYYLSWYQQKPGK
APKLLIYTASILQNGVPSRFSGSGSGTDFTLTISSLQPEDFATYYCLQTY
TTPDFGPGTKVEIKGGGGSQSIKGNHLVKVYDYQEDGSVLLTCDAEAKNI
TWFKDGKMIGFLTEDKKKWNLGSNAKDPRGMYQCKGSQNKSKPLQVYYRM
CQNCIELNAATISGFLFAEIVSIFVLAVGVYFIAKRGRKKLLYIFKQPFM
RPVQTTQEEDGCSCRFPEEEEGGCEL
MSLN5scFv-CD3g(full)
(SEQ ID NO: 65)
GSMALPVTALLLPLALLLHAARPQVQLVQSGAEVEKPGASVKVSCKASGY
TFTDYYMHWVRQAPGQGLEWMGWINPNSGGTNYAQKFQGRVTMTRDTSIS
TAYMELSRLRSDDTAVYYCASGWDFDYWGQGTLVTVSSGGGGSGGGGSGG
GGSGGGGSDIVMTQSPSSLSASVGDRVTITCRASQSIRYYLSWYQQKPGK
APKLLIYTASILQNGVPSRFSGSGSGTDFTLTISSLQPEDFATYYCLQTY
TTPDFGPGTKVEIKGGGGSQSIKGNHLVKVYDYQEDGSVLLTCDAEAKNI
TWFKDGKMIGFLTEDKKKWNLGSNAKDPRGMYQCKGSQNKSKPLQVYYRM
CQNCIELNAATISGFLFAEIVSIFVLAVGVYFIAGQDGVRQSRASDKQTL
LPNDQLYQPLKDREDDQYSHLQGNQLRRN
MSLN5scFv-CD3g(full)-41BB
(SEQ ID NO: 66)
GSMALPVTALLLPLALLLHAARPQVQLVQSGAEVEKPGASVKVSCKASGY
TFTDYYMHWVRQAPGQGLEWMGWINPNSGGTNYAQKFQGRVTMTRDTSIS
TAYMELSRLRSDDTAVYYCASGWDFDYWGQGTLVTVSSGGGGSGGGGSGG
GGSGGGGSDIVMTQSPSSLSASVGDRVTITCRASQSIRYYLSWYQQKPGK
APKLLIYTASILQNGVPSRFSGSGSGTDFTLTISSLQPEDFATYYCLQTY
TTPDFGPGTKVEIKGGGGSQSIKGNHLVKVYDYQEDGSVLLTCDAEAKNI
TWFKDGKMIGFLTEDKKKWNLGSNAKDPRGMYQCKGSQNKSKPLQVYYRM
CQNCIELNAATISGFLFAEIVSIFVLAVGVYFIAGQDGVRQSRASDKQTL
LPNDQLYQPLKDREDDQYSHLQGNQLRRNKRGRKKLLYIFKQPFMRPVQT
TQEEDGCSCRFPEEEEGGCEL
MSLN5scFv-CD3e(full)-41BB
(SEQ ID NO: 67)
GSMALPVTALLLPLALLLHAARPQVQLVQSGAEVEKPGASVKVSCKASGY
TFTDYYMHWVRQAPGQGLEWMGWINPNSGGTNYAQKFQGRVTMTRDTSIS
TAYMELSRLRSDDTAVYYCASGWDFDYWGQGTLVTVSSGGGGSGGGGSGG
GGSGGGGSDIVMTQSPSSLSASVGDRVTITCRASQSIRYYLSWYQQKPGK
APKLLIYTASILQNGVPSRFSGSGSGTDFTLTISSLQPEDFATYYCLQTY
TTPDFGPGTKVEIKGGGGSDGNEEMGGITQTPYKVSISGTTVILTCPQYP
GSEILWQHNDKNIGGDEDDKNIGSDEDHLSLKEFSELEQSGYYVCYPRGS
KPEDANFYLYLRARVCENCMEMDVMSVATIVIVDICITGGLLLLVYYWSK
NRKAKAKPVTRGAGAGGRQRGQNKERPPPVPNPDYEPIRKGQRDLYSGLN
QRRIKRGRKKLLYIFKQPFMRPVQTTQEEDGCSCRFPEEEEGGCEL

Lentivirus Production and Viral Transduction into Primary T Cells

As described in Example 1, lentivirus is produced and is transduced into isolated primary human T-cells. Transduced T-cells and non-transduced control T-cells are expanded and frozen for subsequent analysis. Cells are transduced with lentivirus encoding a CD19-targeting chimeric molecule and with a mesothelin-targeting chimeric molecule as described below, and compared with cells transduced with lentivirus encoding only a CD19-targeting chimeric molecule, or only a mesothelin-targeting chimeric molecule, or untransduced cells. Cells transduced with lentivirus encoding the following constructs are produced and tested in the assays described below:

| | CD19-targeting | Mesothelin-targeting |
|---|---|---|
| Cell ID | chimeric molecule | chimeric molecule |
| Negative Control | None | None |
| CD19 Control | SEQ ID NO: 8 | None |
| Mesothelin Control | None | SEQ ID NO: 67 |
| CD19-e-BB/MESO-g-none | SEQ ID NO: 28 | SEQ ID NO: 65 |
| CD19-e-28/MESO-g-none | SEQ ID NO: 22 | SEQ ID NO: 65 |
| CD19-e-none/MESO-d-BB | SEQ ID NO: 7 | SEQ ID NO: 63 |
| CD19-e-none/MESO-g-BB | SEQ ID NO: 7 | SEQ ID NO: 64 |
| CD19-e-28/MESO-d-BB | SEQ ID NO: 22 | SEQ ID NO: 63 |
| CD19-e-28/MESO-g-BB | SEQ ID NO: 22 | SEQ ID NO: 64 |
| CD19-d-BB/MESO-g-none | SEQ ID NO: 28 | SEQ ID NO: 65 |
| CD19-d-BB/MESO-g-BB | SEQ ID NO: 28 | SEQ ID NO: 64 |
| CD19-g-BB/MESO-d-BB | SEQ ID NO: 31 | SEQ ID NO: 63 |

Cytotoxicity and IL2 Assay

Cytotoxicity and IL2 production induced by the human T-Cells engineered as in this example in response to target tumor cells is assessed as described in Example 1. Nalm6 (CD19+), OVCAR8 (mesothelin+), or a combination of Nalm6 and OVCAR8, transduced with firefly luciferase are used as the target cell line, and are compared to K562 (CD19- and mesothelin-(negative control), transduced with firefly luciferase).

### Example 11: T Cells Expressing Two Different TCARs Show Dual-Specificity

A TCAR with the specificity for CD22 (CD22-65scFv-G4S-CD3eECDTM-41BB, “CD22 TCAR,” SEQ ID NO: 73) as well as a TCAR with specificity for CD19 (CD19scFv-G4S-CD3gECDTM-41BB, “CD19-TCAR,” SEQ ID NO: 72) were cloned into lentiviral CAR expression vectors. It was tested whether T cells expressing TCARs with two different specificities also exerted specific responses to target cells expressing either or both of the target proteins.

Generation of TCAR Lentivirus

TCAR-encoding lentiviral transfer vectors were used to produce the genomic material packaged into the VSVg pseudotyped lentiviral particles. Lentiviral transfer vector DNA encoding the TCAR was mixed with the three packaging components VSVg, gag/pol and rev in combination with Lipofectamine 2000 reagent to transfect Lenti-X 293T cells (Clontech), followed by medium replacement 12-18 h later. 30 hours after medium change, the media was collected, filtered, concentrated using Lenti-X concentrator (Clontech), and stored at −80° C. in aliquots.

Generation of TCAR JNL Cells

The Jurkat NFAT Luciferase (JNL) reporter cell line is based on the acute T cell leukemia line Jurkat. The line was modified to express luciferase under control of the Nuclear Factor of Activated T cells (NFAT) response element. For the transduction with TCARs, 400,000 JNL cells/well of a 12-well plate were transduced with a multiplicity of infection (MOI) of 1.5. Frozen virus-containing supernatant was thawed at room temperature and added to the respective wells. One well each was transduced with CD19-TCAR, CD22-TCAR, or CD19-TCAR plus CD22-TCAR (“CD19/22 dual TCAR”) at MOI=1.5 each. The plates were cultured for 6 days.

Evaluating Functional Expression of Two TCARs on a Single T Cell

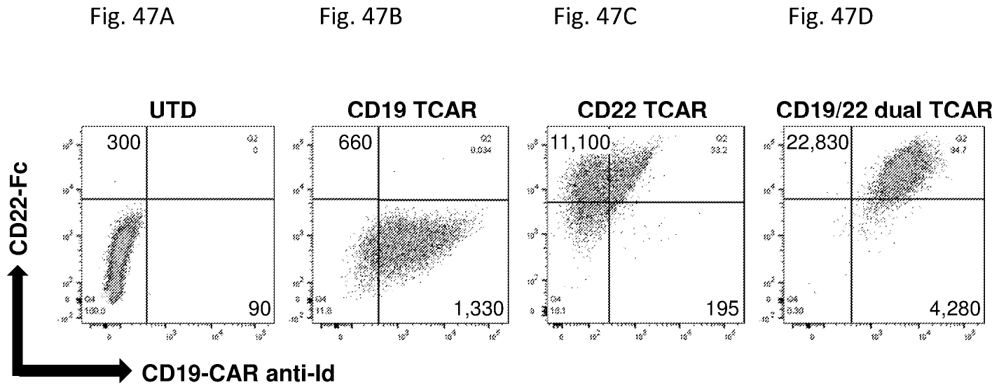

TCAR-expressing T cells were tested for their target binding capability by flow cytometry. Non-transduced (UTD), CD19-TCAR, CD22-TCAR and CD19/22 dual TCAR expressing JNL cells were tested: cells were stained with CD22-Fc for 30 min at 4° C. After a wash, cells were stained with anti-Fc secondary antibody for 30 min at 4° C. After a second wash, cells were stained with CD19-CAR anti-idiotype antibody (Ab) for 30 min at 4° C. Cells were then analyzed on a FACS LSR Fortessa after a last wash. Data was analyzed using FlowJo software. UTD JNL cells did not bind to any of the staining reagents, and low binding of CD22-Fc was interpreted as background binding (FIG. 47A). CD19-TCAR expressing cells showed correct folding and expression of the TCAR as detected by CD19-CAR anti-idiotype Ab staining, whereas CD22-Fc was not bound (FIG. 47B). CD22-TCAR expressing cells bound CD22-Fc but were not stained with the CD19-CAR anti-idiotype Ab (FIG. 47C). In contrast, JNL cells transduced with both viruses, CD19/22 dual TCARs, bound CD22 and showed binding of the CD19-CAR anti-idiotype (FIG. 47D).

Efficacy of TCAR-Redirected JNL Cells

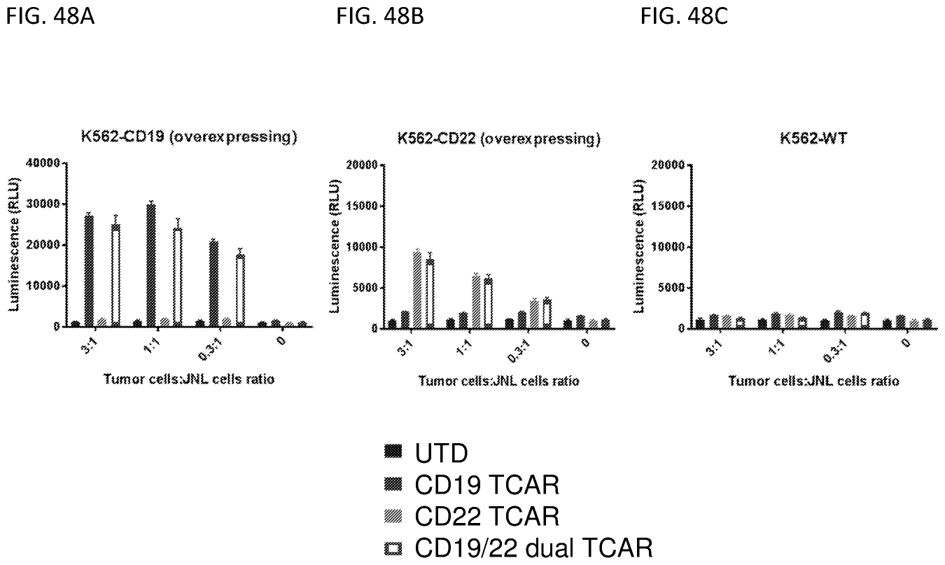

To evaluate the functional ability of TCARs, non-transduced JNL cells and the cells transduced with one or both TCAR-encoding viruses were co-cultured with target cancer cells to read out their activation by quantifying luciferase expression. JNL CART cells were co-cultured with a chronic myelogenous leukemia (CML) cell line K562 overexpressing CD19 or CD22. The parental K562 line served as a negative control. Co-cultures were set up in 384-well plates at effector-to-target (E:T) ratios of 1:3, 1:1 and 1:0.3 and incubated for 24 h, after which the expression of luciferase by the activated JNL TCAR T cells was quantified by britelite plus Reporter Gene Assay System (PerkinElmer, Waltham, Mass.). The amount of light emitted from each well (Luminescence) was a direct read-out of JNL activation by the respective TCAR. CD19/22 dual TCAR cells were activated by both CD19- and CD22-expressing K562 cells, demonstrating their dual specificity (FIGS. 48A and 48B). The extent of activation of the dual TCAR T cells was very similar to the activation of the single TCAR cells by the respective antigen, i.e. CD19 TCAR cells were activated by K562-CD19 and CD22 TCARs were activated by K562-CD22. The single TCAR cells were not activated by the non-cognate antigen (FIGS. 48A and 48B). Also the parental K562 line did not lead to activation of any of the TCARs, proving their specificity. (FIG. 48C).

### CONCLUSIONS

JNL cells transduced with viruses encoding two different TCARs were able to simultaneously express two correctly folded TCARs on the cell surface. This is demonstrated by the capability of CD19/22 dual TCAR cells to bind CD22-Fc as well as to be stained with the CD19-CAR anti-idiotype antibody (FIG. 47D). In addition to the expression on the cell surface, TCARs mediated target-dependent activation of JNLs (FIGS. 48A and 48B). Only CD19/22 dual TCAR cells were activated by both K562-CD19 and CD22, proving the dual specificity of these cells (48A and 48B).

### Example 12: Examine T Cells Expressing Two Different TCARs In Vitro and In Vivo

Human T lymphocytes are taken from a subject and are provided ex vivo, stimulated using anti-CD3/CD28 beads, and transduced with one or two lentiviral vectors encoding TCARs under the control of the EF1a promoter. Two TCARs with different specificities are used in this experiment: one TCAR specific for CD19 and the other TCAR specific for CD22. T cells will be transduced with one vector encoding for either of these TCARs or with both vectors at the same time. In addition, a single bicistronic lentivirus vector is constructed which encodes both the CD19 TCAR and the CD22 TCAR with an intervening P2A site, all under the control of the EF1a promoter, which allows for the generation of dual TCAR cells with the transduction with a single virus. TCAR T cell proliferation, cytokine release and cytotoxicity are assayed against CD19+/CD22− cells, CD19−/CD22+ cells, CD19+/CD22+ cells and a population of cells comprising CD19−/CD22+ cells and CD19+/CD22+ cells, using methods disclosed herein (e.g., as described in WO2014/130657). Cells are further assayed in vivo (proliferation, long term persistence and tumor toxicity, e.g., by methods described in WO2014/130657) by administering the cells intravenously in immune-compromised NOD/SCID/common-gamma chain−/−mice with established tumors. As for the assays in vitro, CD19+/CD22− cells, CD19−/CD22+ cells, CD19+/CD22+ cells and a population of cells comprising CD19−/CD22+ cells and CD19+/CD22+ cells are tested. CART cell persistence, proliferation/expansion and anti-tumor efficacy are monitored. It is investigated whether dual TCARs are capable of rejecting tumors consisting of mixed populations of cancer cells, expressing only one of the respective antigens or both.

### EQUIVALENTS

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

What is claimed is:

1. A system comprising:
a first chimeric membrane protein comprising an extracellular domain comprising a first antigen binding domain and a first extracellular domain derived from the extracellular domain of CD3 gamma, delta, or epsilon, a transmembrane domain, and an intracellular domain comprising a first intracellular co-stimulatory domain derived from a protein other than CD3 gamma, delta or epsilon; and
a second chimeric membrane protein comprising an extracellular domain comprising a second antigen binding domain and a second extracellular domain derived from the extracellular domain of CD3 gamma, delta, or epsilon, a transmembrane domain, and, optionally, an intracellular domain comprising a second intracellular co-stimulatory domain derived from a protein other than CD3 gamma, delta or epsilon;
wherein the first antigen binding domain and the second antigen binding domain are not identical, and wherein the first extracellular domain derived from the extracellular domain of CD3 gamma, delta, or epsilon and the second extracellular domain derived from the extracellular domain of CD3 gamma, delta, or epsilon are not identical.

2. The system of claim 1, wherein the first extracellular domain comprises the extracellular domain of CD3 gamma, delta, or epsilon, or a functional variant thereof, optionally wherein the first extracellular domain comprises the amino acid sequence of SEQ ID NO: 88, 83, or 78 (or a sequence at least about 85%, 90%, 95%, 99% or more identical thereto, and/or having one, two, three or more substitutions, insertions or deletions, e.g., conserved substitutions), optionally wherein the first extracellular domain comprises the amino acid sequence of SEQ ID NO: 88, 83, or 78.

3. The system of claim 1 or 2, wherein the second extracellular domain comprises the extracellular domain of CD3 gamma, delta, or epsilon, or a functional variant thereof, optionally wherein the second extracellular domain comprises the amino acid sequence of SEQ ID NO: 88, 83, or 78 (or a sequence at least about 85%, 90%, 95%, 99% or more identical thereto, and/or having one, two, three or more substitutions, insertions or deletions, e.g., conserved substitutions), optionally wherein the second extracellular domain comprises the amino acid sequence of SEQ ID NO: 88, 83, or 78.

4. The system of any of claims 1-3, wherein:
(i) the first chimeric membrane protein comprises the extracellular domain of CD3 gamma, or a functional variant thereof, and the second chimeric membrane protein comprises the extracellular domain of CD3 delta, or a functional variant thereof;
(ii) the first chimeric membrane protein comprises the amino acid sequence of SEQ ID NO: 88 (or a sequence at least about 85%, 90%, 95%, 99% or more identical thereto, and/or having one, two, three or more substitutions, insertions or deletions, e.g., conserved substitutions), and the second chimeric membrane protein comprises the amino acid sequence of SEQ ID NO: 83 (or a sequence at least about 85%, 90%, 95%, 99% or more identical thereto, and/or having one, two, three or more substitutions, insertions or deletions, e.g., conserved substitutions);
(iii) the first chimeric membrane protein comprises the amino acid sequence of SEQ ID NO: 88, and the second chimeric membrane protein comprises the amino acid sequence of SEQ ID NO: 83;
(iv) the first chimeric membrane protein comprises the extracellular domain of CD3 gamma, or a functional variant thereof, and the second chimeric membrane protein comprises the extracellular domain of CD3 epsilon, or a functional variant thereof;
(v) the first chimeric membrane protein comprises the amino acid sequence of SEQ ID NO: 88 (or a sequence at least about 85%, 90%, 95%, 99% or more identical thereto, and/or having one, two, three or more substitutions, insertions or deletions, e.g., conserved substitutions), and the second chimeric membrane protein comprises the amino acid sequence of SEQ ID NO: 78 (or a sequence at least about 85%, 90%, 95%, 99% or more identical thereto, and/or having one, two, three or more substitutions, insertions or deletions, e.g., conserved substitutions);
(vi) the first chimeric membrane protein comprises the amino acid sequence of SEQ ID NO: 88, and the second chimeric membrane protein comprises the amino acid sequence of SEQ ID NO: 78;
(vii) the first chimeric membrane protein comprises the extracellular domain of CD3 delta, or a functional variant thereof, and the second chimeric membrane protein comprises the extracellular domain of CD3 gamma, or a functional variant thereof;
(viii) the first chimeric membrane protein comprises the amino acid sequence of SEQ ID NO: 83 (or a sequence at least about 85%, 90%, 95%, 99% or more identical thereto, and/or having one, two, three or more substitutions, insertions or deletions, e.g., conserved substitutions), and the second chimeric membrane protein comprises the amino acid sequence of SEQ ID NO: 88 (or a sequence at least about 85%, 90%, 95%, 99% or more identical thereto, and/or having one, two, three or more substitutions, insertions or deletions, e.g., conserved substitutions);
(ix) the first chimeric membrane protein comprises the amino acid sequence of SEQ ID NO: 83, and the second chimeric membrane protein comprises the amino acid sequence of SEQ ID NO: 88;
(x) the first chimeric membrane protein comprises the extracellular domain of CD3 delta, or a functional variant thereof, and the second chimeric membrane protein comprises the extracellular domain of CD3 epsilon, or a functional variant thereof;
(xi) the first chimeric membrane protein comprises the amino acid sequence of SEQ ID NO: 83 (or a sequence at least about 85%, 90%, 95%, 99% or more identical thereto, and/or having one, two, three or more substitutions, insertions or deletions, e.g., conserved substitutions), and the second chimeric membrane protein comprises the amino acid sequence of SEQ ID NO: 78 (or a sequence at least about 85%, 90%, 95%, 99% or more identical thereto, and/or having one, two, three or more substitutions, insertions or deletions, e.g., conserved substitutions);
(xii) the first chimeric membrane protein comprises the amino acid sequence of SEQ ID NO: 83, and the second chimeric membrane protein comprises the amino acid sequence of SEQ ID NO: 78;
(xiii) the first chimeric membrane protein comprises the extracellular domain of CD3 epsilon, or a functional variant thereof, and the second chimeric membrane protein comprises the extracellular domain of CD3 gamma, or a functional variant thereof;
(xiv) the first chimeric membrane protein comprises the amino acid sequence of SEQ ID NO: 78 (or a sequence at least about 85%, 90%, 95%, 99% or more identical thereto, and/or having one, two, three or more substitutions, insertions or deletions, e.g., conserved substitutions), and the second chimeric membrane protein comprises the amino acid sequence of SEQ ID NO: 88 (or a sequence at least about 85%, 90%, 95%, 99% or more identical thereto, and/or having one, two, three or more substitutions, insertions or deletions, e.g., conserved substitutions);
(xv) the first chimeric membrane protein comprises the amino acid sequence of SEQ ID NO: 78, and the second chimeric membrane protein comprises the amino acid sequence of SEQ ID NO: 88;
(xvi) the first chimeric membrane protein comprises the extracellular domain of CD3 epsilon, or a functional variant thereof, and the second chimeric membrane protein comprises the extracellular domain of CD3 delta, or a functional variant thereof;
(xvii) the first chimeric membrane protein comprises the amino acid sequence of SEQ ID NO: 78 (or a sequence at least about 85%, 90%, 95%, 99% or more identical thereto, and/or having one, two, three or more substitutions, insertions or deletions, e.g., conserved substitutions), and the second chimeric membrane protein comprises the amino acid sequence of SEQ ID NO: 83 (or a sequence at least about 85%, 90%, 95%, 99% or more identical thereto, and/or having one, two, three or more substitutions, insertions or deletions, e.g., conserved substitutions); or
(xviii) the first chimeric membrane protein comprises the amino acid sequence of SEQ ID NO: 78, and the second chimeric membrane protein comprises the amino acid sequence of SEQ ID NO: 83.

5. The system of any of claims 1-4, wherein the transmembrane domain of the first chimeric membrane protein comprises the transmembrane domain of CD3 gamma, delta, or epsilon, or a functional variant thereof, optionally wherein the transmembrane domain of the first chimeric membrane protein comprises the amino acid sequence of SEQ ID NO: 89, 84, or 79 (or a sequence at least about 85%, 90%, 95%, 99% or more identical thereto, and/or having one, two, three or more substitutions, insertions or deletions, e.g., conserved substitutions).

6. The system of any of claims 1-4, wherein the transmembrane domain of the first chimeric membrane protein does not comprise a transmembrane domain of CD3 gamma, delta or epsilon.

7. The system of any of claims 1-6, wherein the transmembrane domain of the second chimeric membrane protein comprises the transmembrane domain of CD3 gamma, delta, or epsilon, or a functional variant thereof, optionally wherein the transmembrane domain of the second chimeric membrane protein comprises the amino acid sequence of SEQ ID NO: 89, 84, or 79 (or a sequence at least about 85%, 90%, 95%, 99% or more identical thereto, and/or having one, two, three or more substitutions, insertions or deletions, e.g., conserved substitutions).

8. The system of any of claims 1-6, wherein the transmembrane domain of the second chimeric membrane protein does not comprise a transmembrane domain of CD3 gamma, delta or epsilon.

9. The system of any of claims 1-8, wherein the first chimeric membrane protein comprises the CD3 gamma, delta or epsilon protein, or a functional variant thereof.

10. The system of claim 9, wherein:
(i) the first chimeric membrane protein comprises the amino acid sequence of SEQ ID NO:
90, 85, or 80 (or a sequence at least about 85%, 90%, 95%, 99% or more identical thereto, and/or having one, two, three or more substitutions, insertions or deletions, e.g., conserved substitutions), optionally wherein the first chimeric membrane protein comprises the amino acid sequence of SEQ ID NO: 90, 85, or 80; or
(ii) the first chimeric membrane protein comprises the amino acid sequence of SEQ ID NO: 87, 82, or 77 (or a sequence at least about 85%, 90%, 95%, 99% or more identical thereto, and/or having one, two, three or more substitutions, insertions or deletions, e.g., conserved substitutions), optionally wherein the first chimeric membrane protein comprises the amino acid sequence of SEQ ID NO: 87, 82, or 77.

11. The system of any of claims 1-10, wherein the second chimeric membrane protein comprises the CD3 gamma, delta or epsilon protein, or a functional variant thereof.

12. The system of claim 11, wherein:
(i) the second chimeric membrane protein comprises the amino acid sequence of SEQ ID NO: 90, 85, or 80 (or a sequence at least about 85%, 90%, 95%, 99% or more identical thereto, and/or having one, two, three or more substitutions, insertions or deletions, e.g., conserved substitutions), optionally wherein the second chimeric membrane protein comprises the amino acid sequence of SEQ ID NO: 90, 85, or 80; or
(ii) the second chimeric membrane protein comprises the CD3 gamma, delta or epsilon protein, or a functional variant thereof, optionally wherein the second chimeric membrane protein comprises the amino acid sequence of SEQ ID NO: 87, 82, or 77 (or a sequence at least about 85%, 90%, 95%, 99% or more identical thereto, and/or having one, two, three or more substitutions, insertions or deletions, e.g., conserved substitutions), optionally wherein the second chimeric membrane protein comprises the amino acid sequence of SEQ ID NO: 87, 82, or 77.

13. The system of any of claims 1-9, 11, or 12, wherein the first chimeric membrane protein does not comprise any intracellular domains derived from the CD3 gamma, delta or epsilon protein.

14. The system of any of claims 1-11 or 13, wherein the second chimeric membrane protein does not comprise any intracellular domains derived from the CD3 gamma, delta or epsilon protein.

15. The system of any of claims 1-14, wherein the first antigen binding domain is located N-terminal to said first extracellular domain derived from the extracellular domain of CD3 gamma, delta, or epsilon.

16. The system of any of claims 1-15, wherein the second antigen binding domain is located N-terminal to said second extracellular domain derived from the extracellular domain of CD3 gamma, delta, or epsilon.

17. The system of any of claims 1-16, wherein the first chimeric membrane protein, the second chimeric membrane protein, or both the first and second chimeric membrane proteins comprise a third antigen binding domain located N-terminal to said first and/or second antigen binding domain.

18. The system of any one of claims 1-17, wherein the first antigen binding domain and said first extracellular domain derived from the extracellular domain of CD3 gamma, delta, or epsilon are connected by a first linker and/or the second antigen binding domain and said second extracellular domain derived from the extracellular domain of CD3 gamma, delta, or epsilon are connected by a second linker.

19. The system of claim 18, wherein said first linker and/or second linker comprises, e.g., consists of, (GGGGS)n, e.g., wherein n is an integer from 0 to 10, e.g., wherein n=1, 2, or 4.

20. The system of any of claims 1-19, wherein said second chimeric membrane protein comprises a second intracellular co-stimulatory domain derived from a protein other than CD3 gamma, delta or epsilon.

21. The system of any of claims 1-19, wherein said second chimeric membrane protein does not comprise a second intracellular co-stimulatory domain derived from a protein other than CD3 gamma, delta or epsilon.

22. The system of any of claims 1-19 or 21, wherein the system does not comprise a second intracellular co-stimulatory domain.

23. The system of any of claims 1-20, comprising both the first intracellular co-stimulatory domain and the second intracellular co-stimulatory domain.

24. The system of any of claims 1-23, wherein the first chimeric membrane protein comprises a third intracellular co-stimulatory domain derived form a protein other than CD3 gamma, delta or epsilon located C-terminal to the first intracellular co-stimulatory domain.

25. The system of any of claims 1-24, wherein one or more of said intracellular co-stimulatory domains (e.g., the first intracellular co-stimulatory domain and/or second intracellular co-stimulatory domain, if present, and/or third intracellular co-stimulatory domain, if present) is a functional signaling domain of a protein selected from the group consisting of: an MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TN1-R2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, or a functional variant thereof.

26. The system of any of claims 1-25, wherein one or more of said intracellular co-stimulatory domains (e.g., the first intracellular co-stimulatory domain and/or second intracellular co-stimulatory domain, if present, and/or third intracellular co-stimulatory domain, if present) is a functional signaling domain of 4-1BB, or a functional variant thereof, optionally wherein one or more of said intracellular co-stimulatory domains (e.g., the first intracellular co-stimulatory domain and/or second intracellular co-stimulatory domain, if present, and/or third intracellular co-stimulatory domain, if present) comprises the amino acid sequence of SEQ ID NO: 50 (or a sequence at least about 85%, 90%, 95%, 99% or more identical thereto, and/or having one, two, three or more substitutions, insertions or deletions, e.g., conserved substitutions), optionally wherein one or more of said intracellular co-stimulatory domains (e.g., the first intracellular co-stimulatory domain and/or second intracellular co-stimulatory domain, if present, and/or third intracellular co-stimulatory domain, if present) comprises the amino acid sequence of SEQ ID NO: 50.

27. The system of any of claims 1-26, wherein:
(i) the first chimeric membrane protein comprises the amino acid sequence of SEQ ID NO: 91, 86, or 81 (or a sequence at least about 85%, 90%, 95%, 99% or more identical thereto, and/or having one, two, three or more substitutions, insertions or deletions, e.g., conserved substitutions), optionally wherein the first chimeric membrane protein comprises the amino acid sequence of SEQ ID NO: 91, 86, or 81; or
(ii) the second chimeric membrane protein comprises the amino acid sequence of SEQ ID NO: 91, 86, or 81 (or a sequence at least about 85%, 90%, 95%, 99% or more identical thereto, and/or having one, two, three or more substitutions, insertions or deletions, e.g., conserved substitutions), optionally wherein the first chimeric membrane protein comprises the amino acid sequence of SEQ ID NO: 91, 86, or 81.

28. The system of any of claims 1-27, wherein the first antigen binding domain binds a tumor antigen.

29. The system of any of claims 1-28, wherein the first antigen binding domain binds a B-cell antigen.

30. The system of claim 29, wherein the B-cell antigen bound by the first antigen binding domain is CD5, CD10, CD19, CD20, CD21, CD22, CD23, CD24, CD25, CD27, CD30, CD34, CD37, CD38, CD40, CD53, CD69, CD72, CD73, CD74, CD75, CD77, CD79a, CD79b, CD80, CD81, CD82, CD83, CD84, CD85, CD86, CD123, CD135, CD138, CD179, CD269, Flt3, ROR1, BCMA, FcRn5, FcRn2, CS-1, CXCR4, 5, 7, IL-7/3R, IL7/4/3R, or IL4R.

31. The system of claim 30, wherein the B-cell antigen bound by the first antigen binding domain is CD19, CD20, CD22, FcRn5, FcRn2, BCMA, CS-1, or CD138.

32. The system of any of claims 1-31, wherein the second antigen binding domain binds a tumor antigen.

33. The system of any of claims 1-32, wherein the second antigen binding domain binds a B-cell antigen.

34. The system of any of claims 29-31 and 33, wherein the second antigen binding domain binds a different B-cell antigen than the B-cell antigen bound by the first antigen binding domain.

35. The system of claim 33 or 34, wherein the B-cell antigen bound by the second antigen binding domain is CD5, CD10, CD19, CD20, CD21, CD22, CD23, CD24, CD25, CD27, CD30, CD34, CD37, CD38, CD40, CD53, CD69, CD72, CD73, CD74, CD75, CD77, CD79a, CD79b, CD80, CD81, CD82, CD83, CD84, CD85, CD86, CD123, CD135, CD138, CD179, CD269, Flt3, ROR1, BCMA, FcRn5, FcRn2, CS-1, CXCR4, 5, 7, IL-7/3R, IL7/4/3R, or IL4R.

36. The system of claim 35, wherein the B-cell antigen bound by the second antigen binding domain is CD19, CD20, CD22, FcRn5, FcRn2, BCMA, CS-1, or CD138.

37. The system of any of claims 33-36, wherein:
(i) the first antigen binding domain binds CD19 and the second antigen binding domain binds CD20;
(ii) the first antigen binding domain binds CD19 and the second antigen binding domain binds CD22;
(iii) the first antigen binding domain binds CD20 and the second antigen binding domain binds CD22;
(iv) the first antigen binding domain binds CD20 and the second antigen binding domain binds CD19;
(v) the first antigen binding domain binds CD22 and the second antigen binding domain binds CD19; or
(vi) the first antigen binding domain binds CD22 and the second antigen binding domain binds CD20.

38. The system of claim 37, wherein the first antigen binding domain binds CD19 and the second antigen binding domain binds CD22, optionally wherein:
(i) the first chimeric membrane protein comprises the amino acid sequence of SEQ ID NO: 70 (or a sequence at least about 85%, 90%, 95%, 99% or more identical thereto, and/or having one, two, three or more substitutions, insertions or deletions, e.g., conserved substitutions), and the second chimeric membrane protein comprises the amino acid sequence of SEQ ID NO: 75 or 76 (or a sequence at least about 85%, 90%, 95%, 99% or more identical thereto, and/or having one, two, three or more substitutions, insertions or deletions, e.g., conserved substitutions);
(ii) the first chimeric membrane protein comprises the amino acid sequence of SEQ ID NO: 71 (or a sequence at least about 85%, 90%, 95%, 99% or more identical thereto, and/or having one, two, three or more substitutions, insertions or deletions, e.g., conserved substitutions), and the second chimeric membrane protein comprises the amino acid sequence of SEQ ID NO: 73, 74, 75, or 76 (or a sequence at least about 85%, 90%, 95%, 99% or more identical thereto, and/or having one, two, three or more substitutions, insertions or deletions, e.g., conserved substitutions); or
(iii) the first chimeric membrane protein comprises the amino acid sequence of SEQ ID NO: 72 (or a sequence at least about 85%, 90%, 95%, 99% or more identical thereto, and/or having one, two, three or more substitutions, insertions or deletions, e.g., conserved substitutions), and the second chimeric membrane protein comprises the amino acid sequence of SEQ ID NO: 73 or 74 (or a sequence at least about 85%, 90%, 95%, 99% or more identical thereto, and/or having one, two, three or more substitutions, insertions or deletions, e.g., conserved substitutions).

39. The system of any of claims 1-38, wherein the first or second antigen binding domain binds a solid tumor antigen.

40. The system of claim 39, wherein the solid tumor antigen is EGFRvIII, mesothelin, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRa4, or a peptide of any of these antigens presented on MHC.

41. The system of claim 40, wherein said solid tumor antigen is selected from the group consisting of CLDN6, mesothelin and EGFRvIII.

42. The system of claim 41, wherein:
(i) the first antigen binding domain binds CD19 and the second antigen binding domain binds mesothelin;
(ii) the first antigen binding domain binds CD19 and the second antigen binding domain binds EGFRvIII;
(iii) the first antigen binding domain binds CD19 and the second antigen binding domain binds CLDN6;
(iv) the first antigen binding domain binds mesothelin and the second antigen binding domain binds CD19;
(v) the first antigen binding domain binds EGFRvIII and the second antigen binding domain binds CD19; or
(vi) the first antigen binding domain binds CLDN6 and the second antigen binding domain binds CD19.

43. A nucleic acid construct encoding the system of any one of claims 1-42.

44. The nucleic acid construct of claim 43, wherein said nucleic acid construct is mRNA.

45. The nucleic acid construct of claim 43 or 44, comprising a first nucleic acid molecule encoding the first chimeric membrane protein and a second nucleic acid molecule encoding the second chimeric membrane protein, optionally wherein:
(i) the first and second nucleic acid molecules are disposed on a single nucleic acid molecule, or
(ii) the first and second nucleic acid molecules are disposed on separate nucleic acid molecules.

46. A vector comprising the nucleic acid construct of any of claims 43-45.

47. The vector of claim 46, wherein said vector is a lentiviral, adenoviral, or retroviral vector.

48. The vector of claim 46 or 47, wherein, upon expression of said first and second chimeric membrane proteins, said proteins are expressed as a single mRNA transcript.

49. The vector of claim 48, wherein the nucleic acid sequences encoding said first and second chimeric membrane proteins are separated by a nucleic acid sequence encoding a self-cleavage site or an internal ribosomal entry site.

50. A cell comprising the nucleic acid construct of any of claims 43-45, the vector of any of claims 46-49, or the system of any of claims 1-42.

51. The cell of claim 50, wherein said cell is an NK cell or T cell.

52. The cell of claim 50 or 51, further comprising a first inhibitor, wherein:
(i) the first chimeric membrane protein comprises a first extracellular domain derived from the extracellular domain of CD3 gamma, and the first inhibitor reduces the expression of endogenous CD3 gamma in the cell;
(ii) the first chimeric membrane protein comprises a first extracellular domain derived from the extracellular domain of CD3 delta, and the first inhibitor reduces the expression of endogenous CD3 delta in the cell; or
(iii) the first chimeric membrane protein comprises a first extracellular domain derived from the extracellular domain of CD3 epsilon, and the first inhibitor reduces the expression of endogenous CD3 epsilon in the cell, optionally wherein:
the first inhibitor does not reduce or does not substantially reduce the expression of the first chimeric membrane protein in the cell (e.g., the first inhibitor reduces the expression of the first chimeric membrane protein at a level no more than 2, 5, 10, 15, or 20% compared to the expression of the first chimeric membrane protein in the absence of the first inhibitor).

53. The cell of any one of claims 50-52, further comprising a second inhibitor, wherein:
(i) the second chimeric membrane protein comprises a second extracellular domain derived from the extracellular domain of CD3 gamma, and the second inhibitor reduces the expression of endogenous CD3 gamma in the cell;
(ii) the second chimeric membrane protein comprises a second extracellular domain derived from the extracellular domain of CD3 delta, and the second inhibitor reduces the expression of endogenous CD3 delta in the cell; or
(iii) the second chimeric membrane protein comprises a second extracellular domain derived from the extracellular domain of CD3 epsilon, and the second inhibitor reduces the expression of endogenous CD3 epsilon in the cell, optionally wherein:
the second inhibitor does not reduce or does not substantially reduce the expression of the second chimeric membrane protein in the cell (e.g., the second inhibitor reduces the expression of the second chimeric membrane protein at a level no more than 2, 5, 10, 15, or 20% compared to the expression of the second chimeric membrane protein in the absence of the second inhibitor).

54. The cell of claim 52 or 53, wherein the first or second inhibitor is an agent that mediates RNA interference, e.g., an siRNA or shRNA, or a nucleic acid molecule encoding an siRNA or shRNA.

55. The cell of claim 52 or 53, wherein the first or second inhibitor is a gene editing system (e.g., a CRISPR/Cas9 system, a zinc finger nuclease system, a TALEN system, or a meganuclease system) or a nucleic acid molecule encoding one or more components of the gene editing system.

56. A method of treating a subject with a proliferative disorder, said method comprising administering to the subject the cell of any one of claims 50-55.

57. The method of claim 56, wherein said subject has a tumor and said administration provides said subject with immunity against said tumor.

58. A method of providing an anti-cancer immune response in a subject having a cancer, comprising administering to the subject the cell of any one of claims 50-55.

59. The method of any of claims 56-58, wherein said cell is a T cell or NK cell and is autologous to said subject.

60. The method of any of claims 56-58, wherein said cell is an allogeneic T cell or NK cell.

61. The method of any of claims 56-60, wherein said subject is a human.

62. The method of any of claims 56-61, wherein the subject has a cancer.

63. The method of claim 62, wherein the cancer is chosen from mesothelioma (e.g., malignant pleural mesothelioma), e.g., in a subject who has progressed on at least one prior standard therapy; lung cancer (e.g., non-small cell lung cancer, small cell lung cancer, squamous cell lung cancer, or large cell lung cancer); pancreatic cancer (e.g., pancreatic ductal adenocarcinoma, or metastatic pancreatic ductal adenocarcinoma (PDA), e.g., in a subject who has progressed on at least one prior standard therapy); esophageal adenocarcinoma, ovarian cancer (e.g., serous epithelial ovarian cancer, e.g., in a subject who has progressed after at least one prior regimen of standard therapy), breast cancer, colorectal cancer, bladder cancer or any combination thereof.

64. The method of claim 62, wherein the cancer is chosen from chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, acute myeloid leukemia (AML), or unclassifiable lymphoma.

65. The method of claim 62, wherein the first antigen binding domain binds to a first antigen (e.g., a first tumor antigen) and the second antigen binding domain binds to a second antigen (e.g., a second tumor antigen), wherein the cancer exhibits heterogeneous expression of the first antigen (e.g., a first tumor antigen) and/or the second antigen (e.g., a second tumor antigen), e.g., wherein less than 90%, 80%, 70%, 60%, or 50% of cells in the cancer express the first antigen (e.g., a first tumor antigen) and less than 90%, 80%, 70%, 60%, or 50% of cells in the cancer express the second antigen (e.g., a second tumor antigen).

66. A method of making a cell, comprising introducing the vector of any of claims 46-49 into a cell, e.g., transducing a cell with the vector of any of claims 46-49.

67. The method of claim 66, further comprising introducing a first inhibitor into the cell, wherein:
(i) the first chimeric membrane protein comprises a first extracellular domain derived from the extracellular domain of CD3 gamma, and the first inhibitor reduces the expression of endogenous CD3 gamma in the cell;
(ii) the first chimeric membrane protein comprises a first extracellular domain derived from the extracellular domain of CD3 delta, and the first inhibitor reduces the expression of endogenous CD3 delta in the cell; or
(iii) the first chimeric membrane protein comprises a first extracellular domain derived from the extracellular domain of CD3 epsilon, and the first inhibitor reduces the expression of endogenous CD3 epsilon in the cell, optionally wherein:
the first inhibitor does not reduce or does not substantially reduce the expression of the first chimeric membrane protein in the cell (e.g., the first inhibitor reduces the expression of the first chimeric membrane protein at a level no more than 2, 5, 10, 15, or 20% compared to the expression of the first chimeric membrane protein in the absence of the first inhibitor).

68. The method of claim 66 or 67, further comprising introducing a second inhibitor into the cell, wherein:
(i) the second chimeric membrane protein comprises a second extracellular domain derived from the extracellular domain of CD3 gamma, and the second inhibitor reduces the expression of endogenous CD3 gamma in the cell;
(ii) the second chimeric membrane protein comprises a second extracellular domain derived from the extracellular domain of CD3 delta, and the second inhibitor reduces the expression of endogenous CD3 delta in the cell; or
(iii) the second chimeric membrane protein comprises a second extracellular domain derived from the extracellular domain of CD3 epsilon, and the second inhibitor reduces the expression of endogenous CD3 epsilon in the cell, optionally wherein:
the second inhibitor does not reduce or does not substantially reduce the expression of the second chimeric membrane protein in the cell (e.g., the second inhibitor reduces the expression of the second chimeric membrane protein at a level no more than 2, 5, 10, 15, or 20% compared to the expression of the second chimeric membrane protein in the absence of the second inhibitor).

69. The method of claim 67 or 68, wherein the first or second inhibitor is an agent that mediates RNA interference, e.g., an siRNA or shRNA, or a nucleic acid molecule encoding an siRNA or shRNA.

70. The method of claim 67 or 68, wherein the first or second inhibitor is a gene editing system (e.g., a CRISPR/Cas9 system, a zinc finger nuclease system, a TALEN system, or a meganuclease system) or a nucleic acid molecule encoding one or more components of the gene editing system.

71. The method of any of claims 66-70, wherein the cell is an immune effector cell, e.g., a T cell or an NK cell.


SEQUENCE LISTING (104 sequences)

SEQ ID NO: 1 - protein, 495 aa, Artificial Sequence
  source: /note=“Description of Artificial Sequence Synthetic polypeptide”
GSATMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 476 aa, Artificial Sequence
  source: /note=“Description of Artificial Sequence Synthetic polypeptide”
GSATMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCSLKRGRKKLLYIFKQPFMRPVQTTQEEDGCSCRFPEEEEGGCELMFNAKYVAEATGNFITVMDALKLNYNAKDQLHPLLAELLISINRVTRDDFENRSKLIDWIVRINKLSIGDTLTETQIRELLFDLELAYKSFYALLD

SEQ ID NO: 3 - protein, 295 aa, Artificial Sequence
  source: /note=“Description of Artificial Sequence Synthetic polypeptide”
GSMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGSGGSGSGGGSGSGSSGASADVVSTWVCPICMVSNETQGEFTKDTLPTPICINCGVPADYELTKSSINCSNAIDPNANPRNQFG

SEQ ID NO: 4 - protein, 417 aa, Artificial Sequence
  source: /note=“Description of Artificial Sequence Synthetic polypeptide”
GSATMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCSLKRGRKKLLYIFKQPFMRPVQTTQEEDGCSCRFPEEEEGGCELRIARLEEEVKTLEAQNSELASTANMLEEQVAQLKQKV

SEQ ID NO: 5 - protein, 253 aa, Artificial Sequence
  source: /note=“Description of Artificial Sequence Synthetic polypeptide”
GSMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGSGGSLTDTLQAKTDQLKDEKSALQTKIANLLKEKEKLEFIL

SEQ ID NO: 6 - protein, 281 aa, Artificial Sequence
  source: /note=“Description of Artificial Sequence Synthetic polypeptide”
GSMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSRSKRSRLLHSDYMNMTPRRPGPTRKHYQPYAPPRDFAAYRSGSGSGGSGSGGGSGSGSSGASADVVSTWVCPICMVSNETQGEFTKDTLPTPICINCGVPADYELTKSSINCSNAIDPNANPRNQFG

SEQ ID NO: 7 - protein, 457 aa, Artificial Sequence
  source: /note=“Description of Artificial Sequence Synthetic polypeptide”
GSATMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 8 - protein, 506 aa, Artificial Sequence
  source: /note=“Description of Artificial Sequence Synthetic polypeptide”
GSATMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGSGGSKRGRKKLLYIFKQPFMRPVQTTQEEDGCSCRFPEEEEGGCEL

SEQ ID NO: 9 - protein, 444 aa, Artificial Sequence
  source: /note=“Description of Artificial Sequence Synthetic polypeptide”
GSATMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKRGRKKLLYIFKQPFMRPVQTTQEEDGCSCRFPEEEEGGCEL

SEQ ID NO: 10 - protein, 451 aa, Artificial Sequence
  source: /note=“Description of Artificial Sequence Synthetic polypeptide”
GSMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 451 aa, Artificial Sequence
  source: /note=“Description of Artificial Sequence Synthetic polypeptide”
GSMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCRVKFSRSADAPAFKQGQNQLFNELNLGRREEFDVLDKRRGRDPEMGGKPRRKNPQEGLFNELQKDKMAEAFSEIGMKGERRRGKGHDGLFQGLSTATKDTFDALHMQALPPR

SEQ ID NO: 12 - protein, 381 aa, Artificial Sequence
  source: /note=“Description of Artificial Sequence Synthetic polypeptide”
GSMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCEL

SEQ ID NO: 13 - protein, 455 aa, Artificial Sequence
  source: /note=“Description of Artificial Sequence Synthetic polypeptide”
GSMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDFEPIRKGQRDLFSGLNQRRI

SEQ ID NO: 14 - protein, 488 aa, Artificial Sequence
  source: /note=“Description of Artificial Sequence Synthetic polypeptide”
GSATMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCSLKRGRKKLLYIFKQPFMRPVQTTQEEDGCSCRFPEEEEGGCELMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 15 - protein, 309 aa, Artificial Sequence
  source: /note=“Description of Artificial Sequence Synthetic polypeptide”
GSMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGSGGSILWHEMWHEGLIEASRLYFGERNVKGMFEVLEPLHAMMERGPQTLKETSFNQAYGRDLMEAQEWCRKYMKSGNVKDLLQAWDLYYHVFRRISK

SEQ ID NO: 16 - protein, 309 aa, Artificial Sequence
  source: /note=“Description of Artificial Sequence Synthetic polypeptide”
GSMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDFEPIRKGQRDLFSGLNQRRIGSGSGGSILWHEMWHEGLIEASRLYFGERNVKGMFEVLEPLHAMMERGPQTLKETSFNQAYGRDLMEAQEWCRKYMKSGNVKDLLQAWDLYYHVFRRISK

SEQ ID NO: 17 - protein, 362 aa, Artificial Sequence
  source: /note=“Description of Artificial Sequence Synthetic polypeptide”
GSATMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSPEDANFYLYLRARVCENCMEMDVMSVATIVIVDICITGGLLLLVYYWSKRGRKKLLYIFKQPFMRPVQTTQEEDGCSCRFPEEEEGGCEL

SEQ ID NO: 18 - protein, 362 aa, Artificial Sequence
  source: /note=“Description of Artificial Sequence Synthetic polypeptide”
GSATMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSPEDANFYLYLRARVSENCMEMDVMSVATIVIVDICITGGLLLLVYYWSKRGRKKLLYIFKQPFMRPVQTTQEEDGCSCRFPEEEEGGCEL

SEQ ID NO: 19 - protein, 362 aa, Artificial Sequence
  source: /note=“Description of Artificial Sequence Synthetic polypeptide”
GSATMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSPEDANFYLYLRARVCENSMEMDVMSVATIVIVDICITGGLLLLVYYWSKRGRKKLLYIFKQPFMRPVQTTQEEDGCSCRFPEEEEGGCEL

SEQ ID NO: 20 - protein, 362 aa, Artificial Sequence
  source: /note=“Description of Artificial Sequence Synthetic polypeptide”
GSATMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSPEDANFYLYLRARVSENSMEMDVMSVATIVIVDICITGGLLLLVYYWSKRGRKKLLYIFKQPFMRPVQTTQEEDGCSCRFPEEEEGGCEL

SEQ ID NO: 21 - protein, 448 aa, Artificial Sequence
  source: /note=“Description of Artificial Sequence Synthetic polypeptide”
GSMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSQRRKYRSNKGESPVEPAEPCHYSCPREEEGSTIPIQEDYRKPEPACSP

SEQ ID NO: 22 - protein, 441 aa, Artificial Sequence
  source: /note=“Description of Artificial Sequence Synthetic polypeptide”
GSMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSRSKRSRLLHSDYMNMTPRRPGPTRKHYQPYAPPRDFAAYRS

SEQ ID NO: 23 - protein, 437 aa, Artificial Sequence
  source: /note=“Description of Artificial Sequence Synthetic polypeptide”
GSMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSRRDQRLPPDAHKPPGGGSFRTPIQEEQADAHSTLAKI

SEQ ID NO: 24 - protein, 435 aa, Artificial Sequence
  source: /note=“Description of Artificial Sequence Synthetic polypeptide”
GSMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSTKKKYSSSVHDPNGEYMFMRAVNTAKKSRLTDVTL

SEQ ID NO: 25 - protein, 516 aa, Artificial Sequence
  source: /note=“Description of Artificial Sequence Synthetic polypeptide”
GSMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 26 - protein, 447 aa, Artificial Sequence
  source: /note=“Description of Artificial Sequence Synthetic polypeptide”
GSMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKRGRKKLLYIFKQPFMRPVQTTQEEDGCSCRFPEEEEGGCEL

SEQ ID NO: 27 - protein, 457 aa, Artificial Sequence
  source: /note=“Description of Artificial Sequence Synthetic polypeptide”
GSMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKRGRKKLLYIFKQPFMRPVQTTQEEDGCSCRFPEEEEGGCEL

SEQ ID NO: 28 - protein, 419 aa, Artificial Sequence
  source: /note=“Description of Artificial Sequence Synthetic polypeptide”
GSATMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAKRGRKKLLYIFKQPFMRPVQTTQEEDGCSCRFPEEEEGGCEL

SEQ ID NO: 29 - protein, 422 aa, Artificial Sequence
  source: /note=“Description of Artificial Sequence Synthetic polypeptide”
GSMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAKRGRKKLLYIFKQPFMRPVQTTQEEDGCSCRFPEEEEGGCEL

SEQ ID NO: 30 - protein, 432 aa, Artificial Sequence
  source: /note=“Description of Artificial Sequence Synthetic polypeptide”
GSMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAKRGRKKLLYIFKQPFMRPVQTTQEEDGCSCRFPEEEEGGCEL

SEQ ID NO: 31 - protein, 429 aa, Artificial Sequence
  source: /note=“Description of Artificial Sequence Synthetic polypeptide”
GSATMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAKRGRKKLLYIFKQPFMRPVQTTQEEDGCSCRFPEEEEGGCEL

SEQ ID NO: 32 - protein, 432 aa, Artificial Sequence
  source: /note=“Description of Artificial Sequence Synthetic polypeptide”
GSMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAKRGRKKLLYIFKQPFMRPVQTTQEEDGCSCRFPEEEEGGCEL

SEQ ID NO: 33 - protein, 442 aa, Artificial Sequence
  source: /note=“Description of Artificial Sequence Synthetic polypeptide”
GSMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAKRGRKKLLYIFKQPFMRPVQTTQEEDGCSCRFPEEEEGGCEL

SEQ ID NO: 34 - protein, 342 aa, Artificial Sequence
  source: /note=“Description of Artificial Sequence Synthetic polypeptide”
GSMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSQSFGLLDPKLCYLLDGILFIYGVILTALFLKRGRKKLLYIFKQPFMRPVQTTQEEDGCSCRFPEEEEGGCEL

SEQ ID NO: 35 - protein, 487 aa, Artificial Sequence
  source: /note=“Description of Artificial Sequence Synthetic polypeptide”
GSMALPVTALLLPLALLLHAARPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 441 aa, Artificial Sequence
  source: /note=“Description of Artificial Sequence Synthetic polypeptide”
GSMALPVTALLLPLALLLHAARPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKRGRKKLLYIFKQPFMRPVQTTQEEDGCSCRFPEEEEGGCEL

SEQ ID NO: 37 - protein, 482 aa, Artificial Sequence
  source: /note=“Description of Artificial Sequence Synthetic polypeptide”
GSMALPVTALLLPLALLLHAARPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKTTTPAPRPPTPAPTIASQPLSLRPEASRPAAGGAVHTRGLDTGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKRGRKKLLYIFKQPFMRPVQTTQEEDGCSCRFPEEEEGGCEL

SEQ ID NO: 38 - protein, 447 aa, Artificial Sequence
  source: /note=“Description of Artificial Sequence Synthetic polypeptide”
GSMALPVTALLLPLALLLHAARPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSQRRKYRSNKGESPVEPAEPCHYSCPREEEGSTIPIQEDYRKPEPACSP

SEQ ID NO: 39 - protein, 4 aa, Artificial Sequence
  source: /note=“Description of Artificial Sequence Synthetic peptide”
RGDS

SEQ ID NO: 40 - protein, 21 aa, Artificial Sequence
  source: /note=“Description of Artificial Sequence Synthetic peptide”
  VARIANT (1)..(3): /replace=“ ”
  MISC_FEATURE (1)..(21): /note=“Variant residues given in the sequence have no preference with respect to those in the annotations for variant positions”
GSGEGRGSLLTCGDVEENPGP

SEQ ID NO: 41 - protein, 22 aa, Artificial Sequence
  source: /note=“Description of Artificial Sequence Synthetic peptide”
  VARIANT (1)..(3): /replace=“ ”
  MISC_FEATURE (1)..(22): /note=“Variant residues given in the sequence have no preference with respect to those in the annotations for variant positions”
GSGATNFSLLKQAGDVEENPGP

SEQ ID NO: 42 - protein, 23 aa, Artificial Sequence
  source: /note=“Description of Artificial Sequence Synthetic peptide”
  VARIANT (1)..(3): /replace=“ ”
  MISC_FEATURE (1)..(23): /note=“Variant residues given in the sequence have no preference with respect to those in the annotations for variant positions”
GSGQCTNYALLKLAGDVESNPGP

SEQ ID NO: 43 - protein, 25 aa, Artificial Sequence
  source: /note=“Description of Artificial Sequence Synthetic peptide”
  VARIANT (1)..(3): /replace=“ ”
  MISC_FEATURE (1)..(25): /note=“Variant residues given in the sequence have no preference with respect to those in the annotations for variant positions”
GSGVKQTLNFDLLKLAGDVESNPGP

SEQ ID NO: 44 - protein, 4 aa, Artificial Sequence
  source: /note=“Description of Artificial Sequence Synthetic peptide”
GGGS

SEQ ID NO: 45 - protein, 20 aa, Artificial Sequence
  source: /note=“Description of Artificial Sequence Synthetic peptide”
GGGGSGGGGSGGGGSGGGGS

SEQ ID NO: 46 - protein, 15 aa, Artificial Sequence
  source: /note=“Description of Artificial Sequence Synthetic peptide”
GGGGSGGGGSGGGGS

SEQ ID NO: 47 - DNA, 1184 bp, Artificial Sequence
  source: /note=“Description of Artificial Sequence Synthetic polynucleotide”
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 48 - protein, 21 aa, Artificial Sequence
  source: /note=“Description of Artificial Sequence Synthetic peptide”
MALPVTALLLPLALLLHAARP

SEQ ID NO: 49 - DNA, 63 bp, Artificial Sequence
  source: /note=“Description of Artificial Sequence Synthetic oligonucleotide”
atggccctgcctgtgacagccctgctgctgcctctggctctgctgctgcatgccgctagaccc

SEQ ID NO: 50 - protein, 42 aa, Artificial Sequence
  source: /note=“Description of Artificial Sequence Synthetic polypeptide”
KRGRKKLLYIFKQPFMRPVQTTQEEDGCSCRFPEEEEGGCEL

SEQ ID NO: 51 - DNA, 126 bp, Artificial Sequence
  source: /note=“Description of Artificial Sequence Synthetic polynucleotide”
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 52 - protein, 5 aa, Artificial Sequence
  source: /note=“Description of Artificial Sequence Synthetic peptide”
GGGGS

SEQ ID NO: 53 - protein, 132 aa, Artificial Sequence
  source: /note=“Description of Artificial Sequence Synthetic polypeptide”
DVPDYASLGGPSSPKKKRKVSRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSY

SEQ ID NO: 54 - protein, 108 aa, Artificial Sequence
  source: /note=“Description of Artificial Sequence Synthetic polypeptide”
VQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETS

SEQ ID NO: 55 - protein, 93 aa, Artificial Sequence [sequence omitted]

SEQ ID NO: 56 - protein, 95 aa, Artificial Sequence
  source: /note=“Description of Artificial Sequence Synthetic polypeptide”
ILWHEMWHEGLIEASRLYFGERNVKGMFEVLEPLHAMMERGPQTLKETSFNQAYGRDLMEAQEWCRKYMKSGNVKDLTQAWDLYYHVFRRISKTS

SEQ ID NO: 57 - protein, 95 aa, Artificial Sequence
  source: /note=“Description of Artificial Sequence Synthetic polypeptide”
ILWHEMWHEGLLEASRLYFGERNVKGMFEVLEPLHAMMERGPQTLKETSFNQAYGRDLMEAQEWCRKYMKSGNVKDLTQAWDLYYHVFRRISKTS

SEQ ID NO: 58 - protein, 95 aa, Artificial Sequence
  source: /note=“Description of Artificial Sequence Synthetic polypeptide”
ILWHEMWHEGLEEASRLYFGERNVKGMFEVLEPLHAMMERGPQTLKETSFNQAYGRDLMEAQEWCRKYMKSGNVKDLLQAWDLYYHVFRRISKTS

SEQ ID NO: 59 - protein, 95 aa, Artificial Sequence
  source: /note=“Description of Artificial Sequence Synthetic polypeptide”
  MOD_RES (12)..(12): Any amino acid
  MOD_RES (78)..(78): Any amino acid
ILWHEMWHEGLXEASRLYFGERNVKGMFEVLEPLHAMMERGPQTLKETSFNQAYGRDLMEAQEWCRKYMKSGNVKDLXQAWDLYYHVFRRISKTS

SEQ ID NO: 60 - protein, 95 aa, Artificial Sequence
  source: /note=“Description of Artificial Sequence Synthetic polypeptide”
ILWHEMWHEGLIEASRLYFGERNVKGMFEVLEPLHAMMERGPQTLKETSFNQAYGRDLMEAQEWCRKYMKSGNVKDLLQAWDLYYHVFRRISKTS

SEQ ID NO: 61 - protein, 95 aa, Artificial Sequence
  source: /note=“Description of Artificial Sequence Synthetic polypeptide”
ILWHEMWHEGLLEASRLYFGERNVKGMFEVLEPLHAMMERGPQTLKETSFNQAYGRDLMEAQEWCRKYMKSGNVKDLLQAWDLYYHVFRRISKTS

SEQ ID NO: 62 - protein, 10 aa, Artificial Sequence
  source: /note=“Description of Artificial Sequence Synthetic peptide”
GGGGSGGGGS

SEQ ID NO: 63 - protein, 416 aa, Artificial Sequence
  source: /note=“Description of Artificial Sequence Synthetic polypeptide”
GSMALPVTALLLPLALLLHAARPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKGGGGSFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAKRGRKKLLYIFKQPFMRPVQTTQEEDGCSCRFPEEEEGGCEL

SEQ ID NO: 64 - protein, 426 aa, Artificial Sequence
  source: /note=“Description of Artificial Sequence Synthetic polypeptide”
GSMALPVTALLLPLALLLHAARPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKGGGGSQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAKRGRKKLLYIFKQPFMRPVQTTQEEDGCSCRFPEEEEGGCEL

SEQ ID NO: 65 - protein, 429 aa, Artificial Sequence
  source: /note=“Description of Artificial Sequence Synthetic polypeptide”
GSMALPVTALLLPLALLLHAARPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKGGGGSQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 66 - protein, 471 aa, Artificial Sequence
  source: /note=“Description of Artificial Sequence Synthetic polypeptide”
GSMALPVTALLLPLALLLHAARPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKGGGGSQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNKRGRKKLLYIFKQPFMRPVQTTQEEDGCSCRFPEEEEGGCEL

SEQ ID NO: 67 - protein, 496 aa, Artificial Sequence
  source: /note=“Description of Artificial Sequence Synthetic polypeptide”
GSMALPVTALLLPLALLLHAARPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIKRGRKKLLYIFKQPFMRPVQTTQEEDGCSCRFPEEEEGGCEL

SEQ ID NO: 68 - protein, 50 aa, Artificial Sequence
  source: /note=“Description of Artificial Sequence Synthetic polypeptide”
  MISC_FEATURE (1)..(50): /note=“This sequence may encompass 0-10 ′Gly Gly Gly Gly Ser′ repeating units”
  source: /note=“See specification as filed for detailed description of substitutions and preferred embodiments”
GGGGSGGGGSGGGGSGGGGSGGGGSGGGGSGGGGSGGGGSGGGGSGGGGS

SEQ ID NO: 69 - protein, 40 aa, Artificial Sequence
  source: /note=“Description of Artificial Sequence Synthetic polypeptide”
  MISC_FEATURE (1)..(40): /note=“This sequence may encompass 0-10 ′Gly Gly Gly Ser′ repeating units”
  source: /note=“See specification as filed for detailed description of substitutions and preferred embodiments”
GGGSGGGSGGGSGGGSGGGSGGGSGGGSGGGSGGGSGGGS

SEQ ID NO: 70 - protein, 419 aa, Artificial Sequence
  source: /note=“Description of Artificial Sequence Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKRGRKKLLYIFKQPFMRPVQTTQEEDGCSCRFPEEEEGGCEL

SEQ ID NO: 71 - protein, 394 aa, Artificial Sequence
  source: /note=“Description of Artificial Sequence Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAKRGRKKLLYIFKQPFMRPVQTTQEEDGCSCRFPEEEEGGCEL

SEQ ID NO: 72 - protein, 404 aa, Artificial Sequence
  source: /note=“Description of Artificial Sequence Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAKRGRKKLLYIFKQPFMRPVQTTQEEDGCSCRFPEEEEGGCEL

SEQ ID NO: 73 - protein, 431 aa, Artificial Sequence
  source: /note=“Description of Artificial Sequence Synthetic polypeptide”
EVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSSGGGGSGGGGSGGGGSQSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVLGGGGSQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKRGRKKLLYIFKQPFMRPVQTTQEEDGCSCRFPEEEEGGCEL

SEQ ID NO: 74 - protein, 436 aa, Artificial Sequence
  source: /note=“Description of Artificial Sequence Synthetic polypeptide”
EVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSSGGGGSGGGGSGGGGSQSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVLGGGGSGGGGSQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKRGRKKLLYIFKQPFMRPVQTTQEEDGCSCRFPEEEEGGCEL

SEQ ID NO: 75 - protein, 415 aa, Artificial Sequence
  source: /note=“Description of Artificial Sequence Synthetic polypeptide”
EVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSSGGGGSGGGGSGGGGSQSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVLGGGGSQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAKRGRKKLLYIFKQPFMRPVQTTQEEDGCSCRFPEEEEGGCEL

SEQ ID NO: 76 - protein, 420 aa, Artificial Sequence
  source: /note=“Description of Artificial Sequence Synthetic polypeptide”
EVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSSGGGGSGGGGSGGGGSQSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVLGGGGSGGGGSQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAKRGRKKLLYIFKQPFMRPVQTTQEEDGCSCRFPEEEEGGCEL

SEQ ID NO: 77 - protein, 185 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 78 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 79 - protein, 26 aa, Homo sapiens
VMSVATIVIVDICITGGLLLLVYYWS

SEQ ID NO: 80 - protein, 130 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWS

SEQ ID NO: 81 - protein, 172 aa, Artificial Sequence
  source: /note=“Description of Artificial Sequence Synthetic polypeptide”
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKRGRKKLLYIFKQPFMRPVQTTQEEDGCSCRFPEEEEGGCEL

SEQ ID NO: 82 - protein, 150 aa, Homo sapiens
FKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 83 - protein, 84 aa, Homo sapiens [sequence omitted]

SEQ ID NO: 84 - protein, 21 aa, Homo sapiens
GIIVTDVIATLLLALGVFCFA

SEQ ID NO: 85 - protein, 105 aa, Homo sapiens
FKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFA

SEQ ID NO: 86 - protein, 147 aa, Artificial Sequence
  source: /note=“Description of Artificial Sequence Synthetic polypeptide”
FKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAKRGRKKLLYIFKQPFMRPVQTTQEEDGCSCRFPEEEEGGCEL

SEQ ID NO: 87 - protein, 160 aa, Homo sapiens
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 88 - protein, 94 aa, Homo sapiens
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATIS

SEQ ID NO: 89 - protein, 21 aa, Homo sapiens
GFLFAEIVSIFVLAVGVYFIA

SEQ ID NO: 90 - protein, 115 aa, Homo sapiens
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIA

SEQ ID NO: 91 - protein, 157 aa, Artificial Sequence
  source: /note=“Description of Artificial Sequence Synthetic polypeptide”
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAKRGRKKLLYIFKQPFMRPVQTTQEEDGCSCRFPEEEEGGCEL

SEQ ID NO: 92 - protein, 24 aa, Artificial Sequence
  source: /note=“Description of Artificial Sequence Synthetic peptide”
IYIWAPLAGTCGVLLLSLVITLYC

SEQ ID NO: 93 - DNA, 72 bp, Artificial Sequence
  source: /note=“Description of Artificial Sequence Synthetic oligonucleotide”
atctacatctgggcgcccttggccgggacttgtggggtccttctcctgtcactggttatcaccctttactgc

SEQ ID NO: 94 - protein, 48 aa, Artificial Sequence
  source: /note=“Description of Artificial Sequence Synthetic polypeptide”
QRRKYRSNKGESPVEPAEPCRYSCPREEEGSTIPIQEDYRKPEPACSP

SEQ ID NO: 95 - DNA, 123 bp, Artificial Sequence
  source: /note=“Description of Artificial Sequence Synthetic polynucleotide”
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 96 - protein, 112 aa, Artificial Sequence
  source: /note=“Description of Artificial Sequence Synthetic polypeptide”
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - DNA, 336 bp, Artificial Sequence
  source: /note=“Description of Artificial Sequence Synthetic polynucleotide”
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 98 - protein, 112 aa, Artificial Sequence
  source: /note=“Description of Artificial Sequence Synthetic polypeptide”
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - DNA, 336 bp, Artificial Sequence
  source: /note=“Description of Artificial Sequence Synthetic polynucleotide”
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 100 - protein, 41 aa, Artificial Sequence
  source: /note=“Description of Artificial Sequence Synthetic polypeptide”
RSKRSRLLHSDYMNMTPRRPGPTRKHYQPYAPPRDFAAYRS

SEQ ID NO: 101 - DNA, 123 bp, Artificial Sequence; identical to SEQ ID NO: 95
  source: /note=“Description of Artificial Sequence Synthetic polynucleotide”

SEQ ID NO: 102 - protein, 35 aa, Artificial Sequence
  source: /note=“Description of Artificial Sequence Synthetic polypeptide”
TKKKYSSSVHDPNGEYMFMRAVNTAKKSRLTDVTL

SEQ ID NO: 103 - DNA, 105 bp, Artificial Sequence
  source: /note=“Description of Artificial Sequence Synthetic polynucleotide”
acaaaaaagaagtattcatccagtgtgcacgaccctaacggtgaatacatgttcatgagagcagtgaacacagccaaaaaatccagactcacagatgtgacccta

SEQ ID NO: 104 - protein, 242 aa, Artificial Sequence
  source: /note=“Description of Artificial Sequence Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS